(19) United States
(12) Patent Application Publication
(10) Pub. No.: US 2024/0400721 A1
(43) Pub. Date: Dec. 5, 2024

(54) USES OF DLL3-TARGETING MULTISPECIFIC ANTIGEN-BINDING MOLECULES

(71) Applicant: Chugai Seiyaku Kabushiki Kaisha, Tokyo (JP)

(72) Inventors: Sotaro Naoi, Shizuoka (JP); Shu Feng, Singapore (SG); Tomoyuki Igawa, Tokyo (JP); Shu Wen Samantha Ho, Singapore (SG); Yutaka Matsuda, Shizuoka (JP); Hirofumi Mikami, Shizuoka (JP); Yumiko Kawai, Shizuoka (JP); Toshiaki Tsunenari, Shizuoka (JP)

(73) Assignee: Chugai Seiyaku Kabushiki Kaisha, Tokyo (JP)

(21) Appl. No.: 18/696,717

(22) PCT Filed: Sep. 28, 2022
(86) PCT No.: PCT/JP2022/036063

§ 371 Date: Mar. 28, 2024

(30) Foreign Application Priority Data
Sep. 29, 2021 (WO) PCT/JP2021/035877

(51) Int. Cl. C07K 16/46; A61K 39/00; A61K 45/06; A61P 35/00; C07K 16/28

(52) U.S. Cl. CPC C07K 16/468; A61K 45/06; A61P 35/00; C07K 16/2809; C07K 16/2878; A61K 2039/505; C07K 2317/31; C07K 2317/522; C07K 2317/55; C07K 2317/565

(57) ABSTRACT

The disclosure provides uses of multispecific antigen-binding molecules that targets human DLL3 for the treatment of cancers.

### TECHNICAL FIELD

The present invention relates to multispecific antigen-binding molecules that targets DLL3, pharmaceutical compositions comprising such antigen-binding molecules; and uses of such antigen-binding molecules or such compositions, for therapeutic purposes in the field of cancer diseases.

### BACKGROUND ART

In cancer therapy, the effective and selective destruction of tumor cells while leaving healthy cells and tissues undamaged is a desirable goal. There is a need for having improved treatment for tumorous diseases related to the overexpression of DLL3, such as neuroendocrine carcinomas (NEC), neuroendocrine tumors (NET) and other solid tumors.

Delta-like 3 (DLL3) is a type I membrane protein belonging to Notch ligand family members. DLL3 is a non-canonical Notch ligand, functioning in a cell autonomous manner to inhibit Notch signaling, binding to Notch in cis, thus blocking cell to cell interactions and internalization of Notch in the target cell, a hallmark of canonical Notch signaling. The primary role for DLL3 is in somitogenesis during embryonic development. Mice with DLL3 knockouts show segmental defects in the axial skeleton and cranial and neuronal development. Somitic patterning defects are also seen in humans with certain germline DLL3 mutations, resulting in a condition called spondylocostal dysostosis. There exist previous studies reporting the amplification of the DLL3 gene on chromosome and increased expression of this gene in cancer cell lines (NPL 1) and increased DLL3 expression in some glioma cases (NPL 2). In addition, DLL3 has been proposed previously in methods to diagnose and treat glioma or small cell lung cancer (SCLC), using an ADCC enhanced antibody, antibody-drug conjugate (ADC), and T cell-engaging bispecific molecule using BiTE-Fc format (PTLs 1, 2, and 3).

### CITATION LIST

### Patent Literature

[PTL 1] WO 2011/093097
[PTL 2] WO 2013/126746
[PTL 3] WO 2017/021349

### Non Patent Literature

[NPL 1] Phillips, H. S. (2006) Cancer Cell 9, 157-173.
[NPL 2] Mulledndore, M. E. (2009) Clin Cancer Res 15, 2291-2301.

### SUMMARY OF INVENTION

### Technical Problem

An objective of the present invention is to provide multispecific antigen-binding molecules that enable cancer treatment by recruiting T cells close to DLL3-expressing cells and using the cytotoxicity of T cells against DLL3-expressing cancer cells, methods for producing the multispecific antigen-binding molecules, and therapeutic agents comprising such a multispecific antigen-binding molecule as an active ingredient for inducing cellular cytotoxicity. Another objective of the present invention is to provide pharmaceutical compositions for use in treating or preventing various cancers, which comprise one of the above-mentioned antigen-binding molecules as an active ingredient, and therapeutic methods using the pharmaceutical compositions.

### Solution to Problem

The present invention relates to multispecific antigen-binding molecules that comprise a first antigen-binding moiety and a second antigen-binding moiety, each of which is capable of binding to CD3 and CD137, but does not bind to CD3 and CD137 at the same time (i.e. capable of binding to CD3 and CD137 but not simultaneously); and a third antigen-binding moiety that is capable of binding to DLL3, preferably human DLL3, which induce T-cell dependent cytotoxicity more efficiently whilst circumventing adverse toxicity concerns or side effects that other multispecific antigen-binding molecules may have. The present invention further relates to medical uses of said multispecific antigen-binding molecules and pharmaceutical compositions thereof that can treat various cancers, especially those associated with DLL3 such as DLL3-expressing or DLL3-positive cancer, by comprising the antigen-binding molecule as an active ingredient. In one aspect, said DLL3-expressing or DLL3-positive cancer is selected from the group consisting of pancreatic neuroendocrine tumor (PNET), small-cell type NEC (SCNEC), large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC), neuroblastoma, glioma or glioblastoma (GBM), melanoma, and medullary thyroid cancer.

In one aspect, the multispecific antigen-binding molecule of the present invention have very unique structure format(s), which improve or enhance the efficacy of the multispecific antigen-binding molecules. The new antigen-binding molecules with unique structure formats provide the increased number of antigen-binding domains to give the increased valency and/or specificities to respective antigens on effector cells and target cells with the reduced unwanted adverse effects.

In one particular aspect, the present invention relates to multispecific antigen-binding molecules that comprise a first antigen-binding moiety and a second antigen-binding moiety, each of which is capable of binding to CD3 and CD137, but does not bind to CD3 and CD137 at the same time (i.e. capable of binding to CD3 and CD137 but not simultaneously); and a third antigen-binding moiety that is capable of binding to DLL3, preferably human DLL3, which induce T-cell dependent cytotoxicity efficiently whilst circumventing adverse toxicity concerns or side effects which other multispecific antigen-binding molecules may have. In one aspect, the antigen-biding moieties capable of binding to CD3 and CD137, but does not bind to CD3 and CD137 at the same time are antigen-biding moieties which are capable of binding to human CD3 and human CD137, wherein the first antigen-binding moiety binds to either one of human CD3 and human CD137. Antigen-biding moieties capable of binding to CD3 and CD137, but does not bind to CD3 and CD137 at the same time (i.e. capable of binding to CD3 and CD137 but not simultaneously) may be called “Dual antigen-binding moiety” or in some aspects “Dual-Fabs” as defined more specifically below. In one such aspect, each of the first and second antigen-binding moieties comprises at least one amino acid mutation(s) e.g. cysteine insertion/substitution/mutation which create a disulfide linkage between the first and second antigen-binding moieties to hold them close to each other, and, for example, promote cis-antigen binding to antigen (CD3 and/or CD137) on the same single effector cell as a result of steric hindrance or shorter distance between the two antigen-binding moieties (e.g., Dual-Fabs), thereby improving the safety profile of the trispecific antigen-binding molecule by preventing undesirable crosslinking of two CD3/CD137-expressing immune cells mediated by the two antigen-biding moieties (capable of binding to CD3 and CD137 but not simultaneously) in an DLL3-independent manner. In one specific aspect, said each of the first antigen-binding moiety and the second antigen-binding moiety is a Fab and comprises at least one cysteine residue (via mutation, substitution, or insertion) in the CH1 region, said at least one cysteine residue is capable of forming at least one disulfide bond between the CH1 region of the first antigen-binding moiety and the CH1 region of the second antigen-binding moiety. In another specific aspect, said each of the first antigen-binding moiety and the second antigen-binding moiety comprises one cysteine residue (via mutation, substitution, or insertion) at position 191 according to EU numbering in the CH1 region which is capable of forming one disulfide bond between the CH1 region of the first antigen-binding moiety and the CH1 region of the second antigen-binding moiety. Such disulfide bond in the CH1 region (e.g. position 191 according to EU numbering) linking the first and the second antigen-binding moieties may be called “LINC” herein.

The antigen-binding molecules having such unique structure formats, i.e. a trivalent tri-specific antibody comprising two monovalent Dual-Fabs each capable of binding to CD3 and CD137 but not simultaneously and one monovalent DLL3-binding arm, which may be called “(2+1)” (or “1+2”), were surprisingly found to show superior efficacy compared to other multispecific antibody formats (e.g. BiTE) while exhibiting reduced or minimal off-target side-effects attributed by undesired cross-linking among different cells (e.g., effector cells such as T cells).

The present invention relates to:

[A-1] An antibody for use as a medicament;
or an antibody for use in treating cancer;
or a pharmaceutical formulation comprising an antibody and a pharmaceutically acceptable carrier;
or a pharmaceutical formulation comprising an antibody and a pharmaceutically acceptable carrier for use as a medicament;
or a pharmaceutical formulation comprising an antibody and a pharmaceutically acceptable carrier for use in the treatment of cancer.

[A-2] The antibody or the pharmaceutical formulation of [A-1], wherein the antibody comprises:
(a) a first antigen-binding moiety and a second antigen-binding moiety, each of which comprises an antibody variable region that can be the same or different and is independently selected from the group consisting of:
(a1) an antibody variable region comprising the heavy chain complementarity determining region (CDR) 1 of SEQ ID NO: 20, the heavy chain CDR 2 of SEQ ID NO: 34, the heavy chain CDR 3 of SEQ ID NO: 48, light chain CDR 1 of SEQ ID NO: 63, light chain CDR 2 of SEQ ID NO: 68 and light chain CDR 3 of SEQ ID NO: 73;
(a2) an antibody variable region comprising the heavy chain complementarity determining region (CDR) 1 of SEQ ID NO: 28, the heavy chain CDR 2 of SEQ ID NO: 42, the heavy chain CDR 3 of SEQ ID NO: 56, light chain CDR 1 of SEQ ID NO: 63, light chain CDR 2 of SEQ ID NO: 68 and light chain CDR 3 of SEQ ID NO: 73;
(a3) an antibody variable region comprising heavy chain complementarity determining region (CDR) 1 of SEQ ID NO: 82, heavy chain CDR 2 of SEQ ID NO: 83, heavy chain CDR 3 of SEQ ID NO: 84, light chain CDR 1 of SEQ ID NO: 65, light chain CDR 2 of SEQ ID NO: 70 and light chain CDR 3 of SEQ ID NO: 75; and
(b) a third antigen-binding moiety that binds to human Delta-like 3 (DLL3) and comprises an antibody variable region comprising a heavy chain CDR1 comprising SEQ ID NO: 233, a heavy chain CDR 2 comprising SEQ ID NO: 234, a heavy chain CDR 3 comprising SEQ ID NO: 235, a light chain CDR 1 comprising SEQ ID NO: 237, a light chain CDR 2 comprising SEQ ID NO: 238, and a light chain CDR 3 comprising SEQ ID NO: 239.

[A-3] The antibody or the pharmaceutical formulation of [A-2], wherein each of the first antigen-binding moiety and the second antigen-binding moiety has one, two or more of the following properties:
(i) binds to human CD3;
(ii) binds to human CD137;
(iii) capable of binding to human CD3 and human CD137, wherein each of the first antigen-binding moiety and the second antigen-binding moiety binds to either one of human CD3 and human CD137; and
(iv) capable of binding to human CD3 and human CD137, but does not bind to human CD3 and human CD137 at the same time.

[A-4] The antibody or the pharmaceutical formulation of any one of [A-2] to [A-3], wherein each of the first and second antigen-binding moieties comprises an antibody variable region that can be the same or different and is independently selected from the group consisting of:
(a1) a heavy chain variable region (VH) comprising an amino acid sequence of SEQ ID NO: 6, and a light chain variable region (VL) comprising an amino acid sequence of SEQ ID NO: 58;
(a2) a VH comprising an amino acid sequence of SEQ ID NO: 14, and a VL comprising an amino acid sequence of SEQ ID NO: 58;
(a3) a VH comprising an amino acid sequence of SEQ ID NO: 81, and a VL comprising an amino acid sequence of SEQ ID NO: 60;
(a4) a VH and a VL comprising one or more amino acid substitutions, deletions, additions, and/or insertions in the VH and VL of any one of (a1) to (a3) and has an activity equivalent to that of any of the VH and VL; and
(a5) a VH and a VL having at least 80%, 85%, 90% or 95% sequence identity to the amino acid sequence of the VH and VL defined in any one of (a1) to (a3).

[A-5] The antibody or the pharmaceutical formulation of any one of [A-2] to [A-3], wherein the first antigen-binding moiety and the second antigen-binding moiety each comprises an antibody variable region comprising a heavy chain complementarity determining region (CDR) 1 of SEQ ID NO: 20, a heavy chain CDR 2 of SEQ ID NO: 34, a heavy chain CDR 3 of SEQ ID NO: 48, a light chain CDR 1 of SEQ ID NO: 63, a light chain CDR 2 of SEQ ID NO: 68 and a light chain CDR 3 of SEQ ID NO: 73.

[A-6] The antibody or the pharmaceutical formulation of [A-4], wherein the first antigen-binding moiety and the second antigen-binding moiety each comprises an antibody variable region comprising a heavy chain variable region comprising an amino acid sequence of SEQ ID NO: 6, and a light chain variable region comprising an amino acid sequence of SEQ ID NO: 58.

[A-7] The antibody or the pharmaceutical formulation of any one of [A-2] to [A-6], wherein the third antigen-binding moiety comprises:
(a) an antibody variable region comprising a VH comprising SEQ ID NO: 232 and a VL comprising SEQ ID NO: 236; or
(b) an antibody variable region comprising a VH having at least 80%, 85%, 90% or 95% sequence identity to the amino acid sequence of SEQ ID NO: 232 and a VL having at least 80%, 85%, 90% or 95% sequence identity to the amino acid sequence of SEQ ID NO: 236.

[A-8] The antibody or the pharmaceutical formulation of any one of [A-2] to [A-7], wherein each of the first and second antigen-binding moieties is a Fab that has a cysteine residue at position 191 (EU numbering), and wherein there is a disulfide bond linking the two cysteine residues.

[A-9] The antibody or the pharmaceutical formulation of any one of [A-2] to [A-8], wherein each of the first, second and third antigen binding moieties is a Fab comprising a heavy chain comprising a VH and a CH1 domain and a light chain comprising a VL and a light chain constant (CL) domain, and wherein the C-terminus of the heavy chain of the third antigen binding moiety is fused, directly or via a peptide linker, to the N-terminus of the Fab heavy chain of either the first antigen binding moiety or the second antigen binding moiety.

[A-10-1] The antibody or the pharmaceutical formulation of [A-9], wherein the C-terminus of the heavy chain of the third antigen binding moiety is fused, via a peptide linker, to the N-terminus of the Fab heavy chain of either the first antigen binding moiety or the second antigen binding moiety, and wherein the peptide linker has an amino acid sequence selected from the group consisting of SEQ ID NO: 248, SEQ ID NO: 249, and SEQ ID NO: 259.

[A-10-2] The antibody or the pharmaceutical formulation of [A-10-1], wherein the third antigen binding moiety is a crossover Fab molecule in which the variable regions of the Fab light chain and the Fab heavy chain are exchanged, and wherein each of the first and second antigen binding moiety is a conventional Fab molecule.

[A-11] The antibody or the pharmaceutical formulation of [A-10-2], wherein, in the CL domain of each of the first and second antigen binding moieties, the amino acids at positions 123 and 124 (Kabat numbering) are arginine and lysine, respectively; and wherein, in the CH1 domain of each of the first and second antigen binding moieties, the amino acid at each of positions 147 and 213 (EU numbering) is glutamic acid.

[A-12] The antibody or the pharmaceutical formulation of any one of [A2] to [A-11], further comprising an Fc domain.

[A-13] The antibody or the pharmaceutical formulation of [A-12], wherein the Fc domain comprises a first and a second Fc region subunit, the first Fc-region subunit is selected from the group comprising:
a Fc region polypeptide comprising alanine at each of positions 234 and 235;
a Fc region polypeptide comprising alanine at each of positions 234, 235, and 297; and
a Fc region polypeptide comprising alanine at each of positions 234, 235, and 297, cysteine at position 354, and tryptophan at position 366; and
the second Fc-region subunit is selected from the group comprising:
a Fc region polypeptide comprising alanine at each of positions 234 and 235;
a Fc region polypeptide comprising alanine at each of positions 234, 235, and 297; and
a Fc region polypeptide comprising alanine at each of positions 234, 235, and 297, cysteine at position 349, serine at position 366, alanine at position 368, and valine at position 407,
wherein all positions are by EU numbering.

[A-14] The antibody or the pharmaceutical formulation of [A-2], wherein the antibody comprises five polypeptide chains in a combination selected from the group consisting of (A) to (C) below:
(A) a polypeptide chain comprising the amino acid sequence of SEQ ID NO: 201 (chain 1), a polypeptide chain comprising the amino acid sequence of SEQ ID NO: 206 (chain 2), a polypeptide chain comprising the amino acid sequence of SEQ ID NO: 208 (chain 3), and two polypeptide chains each comprising the amino acid sequence of SEQ ID NO: 214 (chains 4 and 5);
(B) a polypeptide chain comprising the amino acid sequence of SEQ ID NO: 203 (chain 1), a polypeptide chain comprising the amino acid sequence of SEQ ID NO: 206 (chain 2), a polypeptide chain comprising the amino acid sequence of SEQ ID NO: 209 (chain 3), and two polypeptide chains each comprising the amino acid sequence of SEQ ID NO: 214 (chains 4 and 5); and
(C) a polypeptide chain comprising the amino acid sequence of SEQ ID NO: 204 (chain 1), a polypeptide chain comprising the amino acid sequence of SEQ ID NO: 206 (chain 2), a polypeptide chain comprising the amino acid sequence of SEQ ID NO: 209 (chain 3), and two polypeptide chains each comprising the amino acid sequence of SEQ ID NO: 214 (chains 4 and 5).

[A-15] The antibody or the pharmaceutical formulation of [A-2], wherein the antibody comprises five polypeptide chains in the following combination:
a polypeptide chain comprising the amino acid sequence of SEQ ID NO: 201 (chain 1),
a polypeptide chain comprising the amino acid sequence of SEQ ID NO: 206 (chain 2),
a polypeptide chain comprising the amino acid sequence of SEQ ID NO: 208 (chain 3), and two polypeptide chains each comprising the amino acid sequence of SEQ ID NO: 214 (chains 4 and 5).

[A-16] The antibody or the pharmaceutical formulation of [A-2], wherein the antibody comprises five polypeptide chains in the following combination:
a polypeptide chain comprising the amino acid sequence of SEQ ID NO: 203 (chain 1),
a polypeptide chain comprising the amino acid sequence of SEQ ID NO: 206 (chain 2),
a polypeptide chain comprising the amino acid sequence of SEQ ID NO: 209 (chain 3), and two polypeptide chains each comprising the amino acid sequence of SEQ ID NO: 214 (chains 4 and 5).

[A-17] The antibody or the pharmaceutical formulation of [A-2], wherein the antibody comprises five polypeptide chains in the following combination:
a polypeptide chain comprising the amino acid sequence of SEQ ID NO: 204 (chain 1),
a polypeptide chain comprising the amino acid sequence of SEQ ID NO: 206 (chain 2),
a polypeptide chain comprising the amino acid sequence of SEQ ID NO: 209 (chain 3), and two polypeptide chains each comprising the amino acid sequence of SEQ ID NO: 214 (chains 4 and 5).

[A-18] The antibody or the pharmaceutical formulation of any one of [A-14] to [A-17], wherein the five polypeptide chains (chain 1 to chain 5) of the antibody are connected and/or associate with each other as shown in FIG. 1(a).

[A-19] The antibody or the pharmaceutical formulation of any one of [A-14] to [A-17], wherein each of the polypeptide chain 2 and chain 5 associates with the polypeptide chain 1; the polypeptide chain 4 associates with the polypeptide chain 3; and the polypeptide chain 1 associates with the polypeptide chain 3.

[A-20] The antibody or the pharmaceutical formulation of any one of [A-2] to [A-17], for use in combination with an additional therapeutic agent, preferably a chemotherapeutic agent or an immune checkpoint inhibitor.

[A-21] The antibody or the pharmaceutical formulation of [A-20], for use in combination with an immune checkpoint inhibitor, wherein the immune checkpoint inhibitor is a PD-1 axis binding antagonist, preferably an anti-PD-1 antibody or an anti-PD-L1 antibody, more preferably atezolizumab.

[A-22] The antibody or the pharmaceutical formulation of [A-20], for use in combination with a chemotherapeutic agent, wherein the chemotherapeutic agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, an antiangiogenic agent, Etoposide, Irinotecan, Lurbinectedin, Amrubicin, and platinum agents (such as cisplatin and carboplatin).

[A-23] The antibody or the pharmaceutical formulation of any one of [A-20] to [A-22], wherein the immune checkpoint inhibitor or the chemotherapeutic agent is administered concomitantly with the antibody or the pharmaceutical formulation.

[A-24] The antibody or the pharmaceutical formulation of any one of [A-20] to [A-23], wherein the immune checkpoint inhibitor or the chemotherapeutic agent is administered before or after the administration of the antibody or the pharmaceutical formulation.

[A-25] The antibody or the pharmaceutical formulation of any one of [A-2] to [A-24], for use in treating cancer, wherein the cancer is a DLL3-expressing or DLL3-positive cancer.

[A-26] The antibody or the pharmaceutical formulation of [A-25], wherein the cancer is selected from the group consisting of neuroendocrine neoplasm (NEN), neuroendocrine tumor (NET), neuroendocrine carcinoma (NEC), and other solid tumors of non-neuroendocrine origin.

[A-27] The antibody or the pharmaceutical formulation of [A-25], wherein the cancer is selected from the group consisting of pancreatic neuroendocrine tumor (PNET), small-cell type NEC (SCNEC), large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC), neuroblastoma, glioma or glioblastoma (GBM), melanoma, and medullary thyroid cancer.

[A-28] The antibody or the pharmaceutical formulation of [A-25], wherein the cancer is lung cancer, preferably SCLC.

[A-29] The antibody or the pharmaceutical formulation of [A-25], wherein the cancer is glioma or glioblastoma (GBM).

[A-30] The antibody or the pharmaceutical formulation of [A-25], wherein the cancer is neuroendocrine prostate cancer.

[A-31] The antibody or the pharmaceutical formulation of [A-25], wherein the cancer is neuroblastoma.

[A-32] The antibody or the pharmaceutical formulation of any one of [A-2] to [A-31], wherein the antibody is a multispecific antibody.

[A-33] The antibody or the pharmaceutical formulation for use in [A-21], further in combination with a chemotherapeutic agent, wherein the chemotherapeutic agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, an antiangiogenic agent, Etoposide, Irinotecan, Lurbinectedin, Amrubicin, and platinum agents (such as cisplatin and carboplatin).

[A-34] The antibody or the pharmaceutical formulation of [A-22], further in combination with an immune checkpoint inhibitor, wherein the immune checkpoint inhibitor is a PD-1 axis binding antagonist, preferably an anti-PD-1 antibody or an anti-PD-L1 antibody, more preferably atezolizumab.

[B-1] A multispecific antibody which comprises:
(a) a first antigen-binding moiety and a second antigen-binding moiety, each of which comprises an antibody variable region that can be the same or different and is independently selected from the group consisting of:
(a1) an antibody variable region comprising the heavy chain complementarity determining region (CDR) 1 of SEQ ID NO: 20, the heavy chain CDR 2 of SEQ ID NO: 34, the heavy chain CDR 3 of SEQ ID NO: 48, light chain CDR 1 of SEQ ID NO: 63, light chain CDR 2 of SEQ ID NO: 68 and light chain CDR 3 of SEQ ID NO: 73;
(a2) an antibody variable region comprising the heavy chain complementarity determining region (CDR) 1 of SEQ ID NO: 28, the heavy chain CDR 2 of SEQ ID NO: 42, the heavy chain CDR 3 of SEQ ID NO: 56, light chain CDR 1 of SEQ ID NO: 63, light chain CDR 2 of SEQ ID NO: 68 and light chain CDR 3 of SEQ ID NO: 73;
(a3) an antibody variable region comprising heavy chain complementarity determining region (CDR) 1 of SEQ ID NO: 82, heavy chain CDR 2 of SEQ ID NO: 83, heavy chain CDR 3 of SEQ ID NO: 84, light chain CDR 1 of SEQ ID NO: 65, light chain CDR 2 of SEQ ID NO: 70 and light chain CDR 3 of SEQ ID NO: 75; and
(b) a third antigen-binding moiety that binds to human Delta-like 3 (DLL3) and comprises an antibody variable region comprising a heavy chain CDR1 comprising SEQ ID NO: 233, a heavy chain CDR 2 comprising SEQ ID NO: 234, a heavy chain CDR 3 comprising SEQ ID NO: 235, a light chain CDR 1 comprising SEQ ID NO: 237, a light chain CDR 2 comprising SEQ ID NO: 238, and a light chain CDR 3 comprising SEQ ID NO: 239.

[B-2] The multispecific antibody of [B-1], wherein each of the first antigen-binding moiety and the second antigen-binding moiety has one, two or more of the following properties:
(i) binds to human CD3;
(ii) binds to human CD137;
(iii) capable of binding to human CD3 and human CD137, wherein each of the first antigen-binding moiety and the second antigen-binding moiety binds to either one of human CD3 and human CD137; and
(iv) capable of binding to human CD3 and human CD137, but does not bind to human CD3 and human CD137 at the same time.

[B-3] The multispecific antibody of any one of [B-1] to [B-2], wherein each of the first and second antigen-binding moieties comprises an antibody variable region that can be the same or different and is independently selected from the group consisting of:
(a1) a heavy chain variable region (VH) comprising an amino acid sequence of SEQ ID NO: 6, and a light chain variable region (VL) comprising an amino acid sequence of SEQ ID NO: 58;
(a2) a VH comprising an amino acid sequence of SEQ ID NO: 14, and a VL comprising an amino acid sequence of SEQ ID NO: 58;
(a3) a VH comprising an amino acid sequence of SEQ ID NO: 81, and a VL comprising an amino acid sequence of SEQ ID NO: 60;
(a4) a VH and a VL comprising one or more amino acid substitutions, deletions, additions, and/or insertions in the VH and VL of any one of (a1) to (a3) and has an activity equivalent to that of any of the VH and VL; and
(a5) a VH and a VL having at least 80%, 85%, 90% or 95% sequence identity to the amino acid sequence of the VH and VL defined in any one of (a1) to (a3).

[B-4] The multispecific antibody of any one of [B-1] to [B-2], wherein the first antigen-binding moiety and the second antigen-binding moiety each comprises an antibody variable region comprising a heavy chain complementarity determining region (CDR) 1 of SEQ ID NO: 20, a heavy chain CDR 2 of SEQ ID NO: 34, a heavy chain CDR 3 of SEQ ID NO: 48, a light chain CDR 1 of SEQ ID NO: 63, a light chain CDR 2 of SEQ ID NO: 68 and a light chain CDR 3 of SEQ ID NO: 73.

[B-5] The multispecific antibody of [B-3], wherein the first antigen-binding moiety and the second antigen-binding moiety each comprises an antibody variable region comprising a heavy chain variable region comprising an amino acid sequence of SEQ ID NO: 6, and a light chain variable region comprising an amino acid sequence of SEQ ID NO: 58.

[B-6] The multispecific antibody of any one of [B-1] to [B-5], wherein the third antigen-binding moiety comprises:
(a) an antibody variable region comprising a VH comprising SEQ ID NO: 232 and a VL comprising SEQ ID NO: 236; or
(b) an antibody variable region comprising a VH having at least 80%, 85%, 90% or 95% sequence identity to the amino acid sequence of SEQ ID NO: 232 and a VL having at least 80%, 85%, 90% or 95% sequence identity to the amino acid sequence of SEQ ID NO: 236.

[B-7] The multispecific antibody of any one of [B-1] to [B-6], wherein each of the first and second antigen-binding moieties is a Fab that has a cysteine residue at position 191 (EU numbering), and wherein there is a disulfide bond linking the two cysteine residues.

[B-8] The multispecific antibody of any one of [B-1] to [B-7], wherein each of the first, second and third antigen binding moieties is a Fab comprising a heavy chain comprising a VH and a CH1 domain and a light chain comprising a VL and a light chain constant (CL) domain, and wherein the C-terminus of the heavy chain of the third antigen binding moiety is fused, directly or via a peptide linker, to the N-terminus of the Fab heavy chain of either the first antigen binding moiety or the second antigen binding moiety.

[B-9-1] The multispecific antibody of [B-8], wherein the C-terminus of the heavy chain of the third antigen binding moiety is fused, via a peptide linker, to the N-terminus of the Fab heavy chain of either the first antigen binding moiety or the second antigen binding moiety, and wherein the peptide linker has an amino acid sequence selected from the group consisting of SEQ ID NO: 248, SEQ ID NO: 249, and SEQ ID NO: 259.

[B-9-2] The multispecific antibody of [B-9-1], wherein the third antigen binding moiety is a crossover Fab molecule in which the variable regions of the Fab light chain and the Fab heavy chain are exchanged, and wherein each of the first and second antigen binding moiety is a conventional Fab molecule.

[B-10] The multispecific antibody of [B-9-2], wherein, in the CL domain of each of the first and second antigen binding moieties, the amino acids at positions 123 and 124 (Kabat numbering) are arginine and lysine, respectively; and wherein, in the CH1 domain of each of the first and second antigen binding moieties, the amino acid at each of positions 147 and 213 (EU numbering) is glutamic acid.

[B-11] The multispecific antibody of any one of [B-1] to [B-10], further comprising an Fc domain.

[B-12] The multispecific antibody of [B-11], wherein the Fc domain comprises a first and a second Fc region subunit, the first Fc-region subunit is selected from the group comprising:
a Fc region polypeptide comprising alanine at each of positions 234 and 235;
a Fc region polypeptide comprising alanine at each of positions 234, 235, and 297; and
a Fc region polypeptide comprising alanine at each of positions 234, 235, and 297, cysteine at position 354, and tryptophan at position 366; and
the second Fc-region subunit is selected from the group comprising:
a Fc region polypeptide comprising alanine at each of positions 234 and 235;
a Fc region polypeptide comprising alanine at each of positions 234, 235, and 297; and
a Fc region polypeptide comprising alanine at each of positions 234, 235, and 297, cysteine at position 349, serine at position 366, alanine at position 368, and valine at position 407,
wherein all positions are by EU numbering.

[B-13] The multispecific antibody of [B-1], wherein the antibody comprises five polypeptide chains in a combination selected from the group consisting of (A) to (C) below:
(A) a polypeptide chain comprising the amino acid sequence of SEQ ID NO: 201 (chain 1), a polypeptide chain comprising the amino acid sequence of SEQ ID NO: 206 (chain 2), a polypeptide chain comprising the amino acid sequence of SEQ ID NO: 208 (chain 3), and two polypeptide chains each comprising the amino acid sequence of SEQ ID NO: 214 (chains 4 and 5);
(B) a polypeptide chain comprising the amino acid sequence of SEQ ID NO: 203 (chain 1), a polypeptide chain comprising the amino acid sequence of SEQ ID NO: 206 (chain 2), a polypeptide chain comprising the amino acid sequence of SEQ ID NO: 209 (chain 3), and two polypeptide chains each comprising the amino acid sequence of SEQ ID NO: 214 (chains 4 and 5); and
(C) a polypeptide chain comprising the amino acid sequence of SEQ ID NO: 204 (chain 1), a polypeptide chain comprising the amino acid sequence of SEQ ID NO: 206 (chain 2), a polypeptide chain comprising the amino acid sequence of SEQ ID NO: 209 (chain 3), and two polypeptide chains each comprising the amino acid sequence of SEQ ID NO: 214 (chains 4 and 5).

[B-14] The multispecific antibody of [B-1], wherein the antibody comprises five polypeptide chains in the following combination:
a polypeptide chain comprising the amino acid sequence of SEQ ID NO: 201 (chain 1),
a polypeptide chain comprising the amino acid sequence of SEQ ID NO: 206 (chain 2),
a polypeptide chain comprising the amino acid sequence of SEQ ID NO: 208 (chain 3), and two polypeptide chains each comprising the amino acid sequence of SEQ ID NO: 214 (chains 4 and 5).

[B-15] The multispecific antibody of [B-1], wherein the antibody comprises five polypeptide chains in the following combination:
a polypeptide chain comprising the amino acid sequence of SEQ ID NO: 203 (chain 1),
a polypeptide chain comprising the amino acid sequence of SEQ ID NO: 206 (chain 2),
a polypeptide chain comprising the amino acid sequence of SEQ ID NO: 209 (chain 3), and two polypeptide chains each comprising the amino acid sequence of SEQ ID NO: 214 (chains 4 and 5).

[B-16] The multispecific antibody of [B-1], wherein the antibody comprises five polypeptide chains in the following combination:
a polypeptide chain comprising the amino acid sequence of SEQ ID NO: 204 (chain 1),
a polypeptide chain comprising the amino acid sequence of SEQ ID NO: 206 (chain 2),
a polypeptide chain comprising the amino acid sequence of SEQ ID NO: 209 (chain 3), and two polypeptide chains each comprising the amino acid sequence of SEQ ID NO: 214 (chains 4 and 5).

[B-17] The multispecific antibody of any one of [B-13] to [B-16], wherein the five polypeptide chains (chain 1 to chain 5) of the antibody are connected and/or associate with each other as shown in FIG. 1(a).

[B-18] The multispecific antibody of any one of [B-13] to [B-16], wherein each of the polypeptide chain 2 and chain 5 associates with the polypeptide chain 1; the polypeptide chain 4 associates with the polypeptide chain 3; and the polypeptide chain 1 associates with the polypeptide chain 3.

[B-19] The multispecific antibody of any one of [B-1] to [B-18], for use as a medicament.

[B-20] The multispecific antibody of any one of [B-1] to [B-18], for use in the treatment of cancer.

[C-1] A pharmaceutical formulation comprising the multispecific antibody of any one of [B-1] to [B-18] and a pharmaceutically acceptable carrier.

[C-2] A pharmaceutical formulation comprising an immune checkpoint inhibitor and a pharmaceutically acceptable carrier, for use in combination with the multispecific antibody of any one of [B-1] to [B-18], for treatment of cancer.

[C-3] A pharmaceutical formulation comprising a chemotherapeutic agent, and a pharmaceutically acceptable carrier, for use in combination with the multispecific antibody of any one of [B-1] to [B-18], for treatment of cancer.

[C-4] The pharmaceutical formulation of [C-2], wherein the immune checkpoint inhibitor is a PD-1 axis binding antagonist, preferably an anti-PD-1 antibody or an anti-PD-L1 antibody, more preferably atezolizumab.

[C-5] The pharmaceutical formulation of [C-3], wherein the chemotherapeutic agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, an antiangiogenic agent, Etoposide, Irinotecan, Lurbinectedin, Amrubicin, and platinum agents (such as cisplatin and carboplatin).

[C-6] The pharmaceutical formulation of any one of [C-2] to [C-5], wherein the immune checkpoint inhibitor or the chemotherapeutic agent is administered concomitantly with the multispecific antibody.

[C-7] The pharmaceutical formulation of any one of [C-2] to [C-5], wherein the immune checkpoint inhibitor or the chemotherapeutic agent is administered before or after the administration of the multispecific antibody.

[D-1] Use of the multispecific antibody of any one of [B-1] to [B-18] or the pharmaceutical formulation of any one of [C-1] to [C-7] in treating cancer.

[E-1] A method of treating an individual having cancer, comprising administering to the individual an effective amount of the multispecific antibody of any one of [B-1] to [B-18] or the pharmaceutical formulation of any one of [C-1] to [C-7].

[E-2] A method of activating or enhancing a persistent immune response to cancer, wherein the method comprises administering to a subject a therapeutically effective amount of the multispecific antibody of any one of [B-1] to [B-18] or the pharmaceutical formulation of any one of [C-1] to [C-7].

[E-3] A method to increase the efficacy of an immune checkpoint inhibitor, wherein the method comprises administering to a subject a therapeutically effective amount of the multispecific antibody of any one of [B-1] to [B-18] or the pharmaceutical formulation of any one of [C-1] to [C-7], in combination with the immune checkpoint inhibitor.

[E-4] A method of reducing or preventing metastasis in a subject, wherein the method comprises administering to a subject a therapeutically effective amount of the multispecific antibody of any one of [B-1] to [B-18] or the pharmaceutical formulation of any one of [C-1] to [C-7].

[E-5] A method of inhibiting tumor growth in a subject, wherein the method comprises administering to a subject a therapeutically effective amount of the multispecific antibody of any one of [B-1] to [B-18] or the pharmaceutical formulation of any one of [C-1] to [C-7].

[E-6] The method of any one of [E-1] to [E-5], further comprising administering to the subject an additional therapeutic agent, preferably a chemotherapeutic agent, or an immune checkpoint inhibitor.

[E-7] The method of [E-6], wherein the immune checkpoint inhibitor is a PD-1 axis binding antagonist, preferably an anti-PD-1 antibody or an anti-PD-L1 antibody, more preferably atezolizumab.

[E-8] The method of [E-6], wherein the chemotherapeutic agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, an antiangiogenic agent, Etoposide, Irinotecan, Lurbinectedin, Amrubicin, and platinum agents (such as cisplatin and carboplatin).

[E-9] The method of any one of [E-6] to [E-8], wherein the additional therapeutic agent, immune checkpoint inhibitor, or the chemotherapeutic agent is administered concomitantly with the multispecific antibody or the pharmaceutical formulation.

[E-10] The method of any one of [E-6] to [E-8], wherein the additional therapeutic agent, immune checkpoint inhibitor, or the chemotherapeutic agent is administered before or after the administration of the multispecific antibody or the pharmaceutical formulation.

[F-1] Use of the multispecific antibody of any one of [B-1] to [B-18] or the pharmaceutical formulation of any one of [C-1] to [C-3] in the manufacture of a medicament for treatment of cancer.

[F-2] Use of the multispecific antibody of any one of [B-1] to [B-18] or the pharmaceutical formulation of any one of [C-1] to [C-3] in combination with an additional therapeutic agent (preferably a chemotherapeutic agent, or an immune checkpoint inhibitor), in the manufacture of a medicament for treatment of cancer.

[F-3] The use of [F-2], wherein the immune checkpoint inhibitor is a PD-1 axis binding antagonist, preferably an anti-PD-1 antibody or an anti-PD-L1 antibody, more preferably atezolizumab.

[F-4] The use of [F-2], wherein the chemotherapeutic agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, an antiangiogenic agent, Etoposide, Irinotecan, Lurbinectedin, Amrubicin, and platinum agents (such as cisplatin and carboplatin).

[G-1] The pharmaceutical formulation of any one of [C-1] to [C-7], the method or use of any one of [D-1] to [F-4], wherein the cancer is a DLL3-expressing or DLL3-positive cancer.

[G-2] The pharmaceutical formulation of any one of [C-1] to [C-7], the method or use of any one of [D-1] to [F-4], wherein the cancer is selected from the group consisting of neuroendocrine neoplasm (NEN), neuroendocrine tumor (NET), neuroendocrine carcinoma (NEC) or other solid tumors of non-neuroendocrine origin.

[G-3] The pharmaceutical formulation of any one of [C-1] to [C-7], the method or use of any one of [D-1] to [F-4], wherein the cancer is selected from the group consisting of pancreatic neuroendocrine tumor (PNET), small-cell type NEC (SCNEC), large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC), neuroblastoma, glioma or glioblastoma (GBM), melanoma, and medullary thyroid cancer.

[G-4] The pharmaceutical formulation of any one of [C-1] to [C-7], the method or use of any one of [D-1] to [F-4], wherein the cancer is lung cancer, preferably SCLC.

[G-5] The pharmaceutical formulation of any one of [C-1] to [C-7], the method or use of any one of [D-1] to [F-4], wherein the cancer is glioma or glioblastoma (GBM).

[G-6] The pharmaceutical formulation of any one of [C-1] to [C-7], the method or use of any one of [D-1] to [F-4], wherein the cancer is neuroendocrine prostate cancer.

[G-7] The pharmaceutical formulation of any one of [C-1] to [C-7], the method or use of any one of [D-1] to [F-4], wherein the cancer is neuroblastoma.

In another aspect, the present disclosure provides the following:

[1] A multispecific antigen-binding molecule comprising:
a first antigen-binding moiety and a second antigen-binding moiety, each of which have one, two or more of the following properties:
(i) binds to human CD3;
(ii) binds to human CD137;
(iii) capable of binding to human CD3 and human CD137, wherein each of the first antigen-binding moiety and the second antigen-binding moiety binds to either one of human CD3 and human CD137; and
(iv) capable of binding to human CD3 and human CD137, but does not bind to human CD3 and human CD137 at the same time;
and a third antigen-binding moiety that is capable of binding to a third antigen, preferably an antigen expressed on a cancer cell/tissue, more preferably DLL3, even more preferably human DLL3.

[2] The multispecific antigen-binding molecule of [1], wherein the first antigen-binding moiety and the second antigen-binding moiety each comprises an antibody variable region comprising any one of (a1) to (a17) below:
(a1) an antibody variable region comprising heavy chain complementarity determining region (CDR) 1 of SEQ ID NO: 17, heavy chain CDR 2 of SEQ ID NO: 31, heavy chain CDR 3 of SEQ ID NO: 45, light chain CDR 1 of SEQ ID NO: 64, light chain CDR 2 of SEQ ID NO: 69 and light chain CDR 3 of SEQ ID NO: 74;
(a2) an antibody variable region comprising heavy chain complementarity determining region (CDR) 1 of SEQ ID NO: 18, heavy chain CDR 2 of SEQ ID NO: 32, heavy chain CDR 3 of SEQ ID NO: 46, light chain CDR 1 of SEQ ID NO: 63, light chain CDR 2 of SEQ ID NO: 68 and light chain CDR 3 of SEQ ID NO: 73;
(a3) an antibody variable region comprising the heavy chain complementarity determining region (CDR) 1 of SEQ ID NO: 19, the heavy chain CDR 2 of SEQ ID NO: 33, the heavy chain CDR 3 of SEQ ID NO: 47, light chain CDR 1 of SEQ ID NO: 63, light chain CDR 2 of SEQ ID NO: 68 and light chain CDR 3 of SEQ ID NO: 73;
(a4) an antibody variable region comprising the heavy chain complementarity determining region (CDR) 1 of SEQ ID NO: 19, the heavy chain CDR 2 of SEQ ID NO: 33, the heavy chain CDR 3 of SEQ ID NO: 47, light chain CDR 1 of SEQ ID NO: 65, light chain CDR 2 of SEQ ID NO: 70 and light chain CDR 3 of SEQ ID NO: 75;
(a5) an antibody variable region comprising the heavy chain complementarity determining region (CDR) 1 of SEQ ID NO: 20, the heavy chain CDR 2 of SEQ ID NO: 34, the heavy chain CDR 3 of SEQ ID NO: 48, light chain CDR 1 of SEQ ID NO: 63, light chain CDR 2 of SEQ ID NO: 68 and light chain CDR 3 of SEQ ID NO: 73;
(a6) an antibody variable region comprising the heavy chain complementarity determining region (CDR) 1 of SEQ ID NO: 22, the heavy chain CDR 2 of SEQ ID NO: 36, the heavy chain CDR 3 of SEQ ID NO: 50, light chain CDR 1 of SEQ ID NO: 63, light chain CDR 2 of SEQ ID NO: 68 and light chain CDR 3 of SEQ ID NO: 73;
(a7) an antibody variable region comprising the heavy chain complementarity determining region (CDR) 1 of SEQ ID NO: 23, the heavy chain CDR 2 of SEQ ID NO: 37, the heavy chain CDR 3 of SEQ ID NO: 51, light chain CDR 1 of SEQ ID NO: 63, light chain CDR 2 of SEQ ID NO: 68 and light chain CDR 3 of SEQ ID NO: 73;
(a8) an antibody variable region comprising the heavy chain complementarity determining region (CDR) 1 of SEQ ID NO: 23, the heavy chain CDR 2 of SEQ ID NO: 37, the heavy chain CDR 3 of SEQ ID NO: 51, light chain CDR 1 of SEQ ID NO: 66, light chain CDR 2 of SEQ ID NO: 71 and light chain CDR 3 of SEQ ID NO: 76;
(a9) an antibody variable region comprising the heavy chain complementarity determining region (CDR) 1 of SEQ ID NO: 24, the heavy chain CDR 2 of SEQ ID NO: 38, the heavy chain CDR 3 of SEQ ID NO: 52, light chain CDR 1 of SEQ ID NO: 63, light chain CDR 2 of SEQ ID NO: 68 and light chain CDR 3 of SEQ ID NO: 73;
(a10) an antibody variable region comprising the heavy chain complementarity determining region (CDR) 1 of SEQ ID NO: 25, the heavy chain CDR 2 of SEQ ID NO: 39, the heavy chain CDR 3 of SEQ ID NO: 53, light chain CDR 1 of SEQ ID NO: 66, light chain CDR 2 of SEQ ID NO: 71 and light chain CDR 3 of SEQ ID NO: 76;
(a11) an antibody variable region comprising the heavy chain complementarity determining region (CDR) 1 of SEQ ID NO: 26, the heavy chain CDR 2 of SEQ ID NO: 40, the heavy chain CDR 3 of SEQ ID NO: 54, light chain CDR 1 of SEQ ID NO: 66, light chain CDR 2 of SEQ ID NO: 71 and light chain CDR 3 of SEQ ID NO: 76;
(a12) an antibody variable region comprising the heavy chain complementarity determining region (CDR) 1 of SEQ ID NO: 26, the heavy chain CDR 2 of SEQ ID NO: 40, the heavy chain CDR 3 of SEQ ID NO: 54, light chain CDR 1 of SEQ ID NO: 63, light chain CDR 2 of SEQ ID NO: 68 and light chain CDR 3 of SEQ ID NO: 73;
(a13) an antibody variable region comprising the heavy chain complementarity determining region (CDR) 1 of SEQ ID NO: 27, the heavy chain CDR 2 of SEQ ID NO: 41, the heavy chain CDR 3 of SEQ ID NO: 55, light chain CDR 1 of SEQ ID NO: 63, light chain CDR 2 of SEQ ID NO: 68 and light chain CDR 3 of SEQ ID NO: 73;
(a14) an antibody variable region comprising the heavy chain complementarity determining region (CDR) 1 of SEQ ID NO: 28, the heavy chain CDR 2 of SEQ ID NO: 42, the heavy chain CDR 3 of SEQ ID NO: 56, light chain CDR 1 of SEQ ID NO: 63, light chain CDR 2 of SEQ ID NO: 68 and light chain CDR 3 of SEQ ID NO: 73;
(a15) an antibody variable region comprising heavy chain complementarity determining region (CDR) 1 of SEQ ID NO: 82, heavy chain CDR 2 of SEQ ID NO: 83, heavy chain CDR 3 of SEQ ID NO: 84, light chain CDR 1 of SEQ ID NO: 65, light chain CDR 2 of SEQ ID NO: 70 and light chain CDR 3 of SEQ ID NO: 75;
(a16) an antibody variable region that binds to the same epitope of any of the antibody variable region selected from (a1) to (a15); and
(a17) an antibody variable fragment that competes with the binding of any of the antibody variable fragment selected from (a1) to (a15);

[3] The multispecific antigen-binding molecule of any one of [1] to [2], wherein the first antigen-binding moiety and the second antigen-binding moiety each comprises an antibody variable region comprising any one of (a1) to (a18) below:
(a1) an antibody variable region comprising a VH comprising SEQ ID NO: 3 and a VL comprising SEQ ID NO: 59;
(a2) an antibody variable region comprising a VH comprising SEQ ID NO: 4 and a VL comprising SEQ ID NO: 58;
(a3) an antibody variable region comprising a VH comprising SEQ ID NO: 5 and a VL comprising SEQ ID NO:58;
(a4) an antibody variable region comprising a VH comprising SEQ ID NO: 5 and a VL comprising SEQ ID NO: 60;
(a5) an antibody variable region comprising a VH comprising SEQ ID NO: 6 and a VL comprising SEQ ID NO: 58;
(a6) an antibody variable region comprising a VH comprising SEQ ID NO: 8 and a VL comprising SEQ ID NO: 58;
(a7) an antibody variable region comprising a VH comprising SEQ ID NO: 9 and a VL comprising SEQ ID NO: 58;
(a8) an antibody variable region comprising a VH comprising SEQ ID NO: 9 and a VL comprising SEQ ID NO: 61;
(a9) an antibody variable region comprising a VH comprising SEQ ID NO: 10 and a VL comprising SEQ ID NO: 58;
(a10) an antibody variable region comprising a VH comprising SEQ ID NO: 11 and a VL comprising SEQ ID NO: 61;
(a11) an antibody variable region comprising a VH comprising SEQ ID NO: 12 and a VL comprising SEQ ID NO: 61;
(a12) an antibody variable region comprising a VH comprising SEQ ID NO: 12 and a VL comprising SEQ ID NO: 58;
(a13) an antibody variable region comprising a VH comprising SEQ ID NO: 13 and a VL comprising SEQ ID NO: 58;
(a14) an antibody variable region comprising a VH comprising SEQ ID NO: 14 and a VL comprising SEQ ID NO: 58; and
(a15) an antibody variable region comprising a VH comprising SEQ ID NO: 81 and a VL comprising SEQ ID NO: 60;
(a16) an antibody variable region comprising a VH comprising an amino acid sequence selected from the group consisting of SEQ ID NO: 3-6, 8-14 and 81, and/or a VL comprising an amino acid sequence selected from the group consisting of SEQ ID NO: 58-61.
(a17) an antibody variable region that binds to the same epitope of any of the antibody variable region selected from (a1) to (a15); and
(a18) an antibody variable fragment that competes with the binding of any of the antibody variable fragment selected from (a1) to (a15).

[4] The multispecific antigen-binding molecule of any one of [1] to [3], wherein each of the first antigen-binding moiety and the second antigen-binding moiety is a Fab molecule and comprises at least one disulfide bond formed between the CH1 region of the first antigen-binding moiety and the CH1 region of the second antigen-binding moiety.

[4A] The multispecific antigen-binding molecule of [4], wherein each of the first antigen-binding moiety and the second antigen-binding moiety is a Fab molecule and comprises one disulfide bond formed between the amino acid residues at position 191 according to EU numbering in the respective CH1 region of the first antigen-binding moiety and the second antigen-binding moiety.

[5] The multispecific antigen-binding molecule of any one of [1] to [4A], wherein the third antigen binding moiety is fused to either one of the first antigen binding moiety or the second antigen binding moiety.

[5A] The multispecific antigen-binding molecule of [5], wherein the third antigen binding moiety is a Fab or scFv.

[6] The multispecific antigen-binding molecule of any one of [5] to [5A], wherein each of the first, second and third antigen binding moiety is a Fab molecule, wherein the third antigen binding moiety is fused at the C-terminus of the Fab heavy chain (CH1) to the N-terminus of the Fab heavy chain of either one of the first antigen binding moiety or the second antigen binding moiety, optionally via a peptide linker.

[6A] The multispecific antigen-binding molecule of any one of [5] to [6], wherein said peptide linker is selected from the group consisting of the amino acid sequence of SEQ ID NO: 248, SEQ ID NO: 249 or SEQ ID NO: 259.

[6B] The multispecific antigen-binding molecule of any one of [1] to [6A], wherein the first antigen binding moiety is identical to the second antigen binding moiety.

[7] The multispecific antigen-binding molecule of any one of [1] to [6B], wherein the third antigen binding moiety is a crossover Fab molecule in which the variable regions of the Fab light chain and the Fab heavy chain are exchanged, and wherein each of the first and second antigen binding moiety is a conventional Fab molecule.

[8] The multispecific antigen-binding molecule of [7], wherein in the constant domain CL of the light chain of each of the first and second antigen binding moiety, the amino acid(s) at position 123 and/or 124 is/are substituted independently by lysine (K), arginine (R) or histidine (H) (numbering according to Kabat), and wherein in the constant domain CH1 of the heavy chain of each of the first and second antigen binding moiety, the amino acid at position 147 and/or the amino acid at position 213 is substituted independently by glutamic acid (E) or aspartic acid (D) (numbering according to Kabat EU index).

[9] The multispecific antigen-binding molecule of [8], wherein in the constant domain CL of the light chain of each of the first and second antigen binding moiety, the amino acids at position 123 and 124 are arginine (R) and lysine (K) respectively (numbering according to Kabat), and wherein in the constant domain CH1 of the heavy chain of each of the first and second antigen binding moiety the amino acids at position 147 and 213 are glutamic acid (E) (numbering according to Kabat EU index).

[10] The multispecific antigen-binding molecule of any one of [1] to [9], wherein the third antigen-binding moiety capable of binding to DLL3 comprises an antibody variable region comprising any one of (a1) to (a5) below:
(a1) the heavy chain complementarity determining region (CDR) 1 of SEQ ID NO: 233, the heavy chain CDR 2 of SEQ ID NO: 234, the heavy chain CDR 3 of SEQ ID NO: 235, the light chain CDR 1 of SEQ ID NO: 237, the light chain CDR 2 of SEQ ID NO: 238 and the light chain CDR 3 of SEQ ID NO: 239;
(a2) the heavy chain complementarity determining region (CDR) 1 of SEQ ID NO: 276, the heavy chain CDR 2 of SEQ ID NO: 277, the heavy chain CDR 3 of SEQ ID NO: 278, the light chain CDR 1 of SEQ ID NO: 279, the light chain CDR 2 of SEQ ID NO: 280 and the light chain CDR 3 of SEQ ID NO: 281;
(a3) the heavy chain complementarity determining region (CDR) 1 of SEQ ID NO: 285, the heavy chain CDR 2 of SEQ ID NO: 286, the heavy chain CDR 3 of SEQ ID NO: 287, the light chain CDR 1 of SEQ ID NO: 288, the light chain CDR 2 of SEQ ID NO: 289 and the light chain CDR 3 of SEQ ID NO: 290;
(a4) an antibody variable region that binds to the same epitope of any of the antibody variable region selected from (a1) to (a3); and
(a5) an antibody variable fragment that competes with the binding of any of the antibody variable fragment selected from (a1) to (a3).

[11] The multispecific antigen-binding molecule of any one of [1] to [10], wherein the third antigen-binding moiety capable of binding to DLL3 comprises an antibody variable region comprising any one of (a1) to (a6) below:
(a1) a heavy chain variable region comprising an amino acid sequence of SEQ ID NO: 232, and a light chain variable region comprising an amino acid sequence of SEQ ID NO: 236;
(a2) a heavy chain variable region comprising an amino acid sequence of SEQ ID NO: 264, and a light chain variable region comprising an amino acid sequence of SEQ ID NO: 265;
(a3) a heavy chain variable region comprising an amino acid sequence of SEQ ID NO: 266, and a light chain variable region comprising an amino acid sequence of SEQ ID NO: 267;
(a4) a heavy chain variable region comprising an amino acid sequence of SEQ ID NO: 268, and a light chain variable region comprising an amino acid sequence of SEQ ID NO: 269;
(a5) an antibody variable region that binds to the same epitope of any of the antibody variable region selected from (a1) to (a4); and
(a6) an antibody variable fragment that competes with the binding of any of the antibody variable fragment selected from (a1) to (a4).

[12] The multispecific antigen-binding molecule of any one of [1] to [111], further comprises a Fc domain.

[12A] The multispecific antigen-binding molecule of [12], wherein the Fc domain composed of a first and a second Fc region subunit capable of stable association, and wherein the Fc domain exhibits reduced binding affinity to human Fc-gamma receptor, as compared to a native human IgG1 Fc domain.

[12B] The multispecific antigen-binding molecule of [12A], wherein the first Fc-region subunit is selected from the group consisting of:
(a1) a Fc region polypeptide comprising Ala at position 234 and Ala at position 235;
(a2) a Fc region polypeptide comprising Ala at position 234, Ala at position 235, and Ala at position 297;
(a3) a Fc region polypeptide comprising Ala at position 234, Ala at position 235, Ala at position 297, Cys at position 354 and Trp at position 366; and
wherein the second Fc-region subunit is selected from the group consisting of:
(a4) a Fc region polypeptide comprising Ala at position 234 and Ala at position 235;
(a5) a Fc region polypeptide comprising Ala at position 234, Ala at position 235, and Ala at position 297;
(a6) a Fc region polypeptide comprising Ala at position 234, Ala at position 235, Ala at position 297, Cys at position 349, Ser at position 366, Ala at position 368 and Val at position 407; and
wherein the amino acid positions are numbered using EU index numbering.

[12C] The multispecific antigen-binding molecule of any one of [12] to [12B], wherein the Fc domain exhibits enhanced FcRn-binding activity under an acidic pH condition (e.g., pH 5.8) as compared to that of an Fc region of a native IgG.

[12D] The multispecific antigen-binding molecule of [12C], wherein the Fc domain comprises Ala at position 434; Glu, Arg, Ser, or Lys at position 438; and Glu, Asp, or Gln at position 440, according to EU numbering.

[12E] The multispecific antigen-binding molecule of [12D], wherein the Fc domain comprises Ala at position 434; Arg or Lys at position 438; and Glu or Asp at position 440, according to EU numbering.

[12F] The multispecific antigen-binding molecule of [12E], wherein the Fc domain further comprises Ile or Leu at position 428; and/or Ile, Leu, Val, Thr, or Phe at position 436, according to EU numbering.

[12G] The multispecific antigen-binding molecule of any one of [12C] to [12F], wherein the Fc domain comprises a combination of amino acid substitutions selected from the group consisting of:
(a) N434A/Q438R/S440E;
(b) N434A/Q438R/S440D;
(c) N434A/Q438K/S440E;
(d) N434A/Q438K/S440D;
(e) N434A/Y436T/Q438R/S440E;
(f) N434A/Y436T/Q438R/S440D;
(g) N434A/Y436T/Q438K/S440E;
(h) N434A/Y436T/Q438K/S440D;
(i) N434A/Y436V/Q438R/S440E;
(j) N434A/Y436V/Q438R/S440D;
(k) N434A/Y436V/Q438K/S440E;
(l) N434A/Y436V/Q438K/S440D;
(m) N434A/R435H/F436T/Q438R/S440E;
(n) N434A/R435H/F436T/Q438R/S440D;
(o) N434A/R435H/F436T/Q438K/S440E;
(p) N434A/R435H/F436T/Q438K/S440D;
(q) N434A/R435H/F436V/Q438R/S440E;
(r) N434A/R435H/F436V/Q438R/S440D;
(s) N434A/R435H/F436V/Q438K/S440E;
(t) N434A/R435H/F436V/Q438K/S440D;
(u) M428L/N434A/Q438R/S440E;
(v) M428L/N434A/Q438R/S440D;
(w) M428L/N434A/Q438K/S440E;
(x) M428L/N434A/Q438K/S440D;
(y) M428L/N434A/Y436T/Q438R/S440E;
(z) M428L/N434A/Y436T/Q438R/S440D;
(aa) M428L/N434A/Y436T/Q438K/S440E;
(ab) M428L/N434A/Y436T/Q438K/S440D;
(ac) M428L/N434A/Y436V/Q438R/S440E;
(ad) M428L/N434A/Y436V/Q438R/S440D;
(ae) M428L/N434A/Y436V/Q438K/S440E;
(af) M428L/N434A/Y436V/Q438K/S440D;
(ag) L235R/G236R/S239K/M428L/N434A/Y436T/Q438R/S440E; and
(ah) L235R/G236R/A327G/A330S/P331S/M428L/N434A/Y436T/Q438R/S440E, according to EU numbering.

[12H] The multispecific antigen-binding molecule of any one of [12C] to [12G], wherein the Fc domain comprises a combination of amino acid substitutions of M428L/N434A/Q438R/S440E.

[12I] The multispecific antigen-binding molecule of any one of [12] to [12H], wherein the Fc domain is a IgG Fc domain, preferably a human IgG Fc domain, more preferably a human IgG1 Fc domain.

[12J] The multispecific antigen-binding molecule of any one of [12] to [12I], wherein the Fc domain comprises any one of:
(a) a first Fc subunit comprising the amino acid sequence shown in SEQ ID NO: 100 and a second Fc subunit comprising the amino acid sequence shown in SEQ ID NO: 111; and
(b) a first Fc subunit comprising the amino acid sequence shown in SEQ ID NO: 99 and a second Fc subunit comprising the amino acid sequence shown in SEQ ID NO: 109.

[12K] The multispecific antigen-binding molecule of any one of [12] to [12J], wherein each of the first and second antigen-binding moiety is a Fab, wherein the first antigen-binding moiety is fused at the C-terminus of the Fab heavy chain to the N-terminus of the first or second subunit of the Fc domain, and the second antigen-binding moiety is fused at the C-terminus of the Fab heavy chain to the N-terminus of the remaining subunit of the Fc domain.

[12L] The multispecific antigen-binding molecule of [12K], wherein the third antigen binding moiety is fused at the C-terminus to the N-terminus of the Fab heavy chain of either one of the first antigen binding moiety or the second antigen binding moiety, optionally via a peptide linker.

[13] A multispecific antigen-binding molecule comprising five polypeptide chains in any one combination selected from (a1) to (a15) below:
(a1) a polypeptide chain comprising the amino acid sequence of SEQ ID NO: 201 (chain 1), a polypeptide chain comprising the amino acid sequence of SEQ ID NO: 206 (chain 2), a polypeptide chain comprising the amino acid sequence of SEQ ID NO: 208 (chain 3) and two polypeptide chains each comprising the amino acid sequence of SEQ ID NO: 214 (chain 4 & chain 5);
(a2) a polypeptide chain comprising the amino acid sequence of SEQ ID NO: 203 (chain 1), a polypeptide chain comprising the amino acid sequence of SEQ ID NO: 206 (chain 2), a polypeptide chain comprising the amino acid sequence of SEQ ID NO: 209 (chain 3) and two polypeptide chains each comprising the amino acid sequence of SEQ ID NO: 214 (chain 4 & chain 5);
(a3) a polypeptide chain comprising the amino acid sequence of SEQ ID NO: 204 (chain 1), a polypeptide chain comprising the amino acid sequence of SEQ ID NO: 206 (chain 2), a polypeptide chain comprising the amino acid sequence of SEQ ID NO: 209 (chain 3) and two polypeptide chains each comprising the amino acid sequence of SEQ ID NO: 214 (chain 4 & chain 5);
(a4) a polypeptide chain comprising the amino acid sequence of SEQ ID NO: 205 (chain 1), a polypeptide chain comprising the amino acid sequence of SEQ ID NO: 206 (chain 2), a polypeptide chain comprising the amino acid sequence of SEQ ID NO: 209 (chain 3) and two polypeptide chains each comprising the amino acid sequence of SEQ ID NO: 214 (chain 4 & chain 5);
(a5) a polypeptide chain comprising the amino acid sequence of SEQ ID NO: 216 (chain 1), a polypeptide chain comprising the amino acid sequence of SEQ ID NO: 206 (chain 2), a polypeptide chain comprising the amino acid sequence of SEQ ID NO: 229 (chain 3) and two polypeptide chains each comprising the amino acid sequence of SEQ ID NO: 214 (chain 4 & chain 5);
(a6) a polypeptide chain comprising the amino acid sequence of SEQ ID NO: 217 (chain 1), a polypeptide chain comprising the amino acid sequence of SEQ ID NO: 206 (chain 2), a polypeptide chain comprising the amino acid sequence of SEQ ID NO: 210 (chain 3) and two polypeptide chains each comprising the amino acid sequence of SEQ ID NO: 214 (chain 4 & chain 5);
(a7) a polypeptide chain comprising the amino acid sequence of SEQ ID NO: 219 (chain 1), a polypeptide chain comprising the amino acid sequence of SEQ ID NO: 206 (chain 2), a polypeptide chain comprising the amino acid sequence of SEQ ID NO: 211 (chain 3) and two polypeptide chains each comprising the amino acid sequence of SEQ ID NO: 214 (chain 4 & chain 5);
(a8) a polypeptide chain comprising the amino acid sequence of SEQ ID NO: 220 (chain 1), a polypeptide chain comprising the amino acid sequence of SEQ ID NO: 206 (chain 2), a polypeptide chain comprising the amino acid sequence of SEQ ID NO: 211 (chain 3) and two polypeptide chains each comprising the amino acid sequence of SEQ ID NO: 214 (chain 4 & chain 5);
(a9) a polypeptide chain comprising the amino acid sequence of SEQ ID NO: 221 (chain 1), a polypeptide chain comprising the amino acid sequence of SEQ ID NO: 206 (chain 2), a polypeptide chain comprising the amino acid sequence of SEQ ID NO: 211 (chain 3) and two polypeptide chains each comprising the amino acid sequence of SEQ ID NO: 214 (chain 4 & chain 5);
(a10) a polypeptide chain comprising the amino acid sequence of SEQ ID NO: 222 (chain 1), a polypeptide chain comprising the amino acid sequence of SEQ ID NO: 206 (chain 2), a polypeptide chain comprising the amino acid sequence of SEQ ID NO: 230 (chain 3) and two polypeptide chains each comprising the amino acid sequence of SEQ ID NO: 214 (chain 4 & chain 5);
(a11) a polypeptide chain comprising the amino acid sequence of SEQ ID NO: 223 (chain 1), a polypeptide chain comprising the amino acid sequence of SEQ ID NO: 206 (chain 2), a polypeptide chain comprising the amino acid sequence of SEQ ID NO: 212 (chain 3) and two polypeptide chains each comprising the amino acid sequence of SEQ ID NO: 215 (chain 4 & chain 5);
(a12) a polypeptide chain comprising the amino acid sequence of SEQ ID NO: 225 (chain 1), a polypeptide chain comprising the amino acid sequence of SEQ ID NO: 206 (chain 2), a polypeptide chain comprising the amino acid sequence of SEQ ID NO: 213 (chain 3) and two polypeptide chains each comprising the amino acid sequence of SEQ ID NO: 215 (chain 4 & chain 5);
(a13) a polypeptide chain comprising the amino acid sequence of SEQ ID NO: 226 (chain 1), a polypeptide chain comprising the amino acid sequence of SEQ ID NO: 206 (chain 2), a polypeptide chain comprising the amino acid sequence of SEQ ID NO: 213 (chain 3) and two polypeptide chains each comprising the amino acid sequence of SEQ ID NO: 215 (chain 4 & chain 5);
(a14) a polypeptide chain comprising the amino acid sequence of SEQ ID NO: 227 (chain 1), a polypeptide chain comprising the amino acid sequence of SEQ ID NO: 206 (chain 2), a polypeptide chain comprising the amino acid sequence of SEQ ID NO: 213 (chain 3) and two polypeptide chains each comprising the amino acid sequence of SEQ ID NO: 215 (chain 4 & chain 5); and
(a15) a polypeptide chain comprising the amino acid sequence of SEQ ID NO: 228 (chain 1), a polypeptide chain comprising the amino acid sequence of SEQ ID NO: 206 (chain 2), a polypeptide chain comprising the amino acid sequence of SEQ ID NO: 231 (chain 3) and two polypeptide chains each comprising the amino acid sequence of SEQ ID NO: 215 (chain 4 & chain 5);
and wherein, preferably the five polypeptide chains (chain 1 to chain 5) are connected and/or associate with each other according to the orientation shown in FIG. 1(a) (more specifically, each of the polypeptide chain 2 and chain 5 associates with the polypeptide chain 1; the polypeptide chain 4 associates with the polypeptide chain 3; and the polypeptide chain 1 associates with the polypeptide chain 3).

[14] An isolated polynucleotide or plurality of polynucleotides encoding the multispecific antigen-binding molecule of any one of [1] to [13].

[15] A vector encoding the polynucleotide or plurality of polynucleotides of [14].

[16] A host cell comprising the polynucleotide or plurality of polynucleotides of [14], or the vector of [15].

[17] A method of producing the multispecific antigen-binding molecule of any one of to [13], comprising the steps of a) culturing the host cell of [16] under conditions suitable for the expression of the antigen-binding molecule and b) recovering the antigen-binding molecule.

[17A] A multispecific antigen-binding molecule produced by the method of [17].

[18] A pharmaceutical composition comprising the multispecific antigen-binding molecule of any one of [1] to [13] and a pharmaceutically acceptable carrier.

[19] The multispecific antigen-binding molecule of any one of [1] to [13] or the pharmaceutical composition of [18], which induces cytotoxicity, preferably T-cell-dependent cytotoxicity.

[20] The multispecific antigen-binding molecule of any one of [1] to [13] or the pharmaceutical composition of [18], for use as a medicament.

[21] The multispecific antigen-binding molecule of any one of [1] to [13] or the pharmaceutical composition of [18], for use in the treatment or prevention of a disease in an individual in need thereof.

[22] The multispecific antigen-binding molecule or the pharmaceutical composition for use in the treatment/prevention of a disease of [21], wherein the disease is cancer.

[22A] The multispecific antigen-binding molecule or the pharmaceutical composition for use in the treatment/prevention of a disease of [22], wherein the cancer is DLL3-expressing cancer or DLL3-positive cancer, preferably neuroendocrine neoplasm (NEN), neuroendocrine tumor (NET), neuroendocrine carcinoma (NEC), or other solid tumors of non-neuroendocrine origin.

[22B] The multispecific antigen-binding molecule or the pharmaceutical composition for use in the treatment/prevention of a disease of [22] or [22A], wherein the cancer is selected from the group consisting of pancreatic neuroendocrine tumor (PNET), small-cell type NEC (SCNEC), large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC), neuroblastoma, glioma or glioblastoma (GBM), melanoma, and medullary thyroid cancer.

[23] Use of the multispecific antigen binding molecule of any one of [1] to [13] or the pharmaceutical composition of [18], for the manufacture of a medicament for the treatment or prevention of a disease in an individual in need thereof.

[24] A method of treating a disease in an individual, comprising administering to said individual a therapeutically effective amount of a composition comprising the multispecific antigen binding molecule of any one of [1] to [13] or the pharmaceutical composition of [18].

[25] The use of [23] or the method of [24], wherein said disease is cancer, preferably DLL3-positive cancer or DLL3-expressing cancer.

[25A] The use of or the method of [25], wherein the cancer is DLL3-expressing cancer or DLL3-positive cancer, preferably neuroendocrine neoplasm (NEN), neuroendocrine tumor (NET), neuroendocrine carcinoma (NEC), or other solid tumors of non-neuroendocrine origin.

[25B] The use of or the method of [25A], wherein the cancer is selected from the group consisting of pancreatic neuroendocrine tumor (PNET), small-cell type NEC (SCNEC), large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC), neuroblastoma, glioma or glioblastoma (GBM), melanoma, and medullary thyroid cancer.

[26] A method for inducing lysis of a target cell, comprising contacting a target cell with the multispecific antigen binding molecule of any one of [1] to [13] or the pharmaceutical composition of [18] in the presence of a T cell.

[27] A kit comprising the composition of [18]; and a package insert comprising instructions for administering to a subject to treat or delay progression of cancer, preferably DLL3-positive cancer or DLL3-expressing cancer, preferably neuroendocrine neoplasm (NEN), neuroendocrine tumor (NET), neuroendocrine carcinoma (NEC) or other solid tumors of non-neuroendocrine origin.

[27A] The kit of [27], wherein the cancer is selected from the group consisting of pancreatic neuroendocrine tumor (PNET), small-cell type NEC (SCNEC), large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC), neuroblastoma, glioma or glioblastoma (GBM), melanoma, and medullary thyroid cancer.

Another aspect of the present invention relates to:

[28] A multispecific antigen-binding molecule comprising:
a first antigen-binding moiety which has one, two or more of the following properties:
(i) binds to human CD3;
(ii) binds to human CD137;
(iii) capable of binding to human CD3 and human CD137, wherein the first antigen-binding moiety binds to either one of human CD3 or human CD137; and
(iv) capable of binding to human CD3 and human CD137, but does not bind to human CD3 and human CD137 at the same time; and a second antigen-binding moiety that is capable of binding to DLL3, preferably human DLL3.

[29] The multispecific antigen-binding molecule of [28], wherein the first antigen-binding moiety comprises an antibody variable region comprising any one of (a1) to (a17) below:
(a1) the heavy chain complementarity determining region (CDR) 1 of SEQ ID NO: 17, the heavy chain CDR 2 of SEQ ID NO: 31, the heavy chain CDR 3 of SEQ ID NO: 45, the light chain CDR 1 of SEQ ID NO: 64, the light chain CDR 2 of SEQ ID NO: 69 and the light chain CDR 3 of SEQ ID NO: 74;
(a2) the heavy chain complementarity determining region (CDR) 1 of SEQ ID NO: 18, the heavy chain CDR 2 of SEQ ID NO: 32, the heavy chain CDR 3 of SEQ ID NO: 46, the light chain CDR 1 of SEQ ID NO: 63, the light chain CDR 2 of SEQ ID NO: 68 and the light chain CDR 3 of SEQ ID NO: 73;
(a3) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 63, the light chain CDR 2 of SEQ ID NO: 68 and the light chain CDR 3 of SEQ ID NO: 73;
(a4) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 65, the light chain CDR 2 of SEQ ID NO: 70 and the light chain CDR 3 of SEQ ID NO: 75;
(a5) the heavy chain complementarity determining region (CDR) 1 of SEQ ID NO: 20, the heavy chain CDR 2 of SEQ ID NO: 34, the heavy chain CDR 3 of SEQ ID NO: 48, the light chain CDR 1 of SEQ ID NO: 63, the light chain CDR 2 of SEQ ID NO: 68 and the light chain CDR 3 of SEQ ID NO: 73;
(a6) the heavy chain complementarity determining region (CDR) 1 of SEQ ID NO: 22, the heavy chain CDR 2 of SEQ ID NO: 36, the heavy chain CDR 3 of SEQ ID NO: 50, the light chain CDR 1 of SEQ ID NO: 63, the light chain CDR 2 of SEQ ID NO: 68 and the light chain CDR 3 of SEQ ID NO: 73;
(a7) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 63, the light chain CDR 2 of SEQ ID NO: 68 and the light chain CDR 3 of SEQ ID NO: 73;
(a8) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 66, the light chain CDR 2 of SEQ ID NO: 71 and the light chain CDR 3 of SEQ ID NO: 76;
(a9) the heavy chain complementarity determining region (CDR) 1 of SEQ ID NO: 24, the heavy chain CDR 2 of SEQ ID NO: 38, the heavy chain CDR 3 of SEQ ID NO: 52, the light chain CDR 1 of SEQ ID NO: 63, the light chain CDR 2 of SEQ ID NO: 68 and the light chain CDR 3 of SEQ ID NO: 73;
(a10) the heavy chain complementarity determining region (CDR) 1 of SEQ ID NO: 25, the heavy chain CDR 2 of SEQ ID NO: 39, the heavy chain CDR 3 of SEQ ID NO: 53, the light chain CDR 1 of SEQ ID NO: 66, the light chain CDR 2 of SEQ ID NO: 71 and the light chain CDR 3 of SEQ ID NO: 76;
(a11) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 66, the light chain CDR 2 of SEQ ID NO: 71 and the light chain CDR 3 of SEQ ID NO: 76;
(a12) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 63, the light chain CDR 2 of SEQ ID NO: 68 and the light chain CDR 3 of SEQ ID NO: 73;
(a13) the heavy chain complementarity determining region (CDR) 1 of SEQ ID NO: 27, the heavy chain CDR 2 of SEQ ID NO: 41, the heavy chain CDR 3 of SEQ ID NO: 55, the light chain CDR 1 of SEQ ID NO: 63, the light chain CDR 2 of SEQ ID NO: 68 and the light chain CDR 3 of SEQ ID NO: 73;
(a14) the heavy chain complementarity determining region (CDR) 1 of SEQ ID NO: 28, the heavy chain CDR 2 of SEQ ID NO: 42, the heavy chain CDR 3 of SEQ ID NO: 56, the light chain CDR 1 of SEQ ID NO: 63, the light chain CDR 2 of SEQ ID NO: 68 and the light chain CDR 3 of SEQ ID NO: 73;
(a15) the heavy chain complementarity determining region (CDR) 1 of SEQ ID NO: 82, the heavy chain CDR 2 of SEQ ID NO: 83, the heavy chain CDR 3 of SEQ ID NO: 84, the light chain CDR 1 of SEQ ID NO: 65, the light chain CDR 2 of SEQ ID NO: 70 and the light chain CDR 3 of SEQ ID NO: 75;
(a16) an antibody variable region that binds to the same epitope of any of the antibody variable region selected from (a1) to (a15); and
(a17) an antibody variable fragment that competes with the binding of any of the antibody variable fragment selected from (a1) to (a15).

[30] The multispecific antigen-binding molecule of any one of [28] to [29], wherein the first antigen-binding moiety comprises an antibody variable region comprising any one of (a1) to (a18) below:
(a1) a heavy chain variable region comprising an amino acid sequence of SEQ ID NO: 3, and a light chain variable region comprising an amino acid sequence of SEQ ID NO: 59;
(a2) a heavy chain variable region comprising an amino acid sequence of SEQ ID NO: 4, and a light chain variable region comprising an amino acid sequence of SEQ ID NO: 58;
(a3) a heavy chain variable region comprising an amino acid sequence of SEQ ID NO: 5, and a light chain variable region comprising an amino acid sequence of SEQ ID NO: 58;
(a4) a heavy chain variable region comprising an amino acid sequence of SEQ ID NO: 5, and a light chain variable region comprising an amino acid sequence of SEQ ID NO: 60;
(a5) a heavy chain variable region comprising an amino acid sequence of SEQ ID NO: 6, and a light chain variable region comprising an amino acid sequence of SEQ ID NO: 58;
(a6) a heavy chain variable region comprising an amino acid sequence of SEQ ID NO: 8, and a light chain variable region comprising an amino acid sequence of SEQ ID NO: 58;
(a7) a heavy chain variable region comprising an amino acid sequence of SEQ ID NO: 9, and a light chain variable region comprising an amino acid sequence of SEQ ID NO: 58;
(a8) a heavy chain variable region comprising an amino acid sequence of SEQ ID NO: 9, and a light chain variable region comprising an amino acid sequence of SEQ ID NO: 61;
(a9) a heavy chain variable region comprising an amino acid sequence of SEQ ID NO: 10, and a light chain variable region comprising an amino acid sequence of SEQ ID NO: 58;
(a10) a heavy chain variable region comprising an amino acid sequence of SEQ ID NO: 11, and a light chain variable region comprising an amino acid sequence of SEQ ID NO: 61;
(a11) a heavy chain variable region comprising an amino acid sequence of SEQ ID NO: 12, and a light chain variable region comprising an amino acid sequence of SEQ ID NO: 61;
(a12) a heavy chain variable region comprising an amino acid sequence of SEQ ID NO: 12, and a light chain variable region comprising an amino acid sequence of SEQ ID NO: 58;
(a13) a heavy chain variable region comprising an amino acid sequence of SEQ ID NO: 13, and a light chain variable region comprising an amino acid sequence of SEQ ID NO: 58;
(a14) a heavy chain variable region comprising an amino acid sequence of SEQ ID NO: 14, and a light chain variable region comprising an amino acid sequence of SEQ ID NO: 58;
(a15) a heavy chain variable region comprising an amino acid sequence of SEQ ID NO: 81, and a light chain variable region comprising an amino acid sequence of SEQ ID NO: 60;
(a16) a heavy chain variable region comprising an amino acid sequence selected from the group consisting of SEQ ID NO: 3-6, 8-14 and 81, and/or a light chain variable region comprising an amino acid sequence selected from the group consisting of SEQ ID NO: 58-61.
(a17) an antibody variable region that binds to the same epitope of any of the antibody variable region selected from (a1) to (a15); and
(a18) an antibody variable fragment that competes with the binding of any of the antibody variable fragment selected from (a1) to (a15).

In yet another aspect of the present invention relates to:

[31] A multispecific antigen-binding molecule comprising five polypeptide chains in any one of the combinations selected from (a1) to (a15) below:
(a1) a polypeptide chain comprising the amino acid sequence of SEQ ID NO: 201 (chain 1), a polypeptide chain comprising the amino acid sequence of SEQ ID NO: 206 (chain 2), a polypeptide chain comprising the amino acid sequence of SEQ ID NO: 208 (chain 3) and two polypeptide chains each comprising the amino acid sequence of SEQ ID NO: 214 (chain 4 & chain 5);
(a2) a polypeptide chain comprising the amino acid sequence of SEQ ID NO: 203 (chain 1), a polypeptide chain comprising the amino acid sequence of SEQ ID NO: 206 (chain 2), a polypeptide chain comprising the amino acid sequence of SEQ ID NO: 209 (chain 3) and two polypeptide chains each comprising the amino acid sequence of SEQ ID NO: 214 (chain 4 & chain 5);
(a3) a polypeptide chain comprising the amino acid sequence of SEQ ID NO: 204 (chain 1), a polypeptide chain comprising the amino acid sequence of SEQ ID NO: 206 (chain 2), a polypeptide chain comprising the amino acid sequence of SEQ ID NO: 209 (chain 3) and two polypeptide chains each comprising the amino acid sequence of SEQ ID NO: 214 (chain 4 & chain 5);
(a4) a polypeptide chain comprising the amino acid sequence of SEQ ID NO: 205 (chain 1), a polypeptide chain comprising the amino acid sequence of SEQ ID NO: 206 (chain 2), a polypeptide chain comprising the amino acid sequence of SEQ ID NO: 209 (chain 3) and two polypeptide chains each comprising the amino acid sequence of SEQ ID NO: 214 (chain 4 & chain 5);
(a5) a polypeptide chain comprising the amino acid sequence of SEQ ID NO: 216 (chain 1), a polypeptide chain comprising the amino acid sequence of SEQ ID NO: 206 (chain 2), a polypeptide chain comprising the amino acid sequence of SEQ ID NO: 229 (chain 3) and two polypeptide chains each comprising the amino acid sequence of SEQ ID NO: 214 (chain 4 & chain 5);
(a6) a polypeptide chain comprising the amino acid sequence of SEQ ID NO: 217 (chain 1), a polypeptide chain comprising the amino acid sequence of SEQ ID NO: 206 (chain 2), a polypeptide chain comprising the amino acid sequence of SEQ ID NO: 210 (chain 3) and two polypeptide chains each comprising the amino acid sequence of SEQ ID NO: 214 (chain 4 & chain 5);
(a7) a polypeptide chain comprising the amino acid sequence of SEQ ID NO: 219 (chain 1), a polypeptide chain comprising the amino acid sequence of SEQ ID NO: 206 (chain 2), a polypeptide chain comprising the amino acid sequence of SEQ ID NO: 211 (chain 3) and two polypeptide chains each comprising the amino acid sequence of SEQ ID NO: 214 (chain 4 & chain 5);
(a8) a polypeptide chain comprising the amino acid sequence of SEQ ID NO: 220 (chain 1), a polypeptide chain comprising the amino acid sequence of SEQ ID NO: 206 (chain 2), a polypeptide chain comprising the amino acid sequence of SEQ ID NO: 211 (chain 3) and two polypeptide chains each comprising the amino acid sequence of SEQ ID NO: 214 (chain 4 & chain 5);
(a9) a polypeptide chain comprising the amino acid sequence of SEQ ID NO: 221 (chain 1), a polypeptide chain comprising the amino acid sequence of SEQ ID NO: 206 (chain 2), a polypeptide chain comprising the amino acid sequence of SEQ ID NO: 211 (chain 3) and two polypeptide chains each comprising the amino acid sequence of SEQ ID NO: 214 (chain 4 & chain 5);
(a10) a polypeptide chain comprising the amino acid sequence of SEQ ID NO: 222 (chain 1), a polypeptide chain comprising the amino acid sequence of SEQ ID NO: 206 (chain 2), a polypeptide chain comprising the amino acid sequence of SEQ ID NO: 230 (chain 3) and two polypeptide chains each comprising the amino acid sequence of SEQ ID NO: 214 (chain 4 & chain 5);
(a11) a polypeptide chain comprising the amino acid sequence of SEQ ID NO: 223 (chain 1), a polypeptide chain comprising the amino acid sequence of SEQ ID NO: 206 (chain 2), a polypeptide chain comprising the amino acid sequence of SEQ ID NO: 212 (chain 3) and two polypeptide chains each comprising the amino acid sequence of SEQ ID NO: 215 (chain 4 & chain 5);
(a12) a polypeptide chain comprising the amino acid sequence of SEQ ID NO: 225 (chain 1), a polypeptide chain comprising the amino acid sequence of SEQ ID NO: 206 (chain 2), a polypeptide chain comprising the amino acid sequence of SEQ ID NO: 213 (chain 3) and two polypeptide chains each comprising the amino acid sequence of SEQ ID NO: 215 (chain 4 & chain 5);
(a13) a polypeptide chain comprising the amino acid sequence of SEQ ID NO: 226 (chain 1), a polypeptide chain comprising the amino acid sequence of SEQ ID NO: 206 (chain 2), a polypeptide chain comprising the amino acid sequence of SEQ ID NO: 213 (chain 3) and two polypeptide chains each comprising the amino acid sequence of SEQ ID NO: 215 (chain 4 & chain 5);
(a14) a polypeptide chain comprising the amino acid sequence of SEQ ID NO: 227 (chain 1), a polypeptide chain comprising the amino acid sequence of SEQ ID NO: 206 (chain 2), a polypeptide chain comprising the amino acid sequence of SEQ ID NO: 213 (chain 3) and two polypeptide chains each comprising the amino acid sequence of SEQ ID NO: 215 (chain 4 & chain 5); and
(a15) a polypeptide chain comprising the amino acid sequence of SEQ ID NO: 228 (chain 1), a polypeptide chain comprising the amino acid sequence of SEQ ID NO: 206 (chain 2), a polypeptide chain comprising the amino acid sequence of SEQ ID NO: 231 (chain 3) and two polypeptide chains each comprising the amino acid sequence of SEQ ID NO: 215 (chain 4 & chain 5); and wherein, preferably the five polypeptide chains (chain 1 to chain 5) are connected and/or associate with each other according to the orientation shown in FIG. 1(a).

[31A] A multispecific antigen-binding molecule comprising five polypeptide chains in the following combination:
a polypeptide chain comprising the amino acid sequence selected from the group consisting of SEQ ID NO: 201-205 and 216-228 (chain 1); a polypeptide chain comprising the amino acid sequence selected from the group consisting of SEQ ID NO: 206-207 (chain 2); a polypeptide chain comprising the amino acid sequence selected from the group consisting of SEQ ID NO: 208-213 and 229-231 (chain 3); and two polypeptide chains each comprising the amino acid sequence selected from the group consisting of SEQ ID NO: 214 and 215 (chain 4 & chain 5).

[31B] The multispecific antigen-binding molecule of [31] or [31A], wherein each of the polypeptide chain 2 and chain 5 associates with the polypeptide chain 1; the polypeptide chain 4 associates with the polypeptide chain 3; and the polypeptide chain 1 associates with the polypeptide chain 3.

Yet another aspect of the present invention relates to:

[32] An antigen binding molecule capable of binding to DLL3, which comprises an antibody variable region comprising any one of (a1) to (a5) below:
(a1) the heavy chain complementarity determining region (CDR) 1 of SEQ ID NO: 233, the heavy chain CDR 2 of SEQ ID NO: 234, the heavy chain CDR 3 of SEQ ID NO: 235, the light chain CDR 1 of SEQ ID NO: 237, the light chain CDR 2 of SEQ ID NO: 238 and the light chain CDR 3 of SEQ ID NO: 239;
(a2) the heavy chain complementarity determining region (CDR) 1 of SEQ ID NO: 276, the heavy chain CDR 2 of SEQ ID NO: 277, the heavy chain CDR 3 of SEQ ID NO: 278, the light chain CDR 1 of SEQ ID NO: 279, the light chain CDR 2 of SEQ ID NO: 280 and the light chain CDR 3 of SEQ ID NO: 281;
(a3) the heavy chain complementarity determining region (CDR) 1 of SEQ ID NO: 285, the heavy chain CDR 2 of SEQ ID NO: 286, the heavy chain CDR 3 of SEQ ID NO: 287, the light chain CDR 1 of SEQ ID NO: 288, the light chain CDR 2 of SEQ ID NO: 289 and the light chain CDR 3 of SEQ ID NO: 290;
(a4) an antibody variable region that binds to the same epitope of any of the antibody variable region selected from (a1) to (a3); and
(a5) an antibody variable fragment that competes with the binding of any of the antibody variable fragment selected from (a1) to (a3).

[33] An antigen binding molecule capable of binding to DLL3, which comprises an antibody variable region comprising any one of (a1) to (a6) below:
(a1) a heavy chain variable region comprising an amino acid sequence of SEQ ID NO: 232, and a light chain variable region comprising an amino acid sequence of SEQ ID NO: 236;
(a2) a heavy chain variable region comprising an amino acid sequence of SEQ ID NO: 264, and a light chain variable region comprising an amino acid sequence of SEQ ID NO: 265;
(a3) a heavy chain variable region comprising an amino acid sequence of SEQ ID NO: 266, and a light chain variable region comprising an amino acid sequence of SEQ ID NO: 267;
(a4) a heavy chain variable region comprising an amino acid sequence of SEQ ID NO: 268, and a light chain variable region comprising an amino acid sequence of SEQ ID NO: 269;
(a5) an antibody variable region that binds to the same epitope of any of the antibody variable region selected from (a1) to (a4); and
(a6) an antibody variable fragment that competes with the binding of any of the antibody variable fragment selected from (a1) to (a4).

Yet another aspect of the present invention relates to:

[33-1] A multispecific antigen-binding molecule that comprises:
(a) a first antigen-binding moiety and a second antigen-binding moiety, each of which binds to human CD137 and comprises an antibody variable region that can be the same or different and is independently selected from the group consisting of:
(a1) an antibody variable region comprising heavy chain complementarity determining region (CDR) 1 of SEQ ID NO: 17, heavy chain CDR 2 of SEQ ID NO: 31, heavy chain CDR 3 of SEQ ID NO: 45, light chain CDR 1 of SEQ ID NO: 64, light chain CDR 2 of SEQ ID NO: 69 and light chain CDR 3 of SEQ ID NO: 74;
(a2) an antibody variable region comprising heavy chain complementarity determining region (CDR) 1 of SEQ ID NO: 18, heavy chain CDR 2 of SEQ ID NO: 32, heavy chain CDR 3 of SEQ ID NO: 46, light chain CDR 1 of SEQ ID NO: 63, light chain CDR 2 of SEQ ID NO: 68 and light chain CDR 3 of SEQ ID NO: 73;
(a3) an antibody variable region comprising the heavy chain complementarity determining region (CDR) 1 of SEQ ID NO: 19, the heavy chain CDR 2 of SEQ ID NO: 33, the heavy chain CDR 3 of SEQ ID NO: 47, light chain CDR 1 of SEQ ID NO: 63, light chain CDR 2 of SEQ ID NO: 68 and light chain CDR 3 of SEQ ID NO: 73;
(a4) an antibody variable region comprising the heavy chain complementarity determining region (CDR) 1 of SEQ ID NO: 19, the heavy chain CDR 2 of SEQ ID NO: 33, the heavy chain CDR 3 of SEQ ID NO: 47, light chain CDR 1 of SEQ ID NO: 65, light chain CDR 2 of SEQ ID NO: 70 and light chain CDR 3 of SEQ ID NO: 75;
(a5) an antibody variable region comprising the heavy chain complementarity determining region (CDR) 1 of SEQ ID NO: 20, the heavy chain CDR 2 of SEQ ID NO: 34, the heavy chain CDR 3 of SEQ ID NO: 48, light chain CDR 1 of SEQ ID NO: 63, light chain CDR 2 of SEQ ID NO: 68 and light chain CDR 3 of SEQ ID NO: 73;
(a6) an antibody variable region comprising the heavy chain complementarity determining region (CDR) 1 of SEQ ID NO: 22, the heavy chain CDR 2 of SEQ ID NO: 36, the heavy chain CDR 3 of SEQ ID NO: 50, light chain CDR 1 of SEQ ID NO: 63, light chain CDR 2 of SEQ ID NO: 68 and light chain CDR 3 of SEQ ID NO: 73;
(a7) an antibody variable region comprising the heavy chain complementarity determining region (CDR) 1 of SEQ ID NO: 23, the heavy chain CDR 2 of SEQ ID NO: 37, the heavy chain CDR 3 of SEQ ID NO: 51, light chain CDR 1 of SEQ ID NO: 63, light chain CDR 2 of SEQ ID NO: 68 and light chain CDR 3 of SEQ ID NO: 73;
(a8) an antibody variable region comprising the heavy chain complementarity determining region (CDR) 1 of SEQ ID NO: 23, the heavy chain CDR 2 of SEQ ID NO: 37, the heavy chain CDR 3 of SEQ ID NO: 51, light chain CDR 1 of SEQ ID NO: 66, light chain CDR 2 of SEQ ID NO: 71 and light chain CDR 3 of SEQ ID NO: 76;
(a9) an antibody variable region comprising the heavy chain complementarity determining region (CDR) 1 of SEQ ID NO: 24, the heavy chain CDR 2 of SEQ ID NO: 38, the heavy chain CDR 3 of SEQ ID NO: 52, light chain CDR 1 of SEQ ID NO: 63, light chain CDR 2 of SEQ ID NO: 68 and light chain CDR 3 of SEQ ID NO: 73;
(a10) an antibody variable region comprising the heavy chain complementarity determining region (CDR) 1 of SEQ ID NO: 25, the heavy chain CDR 2 of SEQ ID NO: 39, the heavy chain CDR 3 of SEQ ID NO: 53, light chain CDR 1 of SEQ ID NO: 66, light chain CDR 2 of SEQ ID NO: 71 and light chain CDR 3 of SEQ ID NO: 76;
(a11) an antibody variable region comprising the heavy chain complementarity determining region (CDR) 1 of SEQ ID NO: 26, the heavy chain CDR 2 of SEQ ID NO: 40, the heavy chain CDR 3 of SEQ ID NO: 54, light chain CDR 1 of SEQ ID NO: 66, light chain CDR 2 of SEQ ID NO: 71 and light chain CDR 3 of SEQ ID NO: 76;
(a12) an antibody variable region comprising the heavy chain complementarity determining region (CDR) 1 of SEQ ID NO: 26, the heavy chain CDR 2 of SEQ ID NO: 40, the heavy chain CDR 3 of SEQ ID NO: 54, light chain CDR 1 of SEQ ID NO: 63, light chain CDR 2 of SEQ ID NO: 68 and light chain CDR 3 of SEQ ID NO: 73;
(a13) an antibody variable region comprising the heavy chain complementarity determining region (CDR) 1 of SEQ ID NO: 27, the heavy chain CDR 2 of SEQ ID NO: 41, the heavy chain CDR 3 of SEQ ID NO: 55, light chain CDR 1 of SEQ ID NO: 63, light chain CDR 2 of SEQ ID NO: 68 and light chain CDR 3 of SEQ ID NO: 73;
(a14) an antibody variable region comprising the heavy chain complementarity determining region (CDR) 1 of SEQ ID NO: 28, the heavy chain CDR 2 of SEQ ID NO: 42, the heavy chain CDR 3 of SEQ ID NO: 56, light chain CDR 1 of SEQ ID NO: 63, light chain CDR 2 of SEQ ID NO: 68 and light chain CDR 3 of SEQ ID NO: 73; and
(a15) an antibody variable region comprising heavy chain complementarity determining region (CDR) 1 of SEQ ID NO: 82, heavy chain CDR 2 of SEQ ID NO: 83, heavy chain CDR 3 of SEQ ID NO: 84, light chain CDR 1 of SEQ ID NO: 65, light chain CDR 2 of SEQ ID NO: 70 and light chain CDR 3 of SEQ ID NO: 75; and
(b) a third antigen-binding moiety that binds to human Delta-like 3 (DLL3) and comprises an antibody variable region comprising a heavy chain CDR1 comprising SEQ ID NO: 233, a heavy chain CDR 2 comprising SEQ ID NO: 234, a heavy chain CDR 3 comprising SEQ ID NO: 235, a light chain CDR 1 comprising SEQ ID NO: 237, a light chain CDR 2 comprising SEQ ID NO: 238, and a light chain CDR 3 comprising SEQ ID NO: 239.

[33-2] The multispecific antigen-binding molecule of [33-1], wherein each of the first and second antigen-binding moieties comprises an antibody variable region that can be the same or different and is independently selected from the group consisting of:
(a1) a heavy chain variable region comprising an amino acid sequence of SEQ ID NO: 3, and a light chain variable region comprising an amino acid sequence of SEQ ID NO: 59;
(a2) a heavy chain variable region comprising an amino acid sequence of SEQ ID NO: 4, and a light chain variable region comprising an amino acid sequence of SEQ ID NO: 58;
(a3) a heavy chain variable region comprising an amino acid sequence of SEQ ID NO: 5, and a light chain variable region comprising an amino acid sequence of SEQ ID NO: 58;
(a4) a heavy chain variable region comprising an amino acid sequence of SEQ ID NO: 5, and a light chain variable region comprising an amino acid sequence of SEQ ID NO: 60;
(a5) a heavy chain variable region comprising an amino acid sequence of SEQ ID NO: 6, and a light chain variable region comprising an amino acid sequence of SEQ ID NO: 58;
(a6) a heavy chain variable region comprising an amino acid sequence of SEQ ID NO: 8, and a light chain variable region comprising an amino acid sequence of SEQ ID NO: 58;
(a7) a heavy chain variable region comprising an amino acid sequence of SEQ ID NO: 9, and a light chain variable region comprising an amino acid sequence of SEQ ID NO: 58;
(a8) a heavy chain variable region comprising an amino acid sequence of SEQ ID NO: 9, and a light chain variable region comprising an amino acid sequence of SEQ ID NO: 61;
(a9) a heavy chain variable region comprising an amino acid sequence of SEQ ID NO: 10, and a light chain variable region comprising an amino acid sequence of SEQ ID NO: 58;
(a10) a heavy chain variable region comprising an amino acid sequence of SEQ ID NO: 11, and a light chain variable region comprising an amino acid sequence of SEQ ID NO: 61;
(a11) a heavy chain variable region comprising an amino acid sequence of SEQ ID NO: 12, and a light chain variable region comprising an amino acid sequence of SEQ ID NO: 61;
(a12) a heavy chain variable region comprising an amino acid sequence of SEQ ID NO: 12, and a light chain variable region comprising an amino acid sequence of SEQ ID NO: 58;
(a13) a heavy chain variable region comprising an amino acid sequence of SEQ ID NO: 13, and a light chain variable region comprising an amino acid sequence of SEQ ID NO: 58;
(a14) a heavy chain variable region comprising an amino acid sequence of SEQ ID NO: 14, and a light chain variable region comprising an amino acid sequence of SEQ ID NO: 58;
(a15) a heavy chain variable region comprising an amino acid sequence of SEQ ID NO: 81, and a light chain variable region comprising an amino acid sequence of SEQ ID NO: 60; and
(a16) a heavy chain variable region comprising an amino acid sequence selected from the group consisting of SEQ ID NO: 3-6, 8-14 and 81, and/or a light chain variable region comprising an amino acid sequence selected from the group consisting of SEQ ID NO: 58-61.

[33-2A] The multispecific antigen-binding molecule of any one [33-1] to [33-2], wherein the first antigen-binding moiety and the second antigen-binding moiety each comprises an antibody variable region comprising a heavy chain complementarity determining region (CDR) 1 of SEQ ID NO: 20, a heavy chain CDR 2 of SEQ ID NO: 34, a heavy chain CDR 3 of SEQ ID NO: 48, a light chain CDR 1 of SEQ ID NO: 63, a light chain CDR 2 of SEQ ID NO: 68 and a light chain CDR 3 of SEQ ID NO: 73.

[33-2B] The multispecific antigen-binding molecule of [33-2A], wherein the first antigen-binding moiety and the second antigen-binding moiety each comprises an antibody variable region comprising a heavy chain variable region comprising an amino acid sequence of SEQ ID NO: 6, and a light chain variable region comprising an amino acid sequence of SEQ ID NO: 58.

[33-2C] The multispecific antigen-binding molecule of any one of [33-1] to [33-2B], wherein the third antigen-binding moiety comprises an antibody variable region comprising a VH comprising SEQ ID NO: 232 and a VL comprising SEQ ID NO: 236.

[33-3] The multispecific antigen-binding molecule of any one of [33-1] to [33-2C], wherein each of the first and second antigen-binding moieties is a Fab that has a cysteine residue at position 191 (EU numbering), and wherein there is a disulfide bond linking the two cysteine residues.

[33-4] The multispecific antigen-binding molecule of [33-3], wherein each of the first, second and third antigen binding moieties is a Fab comprising a heavy chain comprising a VH and a CH1 domain and a light chain comprising a VL and a light chain constant (CL) domain, and wherein the C-terminus of the heavy chain of the third antigen binding moiety is fused, directly or via a peptide linker, to the N-terminus of the Fab heavy chain of either the first antigen binding moiety or the second antigen binding moiety.

[33-5A] The multispecific antigen-binding molecule of [33-4], wherein the C-terminus of the heavy chain of the third antigen binding moiety is fused, via a peptide linker, to the N-terminus of the Fab heavy chain of either the first antigen binding moiety or the second antigen binding moiety, and wherein the peptide linker has an amino acid sequence selected from the group consisting of SEQ ID NO: 248, SEQ ID NO: 249, and SEQ ID NO: 259.

[33-5B] The multispecific antigen-binding molecule of [33-5A], wherein the third antigen binding moiety is a crossover Fab molecule in which the variable regions of the Fab light chain and the Fab heavy chain are exchanged, and wherein each of the first and second antigen binding moiety is a conventional Fab molecule.

[33-6] The multispecific antigen-binding molecule of [33-5B], wherein, in the CL domain of each of the first and second antigen binding moieties, the amino acids at positions 123 and 124 (Kabat numbering) are arginine and lysine, respectively; and wherein, in the CH1 domain of each of the first and second antigen binding moieties, the amino acid at each of positions 147 and 213 (EU numbering) is glutamic acid.

[33-7] The multispecific antigen-binding molecule of [33-6], further comprising an Fc domain.

[33-8] The multispecific antigen-binding molecule of [33-7], wherein the Fc domain comprises a first and a second Fc region subunit, the first Fc-region subunit is selected from the group comprising:
a Fc region polypeptide comprising alanine at each of positions 234 and 235;
a Fc region polypeptide comprising alanine at each of positions 234, 235, and 297; and
a Fc region polypeptide comprising alanine at each of positions 234, 235, and 297, cysteine at position 354, and tryptophan at position 366; and
the second Fc-region subunit is selected from the group comprising:
a Fc region polypeptide comprising alanine at each of positions 234 and 235;
a Fc region polypeptide comprising alanine at each of positions 234, 235, and 297; and
a Fc region polypeptide comprising alanine at each of positions 234, 235, and 297, cysteine at position 349, serine at position 366, alanine at position 368, and valine at position 407,
wherein all positions are by EU numbering.

Yet another aspect of the present invention relates to:

[34-1] A multispecific antigen-binding molecule that comprises:
(a) a first antigen-binding moiety and a second antigen-binding moiety, each of which binds to human CD3 and comprises an antibody variable region that can be the same or different and is independently selected from the group consisting of (a1) to (a15);
(a1) an antibody variable region comprising a heavy chain complementarity determining region (CDR) 1 comprising SEQ ID NO: 17, a heavy chain CDR 2 comprising SEQ ID NO: 31, a heavy chain CDR 3 comprising SEQ ID NO: 45, a light chain CDR 1 comprising SEQ ID NO: 64, a light chain CDR 2 comprising SEQ ID NO: 69, and a light chain CDR 3 comprising SEQ ID NO: 74;
(a2) an antibody variable region comprising a heavy chain CDR 1 comprising SEQ ID NO: 18, a heavy chain CDR 2 comprising SEQ ID NO: 32, a heavy chain CDR 3 comprising SEQ ID NO: 46, a light chain CDR 1 comprising SEQ ID NO: 63, a light chain CDR 2 comprising SEQ ID NO: 68, and a light chain CDR 3 comprising SEQ ID NO: 73;
(a3) an antibody variable region comprising a heavy chain CDR 1 comprising SEQ ID NO: 19, a heavy chain CDR 2 comprising SEQ ID NO: 33, a heavy chain CDR 3 comprising SEQ ID NO: 47, a light chain CDR 1 comprising SEQ ID NO: 63, a light chain CDR 2 comprising SEQ ID NO: 68, and a light chain CDR 3 comprising SEQ ID NO: 73;
(a4) an antibody variable region comprising a heavy chain CDR 1 comprising SEQ ID NO: 19, a heavy chain CDR 2 comprising SEQ ID NO: 33, a heavy chain CDR 3 comprising SEQ ID NO: 47, a light chain CDR 1 comprising SEQ ID NO: 65, a light chain CDR 2 comprising SEQ ID NO: 70, and a light chain CDR 3 comprising SEQ ID NO: 75;
(a5) an antibody variable region comprising a heavy chain CDR 1 comprising SEQ ID NO: 20, a heavy chain CDR 2 comprising SEQ ID NO: 34, a heavy chain CDR 3 comprising SEQ ID NO: 48, a light chain CDR 1 comprising SEQ ID NO: 63, a light chain CDR 2 comprising SEQ ID NO: 68, and a light chain CDR 3 comprising SEQ ID NO: 73;
(a6) an antibody variable region comprising a heavy chain CDR 1 comprising SEQ ID NO: 22, a heavy chain CDR 2 comprising SEQ ID NO: 36, a heavy chain CDR 3 comprising SEQ ID NO: 50, a light chain CDR 1 comprising SEQ ID NO: 63, a light chain CDR 2 comprising SEQ ID NO: 68, and a light chain CDR 3 comprising SEQ ID NO: 73;
(a7) an antibody variable region comprising a heavy chain CDR 1 comprising SEQ ID NO: 23, a heavy chain CDR 2 comprising SEQ ID NO: 37, a heavy chain CDR 3 comprising SEQ ID NO: 51, a light chain CDR 1 comprising SEQ ID NO: 63, a light chain CDR 2 comprising SEQ ID NO: 68, and a light chain CDR 3 comprising SEQ ID NO: 73;
(a8) an antibody variable region comprising a heavy chain CDR 1 comprising SEQ ID NO: 23, a heavy chain CDR 2 comprising SEQ ID NO: 37, a heavy chain CDR 3 comprising SEQ ID NO: 51, a light chain CDR 1 comprising SEQ ID NO: 66, a light chain CDR 2 comprising SEQ ID NO: 71, and a light chain CDR 3 comprising SEQ ID NO: 76;
(a9) an antibody variable region comprising a heavy chain CDR 1 comprising SEQ ID NO: 24, a heavy chain CDR 2 comprising SEQ ID NO: 38, a heavy chain CDR 3 comprising SEQ ID NO: 52, a light chain CDR 1 comprising SEQ ID NO: 63, a light chain CDR 2 comprising SEQ ID NO: 68, and a light chain CDR 3 comprising SEQ ID NO: 73;
(a10) an antibody variable region comprising a heavy chain CDR 1 comprising SEQ ID NO: 25, a heavy chain CDR 2 comprising SEQ ID NO: 39, a heavy chain CDR 3 comprising SEQ ID NO: 53, a light chain CDR 1 comprising SEQ ID NO: 66, a light chain CDR 2 comprising SEQ ID NO: 71, and a light chain CDR 3 comprising SEQ ID NO: 76;
(a11) an antibody variable region comprising a heavy chain CDR 1 comprising SEQ ID NO: 26, a heavy chain CDR 2 comprising SEQ ID NO: 40, a heavy chain CDR 3 comprising SEQ ID NO: 54, a light chain CDR 1 comprising SEQ ID NO: 66, a light chain CDR 2 comprising SEQ ID NO: 71, and a light chain CDR 3 comprising SEQ ID NO: 76;
(a12) an antibody variable region comprising a heavy chain CDR 1 comprising SEQ ID NO: 26, a heavy chain CDR 2 comprising SEQ ID NO: 40, a heavy chain CDR 3 comprising SEQ ID NO: 54, a light chain CDR 1 comprising SEQ ID NO: 63, a light chain CDR 2 comprising SEQ ID NO: 68, and a light chain CDR 3 comprising SEQ ID NO: 73;
(a13) an antibody variable region comprising a heavy chain CDR 1 comprising SEQ ID NO: 27, a heavy chain CDR 2 comprising SEQ ID NO: 41, a heavy chain CDR 3 comprising SEQ ID NO: 55, a light chain CDR 1 comprising SEQ ID NO: 63, a light chain CDR 2 comprising SEQ ID NO: 68, and a light chain CDR 3 comprising SEQ ID NO: 73;
(a14) an antibody variable region comprising a heavy chain CDR 1 comprising SEQ ID NO: 28, a heavy chain CDR 2 comprising SEQ ID NO: 42, a heavy chain CDR 3 comprising SEQ ID NO: 56, a light chain CDR 1 comprising SEQ ID NO: 63, a light chain CDR 2 comprising SEQ ID NO: 68, and a light chain CDR 3 comprising SEQ ID NO: 73; and
(a15) an antibody variable region comprising a heavy chain CDR 1 comprising SEQ ID NO: 82, a heavy chain CDR 2 comprising SEQ ID NO: 83, a heavy chain CDR 3 comprising SEQ ID NO: 84, a light chain CDR 1 comprising SEQ ID NO: 65, a light chain CDR 2 comprising SEQ ID NO: 70, and a light chain CDR 3 comprising SEQ ID NO: 75; and
(b) a third antigen-binding moiety that binds to human Delta-like 3 (DLL3) and comprises an antibody variable region comprising a heavy chain CDR1 comprising SEQ ID NO: 233, a heavy chain CDR 2 comprising SEQ ID NO: 234, a heavy chain CDR 3 comprising SEQ ID NO: 235, a light chain CDR 1 comprising SEQ ID NO: 237, a light chain CDR 2 comprising SEQ ID NO: 238, and a light chain CDR 3 comprising SEQ ID NO: 239.

[34-2] A multispecific antigen-binding molecule that comprises:
(a) a first antigen-binding moiety and a second antigen-binding moiety, each of which comprises an antibody variable region that can be the same or different and is independently selected from the group consisting of:
(a1) an antibody variable region comprising a heavy chain variable region (VH) comprising SEQ ID NO: 3 and a light chain variable region (VL) comprising SEQ ID NO: 59;
(a2) an antibody variable region comprising a VH comprising SEQ ID NO: 4 and a VL comprising SEQ ID NO: 58;
(a3) an antibody variable region comprising a VH comprising SEQ ID NO: 5 and a VL comprising SEQ ID NO:58;
(a4) an antibody variable region comprising a VH comprising SEQ ID NO: 5 and a VL comprising SEQ ID NO: 60;
(a5) an antibody variable region comprising a VH comprising SEQ ID NO: 6 and a VL comprising SEQ ID NO: 58;
(a6) an antibody variable region comprising a VH comprising SEQ ID NO: 8 and a VL comprising SEQ ID NO: 58;
(a7) an antibody variable region comprising a VH comprising SEQ ID NO: 9 and a VL comprising SEQ ID NO: 58;
(a8) an antibody variable region comprising a VH comprising SEQ ID NO: 9 and a VL comprising SEQ ID NO: 61;
(a9) an antibody variable region comprising a VH comprising SEQ ID NO: 10 and a VL comprising SEQ ID NO: 58;
(a10) an antibody variable region comprising a VH comprising SEQ ID NO: 11 and a VL comprising SEQ ID NO: 61;
(a11) an antibody variable region comprising a VH comprising SEQ ID NO: 12 and a VL comprising SEQ ID NO: 61;
(a12) an antibody variable region comprising a VH comprising SEQ ID NO: 12 and a VL comprising SEQ ID NO: 58;
(a13) an antibody variable region comprising a VH comprising SEQ ID NO: 13 and a VL comprising SEQ ID NO: 58;
(a14) an antibody variable region comprising a VH comprising SEQ ID NO: 14 and a VL comprising SEQ ID NO: 58;
(a15) an antibody variable region comprising a VH comprising SEQ ID NO: 81 and a VL comprising SEQ ID NO: 60; and
(a16) a heavy chain variable region comprising an amino acid sequence selected from the group consisting of SEQ ID NO: 3-6, 8-14 and 81, and/or a light chain variable region comprising an amino acid sequence selected from the group consisting of SEQ ID NO: 58-61;
and
(b) a third antigen-binding moiety comprising an antibody variable region comprising a VH comprising SEQ ID NO: 232 and a VL comprising SEQ ID NO: 236.

[34-3] The multispecific antigen-binding molecule of [34-1], wherein the antibody variable regions of the first and second antigen binding moieties are identical.

[34-4] The multispecific antigen-binding molecule of [34-2], wherein the antibody variable regions of the first and second antigen binding moieties are identical.

[34-5] The multispecific antigen-binding molecule of [34-3], wherein each of the first and second antigen-binding moieties is a Fab that has a cysteine residue at position 191 (EU numbering), and wherein a disulfide bond links these two cysteine residues.

[34-6] The multispecific antigen-binding molecule of [34-4], wherein each of the first and second antigen-binding moieties is a Fab that has a cysteine residue at position 191 (EU numbering), and wherein a disulfide bond links these two cysteine residues.

[34-7] The multispecific antigen-binding molecule of [34-5], wherein each of the first, second and third antigen binding moieties is in the form of a Fab comprising a VH, a VL, a CH1 domain and a light chain constant (CL) domain, and wherein the C-terminus of the CH1 domain of the third antigen-binding moiety is fused, directly or via a peptide linker, to the N-terminus of the VH of either the first antigen binding moiety or the second antigen binding moiety.

[34-8] The multispecific antigen-binding molecule of [34-6], wherein each of the first, second and third antigen binding moieties is in the form of a Fab comprising a VH, a VL, a CH1 domain and a light chain constant (CL) domain, and wherein the C-terminus of the CH1 domain of the third antigen-binding moiety is fused, directly or via a peptide linker, to the N-terminus of the VH of either the first antigen binding moiety or the second antigen binding moiety.

[34-9] The multispecific antigen-binding molecule of [34-7], wherein the fusion is via a peptide linker that comprises an amino acid sequence selected from the group consisting of SEQ ID NO: 248, SEQ ID NO: 249, and SEQ ID NO: 259.

[34-10] The multispecific antigen-binding molecule of [34-8], wherein the fusion is via a peptide linker that comprises an amino acid sequence selected from the group consisting of SEQ ID NO: 248, SEQ ID NO: 249, and SEQ ID NO: 259.

[34-11] The multispecific antigen-binding molecule of [34-9], wherein the third antigen binding moiety is a crossover Fab in which the VH is linked to the CL domain and the VL is linked to the CH1 domain, and wherein each of the first and second antigen binding moieties is a conventional Fab in which the VH is linked to the CH1 domain and the VL is linked to the CL domain.

[34-12] The multispecific antigen-binding molecule of [34-10], wherein the third antigen binding moiety is a crossover Fab in which the VH is linked to the CL domain and the VL is linked to the CH1 domain, and wherein each of the first and second antigen binding moieties is a conventional Fab in which the VH is linked to the CH1 domain and the VL is linked to the CL domain.

[34-13] The multispecific antigen-binding molecule of [34-11], wherein, in the CL domain of each of the first and second antigen binding moieties, the amino acids at positions 123 and 124 (Kabat numbering) are arginine and lysine, respectively; and wherein, in the CH1 domain of each of the first and second antigen binding moieties, the amino acid at each of positions 147 and 213 (EU numbering) is glutamic acid.

[34-14] The multispecific antigen-binding molecule of [34-12], wherein, in the CL domain of each of the first and second antigen binding moieties, the amino acids at positions 123 and 124 (Kabat numbering) are arginine and lysine, respectively; and wherein, in the CH1 domain of each of the first and second antigen binding moieties, the amino acid at each of positions 147 and 213 (EU numbering) is glutamic acid.

[34-15] The multispecific antigen-binding molecule of [34-13], further comprising an Fc domain.

[34-16] The multispecific antigen-binding molecule of [34-14], further comprising an Fc domain.

[34-17] The multispecific antigen-binding molecule of [34-15],
wherein the Fc domain comprises a first and a second Fc region subunit,
wherein the first Fc-region subunit is selected from the group comprising:
an Fc region polypeptide comprising alanine at each of positions 234 and 235;
an Fc region polypeptide comprising alanine at each of positions 234, 235, and 297; and
an Fc region polypeptide comprising alanine at each of positions 234, 235, and 297, cysteine at position 354, and tryptophan at position 366;
wherein the second Fc-region subunit is selected from the group comprising:
an Fc region polypeptide comprising alanine at each of positions 234 and 235;
an Fc region polypeptide comprising alanine at each of positions 234, 235, and 297; and
an Fc region polypeptide comprising alanine at each of positions 234, 235, and 297, cysteine at position 349, serine at position 366, alanine at position 368, and valine at position 407; and
wherein all positions are by EU numbering.

[34-18] The multispecific antigen-binding molecule of [34-16],
wherein the Fc domain comprises a first and a second Fc region subunit,
wherein the first Fc-region subunit is selected from the group comprising:
an Fc region polypeptide comprising alanine at each of positions 234 and 235;
an Fc region polypeptide comprising alanine at each of positions 234, 235, and 297; and
an Fc region polypeptide comprising alanine at each of positions 234, 235, and 297, cysteine at position 354, and tryptophan at position 366;
wherein the second Fc-region subunit is selected from the group comprising:
an Fc region polypeptide comprising alanine at each of positions 234 and 235;
an Fc region polypeptide comprising alanine at each of positions 234, 235, and 297; and
an Fc region polypeptide comprising alanine at each of positions 234, 235, and 297, cysteine at position 349, serine at position 366, alanine at position 368, and valine at position 407; and
wherein all positions are by EU numbering.

### BRIEF DESCRIPTION OF DRAWINGS

FIG. 1 shows design and naming rule of a) Trivalent Ab in DUAL/LINC (1+2) format, b) Bispecific Ab with conventional Ab format, and c) Bispecific Ab with BiTE format.

FIG. 2 shows TDCC activity of antibodies against SK-MEL30 cell lines. a) TDCC comparison among DUAL/LINC and CD3 bispecific formats. b) Linker length effect on cytotoxicity.

FIG. 3 shows in vivo efficacy of antibodies against NCI-H1436 xenograft in huNOG mice model. Y-axis means the tumor volume (mm³) and X-axis means the days after tumor implantation.

FIG. 4 shows results of analyzing CD8 T cell infiltration. Tumors were harvested at indicated time points after antibody injection and T cell infiltration was analyzed using flow cytometer.

FIG. 5 shows results of analyzing exhaustion markers on CD8 T cells. Tumors were harvested on Day 7 after antibody injection and expression of exhaustion markers was analyzed using flow cytometer.

FIG. 6 is a schematic drawing showing the structures of the full-length human DLL3 and human DLL3 ECD fragment proteins. The epitope recognized by each of the anti-DLL3 antibodies is also shown. The EGF domain has six regions, EGF1 to EGF6 from the N-terminal side to the C-terminal side.

FIG. 7 shows T cell-dependent cellular cytotoxicity (TDCC) activity of anti-DLL3-Dual-antibody (DLL3-DualAE05/DualAE05-FF110) against NB-1 cell line (neuroblastoma) and QGP-1 cell line (pancreatic islet cell carcinoma).

FIG. 8 shows in vivo efficacy of tumor volume reduction of anti-DLL3-Dual-LINC antibody (DLL3-DualAE05/DualAE05-FF114) as monotherapy or as combination therapy (Combo) with cisplatin (CDDP) or carboplatin (CBDCA), in DMS53 small cell lung cancer model. CBDCA means monotherapy of CBDCA, and CDDP means monotherapy of CDDP. Y-axis means the tumor volume (mm³) and X-axis means the days after tumor implantation. * P<0.05, ** P<0.01.

FIG. 9 shows in vivo efficacy of tumor volume reduction of anti-DLL3-Dual-antibody (DLL3-DualAE05/DualAE05-FF114) or DLL3CD3BiTE which were administered once in a single dose or multiple times in once every week (QW) schedule in NCI-H1436 xenograft in humanized NOG mice model. Y-axis means the tumor volume (mm³) and X-axis means the days after tumor implantation.

FIG. 10 shows in vivo efficacy of anti-DLL3-Dual-LINC antibody (DLL3-DualAE05/DualAE05-FF114) in combination with steroid or tocilizumab. a) In the premedication experiments with steroid, dexamethasone was intraperitoneally administered 1 and 24 hours, or 1 hour prior to antibody injection to DLL3 expressing PC-10-bearing huNOG mice. The top panel in a) shows tumor suppression by the antibody with or without the steroid. The middle panel in a) shows steroid-mediated inhibition of antibody-induced IFN-gamma release (the left panel) or TNF alpha release (the right panel). The bottom panel in a) shows steroid-mediated inhibition of antibody-induced IL-6 release. b) In the experiments with tocilizumab, tocilizumab was administered one day before or 6 hours after injection of anti-DLL3-Dual-LINC antibody to DLL3 expressing PC-10-bearing huNOG mice. The graph shows tumor suppression by the antibody with or without pre- or post-tocilizumab administration.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

The definitions and detailed description below are provided to facilitate under-standing of the present disclosure illustrated herein.

### Definitions

Amino acids Herein, amino acids are described by one- or three-letter codes or both, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V.

### Alteration of Amino Acids

For amino acid alteration (also described as “amino acid substitution” or “amino acid mutation” within this description) in the amino acid sequence of an antigen-binding molecule, known methods such as site-directed mutagenesis methods (Kunkel et al. (Proc. Natl. Acad. Sci. USA (1985) 82, 488-492)) and overlap extension PCR may be appropriately employed. Furthermore, several known methods may also be employed as amino acid alteration methods for substitution to non-natural amino acids (Annu Rev. Biophys. Biomol. Struct. (2006) 35, 225-249; and Proc. Natl. Acad. Sci. U.S.A. (2003) 100 (11), 6353-6357). For example, it is suitable to use a cell-free translation system (Clover Direct (Protein Express)) containing a tRNA which has a non-natural amino acid bound to a complementary amber suppressor tRNA of one of the stop codons, the UAG codon (amber codon).

In the present specification, the meaning of the term “and/or” when describing the site of amino acid alteration includes every combination where “and” and “or” are suitably combined. Specifically, for example, “the amino acids at positions 33, 55, and/or 96 are substituted” includes the following variation of amino acid alterations: amino acid(s) at (a) position 33, (b) position 55, (c) position 96, (d) positions 33 and 55, (e) positions 33 and 96, (f) positions 55 and 96, and (g) positions 33, 55, and 96.

Furthermore, herein, as an expression showing alteration of amino acids, an expression that shows before and after a number indicating a specific position, one-letter or three-letter codes for amino acids before and after alteration, respectively, may be used appropriately. For example, the alteration N100bL or Asn100bLeu used when substituting an amino acid contained in an antibody variable region indicates substitution of Asn at position 100b (according to Kabat numbering) with Leu. That is, the number shows the amino acid position according to Kabat numbering, the one-letter or three-letter amino-acid code written before the number shows the amino acid before substitution, and the one-letter or three-letter amino-acid code written after the number shows the amino acid after substitution. Similarly, the alteration P238D or Pro238Asp used when substituting an amino acid of the Fc region contained in an antibody constant region indicates substitution of Pro at position 238 (according to EU numbering) with Asp. That is, the number shows the amino acid position according to EU numbering, the one-letter or three-letter amino-acid code written before the number shows the amino acid before substitution, and the one-letter or three-letter amino-acid code written after the number shows the amino acid after substitution.

### Polypeptides

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide as described herein may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

### Percent (%) Amino Acid Sequence Identity

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Recombinant Methods and Compositions

Antibodies and antigen-binding molecules may be produced using recombinant methods and compositions, e.g., as described in U.S. Pat. No. 4,816,567. In one embodiment, isolated nucleic acid encoding an antibody as described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making the multispecific antigen-binding molecule of the present invention is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody described herein, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

Recombinant production of an antigen-binding molecule described herein could be done with methods similar to those described above, by using a host cell comprises (e.g., has been transformed with) one or plural vectors comprising nucleic acid that encodes an amino acid sequence comprising the whole antigen-binding molecule or part of the antigen-binding molecule.

### Antigen-Binding Molecule and Multispecific Antigen-Binding Molecules

The term “antigen-binding molecule”, as used herein, refers to any molecule that comprises an antigen-binding site or any molecule that has binding activity to an antigen, and may further refers to molecules such as a peptide or protein having a length of about five amino acids or more. The peptide and protein are not limited to those derived from a living organism, and for example, they may be a polypeptide produced from an artificially designed sequence. They may also be any of a naturally-occurring polypeptide, synthetic polypeptide, recombinant polypeptide, and such. Scaffold molecules comprising known stable conformational structure such as alpha/beta barrel as scaffold, and in which part of the molecule is made into antigen-binding site, is also one embodiment of the antigen binding molecule described herein.

“Multispecific antigen-binding molecules” refers to antigen-binding molecules that bind specifically to more than one antigen. The term “bispecific” means that the antigen binding molecule is able to specifically bind to at least two distinct antigenic determinants. The term “trispecific” means that the antigen binding molecule is able to specifically bind to at least three distinct antigenic determinants. In certain embodiments, the multispecific antigen binding molecule of the present application is a trispecific antigen binding molecule, i.e. it is capable of specifically binding to three different antigen—capable of binding to either one of CD3 or CD137 but does not bind to both antigens simultaneously, and is capable of specifically binding to DLL3.

In a first aspect, the present disclosure provides a multispecific antigen binding molecule comprising: a first antigen-binding moiety and a second antigen-binding moiety, each of which is capable of binding to CD3 and CD137, but does not bind to CD3 and CD137 at the same time; and a third antigen-binding moiety that is capable of binding to a third antigen, preferably an antigen expressed on a cancer cell/tissue. In certain embodiments, the third antigen bound by the third antigen-binding moiety is DLL3, and preferably is human DLL3.

The first antigen-binding moiety and the second antigen-binding moiety can be a “Dual antigen binding moiety” capable of binding to CD3 and CD137, but not at the same time, which will be described in more detail below. The third antigen-binding moiety can be a “DLL3 antigen binding moiety”, which also will be described in more detail below.

In some embodiments, each of the first antigen-binding moiety and the second antigen-binding moiety is a Fab molecule and comprises at least one disulfide bond formed between the CH1 region of the first antigen-binding moiety and the CH1 region of the second antigen-binding moiety. The disulfide bond may be formed between the amino acid residues at position 191 according to EU numbering in the respective CH1 region of the first antigen-binding moiety and the second antigen-binding moiety.

In some embodiments, the third antigen binding moiety, which may be a Fab or scFv, is fused to either one of the first antigen binding moiety or the second antigen binding moiety. In the case that each of the first, second, and third antigen binding moiety is a Fab molecule, the third antigen binding moiety may be fused at the C-terminus of the Fab heavy chain (CH1) to the N-terminus of the Fab heavy chain of either one of the first antigen binding moiety or the second antigen binding moiety, optionally via a peptide linker. Representative peptide linkers include those consisting of the amino acid sequence of SEQ ID NO: 248, SEQ ID NO: 249, or SEQ ID NO: 259. In certain embodiments, the first antigen binding moiety is identical to the second antigen binding moiety.

In some embodiments, the third antigen binding moiety is a crossover Fab molecule in which the variable regions of the Fab light chain and the Fab heavy chain are exchanged, and wherein each of the first and second antigen binding moiety is a conventional Fab molecule.

In some embodiments, in the constant domain CL of the light chain of each of the first and second antigen binding moiety, the amino acid(s) at position 123 and/or 124 is/are substituted independently by lysine (K), arginine (R) or histidine (H) (numbering according to Kabat), and wherein in the constant domain CH1 of the heavy chain of each of the first and second antigen binding moiety, the amino acid at position 147 and/or the amino acid at position 213 is substituted independently by glutamic acid (E) or aspartic acid (D) (numbering according to Kabat EU index). In other embodiments, in the constant domain CL of the light chain of each of the first and second antigen binding moiety, the amino acids at position 123 and 124 are arginine (R) and lysine (K) respectively (numbering according to Kabat), and wherein in the constant domain CH1 of the heavy chain of each of the first and second antigen binding moiety the amino acids at position 147 and 213 are glutamic acid (E) (numbering according to Kabat EU index).

The multispecific antigen-binding molecule can further comprise a Fc domain, which will be described in detail below. In the case that each of the first and second antigen-binding moiety is a Fab, the first antigen-binding moiety may be fused at the C-terminus of the Fab heavy chain to the N-terminus of the first or second subunit of the Fc domain, and the second antigen-binding moiety may be fused at the C-terminus of the Fab heavy chain to the N-terminus of the remaining subunit of the Fc domain. In some embodiments, the third antigen binding moiety is fused at the C-terminus to the N-terminus of the Fab heavy chain of either one of the first antigen binding moiety or the second antigen binding moiety, optionally via a peptide linker.

In another aspect of the present invention, the present disclosure provides a multispecific antigen-binding molecule comprising: an antigen-binding moiety which is capable of binding to CD3 and CD137, but does not bind to CD3 and CD137 at the same time; and an antigen-binding moiety that is capable of binding to DLL3, preferably human DLL3. In certain embodiments, the antigen-binding moiety which is capable of binding to CD3 and CD137, but does not bind to CD3 and CD137 at the same time, is a “Dual antigen binding moiety” capable of binding to CD3 and CD137, but not at the same time, described in detail below.

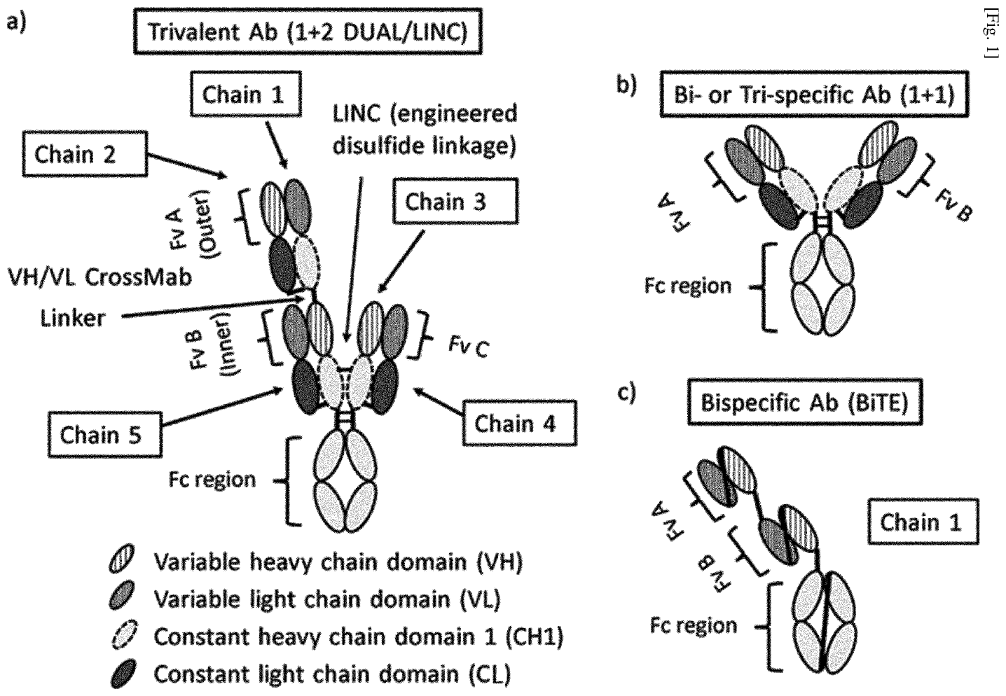

The components of the multispecific antigen binding molecules of the present invention can be fused to each other in a variety of configurations. Exemplary configurations are depicted in FIG. 1 a) read together with Tables 10-1 to 10-3.

According to any of the above embodiments, components of the multispecific antigen binding molecules (e.g. antigen binding moiety, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G4S)n, (SG4)n, (G4S)n or G4(SG4)n peptide linkers, wherein n is generally a number between 1 and 10, typically between 2 and 4.

### Pyroglutamylation

It is known that when an antibody is expressed in cells, the antibody is modified after translation. Examples of the posttranslational modification include cleavage of lysine at the C terminal of the heavy chain by a carboxypeptidase; modification of glutamine or glutamic acid at the N terminal of the heavy chain and the light chain to pyroglutamic acid by pyroglutamylation; glycosylation; oxidation; deamidation; and glycation, and it is known that such posttranslational modifications occur in various antibodies (Journal of Pharmaceutical Sciences, 2008, Vol. 97, p. 2426-2447).

In some embodiments, the multispecific antigen binding molecules of the present invention also includes posttranslational modification. Examples of posttranslational modification includes pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

### Antigen Binding Moiety

As used herein, the term “antigen binding moiety” refers to a polypeptide molecule that specifically binds to an antigen. In one embodiment, an antigen binding moiety is able to direct the entity to which it is attached to a target site, for example to a specific type of tumor cell expressing the cancer antigen (DLL3). In another embodiment an antigen binding moiety is able to activate signaling through its target antigen, for example a T cell receptor complex antigen (in particular CD3) and/or a co-stimulatory receptor (CD137). Antigen binding moieties include antibodies and fragments thereof as further defined herein. Particular antigen binding moieties include an antigen binding domain or an antibody variable region of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: alpha, delta, epsilon, gamma, or mu. Useful light chain constant regions include any of the two isotypes: kappa and lambda.

As used herein, the terms “first”, “second”, and “third” with respect to antigen binding moieties etc., are used for convenience of distinguishing when there is more than one of each type of moiety. Use of these terms is not intended to confer a specific order or orientation of the multispecific antigen binding molecule unless explicitly so stated.

In another aspect, antigen binding moiety of the present invention disclosed herein can be used in novel chimeric antigen receptor (CAR) comprising one or more of the antigen binding moiety disclosed herein. In certain embodiments, a CAR of the invention will comprise a scFv construct, and in a preferred embodiment, will comprise and comprise a heavy and light chain variable region as disclosed herein. In a preferred embodiment, the disclosed chimeric antigen receptors are useful for treating or preventing a proliferative disorder and any recurrence or metastasis thereof.

Antigen-Binding Moiety Capable of Binding to CD3 and CD137 but not at the Same Time

The multispecific antigen binding molecule described herein comprises at least one antigen binding moiety capable of binding to CD3 and CD137, but does not bind to CD3 and CD137 at the same time (also referred to herein as “Dual antigen binding moiety” or “first antigen binding moiety” or “Dual-Fab” or Dual-Ig”). In one embodiment, the antigen binding moiety capable of binding to CD3 and CD137 but does not bind to CD3 and CD137 at the same time, is an antigen binding moiety that binds to human CD3. In another embodiment, the antigen binding moiety capable of binding to CD3 and CD137 but does not bind to CD3 and CD137 at the same time, is an antigen binding moiety that binds to human CD137. In another embodiment, the antigen binding moiety capable of binding to CD3 and CD137 but does not bind to CD3 and CD137 at the same time, is an antigen binding moiety that is capable of binding to human CD3 and human CD137, wherein the antigen-binding moiety binds to either one of human CD3 and human CD137. In a particular embodiment, the multispecific antigen binding molecule comprises two Dual antigen binding moieties (“first antigen binding moiety” and “second antigen binding moiety”, each of which may be called as “Dual-Fab”). In some embodiments, each of the two Dual antigen binding moiety (“first antigen binding moiety” or “second antigen binding moiety” or “Dual-Fab”) provides monovalent binding to CD3 or CD137, but does not bind to CD3 and CD137 at the same time. In a particular embodiment, the multispecific antigen binding molecule comprises not more than two of the Dual antigen binding moiety (“first antigen binding moiety” or “second antigen binding moiety” or “Dual-Fab”).

In certain embodiments, the Dual antigen binding moiety (“first antigen binding moiety” or “second antigen binding moiety” or “Dual-Fab”) is generally a Fab molecule, particularly a conventional Fab molecule. In certain embodiments, the Dual antigen binding moiety (“first antigen binding moiety” or “second antigen binding moiety” or “Dual-Fab”) is a domain comprising antibody light-chain and heavy-chain variable regions (VL and VH). Suitable examples of such domains comprising antibody light-chain and heavy-chain variable regions include “single chain Fv (scFv)”, “single chain antibody”, “Fv”, “single chain Fv 2 (scFv2)”, “Fab”, “F(ab′)2”, etc.

In certain embodiments, the Dual antigen binding moiety (“first antigen binding moiety” or “second antigen binding moiety” or “Dual-Fab”) specifically binds to the whole or a portion of a partial peptide of CD3. In a particular embodiment, CD3 is human CD3 or cynomolgus CD3, most particularly human CD3. In a particular embodiment the first antigen binding moiety is cross-reactive for (i.e. specifically binds to) human and cynomolgus CD3. In some embodiments, the first antigen binding moiety is capable of specific binding to the epsilon subunit of CD3, in particular the human CD3 epsilon subunit of CD3 which is shown in SEQ ID NO: 7 (NP_000724.1) (RefSeq registration numbers are shown within the parentheses). In some embodiments, the Dual antigen binding moiety (“first antigen binding moiety” or “second antigen binding moiety” or “Dual-Fab”) is capable of specific binding to the CD3 epsilon chain expressed on the surface of eukaryotic cells. In some embodiments, the Dual antigen binding moiety (“first antigen binding moiety” or “second antigen binding moiety” or “Dual-Fab”) binds to the CD3 epsilon chain expressed on the surface of T cells.

In certain embodiments, the CD137 is human CD137. In some embodiments, favorable examples of an antigen-binding molecule of the present invention comprises Dual antigen binding moiety (“first antigen binding moiety” or “second antigen binding moiety” or “Dual-Fab”) that binds to the same epitope as the human CD137 epitope bound by the antibody selected from the group consisting of:
antibody that recognize a region comprising the SPCPPNSFSSAGGQRTCD ICRQCKGVFRTRKECSSTSNAECDCTPGFHCLGAGCSMCEQDCKQGQELTK KGC sequence (SEQ ID NO: 21),
antibody that recognize a region comprising the DCTPGFHCLGAGCSMCEQDCK-QGQELTKKGC sequence (SEQ ID NO: 35),
antibody that recognize a region comprising the LQDPCSNCPAGTFCDNNRNQIC-SPCPPNSFSSAGGQRTCDICRQCKGVFRTRKECSSTSNAEC sequence (SEQ ID NO: 49), and
antibody that recognize a region comprising the LQDPCSNCPAGTFCDNNRNQIC sequence (SEQ ID NO: 105) in the human CD137 protein.

In specific embodiments, the Dual antigen binding moiety (“first antigen binding moiety” or “second antigen binding moiety” or “Dual-Fab”) comprises any one of the antibody variable region sequences shown in Table 1 below. In specific embodiments, the Dual antigen binding moiety (“first antigen binding moiety” or “second antigen binding moiety” or “Dual-Fab”) comprises any one of the combinations of the heavy chain variable region and light chain variable region shown in Table 1.

TABLE 1
SEQ ID NOs of the variable regions of the Dual antigen
binding moiety (“first antigen binding moiety”
or “second antigen binding moiety” or “Dual-Fab”)

| | SEQ ID NOs | |
|---|---|---|
| | Heavy chain variable region | Light chain variable region |
| Name | (VH) | (VL) |
| DualAE08 | 3 | 59 |
| DualAE06 | 4 | 58 |
| DualAE17 | 5 | 58 |
| DualAE10 | 5 | 60 |
| DualAE05 | 6 | 58 |
| DualAE19 | 8 | 58 |
| DualAE20 | 9 | 58 |
| DualAE21 | 9 | 61 |
| DualAE22 | 10 | 58 |
| DualAE23 | 11 | 61 |
| DualAE09 | 12 | 61 |
| DualAE18 | 12 | 58 |
| DualAE14 | 13 | 58 |
| DualAE15 | 14 | 58 |
| DualAE16 | 81 | 60 |

In one embodiment the Dual antigen binding moiety (“first antigen binding moiety” or “second antigen binding moiety” or “Dual-Fab”) comprises a heavy chain variable region sequence that is at least about 95%, 96%, 97%, 98%, 99%, or 100% identical to SEQ ID NO: 6 and a light chain variable region sequence that is at least about 95%, 96%, 97%, 98%, 99%, or 100% identical to SEQ ID NO: 58. In one embodiment the Dual antigen binding moiety (“first antigen binding moiety” or “second antigen binding moiety” or “Dual-Fab”) comprises a heavy chain variable region comprising the amino acid sequence of SEQ ID NO: 6 and a light chain variable region comprising the amino acid sequence of SEQ ID NO: 58.

In one embodiment the Dual antigen binding moiety (“first antigen binding moiety” or “second antigen binding moiety” or “Dual-Fab”) comprises a heavy chain variable region sequence that is at least about 95%, 96%, 97%, 98%, 99%, or 100% identical to SEQ ID NO: 14 and a light chain variable region sequence that is at least about 95%, 96%, 97%, 98%, 99%, or 100% identical to SEQ ID NO: 58. In one embodiment the Dual antigen binding moiety (“first antigen binding moiety” or “second antigen binding moiety” or “Dual-Fab”) comprises a heavy chain variable region comprising the amino acid sequence of SEQ ID NO: 14 and a light chain variable region comprising the amino acid sequence of SEQ ID NO: 58.

In one embodiment the Dual antigen binding moiety (“first antigen binding moiety” or “second antigen binding moiety” or “Dual-Fab”) comprises a heavy chain variable region sequence that is at least about 95%, 96%, 97%, 98%, 99%, or 100% identical to SEQ ID NO: 81 and a light chain variable region sequence that is at least about 95%, 96%, 97%, 98%, 99%, or 100% identical to SEQ ID NO: 58. In one embodiment the Dual antigen binding moiety (“first antigen binding moiety” or “second antigen binding moiety” or “Dual-Fab”) comprises a heavy chain variable region comprising the amino acid sequence of SEQ ID NO: 81 and a light chain variable region comprising the amino acid sequence of SEQ ID NO: 58.

In specific embodiments, the Dual antigen binding moiety (“first antigen binding moiety” or “second antigen binding moiety” or “Dual-Fab”) comprises any one of the combinations of HVR sequences shown in Table 2 below.

TABLE 2
SEQ ID NOs of the HVR (CDR) sequences of the Dual antigen binding moiety (“first
antigen binding moiety” or “second antigen binding moiety” or “Dual-Fab”)

| | SEQ ID NOs | | | | | |
|---|---|---|---|---|---|---|
| Name | HVR-H1 | HVR-H2 | HVR-H3 | HVR-L1 | HVR-L2 | HVR-L3 |
| DualAE08 | 17 | 31 | 45 | 64 | 69 | 74 |
| DualAE06 | 18 | 32 | 46 | 63 | 68 | 73 |
| DualAE17 | 19 | 33 | 47 | 63 | 68 | 73 |
| DualAE10 | 19 | 33 | 47 | 65 | 70 | 75 |
| DualAE05 | 20 | 34 | 48 | 63 | 68 | 73 |
| DualAE19 | 22 | 36 | 50 | 63 | 68 | 73 |
| DualAE20 | 23 | 37 | 51 | 63 | 68 | 73 |
| DualAE21 | 23 | 37 | 51 | 66 | 71 | 76 |
| DualAE22 | 24 | 38 | 52 | 63 | 68 | 73 |
| DualAE23 | 25 | 39 | 53 | 66 | 71 | 76 |
| DualAE09 | 26 | 40 | 54 | 66 | 71 | 76 |
| DualAE18 | 26 | 40 | 54 | 63 | 68 | 73 |
| DualAE14 | 27 | 41 | 55 | 63 | 68 | 73 |
| DualAE15 | 28 | 42 | 56 | 63 | 68 | 73 |
| DualAE16 | 82 | 83 | 84 | 65 | 70 | 75 |

In some embodiments, the Dual antigen binding moiety (“first antigen binding moiety” or “second antigen binding moiety” or “Dual-Fab”) each comprises antibody variable region comprising any one of (a1) to (a17) below:
(a1) the heavy chain complementarity determining region (CDR) 1 of SEQ ID NO: 17, the heavy chain CDR 2 of SEQ ID NO: 31, the heavy chain CDR 3 of SEQ ID NO: 45, the light chain CDR 1 of SEQ ID NO: 64, the light chain CDR 2 of SEQ ID NO: 69 and the light chain CDR 3 of SEQ ID NO: 74;
(a2) the heavy chain complementarity determining region (CDR) 1 of SEQ ID NO: 18, the heavy chain CDR 2 of SEQ ID NO: 32, the heavy chain CDR 3 of SEQ ID NO: 46, the light chain CDR 1 of SEQ ID NO: 63, the light chain CDR 2 of SEQ ID NO: 68 and the light chain CDR 3 of SEQ ID NO: 73;
(a3) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 63, the light chain CDR 2 of SEQ ID NO: 68 and the light chain CDR 3 of SEQ ID NO: 73;
(a4) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 65, the light chain CDR 2 of SEQ ID NO: 70 and the light chain CDR 3 of SEQ ID NO: 75;
(a5) the heavy chain complementarity determining region (CDR) 1 of SEQ ID NO: 20, the heavy chain CDR 2 of SEQ ID NO: 34, the heavy chain CDR 3 of SEQ ID NO: 48, the light chain CDR 1 of SEQ ID NO: 63, the light chain CDR 2 of SEQ ID NO: 68 and the light chain CDR 3 of SEQ ID NO: 73;
(a6) the heavy chain complementarity determining region (CDR) 1 of SEQ ID NO: 22, the heavy chain CDR 2 of SEQ ID NO: 36, the heavy chain CDR 3 of SEQ ID NO: 50, the light chain CDR 1 of SEQ ID NO: 63, the light chain CDR 2 of SEQ ID NO: 68 and the light chain CDR 3 of SEQ ID NO: 73;
(a7) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 63, the light chain CDR 2 of SEQ ID NO: 68 and the light chain CDR 3 of SEQ ID NO: 73;
(a8) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 66, the light chain CDR 2 of SEQ ID NO: 71 and the light chain CDR 3 of SEQ ID NO: 76;
(a9) the heavy chain complementarity determining region (CDR) 1 of SEQ ID NO: 24, the heavy chain CDR 2 of SEQ ID NO: 38, the heavy chain CDR 3 of SEQ ID NO: 52, the light chain CDR 1 of SEQ ID NO: 63, the light chain CDR 2 of SEQ ID NO: 68 and the light chain CDR 3 of SEQ ID NO: 73;
(a10) the heavy chain complementarity determining region (CDR) 1 of SEQ ID NO: 25, the heavy chain CDR 2 of SEQ ID NO: 39, the heavy chain CDR 3 of SEQ ID NO: 53, the light chain CDR 1 of SEQ ID NO: 66, the light chain CDR 2 of SEQ ID NO: 71 and the light chain CDR 3 of SEQ ID NO: 76;
(a11) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 66, the light chain CDR 2 of SEQ ID NO: 71 and the light chain CDR 3 of SEQ ID NO: 76;
(a12) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 63, the light chain CDR 2 of SEQ ID NO: 68 and the light chain CDR 3 of SEQ ID NO: 73;
(a13) the heavy chain complementarity determining region (CDR) 1 of SEQ ID NO: 27, the heavy chain CDR 2 of SEQ ID NO: 41, the heavy chain CDR 3 of SEQ ID NO: 55, the light chain CDR 1 of SEQ ID NO: 63, the light chain CDR 2 of SEQ ID NO: 68 and the light chain CDR 3 of SEQ ID NO: 73;
(a14) the heavy chain complementarity determining region (CDR) 1 of SEQ ID NO: 28, the heavy chain CDR 2 of SEQ ID NO: 42, the heavy chain CDR 3 of SEQ ID NO: 56, the light chain CDR 1 of SEQ ID NO: 63, the light chain CDR 2 of SEQ ID NO: 68 and the light chain CDR 3 of SEQ ID NO: 73;
(a15) the heavy chain complementarity determining region (CDR) 1 of SEQ ID NO: 82, the heavy chain CDR 2 of SEQ ID NO: 83, the heavy chain CDR 3 of SEQ ID NO: 84, the light chain CDR 1 of SEQ ID NO: 65, the light chain CDR 2 of SEQ ID NO: 70 and the light chain CDR 3 of SEQ ID NO: 75;
(a16) an antibody variable region that binds to the same epitope of any of the antibody variable region selected from (a1) to (a15); and
(a17) an antibody variable fragment that competes with the binding of any of the antibody variable fragment selected from (a1) to (a15).

In some embodiments, the multispecific antigen binding molecules or the Dual antigen binding moiety (“first antigen binding moiety” or “second antigen binding moiety” or “Dual-Fab”) of the present invention also includes posttranslational modification. Examples of posttranslational modification includes pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

### Antigen-Binding Moiety Capable of Binding to DLL3

The multispecific antigen binding molecule described herein comprises at least one antigen binding moiety capable of binding to Delta-like 3 (DLL3) (also referred to herein as a “DLL3 antigen binding moiety” or “third antigen binding moiety” or “antigen-binding moiety that binds to DLL3”).

In certain embodiments, the multispecific antigen binding molecule comprises one antigen binding moiety capable of binding to DLL3. In certain embodiments, the multispecific antigen binding molecule comprises two antigen binding moieties capable of binding to DLL3 (“DLL3 antigen binding moiety”). In a particular such embodiment, each of these antigen binding moieties specifically binds to the same epitope of DLL3. In an even more particular embodiment, all of these “DLL3 antigen binding moiety” are identical. In one embodiment, the multispecific antigen binding molecule comprises an immunoglobulin molecule capable of specific binding to DLL3 (“DLL3 antigen binding moiety”). In one embodiment the multispecific antigen binding molecule comprises not more than two antigen binding moieties capable of binding to DLL3 (“DLL3 antigen binding moiety”).

In certain embodiments, the DLL3 antigen binding moiety is a crossover Fab molecule, i.e. a DLL3 molecule wherein either the variable or the constant regions of the Fab heavy and light chains are exchanged. In certain embodiments, the DLL3 antigen binding moiety is a crossover Fab molecule in which the variable regions of the Fab light chain and the Fab heavy chain are exchanged.

In some embodiments, the DLL3 antigen binding moiety binds specifically to the extracellular domain of DLL3. In some embodiments, the DLL3 antigen binding moiety binds specifically to an epitope within the extracellular domain of DLL3. In some embodiments, the DLL3 antigen binding moiety binds to the DLL3 protein expressed on the surface of eukaryotic cells. In some embodiments, the DLL3 antigen binding moiety binds to the DLL3 protein expressed on the surface of cancer cells.

In some embodiments, the multispecific antigen-binding molecules or the DLL3 antigen binding moiety bind to an epitope within the extracellular domain (ECD), i.e., the domain from the N-terminus to immediately before the TM region, but not to the TM region or the C-terminal intracellular domain. The multispecific antigen-binding molecules or the DLL3 antigen binding moiety may bind to an epitope within any of the above-mentioned domains/regions within the ECD. In preferred embodiments, the multispecific antigen-binding molecules or the DLL3 antigen binding moiety bind to an epitope within the region from EGF6 to immediately before the TM region. More specifically, the multispecific antigen-binding molecules or the DLL3 antigen binding moiety may bind to an epitope within the regions defined in SEQ ID NO: 89 in human DLL3. In some embodiments, the multispecific antigen-binding molecules or the DLL3 antigen binding moiety bind to the EGF1, EGF2, EGF3, EGF4, EGF5, or EGF6 region or a region from EGF6 to immediately before the TM region of human DLL3, or an epitope within the EGF1, EGF2, EGF3, EGF4, EGF5, or EGF6 region or a region from EGF6 to immediately before the TM region of human DLL3. In some embodiments, the multispecific antigen-binding molecules or the DLL3 antigen binding moiety can be derived from previously reported anti-DLL3 antibodies in which the DLL3 epitopes bound have been characterized (e.g. WO2019131988 and WO2011093097).

In specific embodiments, the multispecific antigen-binding molecules or the DLL3 antigen binding moiety comprises any one of the antibody variable region sequences shown in Table 3 below. In specific embodiments, the multispecific antigen-binding molecules or the DLL3 antigen binding moiety comprises any one of the combinations of the heavy chain variable region and light chain variable region shown in Table 3. In some embodiments, multispecific antigen-binding molecules or the DLL3 antigen binding moiety comprises is a domain that comprises an antibody variable fragment that competes for binding to DLL3 with any one of the antibody variable regions shown in Table 3.

TABLE 3
SEQ ID NOs of the variable regions of the
exemplary DLL3 antigen binding moiety

SEQ ID NOs

| | Heavy chain | Light chain |
|---|---|---|
| | variable region | variable region |
| Name | (VH) | (VL) |
| DL301 | 305 | 313 |
| DL306 | 306 | 314 |
| DL309 | 307 | 315 |
| DL312 | 308 | 316 |
| DLL3-14 | 309 | 317 |
| DLL3-22 | 310 | 318 |
| DLL3-4 | 311 | 319 |
| DLL3-6 | 312 | 320 |
| DLA0106 | 260 | 261 |
| DLA0126 | 262 | 263 |
| DLA0316 | 264 | 265 |
| DLA0379 | 266 | 267 |
| DLA0580 | 268 | 269 |
| DLA0641 | 270 | 271 |
| DLA0769 | 272 | 273 |
| DLA0841 | 274 | 275 |
| D30841AE05 | 297 | 236 |
| D30841AE08 | 298 | 236 |
| D30841AE11 | 298 | 302 |
| D30841AE12 | 299 | 236 |
| D30841AE13 | 232 | 236 |
| D30841AE14 | 300 | 236 |
| D30841AE15 | 301 | 236 |

In one embodiment the DLL3 antigen binding moiety comprises a heavy chain variable region sequence that is at least about 95%, 96%, 97%, 98%, 99% or 100% identical to SEQ ID NO: 232 and a light chain variable region sequence that is at least about 95%, 96%, 97%, 98%, 99% or 100% identical to SEQ ID NO: 236. In one embodiment the DLL3 antigen binding moiety comprises a heavy chain variable region comprising the amino acid sequence of SEQ ID NO: 232 and a light chain variable region comprising the amino acid sequence of SEQ ID NO: 236.

In one embodiment the DLL3 antigen binding moiety comprises a heavy chain variable region sequence that is at least about 95%, 96%, 97%, 98%, 99% or 100% identical to SEQ ID NO: 300 and a light chain variable region sequence that is at least about 95%, 96%, 97%, 98%, 99% or 100% identical to SEQ ID NO: 236. In one embodiment the DLL3 antigen binding moiety comprises a heavy chain variable region comprising the amino acid sequence of SEQ ID NO: 300 and a light chain variable region comprising the amino acid sequence of SEQ ID NO: 236.

In one embodiment the DLL3 antigen binding moiety comprises a heavy chain variable region sequence that is at least about 95%, 96%, 97%, 98%, 99% or 100% identical to SEQ ID NO: 301 and a light chain variable region sequence that is at least about 95%, 96%, 97%, 98%, 99% or 100% identical to SEQ ID NO: 236. In one embodiment the DLL3 antigen binding moiety comprises a heavy chain variable region comprising the amino acid sequence of SEQ ID NO: 301 and a light chain variable region comprising the amino acid sequence of SEQ ID NO: 236.

In one embodiment the DLL3 antigen binding moiety comprises a heavy chain variable region sequence that is at least about 95%, 96%, 97%, 98%, 99% or 100% identical to SEQ ID NO: 274 and a light chain variable region sequence that is at least about 95%, 96%, 97%, 98%, 99% or 100% identical to SEQ ID NO: 275. In one embodiment the DLL3 antigen binding moiety comprises a heavy chain variable region comprising the amino acid sequence of SEQ ID NO: 274 and a light chain variable region comprising the amino acid sequence of SEQ ID NO: 275.

In one embodiment the DLL3 antigen binding moiety comprises a heavy chain variable region sequence that is at least about 95%, 96%, 97%, 98%, 99% or 100% identical to SEQ ID NO: 264 and a light chain variable region sequence that is at least about 95%, 96%, 97%, 98%, 99% or 100% identical to SEQ ID NO: 265. In one embodiment the DLL3 antigen binding moiety comprises a heavy chain variable region comprising the amino acid sequence of SEQ ID NO: 264 and a light chain variable region comprising the amino acid sequence of SEQ ID NO: 265.

In specific embodiments, the DLL3 antigen binding moiety comprises any one of the combinations of HVR sequences shown in Table 4 below. In some embodiments, multispecific antigen-binding molecules or the DLL3 antigen binding moiety comprises is a domain that comprises an antibody variable fragment that competes for binding to DLL3 with any one of the antibody variable regions shown in Table 4, or competes for binding to DLL3 with any antibody variable fragment that comprises the HVR sequence identical with the HVR regions of the antibody variable regions shown in Table 4.

TABLE 4
SEQ ID NOs of the HVR (CDR) sequences of
exemplary DLL3 antigen binding moiety

| | SEQ ID NOs | | | | | |
|---|---|---|---|---|---|---|
| Name | HVR-H1 | HVR-H2 | HVR-H3 | HVR-L1 | HVR-L2 | HVR-L3 |
| DLA0316 | 276 | 277 | 278 | 279 | 280 | 281 |
| DLA0580 | 285 | 286 | 287 | 288 | 289 | 290 |
| DLA0769 | 291 | 292 | 293 | 294 | 295 | 296 |
| DLA0841 | 282 | 283 | 284 | 237 | 238 | 239 |
| D30841AE05 | 233 | 234 | 303 | 237 | 238 | 239 |
| D30841AE08 | 233 | 234 | 235 | 237 | 238 | 239 |
| D30841AE11 | 233 | 234 | 235 | 237 | 238 | 304 |
| D30841AE12 | 233 | 234 | 235 | 237 | 238 | 239 |
| D30841AE13 | 233 | 234 | 235 | 237 | 238 | 239 |
| D30841AE14 | 233 | 234 | 235 | 237 | 238 | 239 |
| D30841AE15 | 233 | 234 | 235 | 237 | 238 | 239 |

In some embodiments, the multispecific antigen binding molecules or the DLL3 antigen binding moiety of the present invention comprises an antibody variable region comprising any one of (a1) to (a5) below:
(a1) the heavy chain complementarity determining region (CDR) 1 of SEQ ID NO: 233, the heavy chain CDR 2 of SEQ ID NO: 234, the heavy chain CDR 3 of SEQ ID NO: 235, the light chain CDR 1 of SEQ ID NO: 237, the light chain CDR 2 of SEQ ID NO: 238 and the light chain CDR 3 of SEQ ID NO: 239;
(a2) the heavy chain complementarity determining region (CDR) 1 of SEQ ID NO: 276, the heavy chain CDR 2 of SEQ ID NO: 277, the heavy chain CDR 3 of SEQ ID NO: 278, the light chain CDR 1 of SEQ ID NO: 279, the light chain CDR 2 of SEQ ID NO: 280 and the light chain CDR 3 of SEQ ID NO: 281;
(a3) the heavy chain complementarity determining region (CDR) 1 of SEQ ID NO: 285, the heavy chain CDR 2 of SEQ ID NO: 286, the heavy chain CDR 3 of SEQ ID NO: 287, the light chain CDR 1 of SEQ ID NO: 288, the light chain CDR 2 of SEQ ID NO: 289 and the light chain CDR 3 of SEQ ID NO: 290;
(a4) an antibody variable region that binds to the same epitope of any of the antibody variable region selected from (a1) to (a3); and
(a5) an antibody variable fragment that competes with the binding of any of the antibody variable fragment selected from (a1) to (a3).

In some embodiments, the multispecific antigen binding molecules or the DLL3 antigen binding moiety of the present invention also includes posttranslational modification. Examples of posttranslational modification includes pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

In another aspect, the DLL3 antigen binding moiety of the present invention can be used in novel chimeric antigen receptor (CAR) incorporating a DLL3 binding domain (DLL3 CAR). In certain embodiments, a DLL3 binding domain (and DLL3 CAR) of the invention will comprise a scFv construct, and in a preferred embodiment, will comprise a heavy and light chain variable region as disclosed herein. In other preferred embodiments, the DLL3 binding domain (and DLL3 CAR) of the invention will comprise a scFv construct or fragment thereof comprising the heavy and light chain variable regions disclosed herein. In a preferred embodiment, the disclosed chimeric antigen receptors are useful for treating or preventing a proliferative disorder and any recurrence or metastasis thereof.

In certain embodiments, the DLL3 protein is expressed on tumor-initiating cells. DLL3 CAR is expressed on cytotoxic lymphocytes (preferably autologous cytotoxic lymphocytes) via genetic modification (e.g., transduction), resulting in DLL3-sensitive lymphocytes that can be used to target and kill DLL3-positive tumor cells. As will be broadly discussed herein, CARs of the invention typically comprise an extracellular domain, a transmembrane domain, and an intracellular signaling domain comprising a DLL3 binding domain that activates certain lymphocytes and produces immune response of DLL3 positive tumor cells. Selected embodiments of the invention comprise immunologically active host cells which exhibit the disclosed CAR and various polynucleotide sequences and vectors encoding the DLL3 CAR of the invention. Other aspects include methods of enhancing the activity of T lymphocytes or natural killer (NK) cells in an individual by introducing a host cell expressing a DLL3 CAR molecule into an individual suffering from cancer and treating the individual. Such aspects include, inter alia, lung cancer (e.g., small cell lung cancer) and melanoma.

### Antigen

As used herein, the term “antigen” refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g. CD3, CD137, DLL3) can be any native form the proteins from any vertebrate source, including mammals such as primates (e.g. humans) and rodents (e.g. mice and rats), unless otherwise indicated. In a particular embodiment the antigen is a human CD3, human CD137 or human DLL3. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

In certain embodiments the multispecific antigen binding molecule described herein binds to an epitope of CD3, CD137 or DLL3 that is conserved among the CD3, CD137 or DLL3 from different species. In certain embodiments the multispecific antigen binding molecule of the present application is a trispecific antigen binding molecule, i.e. it is capable of specifically binding to three different antigens—capable of binding to either one of CD3 or CD137 but does not bind to both antigens simultaneously, and is capable of specifically binding to DLL3.

In certain embodiments, the multispecific antigen binding molecule specifically binds to the whole or a portion of a partial peptide of CD3. In a particular embodiment CD3 is human CD3 or cynomolgus CD3, most particularly human CD3. In a particular embodiment the multispecific antigen binding molecule is cross-reactive for (i.e. specifically binds to) human and cynomolgus CD3. In some embodiments, the multispecific antigen binding molecule is capable of specific binding to the epsilon subunit of CD3, in particular the human CD3 epsilon subunit of CD3 which is shown in SEQ ID NOs: 7 (NP_000724.1) (RefSeq registration numbers are shown within the parentheses). In some embodiments, the multispecific antigen binding molecule is capable of specific binding to the CD3 epsilon chain expressed on the surface of eukaryotic cells. In some embodiments, the multispecific antigen binding molecule binds to the CD3 epsilon chain expressed on the surface of T cells.

In certain embodiments, the CD137 is human CD137. In some embodiments, favorable examples of an antigen-binding molecule of the present invention include antigen-binding molecules that bind to the same epitope as the human CD137 epitope bound by the antibody selected from the group consisting of:
antibody that recognize a region comprising the SPCPPNSFSSAGGQRTCDI-CRQCKGVFRTRKECSSTSNAECDCTPGFHCLGAGCSMCEQDCKQGQELTKKG C sequence (SEQ ID NO: 21),
antibody that recognize a region comprising the DCTPGFHCLGAGCSMCEQDCK-QGQELTKKGC sequence (SEQ ID NO: 35),
antibody that recognize a region comprising the LQDPCSNCPAGTFCDNNRNQIC-SPCPPNSFSSAGGQRTCDICRQCKGVFRTRKECSSTSNAEC sequence (SEQ ID NO: 49), and
antibody that recognize a region comprising the LQDPCSNCPAGTFCDNNRNQIC sequence (SEQ ID NO: 105) in the human CD137 protein.

The term “DLL3”, as used herein, refers to any native DLL3 (Delta-like 3) from any vertebrate source, including mammals such as primates (e.g. humans) and rodents (e.g., mice and rats), unless otherwise indicated. The term encompasses “full-length” unprocessed DLL3 as well as any form of DLL3 that results from processing in the cell. The term also encompasses naturally occurring variants of DLL3, e.g., splice variants or allelic variants. The amino acid sequence of an exemplary human DLL3 is known as NCBI Reference Sequence (RefSeq) NM_016941.3, and the amino acid sequence of an exemplary cynomolgus DLL3 is known as NCBI Reference Sequence XP_005589253.1, and the amino acid sequence of an exemplary mouse DLL3 is known as NCBI Reference Sequence NM_007866.2.

The human DLL3 protein comprises a transmembrane (TM) region and an intracellular domain on the C-terminal side, and a DSL (Notch) domain on the N-terminal side (see, e.g., FIG. 6). In addition, DLL3 has an EGF domain comprising six regions, EGF1 to EGF6 from the N-terminal side to the C-terminal side. In some embodiments, the multispecific antigen-binding molecules or the DLL3 antigen binding moiety of the present invention bind to an epitope within the extracellular domain (ECD), i.e., the domain from the N-terminus to immediately before the TM region, but not to the TM region or the C-terminal intracellular domain. The multispecific antigen-binding molecules or the DLL3 antigen binding moiety of the present invention may bind to an epitope within any of the above-mentioned domains/regions within the ECD. In preferred embodiments, the multispecific antigen-binding molecules or the DLL3 antigen binding moiety of the present invention bind to an epitope within the region from EGF6 to immediately before the TM region. More specifically, the multispecific antigen-binding molecules or the DLL3 antigen binding moiety of the present invention may bind to an epitope within the regions defined in SEQ ID NO: 89 in human DLL3. In some embodiments, the molecules/antibodies of the present invention bind to the EGF1, EGF2, EGF3, EGF4, EGF5, or EGF6 region or a region from EGF6 to immediately before the TM region of human DLL3, or an epitope within the EGF1, EGF2, EGF3, EGF4, EGF5, or EGF6 region or a region from EGF6 to immediately before the TM region of human DLL3.

In human DLL3, the above-mentioned domains/regions have the following amino acid residues (see, e.g., http://www.uniprot.org/uniprot/Q9NYJ7 or WO2013/126746):
Extracellular domain (ECD): amino acid residues at positions 1 to 492;
DSL domain: amino acid residues at positions 176 to 215;
EGF domain: amino acid residues at positions 216 to 465;
EGF1 region: amino acid residues at positions 216 to 249;
EGF2 region: amino acid residues at positions 274 to 310;
EGF3 region: amino acid residues at positions 312 to 351;
EGF4 region: amino acid residues at positions 353 to 389;
EGF5 region: amino acid residues at positions 391 to 427;
EGF6 region: amino acid residues at positions 429 to 465;
The region from EGF6 to immediately before the TM region: amino acid residues at positions 429 to 492;
TM region: amino acid residues at positions 493 to 513; and
C-terminal intracellular domain: amino acid residues at positions 516 to 618 (or 516 to 587 in some isoforms). The amino acid positions mentioned above also refers to the amino acid positions in the amino acid sequence shown in SEQ ID NO: 90.
   Thus, the multispecific antigen-binding molecules or the DLL3 antigen binding moiety of the present invention may bind to an above-mentioned region/domain having the amino acid residues at the above-mentioned positions in human DLL3. That is, the multispecific antigen-binding molecules or the DLL3 antigen binding moiety of the present invention may bind to an epitope within the above-mentioned region/domain having the amino acid residues at the above-mentioned positions in human DLL3.

The DLL3 protein used in the present invention may be a DLL3 protein having the sequence described above or may be a modified protein having a sequence derived from the sequence described above by the modification of one or more amino acids. Examples of the modified protein having a sequence derived from the sequence described above by the modification of one or more amino acids can include polypeptides having 70% or more, preferably 80% or more, more preferably 90% or more, even more preferably 95% or more identity with to the amino acid sequence described above. Alternatively, partial peptides of these DLL3 proteins may be used.

The DLL3 protein used in the present invention is not limited by its origin and is preferably a human or cynomolgus DLL3 protein.

In some embodiments, for the DLL3 protein, DLL3 ECD fragment proteins (or ECD variants) may be used. Depending on the site of truncation, the fragments/variants may comprise, from the N-terminal side to the C-terminal side, the DSL domain to EGF6, EGF1 to EGF6, EGF2 to EGF6, EGF3 to EGF6, EGF4 to EGF6, EGF5 and EGF6, or EGF6. The fragments/variants may further comprise a region spanning from immediately after the EGF6 region to immediately before the TM region. A Flag tag may be attached to the C terminus of the fragments/variants using a technique well-known in the art.

### Antigen Binding Domain

The term “antigen binding domain” refers to the part of an antibody that comprises the area which specifically binds to and is complementary to part or all of an antigen. An antigen binding domain may be provided by, for example, one or more antibody variable domains (also called antibody variable regions). Preferably, the antigen-binding domains contain both the antibody light chain variable region (VL) and antibody heavy chain variable region (VH). Such preferable antigen-binding domains include, for example, “single-chain Fv (scFv)”, “single-domain antibody or VHH”, “single-chain antibody”, “Fv”, “single-chain Fv2 (scFv2)”, “Fab”, and “F (ab′)2”.

### Variable Region

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### HVR or CDR

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Hypervariable regions (HVRs) are also referred to as “complementarity determining regions” (CDRs), and these terms are used herein interchangeably in reference to portions of the variable region that form the antigen binding regions. Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

HVR-H1, HVR-H2, HVR-H3, HVR-L1, HVR-L2, and HVR-L3 are also mentioned as “H-CDR1”, “H-CDR2”, “H-CDR3”, “L-CDR1”, “L-CDR2”, and “L-CDR3”, respectively.

Capable of Binding to CD3 and CD137 but does not Bind to CD3 and CD137 at the Same Time

Whether the antibody variable region of the present invention is “capable of binding to CD3 and CD137” can be determined by a method known in the art.

This can be determined by, for example, an electrochemiluminescence method (ECL method) (BMC Research Notes 2011, 4: 281).

Specifically, for example, a low-molecular antibody composed of a region capable of binding to CD3 and CD137, for example, a Fab region, of a biotin-labeled antigen-binding molecule to be tested, or a monovalent antibody (antibody lacking one of the two Fab regions carried by a usual antibody) thereof is mixed with CD3 or CD137 labeled with sulfo-tag (Ru complex), and the mixture is added onto a streptavidin-immobilized plate. In this operation, the biotin-labeled antigen-binding molecule to be tested binds to streptavidin on the plate. Light is developed from the sulfo-tag, and the luminescence signal can be detected using Sector Imager 600 or 2400 (MSD K.K.) or the like to thereby confirm the binding of the aforementioned region of the antigen-binding molecule to be tested to CD3 or CD137.

Alternatively, this assay may be conducted by ELISA, FACS (fluorescence activated cell sorting), ALPHAScreen (amplified luminescent proximity homogeneous assay screen), the BIACORE method based on a surface plasmon resonance (SPR) phenomenon, etc. (Proc. Natl. Acad. Sci. USA (2006) 103 (11), 4005-4010).

Specifically, the assay can be conducted using, for example, an interaction analyzer Biacore (GE Healthcare Japan Corp.) based on a surface plasmon resonance (SPR) phenomenon. The Biacore analyzer includes any model such as Biacore T100, T200, X100, A100, 4000, 3000, 2000, 1000, or C. Any sensor chip for Biacore, such as a CM7, CM5, CM4, CM3, C1, SA, NTA, L1, HPA, or Au chip, can be used as a sensor chip. Proteins for capturing the antigen-binding molecule of the present invention, such as protein A, protein G, protein L, anti-human IgG antibodies, anti-human IgG-Fab, anti-human L chain antibodies, anti-human Fc antibodies, antigenic proteins, or antigenic peptides, are immobilized onto the sensor chip by a coupling method such as amine coupling, disulfide coupling, or aldehyde coupling. CD3 or CD137 is injected thereon as an analyte, and the interaction is measured to obtain a sensorgram. In this operation, the concentration of CD3 or CD137 can be selected within the range of a few micro M to a few pM according to the interaction strength (e.g., KD) of the assay sample.

Alternatively, CD3 or CD137 may be immobilized instead of the antigen-binding molecule onto the sensor chip, with which the antibody sample to be evaluated is in turn allowed to interact. Whether the antibody variable region of the antigen-binding molecule of the present invention has binding activity against CD3 or CD137 can be confirmed on the basis of a dissociation constant (KD) value calculated from the sensorgram of the interaction or on the basis of the degree of increase in the sensorgram after the action of the antigen-binding molecule sample over the level before the action.

In some embodiments, binding activity or affinity of the antibody variable region of the present invention to the antigen of interest (i.e. CD3 or CD137) are assessed at 37 degrees C. (for CD137) or 25 degrees C. (for CD3) using e.g., Biacore T200 instrument (GE Healthcare) or Biacore 8K instrument (GE Healthcare). Anti-human Fc (e.g., GE Healthcare) is immobilized onto all flow cells of a CM4 sensor chip using amine coupling kit (e.g., GE Healthcare). The antigen binding molecules or antibody variable regions are captured onto the anti-Fc sensor surfaces, then the antigen (CD3 or CD137) is injected over the flow cell. The capture levels of the antigen binding molecules or antibody variable regions may be aimed at 200 resonance unit (RU). Recombinant human CD3 or CD137 may be injected at 2000 to 125 nM prepared by two-fold serial dilution, followed by dissociation. All antigen binding molecules or antibody variable regions and analytes are prepared in ACES pH 7.4 containing 20 mM ACES, 150 mM NaCl, 0.05% Tween 20, 0.005% NaN3. Sensor surface is regenerated each cycle with 3M MgCl2. Binding affinity are determined by processing and fitting the data to 1:1 binding model using e.g., Biacore Insight Evaluation software, version 2.0 (GE Healthcare) or Biacore 8K Evaluation software (GE Healthcare). The KD values are calculated for assessing the specific binding activity or affinity of the antigen binding domains of the present invention.

The ALPHAScreen is carried out by the ALPHA technology using two types of beads (donor and acceptor) on the basis of the following principle: luminescence signals are detected only when these two beads are located in proximity through the biological interaction between a molecule bound with the donor bead and a molecule bound with the acceptor bead. A laser-excited photosensitizer in the donor bead converts ambient oxygen to singlet oxygen having an excited state. The singlet oxygen diffuses around the donor bead and reaches the acceptor bead located in proximity thereto to thereby cause chemiluminescent reaction in the bead, which finally emits light. In the absence of the interaction between the molecule bound with the donor bead and the molecule bound with the acceptor bead, singlet oxygen produced by the donor bead does not reach the acceptor bead. Thus, no chemiluminescent reaction occurs.

One (ligand) of the substances between which the interaction is to be observed is immobilized onto a thin gold film of a sensor chip. The sensor chip is irradiated with light from the back such that total reflection occurs at the interface between the thin gold film and glass. As a result, a site having a drop in reflection intensity (SPR signal) is formed in a portion of reflected light. The other (analyte) of the substances between which the interaction is to be observed is injected on the surface of the sensor chip. Upon binding of the analyte to the ligand, the mass of the immobilized ligand molecule is increased to change the refractive index of the solvent on the sensor chip surface. This change in the refractive index shifts the position of the SPR signal (on the contrary, the dissociation of the bound molecules gets the signal back to the original position). The Biacore system plots on the ordinate the amount of the shift, i.e., change in mass on the sensor chip surface, and displays time-dependent change in mass as assay data (sensorgram). The amount of the analyte bound to the ligand captured on the sensor chip surface (amount of change in response on the sensorgram between before and after the interaction of the analyte) can be determined from the sensorgram. However, since the amount bound also depends on the amount of the ligand, the comparison must be performed under conditions where substantially the same amounts of the ligand are used. Kinetics, i.e., an association rate constant (ka) and a dissociation rate constant (kd), can be determined from the curve of the sensorgram, while affinity (KD) can be determined from the ratio between these constants. Inhibition assay is also preferably used in the BIACORE method. Examples of the inhibition assay are described in Proc. Natl. Acad. Sci. USA (2006) 103 (11), 4005-4010.

The term “does not bind to CD3 and CD137 (4-1BB) at the same time” or “does not bind to CD3 and CD137 (4-1BB) simultaneously” means that the antigen-binding moiety or antibody variable region of the present invention cannot bind to CD137 in a state bound with CD3 whereas the antigen-binding moiety or antibody variable region cannot bind to CD3 in a state bound with CD137. In this context, the phrase “not bind to CD3 and CD137 at the same time” also includes not cross-linking a cell expressing CD3 to a cell expressing CD137, or not binding to CD3 and CD137 each expressed on a different cell, at the same time. This phrase further includes the case where the variable region is capable of binding to both CD3 and CD137 at the same time when CD3 and CD137 are not expressed on cell membranes, as with soluble proteins, or both reside on the same cell, but cannot bind to CD3 and CD137 each expressed on a different cell, at the same time. Such an antibody variable region is not particularly limited as long as the antibody variable region has these functions. Examples thereof can include variable regions derived from an IgG-type antibody variable region by the alteration of a portion of its amino acids so as to bind to the desired antigen. The amino acid to be altered is selected from, for example, amino acids whose alteration does not cancel the binding to the antigen, in an antibody variable region binding to CD3 or CD137.

In this context, the phrase “expressed on different cells” merely means that the antigens are expressed on separate cells. The combination of such cells may be, for example, the same types of cells such as a T cell and another T cell, or may be different types of cells such as a T cell and an NK cell.

Whether the antigen-binding molecule of the present invention does “not bind to CD3 and CD137 at the same time” can be confirmed by: confirming the antigen-binding molecule to have binding activity against both CD3 and CD137; then allowing either CD3 or CD137 to bind in advance to the antigen-binding molecule comprising the variable region having this binding activity; and then determining the presence or absence of its binding activity against the other one by the method mentioned above. Alternatively, this can also be confirmed by determining whether the binding of the antigen-binding molecule to either CD3 or CD137 immobilized on an ELISA plate or a sensor chip is inhibited by the addition of the other one into the solution. In some embodiments, the binding of the antigen-binding molecule of the present invention to either CD3 or CD137 is inhibited by binding of the antigen-binding molecule to the other by at least 50%, preferably 60% or more, more preferably 70% or more, more preferably 80% or more, further preferably 90% or more, or even more preferably 95% or more.

In one aspect, while one antigen (e.g. CD3) is immobilized, the inhibition of the binding of the antigen-binding molecule to CD3 can be determined in the presence of the other antigen (e.g. CD137) by methods known in prior art (i.e. ELISA, BIACORE, and so on). In another aspect, while CD137 is immobilized, the inhibition of the binding of the antigen-binding molecule to CD137 also can be determined in the presence of CD3. When either one of two aspects mentioned above is conducted, the antigen-binding molecule of the present invention is determined not to bind to CD3 and CD137 at the same time if the binding is inhibited by at least 50%, preferably 60% or more, preferably 70% or more, further preferably 80% or more, further preferably 90% or more, or even more preferably 95% or more.

In some embodiments, the concentration of the antigen injected as an analyte is at least 1-fold, 2-fold, 5-fold, 10-fold, 30-fold, 50-fold, or 100-fold higher than the concentration of the other antigen to be immobilized.

In preferable manner, the concentration of the antigen injected as an analyte is 100-fold higher than the concentration of the other antigen to be immobilized and the binding is inhibited by at least 80%.

In one embodiment, the ratio of the KD value for the CD3 (analyte)-binding activity of the antigen-binding molecule to the CD137 (immobilized)-binding activity of the antigen-binding molecule (KD (CD3)/KD (CD137)) is calculated and the CD3 (analyte) concentration which is 10-fold, 50-fold, 100-fold, or 200-fold of the ratio of the KD value (KD(CD3)/KD(CD137) higher than the CD137 (immobilized) concentration can be used for the competition measurement above. (e.g. 1-fold, 5-fold, 10-fold, or 20-fold higher concentration can be selected when the ratio of the KD value is 0.1. Furthermore, 100-fold, 500-fold, 1000-fold, or 2000-fold higher concentration can be selected when the ratio of the KD value is 10.)

In one aspect, while one antigen (e.g. CD3) is immobilized, the attenuation of the binding signal of the antigen-binding molecule to CD3 can be determined in the presence of the other antigen (e.g. CD137) by methods known in prior art (i.e. ELISA, ECL and so on). In another aspect, while CD137 is immobilized, the attenuation of the binding signal of the antigen-binding molecule to CD137 also can be determined in the presence of CD3. When either one of two aspects mentioned above is conducted, the antigen-binding molecule of the present invention is determined not to bind to CD3 and CD137 at the same time if the binding signal is attenuated by at least 50%, preferably 60% or more, preferably 70% or more, further preferably 80% or more, further preferably 90% or more, or even more preferably 95% or more.

In some embodiments, the concentration of the antigen injected as an analyte is at least 1-fold, 2-fold, 5-fold, 10-fold, 30-fold, 50-fold, or 100-fold higher than the concentration of the other antigen to be immobilized.

In preferable manner, the concentration of the antigen injected as an analyte is 100-fold higher than the concentration of the other antigen to be immobilized and the binding is inhibited by at least 80%.

In one embodiment, the ratio of the KD value for the CD3 (analyte)-binding activity of the antigen-binding molecule to the CD137 (immobilized)-binding activity of the antigen-binding molecule (KD (CD3)/KD (CD137)) is calculated and the CD3 (analyte) concentration which is 10-fold, 50-fold, 100-fold, or 200-fold of the ratio of the KD value (KD(CD3)/KD(CD137) higher than the CD137 (immobilized) concentration can be used for the measurement above. (e.g. 1-fold, 5-fold, 10-fold, or 20-fold higher concentration can be selected when the ratio of the KD value is 0.1. Furthermore, 100-fold, 500-fold, 1000-fold, or 2000-fold higher concentration can be selected when the ratio of the KD value is 10.)

Specifically, in the case of using, for example, the ECL method, a biotin-labeled antigen-binding molecule to be tested, CD3 labeled with sulfo-tag (Ru complex), and an unlabeled CD137 are prepared. When the antigen-binding molecule to be tested is capable of binding to CD3 and CD137, but does not bind to CD3 and CD137 at the same time, the luminescence signal of the sulfo-tag is detected in the absence of the unlabeled CD137 by adding the mixture of the antigen-binding molecule to be tested and labeled CD3 onto a streptavidin-immobilized plate, followed by light development. By contrast, the luminescence signal is decreased in the presence of unlabeled CD137. This decrease in luminescence signal can be quantified to determine relative binding activity. This analysis may be similarly conducted using the labeled CD137 and the unlabeled CD3.

In the case of the ALPHAScreen, the antigen-binding molecule to be tested interacts with CD3 in the absence of the competing CD137 to generate signals of 520 to 620 nm. The untagged CD137 competes with CD3 for the interaction with the antigen-binding molecule to be tested. Decrease in fluorescence caused as a result of the competition can be quantified to thereby determine relative binding activity. The polypeptide biotinylation using sulfo-NHS-biotin or the like is known in the art. CD3 can be tagged with GST by an appropriately adopted method which involves, for example: fusing a polynucleotide encoding CD3 in flame with a polynucleotide encoding GST; and allowing the resulting fusion gene to be expressed by cells or the like harboring vectors capable of expression thereof, followed by purification using a glutathione column. The obtained signals are preferably analyzed using, for example, software GRAPHPAD PRISM (GraphPad Software, Inc., San Diego) adapted to a one-site competition model based on nonlinear regression analysis. This analysis may be similarly conducted using the tagged CD137 and the untagged CD3.

Alternatively, a method using fluorescence resonance energy transfer (FRET) may be used. FRET is a phenomenon in which excitation energy is transferred directly between two fluorescent molecules located in proximity to each other by electron resonance. When FRET occurs, the excitation energy of a donor (fluorescent molecule having an excited state) is transferred to an acceptor (another fluorescent molecule located near the donor) so that the fluorescence emitted from the donor disappears (to be precise, the lifetime of the fluorescence is shortened) and instead, the fluorescence is emitted from the acceptor. By use of this phenomenon, whether or not bind to CD3 and CD137 at the same time can be analyzed. For example, when CD3 carrying a fluorescence donor and CD137 carrying a fluorescence acceptor bind to the antigen-binding molecule to be tested at the same time, the fluorescence of the donor disappears while the fluorescence is emitted from the acceptor. Therefore, change in fluorescence wavelength is observed. Such an antibody is confirmed to bind to CD3 and CD137 at the same time. On the other hand, if the mixing of CD3, CD137, and the antigen-binding molecule to be tested does not change the fluorescence wavelength of the fluorescence donor bound with CD3, this antigen-binding molecule to be tested can be regarded as antigen binding domain that is capable of binding to CD3 and CD137, but does not bind to CD3 and CD137 at the same time.

For example, a biotin-labeled antigen-binding molecule to be tested is allowed to bind to streptavidin on the donor bead, while CD3 tagged with glutathione S transferase (GST) is allowed to bind to the acceptor bead. The antigen-binding molecule to be tested interacts with CD3 in the absence of the competing second antigen to generate signals of 520 to 620 nm. The untagged second antigen competes with CD3 for the interaction with the antigen-binding molecule to be tested. Decrease in fluorescence caused as a result of the competition can be quantified to thereby determine relative binding activity. The polypeptide biotinylation using sulfo-NHS-biotin or the like is known in the art. CD3 can be tagged with GST by an appropriately adopted method which involves, for example: fusing a polynucleotide encoding CD3 in flame with a polynucleotide encoding GST; and allowing the resulting fusion gene to be expressed by cells or the like harboring vectors capable of expression thereof, followed by purification using a glutathione column. The obtained signals are preferably analyzed using, for example, software GRAPHPAD PRISM (GraphPad Software, Inc., San Diego) adapted to a one-site competition model based on nonlinear regression analysis.

The tagging is not limited to the GST tagging and may be carried out with any tag such as, but not limited to, a histidine tag, MBP, CBP, a Flag tag, an HA tag, a V5 tag, or a c-myc tag. The binding of the antigen-binding molecule to be tested to the donor bead is not limited to the binding using biotin-streptavidin reaction. Particularly, when the antigen-binding molecule to be tested comprises Fc, a possible method involves allowing the antigen-binding molecule to be tested to bind via an Fc-recognizing protein such as protein A or protein G on the donor bead.

Also, the case where the variable region is capable of binding to CD3 and CD137 at the same time when CD3 and CD137 are not expressed on cell membranes, as with soluble proteins, or both reside on the same cell, but cannot bind to CD3 and CD137 each expressed on a different cell, at the same time can also be assayed by a method known in the art.

Specifically, the antigen-binding molecule to be tested has been confirmed to be positive in ECL-ELISA for detecting binding to CD3 and CD137 at the same time is also mixed with a cell expressing CD3 and a cell expressing CD137. The antigen-binding molecule to be tested can be shown to be incapable of binding to CD3 and CD137 expressed on different cells, at the same time unless the antigen-binding molecule and these cells bind to each other at the same time. This assay can be conducted by, for example, cell-based ECL-ELISA. The cell expressing CD3 is immobilized onto a plate in advance. After binding of the antigen-binding molecule to be tested thereto, the cell expressing CD137 is added to the plate. A different antigen expressed only on the cell expressing CD137 is detected using a sulfo-tag-labeled antibody against this antigen. A signal is observed when the antigen-binding molecule binds to the two antigens respectively expressed on the two cells, at the same time. No signal is observed when the antigen-binding molecule does not bind to these antigens at the same time.

Alternatively, this assay may be conducted by the ALPHAScreen method. The antigen-binding molecule to be tested is mixed with a cell expressing CD3 bound with the donor bead and a cell expressing CD137 bound with the acceptor bead. A signal is observed when the antigen-binding molecule binds to the two antigens expressed on the two cells respectively, at the same time. No signal is observed when the antigen-binding molecule does not bind to these antigens at the same time.

Alternatively, this assay may also be conducted by an Octet interaction analysis method. First, a cell expressing CD3 tagged with a peptide tag is allowed to bind to a biosensor that recognizes the peptide tag. A cell expressing CD137 and the antigen-binding molecule to be tested are placed in wells and analyzed for interaction. A large wavelength shift caused by the binding of the antigen-binding molecule to be tested and the cell expressing CD137 to the biosensor is observed when the antigen-binding molecule binds to the two antigens expressed on the two cells respectively, at the same time. A small wavelength shift caused by the binding of only the antigen-binding molecule to be tested to the biosensor is observed when the antigen-binding molecule does not bind to these antigens at the same time.

Instead of these methods based on the binding activity, assay based on biological activity may be conducted. For example, a cell expressing CD3 and a cell expressing CD137 are mixed with the antigen-binding molecule to be tested, and cultured. The two antigens expressed on the two cells respectively are mutually activated via the antigen-binding molecule to be tested when the antigen-binding molecule binds to these two antigens at the same time. Therefore, change in activation signal, such as increase in the respective downstream phosphorylation levels of the antigens, can be detected. Alternatively, cytokine production is induced as a result of the activation. Therefore, the amount of cytokines produced can be measured to thereby confirm whether or not to bind to the two cells at the same time. Alternatively, cytotoxicity against a cell expressing CD137 is induced as a result of the activation. Alternatively, the expression of a reporter gene is induced by a promoter which is activated at the downstream of the signal transduction pathway of CD137 or CD3 as a result of the activation. Therefore, the cytotoxicity or the amount of reporter proteins produced can be measured to thereby confirm whether or not to bind to the two cells at the same time.

### At Least One Disulfide Bond

In one aspect of the present invention, each of the first antigen-binding moiety and the second antigen-binding moiety comprises at least one cysteine residue (via mutation, substitution or insertion), preferably in the CH1 region, and said at least one cysteine residue is capable of forming at least one disulfide bond between the first antigen-binding moiety and the second antigen-binding moiety. In certain embodiments, the cysteine residue is present within a CH1 region of an antibody heavy chain constant region, and for example, it is present at a position selected from the group consisting of positions 119, 122, 123, 131, 132, 133, 134, 135, 136, 137, 139, 140, 148, 150, 155, 156, 157, 159, 160, 161, 162, 163, 165, 167, 174, 176, 177, 178, 190, 191, 192, 194, 195, 197, 213, and 214 according to EU numbering in the CH1 region. In one embodiment, each of the first antigen-binding moiety and the second antigen-binding moiety comprises one cysteine residue (via mutation, substitution or insertion) at position 191 according to EU numbering in the CH1 region which is capable of forming one disulfide bond between the CH1 region of the first antigen-binding moiety and the CH1 region of the second antigen-binding moiety.

In an embodiment of the above aspects, “at least one bond” to be formed linking the first antigen-binding moiety and the second antigen-binding moiety as described above can hold the two antigen binding moiety (i.e., the first antigen-binding moiety and the second antigen-binding moiety as described above) spatially close positions. By virtue of the linkage between the first antigen-binding moiety and the second antigen-binding moiety via the disulfide bond(s), the antigen-binding molecule of the present invention is capable of holding two antigen-binding moieties at closer positions than a control antigen-binding molecule, which differs from the antigen-binding molecule of the present invention only in that the control antigen-binding molecule does not have the additional bond(s) introduced between the two antigen-binding moieties. In some embodiments, the term “spatially close positions” or “closer positions” includes the meaning that the first antigen-binding domain and the second antigen-binding domain as described above hold in shortened distance and/or reduced flexibility.

As the results, the two antigen binding moieties (i.e., the first antigen-binding moiety and the second antigen-binding moiety as described above) of the antigen-binding molecule of the present invention binds to the antigens expressed on the same single cell. In other words, the respective two antigen-binding moieties (i.e., the first antigen-binding moiety and the second antigen-binding moiety as described above) of the antigen-binding molecule of the present invention do not bind to antigens expressed on different cells so as to cause a cross-linking the different cells. In the present application, such antigen-binding manner of the antigen-binding molecule of the present invention can be called as “cis-binding”, whereas the antigen-binding manner of an antigen-binding molecule which respective two antigen-binding moiety of the antigen-binding molecule bind to antigens expressed on different cells so as to cause a cross-linking the different cells can be called as “trans-binding”. In some embodiments, the antigen-binding molecule of the present invention predominantly binds to the antigens expressed on the same single cell in “cis-biding” manner.

In an embodiment of the above aspects, by virtue of the disulfide linkage between the first antigen-binding moiety and the second antigen-binding moiety via the disulfide bond(s) as described above, the antigen-binding molecule of the present invention is capable of reducing and/or preventing unwanted cross-linking and activation of immune cells (e.g., T-cells, NK cells, DC cells, or the like). That is, in some embodiments of the present invention, the first antigen-binding moiety of the antigen-binding molecule of the present invention binds to any signaling molecule expressed on an immune cell such as T-cell (e.g., the first antigen), and the second antigen-binding domain of the antigen-binding molecule of the present invention also binds to any signaling molecule expressed on an immune cell such as T-cell (e.g., the first antigen or the second antigen which is different from the first antigen). Thus, the first antigen-binding domain and the second antigen-binding domain of the antigen binding-molecule of the present invention can bind to either of the first or second signaling molecule expressed on the same single immune cell such as T cell (i.e., cis-binding manner) or on different immune cell such as T cells (i.e., trans-biding manner). When the first antigen-binding domain and the second antigen-binding domain bind to the signaling molecule expressed on different immune cells such as T-cells in trans-binging manner, those different immune cells such as T-cells are cross-linked, and, in certain situation, such crosslinking of immune cells such as T-cells may cause unwanted activation of the immune cells such as T-cells.

On the other hand, in the case of another embodiment of the antigen-binding molecule of the present invention, that is, an antigen-binding molecule comprising the first antigen-binding moiety and the second antigen-binding moiety, which are linked with each other via at least one disulfide bond in the CH1 region (position 191 according to EU numbering), both of the first antigen-binding moiety and the second antigen-binding moiety can binds to the signaling molecules expressed on the same single immune cells such as T cell in “cis-biding” manner, so that the crosslinking of different immune cells such as T-cells via the antigen-binding molecule can be reduced to avoid unwanted activation of immune cells.

In the instant application, the above-described feature, the at least one disulfide bond in the CH1 region (e.g. position 191 according to EU numbering) linking the first antigen-binding moiety and the second antigen-binding moiety may be described with the abbreviated term “LINC”. Using this abbreviation, in some embodiments, the above-described antigen-binding molecule of the present invention having said at least one disulfide bond may be indicated as, e.g., “LINC format”, “Dual/LINC” or “DLL3-Dual/LINC” or the like. Likewise, antigen-binding molecules of which the first antigen-binding moiety and the second antigen-binding moiety that are not linked/yet to be linked with each other via at least one disulfide bond in the CH1 region (e.g. position 191 according to EU numbering) may be described with the abbreviated term “UnLINC”.

### Fab Molecule

A “Fab molecule” refers to a protein consisting of the VH and CH1 domain of the heavy chain (the “Fab heavy chain”) and the VL and CL domain of the light chain (the “Fab light chain”) of an immunoglobulin.

### Fused

By “fused” is meant that the components (e.g. a Fab molecule and an Fc domain subunit) are linked by peptide bonds, either directly or via one or more peptide linkers.

### “Crossover” Fab

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

### “Conventional” Fab

In contrast thereto, by a “conventional” Fab molecule is meant a Fab molecule in its natural format, i.e. comprising a heavy chain composed of the heavy chain variable and constant regions (VH-CH1), and a light chain composed of the light chain variable and constant regions (VL-CL). The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called alpha (IgA), delta (IgD), epsilon (IgE), gamma (IgG), or mu (IgM), some of which may be further divided into subtypes, e.g. gamma1 (IgG1), gamma2 (IgG2), gamma3 (IgG3), gamma4 (IgG4), alpha1 (IgA1) and alpha2 (IgA2). The light chain of an immunoglobulin may be assigned to one of two types, called kappa and lambda, based on the amino acid sequence of its constant domain. An immunoglobulin essentially consists of two Fab molecules and an Fc domain, linked via the immunoglobulin hinge region.

### Affinity

“Affinity” refers to the strength of the sum total of noncovalent interactions between a single binding site of a molecule (e.g., an antigen-binding molecule or antibody) and its binding partner (e.g., an antigen). Unless indicated otherwise, as used herein, “binding affinity” refers to intrinsic binding affinity which reflects a 1:1 interaction between members of a binding pair (e.g., antigen-binding molecule and antigen, or antibody and antigen). The affinity of a molecule X for its partner Y can generally be represented by the dissociation constant (KD), which is the ratio of dissociation and association rate constants (koff and kon, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

### Methods to Determine Affinity

In certain embodiments, the antigen-binding molecule or antibody provided herein has a dissociation constant (KD) of 1 micro M or less, 120 nM or less, 100 nM or less, 80 nM or less, 70 nM or less, 50 nM or less, 40 nM or less, 30 nM or less, 20 nM or less, 10 nM or less, 2 nM or less, 1 nM or less, 0.1 nM or less, 0.01 nM or less, or 0.001 nM or less (e.g., 10⁻⁸ M or less, 10⁻⁸ M to 10⁻¹³ M, 10⁻⁹ M to 10⁻¹³ M) for its antigen. In certain embodiments, the KD value of the antibody/antigen-binding molecule for CD3, CD137 or DLL3 falls within the range of 1-40, 1-50, 1-70, 1-80, 30-50, 30-70, 30-80, 40-70, 40-80, or 60-80 nM.

In one embodiment, KD is measured by a radiolabeled antigen-binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro l/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE(registered trademark)-3000 (BIAcore, Inc., Piscataway, NJ) is performed at 25 degrees C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 micro g/ml (˜0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min. Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

According to the methods for measuring the affinity of the antigen-binding molecule or the antibody described above, persons skilled in art can carry out affinity measurement for other antigen-binding molecules or antibodies, towards various kind of antigens.

### Antibody

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

### Antibody Fragment

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

### Class of Antibody

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

Unless otherwise indicated, amino acid residues in the light chain constant region are numbered herein according to Kabat et al., and numbering of amino acid residues in the heavy chain constant region is according to the EU numbering system, also called the EU index, as described in Kabat et al., Sequences of Proteins of Immunological Interest, 5th Ed. Public Health Service, National Institutes of Health, Bethesda, MD, 1991.

### Framework

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

Human consensus framework A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

### Chimeric Antibody

The term “chimeric” antibody refers to an antibody in which a portion of the heavy and/or light chain is derived from a particular source or species, while the remainder of the heavy and/or light chain is derived from a different source or species. Similarly, the term “chimeric antibody variable domain” refers to an antibody variable region in which a portion of the heavy and/or light chain variable region is derived from a particular source or species, while the remainder of the heavy and/or light chain variable region is derived from a different source or species.

### Humanized Antibody

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A “humanized antibody variable region” refers to the variable region of a humanized antibody.

### Human Antibody

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “human antibody variable region” refers to the variable region of a human antibody.

### Polynucleotide (Nucleic Acid)

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, alpha-anomeric sugars, epimeric sugars such as arabinose, xyloses or lyxoses, pyranose sugars, furanose sugars, sedo-heptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

### Isolated (Nucleic Acid)

An “isolated” nucleic acid molecule is one which has been separated from a component of its natural environment. An isolated nucleic acid molecule further includes a nucleic acid molecule contained in cells that ordinarily contain the nucleic acid molecule, but the nucleic acid molecule is present extrachromosomally or at a chromosomal location that is different from its natural chromosomal location.

### Vector

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Vectors could be introduced into host cells using virus or electroporation. However, introduction of vectors is not limited to in vitro method. For example, vectors could also be introduced into a subject using in vivo method directly.

In another aspects of the present invention, the vectors comprising nucleic acid molecule encodes the antigen-binding moiety capable of binding to CD3 and CD137 but not at the same time, antigen-binding moiety capable of binding to DLL3, antigen-binding molecules or antibodies of the present disclosure may be introduced to subjects, to express the antigen-binding moieties, antigen-binding molecules or antibodies of the present disclosure directly within the subject. An example of vectors that is possible to be used is adenovirus, but not limited to. It is also possible to administer the nucleic acid molecule encoding the antigen-binding moieties, antigen-binding molecules or antibodies of the present disclosure directly into a subject, or transfer the nucleic acid molecule encodes the antigen-binding moieties, antigen-binding molecules or antibodies of the present disclosure via electroporation to a subject, or administer cells comprises nucleic acid molecule encodes the antigen-binding moieties, antigen-binding molecules or antibodies of the present disclosure to be expressed and secreted into a subject, to express and secrete the antigen-binding moieties, antigen-binding molecules or antibodies of the present disclosure in the subject continuously.

### Host Cell

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### Specificity

“Specific” means that a molecule that binds specifically to one or more binding partners does not show any significant binding to molecules other than the partners. Furthermore, “specific” is also used when an antigen-binding site is specific to a particular epitope of multiple epitopes contained in an antigen. If an antigen-binding molecule binds specifically to an antigen, it is also described as “the antigen-binding molecule has/shows specificity to/towards the antigen”. When an epitope bound by an antigen-binding site is contained in multiple different antigens, an antigen-binding molecule containing the antigen-binding site can bind to various antigens that have the epitope.

### Antibody Fragment

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

### Variable Fragment (Fv)

Herein, the term “variable fragment (Fv)” refers to the minimum unit of an antibody-derived antigen-binding site that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen.

scFv, Single-Chain Antibody, and sc(Fv)₂

Herein, the terms “scFv”, “single-chain antibody”, and “sc(Fv)₂” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen-binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

scFv is an single chain low molecule weight antibody in which VH and VL forming Fv are linked together by a peptide linker (Proc. Natl. Acad. Sci. U.S.A. (1988) 85(16), 5879-5883). VH and VL can be retained in close proximity by the peptide linker.

sc(Fv)₂ is a single chain antibody in which four variable regions of two VL and two VH are linked by linkers such as peptide linkers to form a single chain (J Immunol. Methods (1999) 231(1-2), 177-189). The two VH and two VL may be derived from different monoclonal antibodies. Such sc(Fv)₂ preferably includes, for example, a bispecific sc(Fv)₂ that recognizes two epitopes present in a single antigen as disclosed in the Journal of Immunology (1994) 152(11), 5368-5374. sc(Fv)₂ can be produced by methods known to those skilled in the art. For example, sc(Fv)₂ can be produced by linking scFv by a linker such as a peptide linker.

Herein, an sc(Fv)₂ includes two VH units and two VL units which are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Examples of the form are listed below.
[VL]-linker-[VH]-linker-[VH]-linker-[VL]
[VH]-linker-[VL]-linker-[VL]-linker-[VH]
[VH]-linker-[VH]-linker-[VL]-linker-[VL]
[VL]-linker-[VL]-linker-[VH]-linker-[VH]
[VL]-linker-[VH]-linker-[VL]-linker-[VH]

The molecular form of sc(Fv)₂ is also described in detail in WO 2006/132352. According to these descriptions, those skilled in the art can appropriately prepare desired sc(Fv)₂ to produce the polypeptide complexes disclosed herein.

Furthermore, the antigen-binding molecules or antibodies of the present disclosure may be conjugated with a carrier polymer such as PEG or an organic compound such as an anticancer agent. Alternatively, a sugar chain addition sequence is preferably inserted into the antigen-binding molecules or antibodies such that the sugar chain produces a desired effect.

The linkers to be used for linking the variable regions of an antibody comprise arbitrary peptide linkers that can be introduced by genetic engineering, synthetic linkers, and linkers disclosed in, for example, Protein Engineering, 9(3), 299-305, 1996. However, peptide linkers are preferred in the present disclosure. The length of the peptide linkers is not particularly limited, and can be suitably selected by those skilled in the art according to the purpose. The length is preferably five amino acids or more (without particular limitation, the upper limit is generally 30 amino acids or less, preferably 20 amino acids or less), and particularly preferably 15 amino acids. When sc(Fv)₂ contains three peptide linkers, their length may be all the same or different.

For example, such peptide linkers include:

| | Ser, |
|---|---|
| | Gly-Ser, |
| | Gly-Gly-Ser, |
| | Ser-Gly-Gly, |
| (SEQ ID NO: 91) | |
| | Gly-Gly-Gly-Ser, |
| (SEQ ID NO: 92) | |
| | Ser-Gly-Gly-Gly, |
| (SEQ ID NO: 93) | |
| | Gly-Gly-Gly-Gly-Ser, |
| (SEQ ID NO: 94) | |
| | Ser-Gly-Gly-Gly-Gly, |
| (SEQ ID NO: 95) | |
| | Gly-Gly-Gly-Gly-Gly-Ser, |
| (SEQ ID NO: 96) | |
| | Ser-Gly-Gly-Gly-Gly-Gly, |
| (SEQ ID NO: 97) | |
| | Gly-Gly-Gly-Gly-Gly-Gly-Ser, |
| (SEQ ID NO: 98) | |
| | Ser-Gly-Gly-Gly-Gly-Gly-Gly, |
| (SEQ ID NO: 93) | |
| | (Gly-Gly-Gly-Gly-Ser)n, |
| | and |
| (SEQ ID NO: 94) | |
| | (Ser-Gly-Gly-Gly-Gly)n, |

where n is an integer of 1 or larger. The length or sequences of peptide linkers can be selected accordingly by those skilled in the art depending on the purpose.

Synthetic linkers (chemical crosslinking agents) are routinely used to crosslink peptides, and examples include:
N-hydroxy succinimide (NHS),
disuccinimidyl suberate (DSS),
bis(sulfosuccinimidyl) suberate (BS3),
dithiobis(succinimidyl propionate) (DSP),
dithiobis(sulfosuccinimidyl propionate) (DTSSP),
ethylene glycol bis(succinimidyl succinate) (EGS),
ethylene glycol bis(sulfosuccinimidyl succinate) (sulfo-EGS),
disuccinimidyl tartrate (DST), disulfosuccinimidyl tartrate (sulfo-DST),
bis[2-(succinimidoxycarbonyloxy)ethyl] sulfone (BSOCOES), and
bis[2-(sulfosuccinimidoxycarbonyloxy)ethyl] sulfone (sulfo-BSOCOES). These crosslinking agents are commercially available.

In general, three linkers are required to link four antibody variable regions together. The linkers to be used may be of the same type or different types.

### Fab, F(ab′)₂, and Fab′

“Fab” consists of a single light chain, and a CH1 domain and variable region from a single heavy chain. The heavy chain of Fab molecule cannot form disulfide bonds with another heavy chain molecule.

“F(ab′)₂” or “Fab” is produced by treating an immunoglobulin (monoclonal antibody) with a protease such as pepsin and papain, and refers to an antibody fragment generated by digesting an immunoglobulin (monoclonal antibody) near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)₂” consists of two light chains and two heavy chains comprising the constant region of a CH1 domain and a portion of CH2 domains so that disulfide bonds are formed between the two heavy chains. The F(ab′)₂ disclosed herein can be preferably produced as follows. A whole monoclonal antibody or such comprising a desired antigen-binding site is partially digested with a protease such as pepsin; and Fc fragments are removed by adsorption onto a Protein A column. The protease is not particularly limited, as long as it can cleave the whole antibody in a selective manner to produce F(ab′)₂ under an appropriate setup enzyme reaction condition such as pH. Such proteases include, for example, pepsin and ficin.

### Single-Domain Antibody

In the present specification, the term “single-domain antibody” is not limited by its structure as long as the domain can exert antigen binding activity by itself. It is known that a general antibody, for example, an IgG antibody, exhibits antigen binding activity in a state where a variable region is formed by the pairing of VH and VL, whereas the own domain structure of the single-domain antibody can exert antigen binding activity by itself without pairing with another domain. Usually, the single-domain antibody has a relatively low molecular weight and exists in the form of a monomer.

Examples of the single-domain antibody include, but are not limited to, antigen binding molecules congenitally lacking a light chain, such as VHH of an animal of the family Camelidae and shark VNAR, and antibody fragments containing the whole or a portion of an antibody VH domain or the whole or a portion of an antibody VL domain. Examples of the single-domain antibody which is an antibody fragment containing the whole or a portion of an antibody VH or VL domain include, but are not limited to, artificially prepared single-domain antibodies originating from human antibody VH or human antibody VL as described in U.S. Pat. No. 6,248,516 B1, etc. In some embodiments of the present invention, one single-domain antibody has three CDRs (CDR1, CDR2 and CDR3).

The single-domain antibody can be obtained from an animal capable of producing the single-domain antibody or by the immunization of the animal capable of producing the single-domain antibody. Examples of the animal capable of producing the single-domain antibody include, but are not limited to, animals of the family Camelidae, and transgenic animals harboring a gene capable of raising the single-domain antibody. The animals of the family Camelidae include camels, lamas, alpacas, one-hump camels and guanacos, etc. Examples of the transgenic animals harboring a gene capable of raising the single-domain antibody include, but are not limited to, transgenic animals described in International Publication No. WO2015/143414 and U.S. Patent Publication No. US2011/0123527 A1. The framework sequences of the single-domain antibody obtained from the animal may be converted to human germline sequences or sequences similar thereto to obtain a humanized single-domain antibody. The humanized single-domain antibody (e.g., humanized VHH) is also one embodiment of the single-domain antibody of the present invention.

Alternatively, the single-domain antibody can be obtained by ELISA, panning, or the like from a polypeptide library containing single-domain antibodies. Examples of the polypeptide library containing single-domain antibodies include, but are not limited to, naive antibody libraries obtained from various animals or humans (e.g., Methods in Molecular Biology 2012 911 (65-78); and Biochimica et Biophysica Acta—Proteins and Proteomics 2006 1764: 8 (1307-1319)), antibody libraries obtained by the immunization of various animals (e.g., Journal of Applied Microbiology 2014 117: 2 (528-536)), and synthetic antibody libraries prepared from antibody genes of various animals or humans (e.g., Journal of Biomolecular Screening 2016 21: 1 (35-43); Journal of Biological Chemistry 2016 291:24 (12641-12657); and AIDS 2016 30: 11 (1691-1701)).

### Fc Region

The term “Fc region” or “Fc domain” refers to a region comprising a fragment consisting of a hinge or a portion thereof and CH2 and CH3 domains in an antibody molecule. The Fc region of IgG class means, but is not limited to, a region from, for example, cysteine 226 (EU numbering (also referred to as EU index herein)) to the C terminus or proline 230 (EU numbering) to the C terminus. The Fc region can be preferably obtained by the partial digestion of, for example, an IgG1, IgG2, IgG3, or IgG4 monoclonal antibody with a proteolytic enzyme such as pepsin followed by the re-elution of a fraction adsorbed on a protein A column or a protein G column. Such a proteolytic enzyme is not particularly limited as long as the enzyme is capable of digesting a whole antibody to restrictively form Fab or F(ab′)2 under appropriately set reaction conditions (e.g., pH) of the enzyme. Examples thereof can include pepsin and papain.

An Fc region derived from, for example, naturally occurring IgG can be used as the “Fc region” of the present invention. In this context, the naturally occurring IgG means a polypeptide that contains an amino acid sequence identical to that of IgG found in nature and belongs to a class of an antibody substantially encoded by an immunoglobulin gamma gene. The naturally occurring human IgG means, for example, naturally occurring human IgG1, naturally occurring human IgG2, naturally occurring human IgG3, or naturally occurring human IgG4. The naturally occurring IgG also includes variants or the like spontaneously derived therefrom. A plurality of allotype sequences based on gene polymorphism are described as the constant regions of human IgG1, human IgG2, human IgG3, and human IgG4 antibodies in Sequences of proteins of immunological interest, NIH Publication No. 91-3242, any of which can be used in the present invention. Particularly, the sequence of human IgG1 may have DEL or EEM as an amino acid sequence of EU numbering positions 356 to 358.

In some embodiments, the Fc domain of the multispecific antigen binding molecule consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the multispecific antigen binding molecule described herein comprises not more than one Fc domain.

In one embodiment described herein, the Fc domain of the multispecific antigen binding molecule is an IgG Fc domain. In a particular embodiment the Fc domain is an IgG1 Fc domain. In a further particular embodiment the Fc domain is a human IgG1 Fe region.

In certain embodiments, the Fc domain of the multispecific antigen binding molecule is composed of a first and a second Fc region subunit capable of stable association, and the Fc domain exhibits reduced binding affinity to human Fc gamma receptor, as compared to a native human IgG1 Fc domain.

In certain embodiments, the Fc domain of the multispecific antigen binding molecule described herein comprises a modification promoting the association of the first and the second subunit of the Fc domain. In a specific embodiment said modification is a so-called “knob-into-hole” modification, comprising a “knob” modification in one of the two subunits of the Fc domain and a “hole” modification in the other one of the two subunits of the Fc domain, which will be described in more detail below.

In a specific embodiment, in the Fc domain composed of a first and a second Fc region subunit capable of stable association and exhibiting reduced binding affinity to human Fc-gamma receptor as compared to a native human IgG1 Fc domain, the first Fc-region subunit is selected from the group consisting of:
(a1) a Fc region polypeptide comprising the mutations L234A, L235A;
(a2) a Fc region polypeptide comprising the mutations L234A, L235A, N297A;
(a3) a Fc region polypeptide comprising the mutations L234A, L235A, N297A, S354C, T366W; and
the second Fc-region subunit is selected from the group consisting of:
(a4) a Fc region polypeptide comprising the mutations L234A, L235A;
(a5) a Fc region polypeptide comprising the mutations L234A, L235A, N297A; and
(a6) a Fc region polypeptide comprising the mutations L234A, L235A, N297A, Y349C, T366S, L368A, Y407V (the amino acid positions are numbered using EU index numbering).

In certain embodiments, the Fc domain of the multispecific antigen binding molecule described herein exhibits enhanced FcRn-binding activity under an acidic pH condition (e.g., pH 5.8) as compared to that of an Fc region of a native IgG. Such Fc domain comprises, for example, Ala at position 434; Glu, Arg, Ser, or Lys at position 438; and Glu, Asp, or Gln at position 440, according to EU numbering. In some embodiments, the Fc domain comprises Ala at position 434; Arg or Lys at position 438; and Glu or Asp at position 440, according to EU numbering. In some embodiments, the Fc domain further comprises Ile or Leu at position 428; and/or Ile, Leu, Val, Thr, or Phe at position 436, according to EU numbering. In some embodiments, the Fc domain comprises a combination of amino acid substitutions selected from the group consisting of:
(a) N434A/Q438R/S440E;
(b) N434A/Q438R/S440D;
(c) N434A/Q438K/S440E;
(d) N434A/Q438K/S440D;
(e) N434A/Y436T/Q438R/S440E;
(f) N434A/Y436T/Q438R/S440D;
(g) N434A/Y436T/Q438K/S440E;
(h) N434A/Y436T/Q438K/S440D;
(i) N434A/Y436V/Q438R/S440E;
(j) N434A/Y436V/Q438R/S440D;
(k) N434A/Y436V/Q438K/S440E;
(l) N434A/Y436V/Q438K/S440D;
(m) N434A/R435H/F436T/Q438R/S440E;
(n) N434A/R435H/F436T/Q438R/S440D;
(o) N434A/R435H/F436T/Q438K/S440E;
(p) N434A/R435H/F436T/Q438K/S440D;
(q) N434A/R435H/F436V/Q438R/S440E;
(r) N434A/R435H/F436V/Q438R/S440D;
(s) N434A/R435H/F436V/Q438K/S440E;
(t) N434A/R435H/F436V/Q438K/S440D;
(u) M428L/N434A/Q438R/S440E;
(v) M428L/N434A/Q438R/S440D;
(w) M428L/N434A/Q438K/S440E;
(x) M428L/N434A/Q438K/S440D;
(y) M428L/N434A/Y436T/Q438R/S440E;
(z) M428L/N434A/Y436T/Q438R/S440D;
(aa) M428L/N434A/Y436T/Q438K/S440E;
(ab) M428L/N434A/Y436T/Q438K/S440D;
(ac) M428L/N434A/Y436V/Q438R/S440E;
(ad) M428L/N434A/Y436V/Q438R/S440D;
(ae) M428L/N434A/Y436V/Q438K/S440E;
(af) M428L/N434A/Y436V/Q438K/S440D;
(ag) L235R/G236R/S239K/M428L/N434A/Y436T/Q438R/S440E; and
(ah) L235R/G236R/A327G/A330S/P331S/M428L/N434A/Y436T/Q438R/S440E, according to EU numbering.

In some embodiments, the Fc domain of the multispecific antigen binding molecule comprises a combination of amino acid substitutions of M428L/N434A/Q438R/S440E. In some embodiments, the Fc domain is a IgG Fc domain, preferably a human IgG Fc domain, more preferably a human IgG1 Fc domain. In certain embodiments, the Fc domain of the multispecific antigen binding molecule comprises any of: (a) a first Fc subunit comprising the amino acid sequence shown in SEQ ID NO: 100 and a second Fc subunit comprising the amino acid sequence shown in SEQ ID NO: 111; and (b) a first Fc subunit comprising the amino acid sequence shown in SEQ ID NO: 99 and a second Fc subunit comprising the amino acid sequence shown in SEQ ID NO: 109.

Fc Region with a Reduced Fc Gamma Receptor-Binding Activity

Herein, “a reduced Fc gamma receptor-binding activity” means, for example, that based on the above-described analysis method the competitive activity of a test antigen-binding molecule or antibody is 50% or less, preferably 45% or less, 40% or less, 35% or less, 30% or less, 20% or less, or 15% or less, and particularly preferably 10% or less, 9% or less, 8% or less, 7% or less, 6% or less, 5% or less, 4% or less, 3% or less, 2% or less, or 1% or less than the competitive activity of a control antigen-binding molecule or antibody.

Antigen-binding molecules or antibodies comprising the Fc domain of a monoclonal IgG1, IgG2, IgG3, or IgG4 antibody can be appropriately used as control antigen-binding molecules or antibodies. The Fc domain structures are shown in SEQ ID NOs: 85 (A is added to the N terminus of RefSeq accession number AAC82527.1), 86 (A is added to the N terminus of RefSeq accession number AAB59393.1), 87 (A is added to the N terminus of RefSeq accession number CAA27268.1), and 88 (A is added to the N terminus of RefSeq accession number AAB59394.1). Furthermore, when an antigen-binding molecule or antibody comprising an Fc domain mutant of an antibody of a particular isotype is used as a test substance, the effect of the mutation of the mutant on the Fc gamma receptor-binding activity is assessed using as a control an antigen-binding molecule or antibody comprising an Fc domain of the same isotype. As described above, antigen-binding molecules or antibodies comprising an Fc domain mutant whose Fc gamma receptor-binding activity has been judged to be reduced are appropriately prepared.

Such known mutants include, for example, mutants having a deletion of amino acids 231A-238S (EU numbering) (WO 2009/011941), as well as mutants C226S, C229S, P238S, (C220S) (J. Rheumatol (2007) 34, 11); C226S and C229S (Hum. Antibod. Hybridomas (1990) 1(1), 47-54); C226S, C229S, E233P, L234V, and L235A (Blood (2007) 109, 1185-1192).

Specifically, the preferred antigen-binding molecules or antibodies include those comprising an Fc domain with a mutation (such as substitution) of at least one amino acid selected from the following amino acid positions: 220, 226, 229, 231, 232, 233, 234, 235, 236, 237, 238, 239, 240, 264, 265, 266, 267, 269, 270, 295, 296, 297, 298, 299, 300, 325, 327, 328, 329, 330, 331, or 332 (EU numbering), in the amino acids forming the Fc domain of an antibody of a particular isotype. The isotype of antibody from which the Fc domain originates is not particularly limited, and it is possible to use an appropriate Fc domain derived from a monoclonal IgG1, IgG2, IgG3, or IgG4 antibody. It is preferable to use Fe domains derived from IgG1 antibodies.

The preferred antigen-binding molecules or antibodies include, for example, those comprising an Fc domain which has any one of the substitutions shown below, whose positions are specified according to EU numbering (each number represents the position of an amino acid residue in the EU numbering; and the one-letter amino acid symbol before the number represents the amino acid residue before substitution, while the one-letter amino acid symbol after the number represents the amino acid residue after the substitution) in the amino acids forming the Fc domain of IgG1 antibody:
(a) L234F, L235E, P331S;
(b) C226S, C229S, P238S;
(c) C226S, C229S; or
(d) C226S, C229S, E233P, L234V, L235A;
as well as those having an Fc domain which has a deletion of the amino acid sequence at positions 231 to 238.

Furthermore, the preferred antigen-binding molecules or antibodies also include those comprising an Fc domain that has any one of the substitutions shown below, whose positions are specified according to EU numbering in the amino acids forming the Fc domain of an IgG2 antibody:
(e) H268Q, V309L, A330S, and P331S;
(f) V234A;
(g) G237A;
(h) V234A and G237A;
(i) A235E and G237A; or
(j) V234A, A235E, and G237A. Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG3 antibody:
(k) F241A;
(l) D265A; or
(m) V264A. Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG4 antibody:
(n) L235A, G237A, and E318A;
(o) L235E; or
(p) F234A and L235A. Each number represents the position of an amino acid residue in EU numbering; and the one-letter amino acid symbol before the number represents the amino acid residue before substitution, while the one-letter amino acid symbol after the number represents the amino acid residue after the substitution.

The other preferred antigen-binding molecules or antibodies include, for example, those comprising an Fc domain in which any amino acid at position 233, 234, 235, 236, 237, 327, 330, or 331 (EU numbering) in the amino acids forming the Fc domain of an IgG1 antibody is substituted with an amino acid of the corresponding position in EU numbering in the corresponding IgG2 or IgG4.

The preferred antigen-binding molecules or antibodies also include, for example, those comprising an Fc domain in which any one or more of the amino acids at positions 234, 235, and 297 (EU numbering) in the amino acids forming the Fc domain of an IgG1 antibody is substituted with other amino acids. The type of amino acid after substitution is not particularly limited; however, the antigen-binding molecules or antibodies comprising an Fc domain in which any one or more of the amino acids at positions 234, 235, and 297 are substituted with alanine are particularly preferred.

The preferred antigen-binding molecules or antibodies also include, for example, those comprising an Fc domain in which an amino acid at position 265 (EU numbering) in the amino acids forming the Fc domain of an IgG1 antibody is substituted with another amino acid. The type of amino acid after substitution is not particularly limited; however, antigen-binding molecules or antibodies comprising an Fc domain in which an amino acid at position 265 is substituted with alanine are particularly preferred.

### Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RITA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

### Fc Gamma Receptor

Fc gamma receptor refers to a receptor capable of binding to the Fc domain of monoclonal IgG1, IgG2, IgG3, or IgG4 antibodies, and includes all members belonging to the family of proteins substantially encoded by an Fc gamma receptor gene. In human, the family includes Fc gamma RI (CD64) including isoforms Fc gamma RIa, Fc gamma RIb and Fc gamma RIc; Fc gamma RII (CD32) including isoforms Fc gamma RIIa (including allotype H131 and R131), Fc gamma RIIb (including Fc gamma RIIb-1 and Fc gamma RIIb-2), and Fc gamma RIIc; and Fc gamma RIII (CD16) including isoform Fc gamma RIIIa (including allotype V158 and F158) and Fc gamma RIIIb (including allotype Fc gamma RIIIb-NA1 and Fc gamma RIIIb-NA2); as well as all unidentified human Fc gamma receptors, Fc gamma receptor isoforms, and allotypes thereof. However, Fc gamma receptor is not limited to these examples. Without being limited thereto, Fc gamma receptor includes those derived from humans, mice, rats, rabbits, and monkeys. Fc gamma receptor may be derived from any organisms. Mouse Fc gamma receptor includes, without being limited to, Fc gamma RI (CD64), Fc gamma RII (CD32), Fc gamma RIII (CD16), and Fc gamma RIII-2 (CD16-2), as well as all unidentified mouse Fc gamma receptors, Fc gamma receptor isoforms, and allotypes thereof. Such preferred Fc gamma receptors include, for example, human Fe gamma RI (CD64), Fe gamma RIIA (CD32), Fe gamma RIIB (CD32), Fe gamma RIIIA (CD16), and/or Fe gamma RIIIB (CD16). The polynucleotide sequence and amino acid sequence of Fe gamma RI are shown in RefSeq accession number NM_000566.3 and RefSeq accession number NP_000557.1, respectively; the polynucleotide sequence and amino acid sequence of Fe gamma RIIA are shown in RefSeq accession number BC020823.1 and RefSeq accession number AAH20823.1, respectively; the polynucleotide sequence and amino acid sequence of Fe gamma RIIB are shown in RefSeq accession number BC146678.1 and RefSeq accession number AAI46679.1, respectively; the polynucleotide sequence and amino acid sequence of Fe gamma RIIIA are shown in RefSeq accession number BC033678.1 and RefSeq accession number AAH33678.1, respectively; and the polynucleotide sequence and amino acid sequence of Fe gamma RIIIB are shown in RefSeq accession number BC128562.1 and RefSeq accession number AAI28563.1, respectively. Whether an Fe gamma receptor has binding activity to the Fc domain of a monoclonal IgG1, IgG2, IgG3, or IgG4 antibody can be assessed by ALPHA screen (Amplified Luminescent Proximity Homogeneous Assay), surface plasmon resonance (SPR)-based BIACORE method, and others (Proc. Natl. Acad. Sci. USA (2006) 103(11), 4005-4010), in addition to the above-described FACS and ELISA formats.

Meanwhile, “Fc ligand” or “effector ligand” refers to a molecule and preferably a polypeptide that binds to an antibody Fc domain, forming an Fc/Fc ligand complex. The molecule may be derived from any organisms. The binding of an Fc ligand to Fc preferably induces one or more effector functions. Such Fc ligands include, but are not limited to, Fc receptors, Fe gamma receptor, Fe alpha receptor, Fc beta receptor, FcRn, C1q, and C3, mannan-binding lectin, mannose receptor, Staphylococcus Protein A, Staphylococcus Protein G, and viral Fe gamma receptors. The Fc ligands also include Fc receptor homologs (FcRH) (Davis et al., (2002) Immunological Reviews 190, 123-136), which are a family of Fc receptors homologous to Fe gamma receptor. The Fc ligands also include unidentified molecules that bind to Fc.

### Fc Gamma Receptor-Binding Activity

The impaired binding activity of Fc domain to any of the Fe gamma receptors Fe gamma RI, Fe gamma RIIA, Fe gamma RIIB, Fe gamma RIIIA, and/or Fe gamma RIIIB can be assessed by using the above-described FACS and ELISA formats as well as ALPHA screen (Amplified Luminescent Proximity Homogeneous Assay) and surface plasmon resonance (SPR)-based BIACORE method (Proc. Natl. Acad. Sci. USA (2006) 103(11), 4005-4010).

ALPHA screen is performed by the ALPHA technology based on the principle described below using two types of beads: donor and acceptor beads. A luminescent signal is detected only when molecules linked to the donor beads interact biologically with molecules linked to the acceptor beads and when the two beads are located in close proximity. Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled antigen-binding molecule or antibody is immobilized to the donor beads and glutathione S-transferase (GST)-tagged Fc gamma receptor is immobilized to the acceptor beads. In the absence of an antigen-binding molecule or antibody comprising a competitive mutant Fc domain, Fc gamma receptor interacts with an antigen-binding molecule or antibody comprising a wild-type Fc domain, inducing a signal of 520 to 620 nm as a result. The antigen-binding molecule or antibody having a non-tagged mutant Fc domain competes with the antigen-binding molecule or antibody comprising a wild-type Fc domain for the interaction with Fc gamma receptor. The relative binding affinity can be determined by quantifying the reduction of fluorescence as a result of competition. Methods for biotinylating the antigen-binding molecules or antibodies such as antibodies using Sulfo-NHS-biotin or the like are known. Appropriate methods for adding the GST tag to an Fc gamma receptor include methods that involve fusing polypeptides encoding Fc gamma receptor and GST in-frame, expressing the fused gene using cells introduced with a vector carrying the gene, and then purifying using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto the gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the BIACORE methods. Examples of such inhibition assay are described in Proc. Natl. Acad. Sci. USA (2006) 103(11), 4005-4010.

### Production and Purification of Multispecific Antibodies

Multispecific antigen binding molecules described herein comprise two types of antigen binding moieties having different binding specificities (e.g. the “first antigen binding moiety” and the “second antigen binding moiety” both capable of binding to CD3 and CD137, and the “third antigen-binding moiety” capable of binding to a different antigen), each of which is eventually fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of multispecific antigen binding molecules in recombinant production, it will thus be advantageous to introduce in the Fc domain of the multispecific antigen binding molecule a modification promoting the association of the desired polypeptides.

Accordingly, in particular embodiments the Fc domain of the multispecific antigen binding molecule described herein comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

In a specific embodiment said modification is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the multispecific antigen binding molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A).

In yet a further embodiment, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter, J Immunol Methods 248, 7-15 (2001)).

In other embodiments, other techniques for promoting the association among H chains and between L and H chains having the desired combinations can be applied to the multispecific antigen-binding molecules of the present invention.

For example, techniques for suppressing undesired H-chain association by introducing electrostatic repulsion at the interface of the second constant region or the third constant region of the antibody H chain (CH2 or CH3) can be applied to multispecific antibody association (WO2006/106905).

In the technique of suppressing unintended H-chain association by introducing electrostatic repulsion at the interface of CH2 or CH3, examples of amino acid residues in contact at the interface of the other constant region of the H chain include regions corresponding to the residues at EU numbering positions 356, 439, 357, 370, 399, and 409 in the CH3 region.

More specifically, examples include an antibody comprising two types of H-chain CH3 regions, in which one to three pairs of amino acid residues in the first H-chain CH3 region, selected from the pairs of amino acid residues indicated in (1) to (3) below, carry the same type of charge: (1) amino acid residues comprised in the H chain CH3 region at EU numbering positions 356 and 439; (2) amino acid residues comprised in the H-chain CH3 region at EU numbering positions 357 and 370; and (3) amino acid residues comprised in the H-chain CH3 region at EU numbering positions 399 and 409.

Furthermore, the antibody may be an antibody in which pairs of the amino acid residues in the second H-chain CH3 region which is different from the first H-chain CH3 region mentioned above, are selected from the aforementioned pairs of amino acid residues of (1) to (3), wherein the one to three pairs of amino acid residues that correspond to the aforementioned pairs of amino acid residues of (1) to (3) carrying the same type of charges in the first H-chain CH3 region mentioned above carry opposite charges from the corresponding amino acid residues in the first H-chain CH3 region mentioned above.

Each of the amino acid residues indicated in (1) to (3) above come close to each other during association. Those skilled in the art can find out positions that correspond to the above-mentioned amino acid residues of (1) to (3) in a desired H-chain CH3 region or H-chain constant region by homology modeling and such using commercially available software, and amino acid residues of these positions can be appropriately subjected to modification.

In the antibodies mentioned above, “charged amino acid residues” are preferably selected, for example, from amino acid residues included in either one of the following groups:
(a) glutamic acid (E) and aspartic acid (D); and
(b) lysine (K), arginine (R), and histidine (H).

In the above-mentioned antibodies, the phrase “carrying the same charge” means, for example, that all of the two or more amino acid residues are selected from the amino acid residues included in either one of groups (a) and (b) mentioned above. The phrase “carrying opposite charges” means, for example, that when at least one of the amino acid residues among two or more amino acid residues is selected from the amino acid residues included in either one of groups (a) and (b) mentioned above, the remaining amino acid residues are selected from the amino acid residues included in the other group.

In a preferred embodiment, the antibodies mentioned above may have their first H-chain CH3 region and second H-chain CH3 region crosslinked by disulfide bonds.

In the present invention, amino acid residues subjected to modification are not limited to the above-mentioned amino acid residues of the antibody variable regions or the antibody constant regions. Those skilled in the art can identify the amino acid residues that form an interface in mutant polypeptides or heteromultimers by homology modeling and such using commercially available software; and amino acid residues of these positions can then be subjected to modification so as to regulate the association.

In addition, other known techniques can also be used for formation of multispecific antibodies of the present invention. Association of polypeptides having different sequences can be induced efficiently by complementary association of CH3 using a strand-exchange engineered domain CH3 produced by changing part of one of the H-chain CH3s of an antibody to a corresponding IgA-derived sequence and introducing a corresponding IgA-derived sequence into the complementary portion of the other H-chain CH3 (Protein Engineering Design & Selection, 23; 195-202, 2010). This known technique can also be used to efficiently form multispecific antibodies of interest.

In addition, technologies for antibody production using association of antibody CH1 and CL and association of VH and VL as described in WO 2011/028952, WO2014/018572, and Nat Biotechnol. 2014 February; 32(2):191-8; technologies for producing bispecific antibodies using separately prepared monoclonal antibodies in combination (Fab Arm Exchange) as described in WO2008/119353 and WO2011/131746; technologies for regulating association between antibody heavy-chain CH3s as described in WO2012/058768 and WO2013/063702; technologies for producing multispecific antibodies composed of two types of light chains and one type of heavy chain as described in WO2012/023053; technologies for producing multispecific antibodies using two bacterial cell strains that individually express one of the chains of an antibody comprising a single H chain and a single L chain as described by Christoph et al. (Nature Biotechnology Vol. 31, p 753-758 (2013)); and such may be used for the formation of multispecific antibodies.

Alternatively, even when a multispecific antibody of interest cannot be formed efficiently, a multispecific antibody of the present invention can be obtained by separating and purifying the multispecific antibody of interest from the produced antibodies. For example, a method for enabling purification of two types of homomeric forms and the heteromeric antibody of interest by ion-exchange chromatography by imparting a difference in isoelectric points by introducing amino acid substitutions into the variable regions of the two types of H chains has been reported (WO2007114325). To date, as a method for purifying heteromeric antibodies, methods using Protein A to purify a heterodimeric antibody comprising a mouse IgG2a H chain that binds to Protein A and a rat IgG2b H chain that does not bind to Protein A have been reported (WO98050431 and WO95033844). Furthermore, a heterodimeric antibody can be purified efficiently on its own by using H chains comprising substitution of amino acid residues at EU numbering positions 435 and 436, which is the TgG-Protein A binding site, with Tyr, His, or such which are amino acids that yield a different Protein A affinity, or using H chains with a different protein A affinity, to change the interaction of each of the H chains with Protein A, and then using a Protein A column.

Furthermore, an Fc region whose Fc region C-terminal heterogeneity has been improved can be appropriately used as an Fc region of the present invention. More specifically, the present invention provides Fc regions produced by deleting glycine at position 446 and lysine at position 447 as specified by EU numbering from the amino acid sequences of two polypeptides constituting an Fc region derived from IgG1, IgG2, IgG3, or IgG4.

Multispecific antigen binding molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the multispecific antigen binding molecule binds. For example, for affinity chromatography purification of multispecific antigen binding molecules of the invention, a matrix with protein A or protein G may be used. Sequential Protein A or G affinity chromatography and size exclusion chromatography can be used to isolate a multispecific antigen binding molecule. The purity of the multispecific antigen binding molecule can be determined by any of a variety of well known analytical methods including gel electrophoresis, high pressure liquid chromatography, and the like.

### Antibody-Dependent Cell-Mediated Cytotoxicity

“Antibody-dependent cell-mediated cytotoxicity” or “ADCC” refers to a form of cytotoxicity in which secreted Ig bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express Fc gamma RIII only, whereas monocytes express Fc gamma RI, Fc gamma RII, and Fc gamma RIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

### Complement Dependent Cytotoxicity

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

### T Cell Dependent Cellular Cytotoxicity

“T cell dependent cellular cytotoxicity” or “TDCC” refers to a form of cytotoxicity in which an antigen-binding molecule binds to both an antigen expressed on the target cell, and another antigen expressed on T cell, that redirect T cell near to the target cell, as cytotoxicity against the target cell is induced due to the T cell. The method to assess T cell dependent cellular cytotoxicity, an in vitro TDCC assay, is also described in the “Measurement of T cell dependent cellular cytotoxicity” section of this description.

### Measurement of T Cell Dependent Cellular Cytotoxicity

In the embodiment that the antigen-binding molecule binds to both DLL3 and CD3/CD137, the methods described below are preferably used as a method for assessing or determining T cell dependent cellular cytotoxicity (TDCC) caused by contacting an antigen-binding molecule of the present disclosure with DLL3-expressing cells to which the antigen-binding site in the antigen-binding molecules of the present disclosure binds. The methods for assessing or determining the cytotoxic activity in vitro include methods for determining the activity of cytotoxic T-cells or the like. Whether an antigen-binding molecule of the present disclosure has the activity of inducing T-cell mediated cellular cytotoxicity can be determined by known methods (see, for example, Current protocols in Immunology, Chapter 7. Immunologic studies in humans, Editor, John E, Coligan et al., John Wiley & Sons, Inc., (1993)). In the cytotoxicity assay, an antigen-binding molecule which is able to bind to an antigen different from DLL3 and which is not expressed in the cells, and CD3/CD137, is used as a control antigen-binding molecule. The control antigen-binding molecule is assayed in the same manner. Then, the activity is assessed by testing whether an antigen-binding molecule of the present disclosure exhibits a stronger cytotoxic activity than that of a control antigen-binding molecule.

Meanwhile, the in vivo anti-tumor efficacy is assessed or determined, for example, by the following procedure. Cells expressing the antigen to which the antigen-binding site in an antigen-binding molecule of the present disclosure binds are transplanted intracutaneously or subcutaneously to a nonhuman animal subject. Then, from the day of transplantation or thereafter, a test antigen-binding molecule is administered into vein or peritoneal cavity every day or at intervals of several days. The tumor size is measured over time. Difference in the change of tumor size can be defined as the cytotoxic activity. As in an in vitro assay, a control antigen-binding molecule is administered. The antigen-binding molecule of the present disclosure can be judged to have cytotoxic activity when the tumor size is smaller in the group administered with the antigen-binding molecule of the present disclosure than in the group administered with the control antigen-binding molecule.

An MTT method and measurement of isotope-labeled thymidine uptake into cells are preferably used to assess or determine the effect of contact with an antigen-binding molecule of the present disclosure to suppress the growth of cells expressing an antigen to which the antigen-binding site in the antigen-binding molecule binds. Meanwhile, the same methods described above for assessing or determining the in vivo cytotoxic activity can be used preferably to assess or determine the activity of suppressing cell growth in vivo.

The TDCC of an antibody or antigen-binding molecule of the disclosure can be evaluated by any suitable method known in the art. For example, TDCC can be measured by lactate dehydrogenase (LDH) release assay. In this assay, target cells (e.g. DLL3-expressing cells) are incubated with T cells (e.g. PBMCs) in the presence of a test antibody or antigen-binding molecule, and the activity of LDH that has been released from target cells killed by T cells is measured using a suitable reagent. Typically, the cytotoxic activity is calculated as a percentage of the LDH activity resulting from the incubation with the antibody or antigen-binding molecule relative to the LDH activity resulting from 100% killing of target cells (e.g. lysed by treatment with Triton-X). If the cytotoxic activity calculated as mentioned above is higher, the test antibody or antigen-binding molecule is determined to have higher TDCC.

Additionally or alternatively, for example, TDCC can also be measured by real-time cell growth inhibition assay. In this assay, target cells (e.g. DLL3-expressing cells) are incubated with T cells (e.g. PBMCs) in the presence of a test antibody or antigen-binding molecule on a 96-well plate, and the growth of the target cells is monitored by methods known in the art, for example, by using a suitable analyzing instrument (e.g. xCELLigence Real-Time Cell Analyzer). The rate of cell growth inhibition (CGI: %) is determined from the cell index value according to the formulation given as CGI (%)=100−(CIAb×100/CINoAb). “CIAb” represents the cell index value of wells with the antibody or antigen-binding molecule on a specific experimental time and “CINoAb” represents the average cell index value of wells without the antibody or antigen-binding molecule. If the CGI rate of the antibody or antigen-binding molecule is high, i.e., has a significantly positive value, it can be said that the antibody or antigen-binding molecule has TDCC activity.

In one aspect, an antibody or antigen-binding molecule of the disclosure has T cell activation activity. T cell activation can be assayed by methods known in the art, such as a method using an engineered T cell line that expresses a reporter gene (e.g. luciferase) in response to its activation (e.g. Jurkat/NFAT-RE Reporter Cell Line (T Cell Activation Bioassay, Promega)). In this method, target cells (e.g. DLL3-expressing cells) are cultured with T cells in the presence of a test antibody or antigen-binding molecule, and then the level or activity of the expression product of the reporter gene is measured by appropriate methods as an index of T cell activation. When the reporter gene is a luciferase gene, luminescence arising from reaction between luciferase and its substrate may be measured as an index of T cell activation. If T cell activation measured as described above is higher, the test antibody or antigen-binding molecule is determined to have higher T cell activation activity.

### Pharmaceutical Composition

In one aspect, the present disclosure provides a pharmaceutical composition or pharmaceutical formulation comprising the antigen-binding molecule or antibody of the disclosure. In certain embodiments, the pharmaceutical composition or pharmaceutical formulation of the disclosure induces T-cell-dependent cytotoxicity, in another word, the pharmaceutical composition or pharmaceutical formulation of the disclosure is a therapeutic agent for inducing cellular cytotoxicity. In certain embodiments, the pharmaceutical composition or pharmaceutical formulation of the disclosure is a pharmaceutical composition or pharmaceutical formulation for treatment and/or prevention of cancer. In certain embodiments, the pharmaceutical composition or pharmaceutical formulation of the disclosure is a pharmaceutical composition or pharmaceutical formulation used for treatment and/or prevention of DLL3-positive or DLL3-expressing cancers including lung cancer (including small cell lung cancer) and melanoma, or includes a neuroendocrine Neoplasm (NEN), a neuroendocrine tumor (NET) or a neuroendocrine carcinoma (NEC) that expresses DLL3, or other solid tumors of non-neuroendocrine origin that expresses DLL3. In some preferred embodiment, examples of cancer includes pancreatic neuroendocrine tumor (PNET), small-cell type NEC (SCNEC) or large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC); or other solid tumors such as neuroblastoma, glioma or glioblastoma (GBM), melanoma, medullary thyroid cancer. In a preferred embodiment, the tumor or cancer disease is lung cancer, preferably SCLC. In another preferred embodiment, the tumor or cancer disease is glioma or glioblastoma (GBM). In another preferred embodiment, the tumor or cancer disease is neuroendocrine prostate cancer. In certain embodiments, the pharmaceutical composition or pharmaceutical formulation of the disclosure is cell growth-suppressing agent. In certain embodiments, the pharmaceutical composition or pharmaceutical formulation of the disclosure is anticancer agent.

A pharmaceutical composition or pharmaceutical formulation of the present disclosure, a therapeutic agent for inducing cellular cytotoxicity, a cell growth-suppressing agent, or an anticancer agent of the present disclosure may be formulated with different types of antigen-binding molecules or antibodies, if needed. For example, the cytotoxic action against cells expressing an antigen can be enhanced by a cocktail of multiple antigen-binding molecules or antibodies of the disclosure.

Pharmaceutical compositions or pharmaceutical formulation of an antigen-binding molecule or antibody as described herein are prepared by mixing such antigen-binding molecule or antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

If necessary, the antigen-binding molecules or antibodies of the present disclosure may be encapsulated in microcapsules (microcapsules made from hydroxymethylcellulose, gelatin, poly[methylmethacrylate], and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for preparing agents as sustained-release agents are known, and these can be applied to the antigen-binding molecules of the present disclosure (J. Biomed. Mater. Res. (1981) 15, 267-277; Chemtech. (1982) 12, 98-105; U.S. Pat. No. 3,773,719; European Patent Application (EP) Nos. EP58481 and EP133988; Biopolymers (1983) 22, 547-556).

The pharmaceutical compositions, cell growth-suppressing agents, or anticancer agents of the present disclosure may be administered either orally or parenterally to patients. Parental administration is preferred. Specifically, such administration methods include injection, nasal administration, transpulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present disclosure can be administered locally or systemically by injection. Furthermore, appropriate administration methods can be selected according to the patient's age and symptoms. The administered dose can be selected, for example, from the range of 0.0001 mg to 1,000 mg per kg of body weight for each administration. Alternatively, the dose can be selected, for example, from the range of 0.001 mg/body to 100,000 mg/body per patient. However, the dose of a pharmaceutical composition of the present disclosure is not limited to these doses.

Preferably, a pharmaceutical composition or pharmaceutical formulations of the present disclosure comprises an antigen-binding molecule or antibody as described herein. In one aspect, the composition is a pharmaceutical composition for use in inducing cellular cytotoxicity. In another aspect, the composition is a pharmaceutical composition for use in treating or preventing cancer. Preferably, the cancer is lung cancer (including small cell lung cancer) and melanoma or includes a neuroendocrine Neoplasm (NEN), a neuroendocrine tumor (NET) or a neuroendocrine carcinoma (NEC) that expresses DLL3, or other solid tumors of non-neuroendocrine origin that expresses DLL3. In some preferred embodiment, examples of cancer includes pancreatic neuroendocrine tumor (PNET), small-cell type NEC (SCNEC) or large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC); or other solid tumors such as neuroblastoma, glioma or glioblastoma (GBM), melanoma, medullary thyroid cancer. In a preferred embodiment, the tumor or cancer disease is lung cancer, preferably SCLC. In another preferred embodiment, the tumor or cancer disease is glioma or glioblastoma (GBM). In another preferred embodiment, the tumor or cancer disease is neuroendocrine prostate cancer. The pharmaceutical composition of the present disclosure can be used for treating or preventing cancer. Thus, the present disclosure provides a method for treating or preventing cancer, in which the antigen-binding molecule or antibody as described herein is administered to a patient in need thereof.

The present disclosure also provides methods for damaging cells expressing DLL3 or for suppressing the cell growth by contacting the cells expressing DLL3 with an antigen-binding molecule of the present disclosure that binds to DLL3. Cells to which an antigen-binding molecule of the present disclosure binds are not particularly limited, as long as they express DLL3. Specifically, in the present disclosure, the preferred DLL3-expressing cells include lung cancer (including small cell lung cancer) and melanoma, or includes a neuroendocrine Neoplasm (NEN), a neuroendocrine tumor (NET) or a neuroendocrine carcinoma (NEC) that expresses DLL3, or other solid tumors of non-neuroendocrine origin that expresses DLL3. In some preferred embodiment, examples of cancer includes pancreatic neuroendocrine tumor (PNET), small-cell type NEC (SCNEC) or large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC); or other solid tumors such as neuroblastoma, glioma or glioblastoma (GBM), melanoma, medullary thyroid cancer. In a preferred embodiment, the tumor or cancer disease is lung cancer, preferably SCLC. In another preferred embodiment, the tumor or cancer disease is glioma or glioblastoma (GBM). In another preferred embodiment, the tumor or cancer disease is neuroendocrine prostate cancer.

In the present disclosure, “contact” can be carried out, for example, by adding an antigen-binding molecule of the present disclosure to culture media of cells expressing DLL3 cultured in vitro. In this case, an antigen-binding molecule to be added can be used in an appropriate form, such as a solution or solid prepared by lyophilization or the like. When the antigen-binding molecule of the present disclosure is added as an aqueous solution, the solution may be a pure aqueous solution containing the antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The added concentration is not particularly limited; however, the final concentration in a culture medium is preferably in a range of 1 pg/ml to 1 g/ml, more preferably 1 ng/ml to 1 mg/ml, and still more preferably 1 micro g/ml to 1 mg/ml.

In another embodiment of the present disclosure, “contact” can also be carried out by administration to nonhuman animals transplanted with DLL3-expressing cells in vivo or to animals having cancer cells expressing DLL3 endogenously. The administration method may be oral or parenteral. Parenteral administration is particularly preferred. Specifically, the parenteral administration method includes injection, nasal administration, pulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present disclosure can be administered locally or systemically by injection. Furthermore, an appropriate administration method can be selected according to the age and symptoms of an animal subject. When the antigen-binding molecule is administered as an aqueous solution, the solution may be a pure aqueous solution containing the antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The administered dose can be selected, for example, from the range of 0.0001 to 1,000 mg per kg of body weight for each administration. Alternatively, the dose can be selected, for example, from the range of 0.001 to 100,000 mg/body for each patient. However, the dose of an antigen-binding molecule of the present disclosure is not limited to these examples.

The present disclosure also provides kits for use in a method of the present disclosure, which contain an antigen-binding molecule of the present disclosure or an antigen-binding molecule produced by a method of the present disclosure. The kits may be packaged with an additional pharmaceutically acceptable carrier or medium, or instruction manual describing how to use the kits, etc.

In another aspect of the invention, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Package Insert

The term “package insert” is used to refer to instructions customarily included in commercial packages of therapeutic products, that contain information about the in-dications, usage, dosage, administration, combination therapy, contraindications and/or warnings concerning the use of such therapeutic products.

### Pharmaceutical Formulation

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

### Pharmaceutically Acceptable Carrier

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

### Treatment

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antigen-binding molecules or antibodies of the present disclosure are used to delay development of a disease or to slow the progression of a disease.

### Cancer

The terms “cancer” and “cancerous” refer to or describe the physiological condition in mammals that is typically characterized by unregulated cell growth/proliferation.

In certain embodiments, the cancer is a DLL3-expressing or DLL3-positive cancer which includes a neuroendocrine Neoplasm (NEN), a neuroendocrine tumor (NET) or a neuroendocrine carcinoma (NEC) that expresses DLL3, or other solid tumors of non-neuroendocrine origin that expresses DLL3. In some embodiments, examples of cancer includes pancreatic neuroendocrine tumor (PNET), small-cell type NEC (SCNEC) or large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC); or other solid tumors such as neuroblastoma, glioma or glioblastoma (GBM), melanoma, medullary thyroid cancer. In a preferred embodiment of the invention, the tumor or cancer disease is lung cancer, preferably SCLC. In another preferred embodiment of the invention, the tumor or cancer disease is glioma or glioblastoma (GBM). In another preferred embodiment of the invention, the tumor or cancer disease is neuroendocrine prostate cancer.

### Tumor

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

### Other Agents and Treatments

The multispecific antigen binding molecules described herein may be administered in combination with one or more other agents in therapy. For instance, a multispecific antigen binding molecules as described herein may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunotherapeutic agent, a cytostatic agent, an inhibitor of cell adhesion, a cytotoxic agent, an activator of cell apoptosis, or an agent that increases the sensitivity of cells to apoptotic inducers.

In certain embodiments of the above embodiments, the additional therapeutic agent is an immunotherapeutic agent. In some embodiments, the immunotherapeutic agent is an agent that modulates an immune response. In some embodiments, the immunotherapeutic agent is an agent that enhances an anti-tumor immune response. In some embodiments, the immunotherapeutic agent is an agent that increases cell-mediated immunity. In some embodiments, the immunotherapeutic agent is an agent that increases T cell activity. In some embodiments, the immunotherapeutic agent is an agent that increases cytolytic T Cell (CTL) activity. In some embodiments, the immunotherapeutic agent is an agent that increases NK cell activity. In some embodiments, the immunotherapeutic agent is an agent that suppresses suppression of an immune response. In some embodiments, the immunotherapeutic agent is an agent that inhibits a suppressor cell or suppresses the activity of a cell. In some embodiments, the immunotherapeutic agent is an agent that inhibits Treg activity. In some embodiments, the immunotherapeutic agent is an agent that inhibits MDSC activity. In some embodiments, the immunotherapeutic agent is an agent that inhibits the activity of an inhibitory immune checkpoint receptor. In some embodiments, the immunotherapeutic agent is an agent that inhibits the activity of PD-1. In some embodiments, the immunotherapeutic agent is an agent that inhibits the activity of PD-L1 and/or PD-L2. In some embodiments, the immunotherapeutic agent is an agent that inhibits the activity of CTLA-4. In some embodiments, the immunotherapeutic agent is an agent that inhibits the activity of CD80 and/or CD 86. In some embodiments, the immunotherapeutic agent is an agent that inhibits the activity of TIGIT. In some embodiments, the immunotherapeutic agent is an agent that inhibits the activity of KIR. In some embodiments, the immunotherapeutic agent is an agent that inhibits the activity of IDO 1. In some embodiments, the immunotherapeutic agent is an agent that enhances or stimulates the activity of activating an immune checkpoint receptor. In some embodiments, the immunotherapeutic agent is an agent that enhances or stimulates the activity of GITR. In some embodiments, the immunotherapeutic agent is an agent that enhances or stimulates the activity of OX 40. In some embodiments, the immunotherapeutic agent is an agent that enhances or stimulates the activity of CD 40.

In some embodiments described herein, the immunotherapeutic agent is a PD-1 antagonist, a PD-L1 antagonist, a PD-L2 antagonist, a CTLA-4 antagonist, a CD80 antagonist, a CD86 antagonist, a TIGIT antagonist, a KIR antagonist, a Tim-3 antagonist, a LAG3 antagonist, a CD96 antagonist, a CD20 antagonist, or an IDO1 antagonist. In some embodiments, the PD-1 antagonist is an antibody that specifically binds to PD-1. In some embodiments, the antibody that binds PD-1 is pembrolizumab, pidilizumab (pidilizumab) (CT-011), or nimotuzumab (nivolumab). In some embodiments, the PD-L1 antagonist is an antibody that specifically binds to PD-L1. In some embodiments, the antibody that binds PD-L1 is atezolizumab, durvalumab (MEDI4736), or BMS-936559. In some embodiments, the CTLA-4 antagonist is an antibody that specifically binds CTLA-4. In some embodiments, the antibody that binds to CTLA-4 is ipilimumab or tremelimumab (tremelimumab) (CP-675,206). In some embodiments, the KIR antagonist is an antibody that specifically binds KIR. In some embodiments, the antibody that binds KIR is rituximab (lirilumab). In some embodiments of the methods described herein, the immunotherapeutic agent is a CD28 agonist, a 4-1BB agonist, an OX40 agonist, a CD27 agonist, a CD80 agonist, a CD86 agonist, a CD40 agonist, or a GITR agonist.

The multispecific antigen binding molecules described herein may be administered in combination with steroid or other antibody pharmaceuticals such as tocilizumab, where appropriate or necessary, to control cytokine release syndrome (CRS), a common adverse event of T cell-associated therapeutics such as T cell engagers and CAR-T cells.

### Chemotherapeutic Agent

In certain embodiments of the above embodiments, the additional therapeutic agent is a chemotherapeutic agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. A “chemotherapeutic agent” refers to a chemical compound useful in the treatment of cancer. Examples of chemotherapeutic agents include alkylating agents such as thiotepa and cyclophosphamide (CYTOXAN (registered trademark)); alkyl sulfonates such as busulfan, improsulfan and piposulfan; aziridines such as benzodopa, carboquone, meturedopa, and uredopa; ethylenimines and methylol melamines including altretamine, triethylenemelamine, triethylenephosphoramide, tri-ethylenethiophosphoramide and trimethylomelamine; acetogenins (especially bullatacin and bullatacinone); delta-9-tetrahydrocannabinol (dronabinol, MARINOL (registered trademark)); beta-lapachone; lapachol; colchicines; betulinic acid; a camptothecin (including the synthetic analogue topotecan (HYCAMTIN (registered trademark)), CPT-11 (irinotecan, CAMPTOSAR (registered trademark)), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, anthramycin, azaserine, bleomycins, cactinomycin, carubicin, carminomycin, carzinophilin, chromomycins, dactinomycin, daunorubicin, detorubicin, 6-diazo-5-oxo-L-norleucine, doxorubicin (including ADRIAMYCIN (registered trademark), morpholino-doxorubicin, cyanomorpholino-doxorubicin, 2-pyrrolino-doxorubicin, doxorubicin HCl liposome injection (DOXIL (registered trademark)), liposomal doxorubicin TLC D-99 (MYOCET (registered trademark)), peglylated liposomal doxorubicin (CAELYX (registered trademark)), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR (registered trademark)), tegafur (UFTORAL (registered trademark)), capecitabine (XELODA (registered trademark)), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatrexate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitraerine; pentostatin; phenamet; pirarubicin; losoxantrone; 2-ethylhydrazide; procarbazine; PSK (registered trademark) polysaccharide complex (JHS Natural Products, Eugene, OR); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE (registered trademark), FILDESIN (registered trademark)); dacarbazine; mannomustine; mitobronitol; mitolactol; pipobroman; gacytosine; arabinoside (“Ara-C”); thiotepa; taxoid, e.g., paclitaxel (TAXOL (registered trademark)), albumin-engineered nanoparticle formulation of paclitaxel (ABRAXANE™), and docetaxel (TAXOTERE (registered trademark)); chlorambucil; 6-thioguanine; mercaptopurine; methotrexate; platinum agents such as cisplatin, oxaliplatin (e.g., ELOXATIN (registered trademark)), and carboplatin; vincas, which prevent tubulin polymerization from forming microtubules, including vinblastine (VELBAN (registered trademark)), vincristine (ONCOVIN (registered trademark)), vindesine (ELDISINE (registered trademark), FILDESIN (registered trademark)), and vinorelbine (NAVELBINE (registered trademark)); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN (registered trademark)); bisphosphonates such as clodronate (for example, BONEFOS (registered trademark) or OSTAC (registered trademark)), etidronate (DIDROCAL (registered trademark)), NE-58095, zoledronic acid/zoledronate (ZOMETA (registered trademark)), alendronate (FOSAMAX (registered trademark)), pamidronate (AREDIA (registered trademark)), tiludronate (SKELID (registered trademark)), or risedronate (ACTONEL (registered trademark)); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE (registered trademark) vaccine and gene therapy vaccines, for example, ALLOVECTIN (registered trademark) vaccine, LEUVECTIN (registered trademark) vaccine, and VAXID (registered trademark) vaccine; topoisomerase 1 inhibitor (e.g., LURTOTECAN (registered trademark)); rmRH (e.g., ABARELIX (registered trademark)); BAY439006 (sorafenib; Bayer); SU-11248 (sunitinib, SUTENT (registered trademark), Pfizer); perifosine, COX-2 inhibitor (e.g. celecoxib or etoricoxib), proteosome inhibitor (e.g. PS341); bortezomib (VELCADE (registered trademark)); CCI-779; tipifarnib (R11577); sorafenib, ABT510; Bcl-2 inhibitor such as oblimersen sodium (GENASENSE (registered trademark)); pixantrone; EGFR inhibitors (see definition below); tyrosine kinase inhibitors (see definition below); serine-threonine kinase inhibitors such as rapamycin (sirolimus, RAPAMUNE (registered trademark)); farnesyltransferase inhibitors such as lonafarnib (SCH 6636, SARASAR™); Lurbinectedin or Amrubicin; and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein may also include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX (registered trademark)), 4-hydroxytamoxifen, toremifene (FARESTON (registered trademark)), idoxifene, droloxifene, raloxifene (EVISTA (registered trademark)), trioxifene, keoxifene, and selective estrogen receptor modulators (SERMs) such as SERM3; pure anti-estrogens without agonist properties, such as fulvestrant (FASLODEX (registered trademark)), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN (registered trademark)), and nonsteroidal aromatase inhibitors such as anastrazole (ARIMIDEX (registered trademark)), letrozole (FEMARA (registered trademark)) and aminoglutethimide, and other aromatase inhibitors include vorozole (RIVISOR (registered trademark)), megestrol acetate (MEGASE (registered trademark)), fadrozole, and 4(5)-imidazoles; lutenizing hormone-releasing hormone agonists, including leuprolide (LUPRON (registered trademark) and ELIGARD (registered trademark)), goserelin, buserelin, and triptorelin; sex steroids, including progestins such as megestrol acetate and medroxyprogesterone acetate, estrogens such as diethylstilbestrol and premarin, and androgens/retinoids such as fluoxymesterone, all transretinoic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

Such other agents are suitably present in combination in amounts that are effective for the purpose intended. The effective amount of such other agents depends on the amount of multispecific antigen binding molecules used, the type of disorder or treatment, and other factors discussed above. The multispecific antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the multispecific antigen binding molecules described herein can occur prior to, simultaneously, and/or following, administration of the additional therapeutic agent and/or adjuvant. Multispecific antigen binding molecules as described herein can also be used in combination with radiation therapy.

All documents cited herein are incorporated herein by reference.

The following are examples of methods and compositions of the present disclosure. It is understood that various other embodiments may be practiced, given the general description provided above.

### EXAMPLES

### Example 1

### Affinity Matured Variant Screening of Parental Dual-Fab H183L072 for Improvement in In Vitro Cytotoxicity on Tumor Cells

### 1.1 Sequence of Affinity Matured Variants

Concept of providing an immunoglobulin variable (Fab) region that binds CD3 and CD137, but does not bind to CD3 and CD137 at same time (Dual-Fab) is disclosed in WO2019111871 (incorporated herein by reference). To increase the binding affinity of parental Dual-Fab H183L072 (Heavy chain: SEQ ID NO: 1; Light chain: SEQ ID NO: 57) disclosed in WO2019111871, more than 1,000 Dual-Fab variants were generated using H183L072 as a template to introduce single or multiple mutations on a variable region. Antibodies were expressed using Expi293 (Invitrogen) and purified by Protein A purification followed by gel filtration, when gel filtration was necessary. The sequences of 22 representative Dual-Fab variants with multiple mutations are listed in Table 6 and Tables 8-1 to 8-6 and binding affinity and kinetics towards CD3 and CD137 were evaluated at 25 degrees C. and/or 37 degrees C. using Biacore T200 instrument (GE Healthcare) as described below in Example 1.2.2 (Table 9).

### 1.2. Binding Kinetics Information of Affinity Matured Variants

### 1.2.1 Expression and Purification of Human CD3 and CD137

The gamma and epsilon subunits of the human CD3 complex (human CD3eg linker) were linked by a 29-mer linker and a Flag-tag was fused to the C-terminal end of the gamma subunit (SEQ ID NO: 102, Table 5 and 7). This construct was expressed transiently using FreeStyle293F cell line (Thermo Fisher). Conditioned media expressing human CD3eg linker was concentrated using a column packed with Q HP resins (GE healthcare) then applied to FLAG-tag affinity chromatography. Fractions containing human CD3eg linker were collected and subsequently subjected to a Superdex 200 gel filtration column (GE healthcare) equilibrated with 1×D-PBS. Fractions containing human CD3eg linker were then pooled. Human CD137 extracellular domain (ECD) (SEQ ID NO: 103, Table 5 and 7) with hexahistidine (His-tag) and biotin acceptor peptide (BAP) on its C-terminus was expressed transiently using FreeStyle293F cell line (Thermo Fisher). Conditioned media expressing human CD137 ECD was applied to a HisTrap HP column (GE healthcare) and eluted with buffer containing imidazole (Nacalai). Fractions containing human CD137 ECD were collected and subsequently subjected to a Superdex 200 gel filtration column (GE healthcare) equilibrated with 1×D-PBS. Fractions containing human CD137 ECD were then pooled and stored at −80 degrees C.

### 1.2.2 Affinity Measurement Towards Human CD3 and CD137

Binding affinity of Dual-Fab antibodies (Dual-Ig) to human CD3 were assessed at 25 degrees C. using Biacore 8K instrument (GE Healthcare). Anti-human Fc (GE Healthcare) was immobilized onto all flow cells of a CM4 sensor chip using amine coupling kit (GE Healthcare). Antibodies were captured onto the anti-Fc sensor surfaces, then recombinant human CD3 or CD137 was injected over the flow cell. All antibodies and analytes were prepared in ACES pH 7.4 containing 20 mM ACES, 150 mM NaCl, 0.05% Tween 20, 0.005% NaN₃. Sensor surface was regenerated each cycle with 3M MgCl₂. Binding affinity were determined by processing and fitting the data to 1:1 binding model using Biacore Insight Evaluation software, version 2.0 (GE Healthcare). CD137 binding affinity assay was conducted in the same conditions except that assay temperature was set at 37 degrees C. Binding affinity of Dual-Fab antibodies to recombinant human CD3 and CD137 are shown in Tables 9-1 and 9-2 (the expression E used to express the Kₒₙ, K_{off}, and KD values in the table means “10 to the power of” and, for instance, 3.54E+04=3.54*10⁴). As illustrated in Tables 9-1 and 9-2, the Dual Fab variants showed different binding kinetics towards CD3 and CD137 as compared H183/L072.

TABLE 5
Annotation of SEQ ID NOs for Table 7 (SEQ ID NOs for human CD3
and CD137 antigen used for affinity measurement in Table 9)

| Antigen name | SEQ List |
|---|---|
| Human CD3eg linker | 102 |
| Human CD137 ECD | 103 |

TABLE 6
Annotation of SEQ ID NOs for and Tables 8-1 to 8-6 (antibody naming
and SEQ ID NOs for variable region including CDR 1, 2, and 3)

| | | | | | VHR_{—} | VHR_{—} | VHR_{—} | | VLR_{—} | VLR_{—} | VLR_{—} |
|---|---|---|---|---|---|---|---|---|---|---|---|
| DualAE No. | Ab name | VHR name | VLR name | VHR | CDR1 | CDR2 | CDR3 | VLR | CDR1 | CDR2 | CDR3 |
| Parent | H183/L072 | dBBDu183H | dBBDu072L | 1 | 15 | 29 | 43 | 57 | 62 | 67 | 72 |
| DualAE01 | H0868L0581 | dBBDu183H0868 | dBBDu072L0581 | 2 | 16 | 30 | 44 | 58 | 63 | 68 | 73 |
| DualAE08 | H1550L0918 | dBBDu183H1550 | dBBDu072L0918 | 3 | 17 | 31 | 45 | 59 | 64 | 69 | 74 |
| DualAE06 | H1571L0581 | dBBDu183H1571 | dBBDu072L0581 | 4 | 18 | 32 | 46 | 58 | 63 | 68 | 73 |
| DualAE17 | H1610L0581 | dBBDu183H1610 | dBBDu072L0581 | 5 | 19 | 33 | 47 | 58 | 63 | 68 | 73 |
| DualAE10 | H1610L0939 | dBBDu183H1610 | dBBDu072L0939 | 5 | 19 | 33 | 47 | 60 | 65 | 70 | 75 |
| DualAE05 | H1643L0581 | dBBDu183H1643 | dBBDu072L0581 | 6 | 20 | 34 | 48 | 58 | 63 | 68 | 73 |
| DualAE19 | H1647L0581 | dBBDu183H1647 | dBBDu072L0581 | 8 | 22 | 36 | 50 | 58 | 63 | 68 | 73 |
| DualAE20 | H1649L0581 | dBBDu183H1649 | dBBDu072L0581 | 9 | 23 | 37 | 51 | 58 | 63 | 68 | 73 |
| DualAE21 | H1649L0943 | dBBDu183H1649 | dBBDu072L0943 | 9 | 23 | 37 | 51 | 61 | 66 | 71 | 76 |
| DualAE22 | H1651L0581 | dBBDu183H1651 | dBBDu072L0581 | 10 | 24 | 38 | 52 | 58 | 63 | 68 | 73 |
| DualAE23 | H1652L0943 | dBBDu183H1652 | dBBDu072L0943 | 11 | 25 | 39 | 53 | 61 | 66 | 71 | 76 |
| DualAE09 | H1673L0943 | dBBDu183H1673 | dBBDu072L0943 | 12 | 26 | 40 | 54 | 61 | 66 | 71 | 76 |
| DualAE18 | H1673L0581 | dBBDu183H1673 | dBBDu072L0581 | 12 | 26 | 40 | 54 | 58 | 63 | 68 | 73 |
| DualAE14 | H2591L0581 | dBBDu183H2591 | dBBDu072L0581 | 13 | 27 | 41 | 55 | 58 | 63 | 68 | 73 |
| DualAE15 | H2594L0581 | dBBDu183H2594 | dBBDu072L0581 | 14 | 28 | 42 | 56 | 58 | 63 | 68 | 73 |
| DualAE16 | H1644L0939 | dBBDu183H1644 | dBBDu072L0939 | 81 | 82 | 83 | 84 | 60 | 65 | 70 | 75 |
| DualAE02 | H0888L0581 | dBBDu183H0888 | dBBDu072L0581 | 101 | 114 | 127 | 140 | 58 | 63 | 68 | 73 |
| DualAE24 | H1595L0581 | dBBDu183H1595 | dBBDu072L0581 | 104 | 117 | 130 | 143 | 58 | 63 | 68 | 73 |
| DualAE07 | H1573L0581 | dBBDu183H1573 | dBBDu072L0581 | 106 | 119 | 132 | 145 | 58 | 63 | 68 | 73 |
| DualAE25 | H1579L0581 | dBBDu183H1579 | dBBDu072L0581 | 107 | 120 | 133 | 146 | 58 | 63 | 68 | 73 |
| DualAE26 | H1572L0581 | dBBDu183H1572 | dBBDu072L0581 | 110 | 123 | 136 | 149 | 58 | 63 | 68 | 73 |
| DualAE27 | H0883 | dBBDu183H0883 | dBBDu072L | 113 | 126 | 139 | 152 | 57 | 62 | 67 | 72 |
| | CD3ε | CD3εVH | CD3εVL | 77 | | | | 78 | | | |
| | CD137 | CD137VH | CD137VL | 79 | | | | 80 | | | |

Full length amino acid sequence of antigen

| Antigen | SEQ | |
|---|---|---|
| name | List | Amino Acid Sequence |
| Human | 102 | QDGNEEMGGITQTPYKVSISGTTVILTCPQYPGSE |
| CD3eg | | ILWQHNDKNIGGDEDDKNIGSDEDHLSLKEFSELE |
| linker | | QSGYYVCYPRGSKPEDANFYLYLRARVGSADDAKK |
| | | DAAKKDDAKKDDAKKDGSQSIKGNHLVKVYDYQED |
| | | GSVLLTCDAEAKNITWFKDGKMIGFLTEDKKKWNL |
| | | GSNAKDPRGMYQCKGSQNKSKPLQVYYRMDYKDDD |
| | | DK |
| Human | 103 | LQDPCSNCPAGTFCDNNRNQICSPCPPNSFSSAGG |
| CD137 | | QRTCDICRQCKGVFRTRKECSSTSNAECDCTPGFH |
| ECD | | CLGAGCSMCEQDCKQGQELTKKGCKDCCFGTFNDQ |
| | | KRGICRPWTNCSLDGKSVLVNGTKERDVVCGPSPA |
| | | DLSPGASSVTPPAPAREPGHSPQHHHHHHGGGGSG |
| | | LNDIFEAQKIEWHE |

TABLE 8
Full length amino acid sequence of variable region of antibody followed by amino acid sequence
of CDR 1,2 and 3 regions as annotated in Table 6

| | SEQ | |
|---|---|---|
| SEQ list | number | Amino Acid Sequence |
| dBBDu183H | - | QVQLVESGGGLVQPGRSLRLSCAASGFTFSNAWMHWVRQAPGKGLEWVAQIKDKGNAYAAYYA |
| | | PSVKGRFTISRDDSKNSIYLQMNSLKTEDTAVYYCHYVHYASASTVLPAFGVDAWGQGTTVTVSS |
| dBBDu183H0868 | 2 | QVQLVESGGGLVQPGRSLRLSCAASGFKFSNVWMHWVRQAPGKGLEWVAQIKDKYNAYAAYYA |
| | | PSVKGRFTISRDDSKNSIYLQMNSLKTEDTAVYYCHYVHYASASTLLPAFGVDAWGQGTTVTVSS |
| dBBDu183H1550 | 3 | QVQLVESGGGLVQPGRSLRLSCAASGFKFSNVWMHWVRQAPGKGLEWVAQIKDKYNAYAAYYA |
| | | PSVKGRFTISRDDSKNSIYLQMNSLKTEDTAVYYCHYIHYASASTLLPAFGVDAWGQGTTVTVSS |
| dBBDu183H1571 | 4 | QVQLVESGGGLVQPGRSLRLSCAASGFKFSNVWFHWVRQAPGKGLEWVAQIKDKYNAYATYYAP |
| | | SVKGRFTISRDDSKNSIYLQMNSLKTEDTAVYYCHYVHYASASTLLPAFGVDAWGQGTTVTVSS |
| dBBDu183H1610 | 5 | QVQLVESGGGLVQPGRSLRLSCAASGFVFSNVWMHWVRQAPGKGLEWVAQIKDKWNAYAAYYA |
| | | PSVKGRFTISRDDSKNSIYLQMNSLKTEDTAVYYCHYIHYASASTLLPAEGIDAWGQGTTVTVSS |
| dBBDu183H1643 | 6 | QVQLVESGGGLVQPGRSLRLSCAASGFKFSNVWFHWVRQAPGKGLEWVAQIKDYYNAYAAYYAP |
| | | SVKGRFTISRDDSKNSIYLQMNSLKTEDTAVYYCHYVHYASASTLLPAEGVDAWGQGTTVTVSS |
| dBBDu183H1647 | 8 | QVQLVESGGGLVQPGRSLRLSCAASGFKFSNTWFHWVRQAPGKGLEWVAQIKDYYNDYAAYYAP |
| | | SVKGRFTISRDDSKNSIYLQMNSLKTEDTAVYYCHYVHYASASTLLPAEGVDAWGQGTTVTVSS |
| dBBDu183H1649 | 9 | QVQLVESGGGLVQPGRSLRLSCAASGFVFSNVWFHWVRQAPGKGLEWVAQIKDKYNAYADYYAP |
| | | SVKERFTISRDDSKNSIYLQMNSLKTEDTAVYYCHYVHYASASTLLPAEGVDAWGQGTTVTVSS |
| dBBDu183H1651 | 10 | QVQLVESGGGLVQPGRSLRLSCAASGFVFSNVWFHWVRQAPGKGLEWVAQIKDKYNAYADYYAP |
| | | SVEGRFTISRDDSKNSIYLQMNSLKTEDTAVYYCHYVHYASASTLLPAEGVDAWGQGTTVTVSS |
| dBBDu183H1652 | 11 | QVQLVESGGGLVQPGRSLRLSCAASGFVFSNVWFHWVRQAPGKGLEWVAQIKDYYNAYADYYAP |
| | | SVEGRFTISRDDSKNSIYLQMNSLKTEDTAVYYCHYVHYASASTLLPAEGVDAWGQGTTVTVSS |
| dBBDu183H1673 | 12 | QVQLVESGGGLVQPGRSLRLSCAASGFVFSNVWFHWVRQAPGKGLEWVAQIKDKWNAYADYYA |
| | | PSVKERFTISRDDSKNSIYLQMNSLKTEDTAVYYCHYIHYASASTLLPAEGIDAWGQGTTVTVSS |
| dBBDu183H2591 | 13 | QVQLVESGGGLVQPGRSLRLSCAASGFKFSNVWFHWVRQAPGKGLEWVAQIKDYYNAYAGYYHP |
| | | SVKGRFTISRDDSKNSIYLQMNSLKTEDTAVYYCHYVHYAAASTLLPAEGVDAWGQGTTVTVSS |
| dBBDu183H2594 | 14 | QVQLVESGGGLVQPGRSLRLSCAASGFKFSNVWFHWVRQAPGKGLEWVAQIKDYYNAYAGYYHP |
| | | SVKGRFTISRDDSKNSIYLQMNSLKTEDTAVYYCHYVHYAAASQLLPAEGVDAWGQGTTVTVSS |
| dBBDu183H_VHR_CDRI | 15 | NAWMH |
| dBBDu183H0868_VHR_CDR1 | 16 | NVWMH |
| dBBDu183H1550_VHR_CDR1 | 17 | NVWMH |
| dBBDu183H1571_VHR_CDR1 | 18 | NVWFH |
| dBBDu183H1610_VHR_CDR1 | 19 | NVWMH |
| dBBDu183H1643_VHR_CDR1 | 20 | NVWFH |
| dBBDu183H1647_VHR_CDR1 | 22 | NTWFH |
| dBBDu183H1649_VHR_CDR1 | 23 | NVWFH |
| dBBDu183H1651_VHR_CDR1 | 24 | NVWFH |
| dBBDu183H1652_VHR_CDR1 | 25 | NVWFH |
| dBBDu183H1673_VHR_CDR1 | 26 | NVWFH |
| dBBDu183H2591_VHR_CDR1 | 27 | NVWFH |
| dBBDu183H2594_VHR_CDR1 | 28 | NVWFH |
| dBBDu183H_VHR_CDR2 | 29 | QIKDKGNAYAAYYAPSVKG |
| dBBDu183H0868_VHR_CDR2 | 30 | QIKDKYNAYAAYYAPSVKG |
| dBBDu183H1550_VHR_CDR2 | 31 | QIKDKYNAYAAYYAPSVKG |
| dBBDu183H1571_VHR_CDR2 | 32 | QIKDKYNAYATYYAPSVKG |
| dBBDu183H1610_VHR_CDR2 | 33 | QIKDKWNAYAAYYAPSVKG |
| dBBDu183H1643_VHR_CDR2 | 34 | QIKDYYNAYAAYYAPSVKG |
| dBBDu183H1647_VHR_CDR2 | 36 | QIKDYYNDYAAYYAPSVKG |
| dBBDu183H1649_VHR_CDR2 | 37 | QIKDKYNAYADYYAPSVKE |
| dBBDu183H1651_VHR_CDR2 | 38 | QIKDKYNAYADYYAPSVEG |
| dBBDu183H1652_VHR_CDR2 | 39 | QIKDYYNAYADYYAPSVEG |
| dBBDu183H1673_VHR_CDR2 | 40 | QIKDKWNAYADYYAPSVKE |
| dBBDu183H2591_VHR_CDR2 | 41 | QIKDYYNAYAGYYHPSVKG |
| dBBDu183H2594_VHR_CDR2 | 42 | QIKDYYNAYAGYYHPSVKG |
| dBBDu183H_VHR_CDR3 | 43 | VHYASASTVLPAFGVDA |
| dBBDu183H0868_VHR_CDR3 | 44 | VHYASASTLLPAFGVDA |
| dBBDu183H1550_VHR_CDR3 | 45 | IHYASASTLLPAFGVDA |
| dBBDu183H1571_VHR_CDR3 | 46 | VHYASASTLLPAFGVDA |
| dBBDu183H1610_VHR_CDR3 | 47 | IHYASASTLLPAEGIDA |
| dBBDu183H1643_VHR_CDR3 | 48 | VHYASASTLLPAEGVDA |
| dBBDu183H1647_VHR_CDR3 | 50 | VHYASASTLLPAEGVDA |
| dBBDu183H1649_VHR_CDR3 | 51 | VHYASASTLLPAEGVDA |
| dBBDu183H1651_VHR_CDR3 | 52 | VHYASASTLLPAEGVDA |
| dBBDu183H1652_VHR_CDR3 | 53 | VHYASASTLLPAEGVDA |
| dBBDu183H1673_VHR_CDR3 | 54 | IHYASASTLLPAEGIDA |
| dBBDu183H2591_VHR_CDR3 | 55 | VHYAAASTLLPAEGVDA |
| dBBDu183H2594_VHR_CDR3 | 56 | VHYAAASQLLPAEGVDA |
| dBBDu072L | 57 | DIVMTQSPLSLPVTPGEPASISCQASQELVHMNRNTYLHWYQQKPGQAPRLLIYKVSNRFPGVPD |
| | | RFSGSGSGTDFTLKISRVEAEDVGVYYCAQGTSVPFTFGQGTKLEIK |
| dBBDu072L0581 | 58 | DIVMTQSPLSLPVTPGEPASISCQPSQEVVHMNRNTYLHWYQQKPGQAPRLLIYKVSNRFPGVPD |
| | | RFSGSGSGTDFTLKISRVEAEDVGVYYCAQGTSHPFTFGQGTKLEIK |
| dBBDu072L0918 | 59 | DIVMTQSPLSLPVTPGEPASISCQPSQEVVHMNNVVYLHWYQQKPGQAPRLLIYKVSNRFPGVPD |
| | | RFSGSGSGTDFTLKISRVEAEDVGVYYCAQGTSHPFTFGQGTKLEIK |
| dBBDu072L0939 | 60 | DIVMTQSPLSLPVTPGEPASISCQPSQEVVHMNRNTYLHWYQQKPGQAPRLLIYKVSNVFPGVPD |
| | | RFSGSGSGTDFTLKISRVEAEDVGVYYCAQGTHHPFTFGQGTKLEIK |
| dBBDu072L0943 | 61 | DIVMTQSPLSLPVTPGEPASISCQPSEEVVHMNRNTYLHWYQQKPGQAPRLLIYKVSNLFPGVPD |
| | | RFSGSGSGTDFTLKISRVEAEDVGVYYCAQGTHHPFTFGQGTKLEIK |
| dBBDu072L_VLR_CDR1 | 62 | QASQELVHMNRNTYLH |
| dBBDu072L0581_VLR_CDR1 | 63 | QPSQEVVHMNRNTYLH |
| dBBDu072L0918_VLR_CDR1 | 64 | QPSQEVVHMNNVVYLH |
| dBBDu072L0939_VLR_CDR1 | 65 | QPSQEVVHMNRNTYLH |
| dBBDu072L0943_VLR_CDRI | 66 | QPSEEVVHMNRNTYLH |
| dBBDu072L_VLR_CDR2 | 67 | KVSNRFP |
| dBBDu072L0581_VLR_CDR2 | 68 | KVSNRFP |
| dBBDu072L0918_VLR_CDR2 | 69 | KVSNRFP |
| dBBDu072L0939_VLR_CDR2 | 70 | KVSNVFP |
| dBBDu072L0943_VLR_CDR2 | 71 | KVSNLFP |
| dBBDu072L_VLR_CDR3 | 72 | AQGTSVPFT |
| dBBDu072L0581_VLR_CDR3 | 73 | AQGTSHPFT |
| dBBDu072L0918_VLR_CDR3 | 74 | AQGTSHPFT |
| dBBDu072L0939_VLR_CDR3 | 75 | AQGTHHPFT |
| dBBDu072L0943_VLR_CDR3 | 76 | AQGTHHPFT |
| CD3&VH | 77 | QVQLVESGGGVVQPGGSLRLSCAASGFTFSNAWMHWVRQAPGKGLEWVAQIKDKSQNYATYV |
| | | AESVKGRFTISRADSKNSIYLQMNSLKTEDTAVYYCRYVHYAAGYGVDIWGQGTTVTVSS |
| CD3EVL | 78 | DIVMTQSPLSLPVTPGEPASISCRSSQPLVHSNRNTYLHWYQQKPGQAPRLLIYKVSNRFSGVPDRF |
| | | SGSGSGTDFTLKISRVEAEDVGVYYCGQGTQVPYTFGQGTKLEIK |
| CD137VH | 79 | QVQLQQWGAGLLKPSETLSLTCAVYGGSFSGYYWSWIRQSPEKGLEWIGEINHGGYVTYNPSLESR |
| | | VTISVDTSKNQFSLKLSSVTAADTAVYYCARDYGPGNYDWYFDLWGRGTLVTVSS |
| CD137VL | 80 | EIVLTQSPATLSLSPGERATLSCRASQSVSSYLAWYQQKPGQAPRLLIYDASNRATGIPARFSGSGSGT |
| | | DFTLTISSLEPEDFAVYYCQQRSNWPPALTFGGGTKVEIK |
| dBBDu183H1644 | 81 | QVQLVESGGGLVQPGRSLRLSCAASGFVFSNVWFHWVRQAPGKGLEWVAQIKDYYNAYAAYYAP |
| | | SVKGRFTISRDDSKNSIYLQMNSLKTEDTAVYYCHYVHYASASTLLPAEGVDAWGQGTTVTVSS |
| dBBDu183H1644_VHR_CDR1 | 82 | NVWFH |
| dBBDu183H1644_VHR_CDR2 | 83 | QIKDYYNAYAAYYAPSVKG |
| dBBDu183H1644_VHR_CDR3 | 84 | VHYASASTLLPAEGVDA |
| dBBDu183H0888 | 101 | QVQLVESGGGLVQPGRSLRLSCAASGFKFSNVWMHWVRQAPGKGLEWVAQIKDKWNAYAAYYA |
| | | PSVKGRFTISRDDSKNSIYLQMNSLKTEDTAVYYCHYIHYASASTLLPAFGIDAWGQGTTVTVSS |
| dBBDu183H1595 | 104 | QVQLVESGGGLVQPGRSLRLSCAASGFKFSNTWMHWVRQAPGKGLEWVAQIKDKYNAYAAYYA |
| | | PSVKGRFTISRDDSKNSIYLQMNSLKTEDTAVYYCHYIHYASASTLLPAFGVDAWGQGTTVTVSS |
| dBBDu183H1573 | 106 | QVQLVESGGGLVQPGRSLRLSCAASGFKFSNVWFHWVRQAPGKGLEWVAQIKDYYNAYAAYYAP |
| | | SVKGRFTISRDDSKNSIYLQMNSLKTEDTAVYYCHYVHYASASTLLPAFGVDAWGQGTTVTVSS |
| dBBDu183H1579 | 107 | QVQLVESGGGLVQPGRSLRLSCAASGFKFSHVWFHWVRQAPGKGLEWVAQIKDKYNAYAAYYAP |
| | | SVKGRFTISRDDSKNSIYLQMNSLKTEDTAVYYCHYVHYASASTLLPAFGVDAWGQGTTVTVSS |
| dBBDu183H1572 | 110 | QVQLVESGGGLVQPGRSLRLSCAASGFKFSNVWFHWVRQAPGKGLEWVAQIKDKYNAYAAYYAP |
| | | SVKGRFTISRDDSKNSIYLQMNSLKTEDTAVYYCHYVHYASASTLLPAEGVDAWGQGTTVTVSS |
| dBBDu183H0883 | 113 | QVQLVESGGGLVQPGRSLRLSCAASGFTFSNAWMHWVRQAPGKGLEWVAQIKDKGNAYAAYYA |
| | | PSVKGRFTISRDDSKNSIYLQMNSLKTEDTAVYYCRYVHYASASTLLPAFGVDAWGQGTTVTVSS |
| dBBDu183H0888_VHR_CDR1 | 114 | NVWMH |
| dBBDu183H1595_VHR_CDR1 | 117 | NTWMH |
| dBBDu183H1573_VHR_CDR1 | 119 | NVWFH |
| dBBDu183H1579_VHR_CDR1 | 120 | HVWFH |
| dBBDu183H1572_VHR_CDRI | 123 | NVWFH |
| dBBDu183H0883_VHR_CDR1 | 126 | NAWMH |
| dBBDu183H0888_VHR_CDR2 | 127 | QIKDKWNAYAAYYAPSVKG |
| dBBDu183H1595_VHR_CDR2 | 130 | QIKDKYNAYAAYYAPSVKG |
| dBBDu183H1573_VHR_CDR2 | 132 | QIKDYYNAYAAYYAPSVKG |
| dBBDu183H1579_VHR_CDR2 | 133 | QIKDKYNAYAAYYAPSVKG |
| dBBDu183H1572_VHR_CDR2 | 136 | QIKDKYNAYAAYYAPSVKG |
| dBBDu183H0883_VHR_CDR2 | 139 | QIKDKGNAYAAYYAPSVKG |
| dBBDu183H0888_VHR_CDR3 | 140 | IHYASASTLLPAFGIDA |
| dBBDu183II1595_VIIR_CDR3 | 143 | IHYASASTLLPAFGVDA |
| dBBDu183H1573_VHR_CDR3 | 145 | VHYASASTLLPAFGVDA |
| dBBDu183H1579_VHR_CDR3 | 146 | VHYASASTLLPAFGVDA |
| dBBDu183H1572_VHR_CDR3 | 149 | VHYASASTLLPAEGVDA |
| dBBDu183H0883_VHR_CDR3 | 152 | VHYASASTLLPAFGVDA |

TABLE 8-6
Binding kinetics of Dual variants towards human CD3 and human CD137

| | CD3 (25° C.) | | | CD137 (37° C.) | | |
|---|---|---|---|---|---|---|
| Antibody name | ka (M−1s−1) | kd (s−1) | KD (M) | ka (M−1s−1) | kd (s−1) | KD (M) |
| H183L072 | 3.54E+04 | 1.20E−02 | 3.40E−07 | 3.47E+03 | 1.96E−02 | 5.66E−06 |
| H0868L0581 | 1.23E+05 | 1.94E−02 | 1.57E−07 | 1.22E+04 | 1.36E−03 | 1.11E−07 |
| H1550L0918 | 7.20E+04 | 3.16E−03 | 4.38E−08 | 1.09E+04 | 5.79E−03 | 5.30E−07 |
| H1571L0581 | 1.42E+05 | 1.56E−02 | 1.10E−07 | 1.21E+04 | 1.05E−03 | 8.68E−08 |
| H1610L0581 | 6.80E+04 | 1.42E−03 | 2.09E−08 | 1.07E+04 | 1.10E−03 | 1.03E−07 |
| H1610L0939 | 5.00E+04 | 2.53E−03 | 5.07E−08 | 1.30E+04 | 8.01E−04 | 6.18E−08 |
| H1643L0581 | 9.46E+04 | 2.51E−02 | 2.65E−07 | 1.23E+04 | 6.06E−04 | 4.94E−08 |
| H1644L0939 | 5.58E+04 | 8.08E−02 | 1.45E−06 | 1.21E+04 | 4.44E−04 | 3.68E−08 |
| H1647L0581 | 4.43E+04 | 1.01E−01 | 2.28E−06 | 9.98E+03 | 6.47E−04 | 6.48E−08 |
| H1649L0581 | 7.50E+04 | 3.36E−02 | 4.49E−07 | 1.29E+04 | 5.53E−04 | 4.28E−08 |
| H1649L0943 | 6.10E+04 | 4.81E−02 | 7.89E−07 | 1.43E+04 | 4.68E−04 | 3.28E−08 |
| H1651L0581 | 7.18E+04 | 3.71E−02 | 5.17E−07 | 1.40E+04 | 6.03E−04 | 4.32E−08 |
| H1652L0943 | 6.23E+04 | 6.36E−02 | 1.02E−06 | 1.29E+04 | 4.70E−04 | 3.64E−08 |
| H1673L0581 | 7.96E+04 | 1.06E−03 | 1.33E−08 | 1.19E+04 | 9.60E−04 | 8.04E−08 |
| H1673L0943 | 5.50E+04 | 1.16E−03 | 2.10E−08 | 1.22E+04 | 7.22E−04 | 5.91E−08 |
| H2591L0581 | 1.02E+05 | 5.35E−02 | 5.25E−07 | 2.04E+04 | 7.42E−04 | 3.63E−08 |
| H2594L0581 | 9.83E+04 | 5.84E−02 | 5.93E−07 | 2.09E+04 | 1.63E−03 | 7.81E−08 |

TABLE 9-2
Binding kinetics of Dual variants towards human CD3 and human CD137

| | CD3 (25° C.) | | | CD137 (37° C.) | | |
|---|---|---|---|---|---|---|
| Antibody Name | ka (1/Ms) | kd (1/s) | KD (M) | ka (1/Ms) | kd (1/s) | KD (M) |
| H0888L0581 | 9.50E+04 | 1.92E−03 | 2.02E−08 | 1.50E+04 | 3.11E−03 | 2.08E−07 |
| H1595L0581 | 1.16E+05 | 6.58E−03 | 5.70E−08 | 1.38E+04 | 2.69E−03 | 1.95E−07 |
| H1573L0581 | 1.21E+05 | 1.88E−02 | 1.56E−07 | 1.46E+04 | 1.03E−03 | 7.06E−08 |
| H1579L0581 | 1.24E+05 | 3.40E−02 | 2.73E−07 | 1.48E+04 | 4.06E−03 | 2.75E−07 |
| H1572L0581 | 9.77E+04 | 2.80E−02 | 2.86E−07 | 1.39E+04 | 7.22E−04 | 5.21E−08 |

| | | | | |
|---|---|---|---|---|
| H0883 | 9.07E+04 | 9.99E−03 | 1.10E−07 | n.d. |

### Example 2

### Bi-Specific and Tri-Specific Antibody Sequences and Preparation

### 2.1. Design and Preparation of Bi- or Tri-Specific Ab (1+1) Format

To evaluate the efficacy of Dual-Ig variants (DualAE05 etc.) prepared in Example 1, bi-specific and tri-specific antibodies were generated with one arm recognizing tumor antigen DLL3 (from anti-DLL3 antibody D30841AE13) and the other arm recognizing effector cells, predominantly T-cells. DLL3/CD3 epsilon (1+1) and DLL3/DualAE05 (1+1) antibodies were generated by using Fab-arm exchange (FAE) according to the method described in Proc Natl Acad Sci USA. 2013 Mar. 26; 110(13):5145-5150. The (1+1) format is depicted in FIG. 1(b). The target antigen of each Fv region and naming rule of each binding domain in the bi- and trispecific antibodies in the (1+1) format are shown in Table 10-2 and the SEQ ID NOs are shown in Table 12.

### 2.2. Design and Preparation of Trivalent Ab (1+2) Format Comprising One Monovalent DLL3-Binding Arm and Two Dual-Fabs

Target antigen expression in solid tumors are likely to be highly heterogenous and regions of tumors with low antigen expression may not provide sufficient cross-linking of CD3 or CD137. In particular, CD137 receptor clustering is critical for efficient agonistic activity (Trends Biohem Sci. 2002 January; 27(1)19-26). To improve cytotoxicity mediated by CD3 binding and activation, a new trivalent tri-specific antibody format i.e. DLL3-DUAL/LINC, 1+2) was designed to increase number of binding domains to CD137 molecules. Specifically, the new antibody format is a trivalent tri-specific antibody with “1+2” format which comprises two monovalent Dual-Fabs each capable of binding to one CD3 or CD137 but not simultaneously (FvB and FvC of FIG. 1a), and one monovalent DLL3-binding arm (FvA of FIG. 1a), wherein one disulfide bond (“LINC”) is introduced/engineered between the two Dual-Fabs by introducing a cysteine substitution e.g. at the 191 position (S191C with Kabat numbering) of the CH1 domain of each of the two Dual-Fabs (FIG. 1(a) and Table 10-1). Without wishing to be bound by a theory, we envisioned that such engineered disulfide bond (“LINC”) would restrict the antigen (CD3 or CD137) binding orientation of the two Dual-Fabs to cis antigen-binding (i.e. binding to two antigens on the same cell) as a results of steric hindrance or shorter distance between the two Dual-Fabs, thereby improving the safety profile of the trispecific Ab by preventing undesirable crosslinking of two CD3/CD137-expressing immune cells mediated by the two Dual-Fabs in an DLL3-independent manner. Specifically, DLL3-Dual/Dual antibody variants (DLL3-Dual/LINC, 1+2) having monovalent tumor antigen binding ability towards DLL3, bivalent CD3 and bivalent CD137 binding properties attributed to two Dual-Fabs were prepared (FIG. 1(a) and Table 10-1). CrossMab technology (WO2017055539) was also utilized. Fc region was Fc-gamma R silent and deglycosylated. The target antigen of each Fv region and naming rule of each binding domain in the trispecific antibodies are shown in Table 10-1 and the SEQ ID NOs are shown in Table 11-1 and 12. As for a negative control, Ctrl (VHR IC17HdK and VLR IC17L) which does not bind to DLL3 was used in place of the monovalent DLL3-binding arm (Table 12). All antibodies were expressed as trivalent form by transient expression in Expi293 cells (Invitrogen) and purified according to Example 1.1. To improve the purities, antibodies were further purified by passing through an affinity column that specifically binds the UnLINC format (i.e., trivalent 1+2 antibody without the engineered disulfide bond) but not the LINC format (i.e., trivalent 1+2 antibody with the engineered disulfide bond).

### 2.3. Generation of Bispecific Ab (BiTE)

In order to compare the efficacy of Trivalent Ab (DLL3-DUAL/LINC, 2+1) with other DLL3/CD3 bispecific format antibodies, an antibody with half-life extended BiTE format was generated, named as DLL3CD3BiTE. DLL3CD3BiTE was composed of two single-chain variable fragments (ScFv), one directed against the tumor-associated antigen DLL3 fused to one that is directed against CD3 (FIG. 1(c)). The target antigen of each Fv region in the BiTE format bispecific antibody is shown in Table 10-3, and the SEQ ID NO is shown in Table 11-2 and Table 14.

TABLE 10-1
Antibody name, targeted arm and Fc information

| Trivalent Ab | | | | | | | | |
|---|---|---|---|---|---|---|---|---|
| (1 + 2 LINC) | | | | | | | | |
| Antibody Name | Format | Fv A | Linker | Fv B | Fv C | FC | Fc (knob) | Fc (hole) |
| DLL3-DualAE05/ | (1 + 2) | Anti-DLL3 | Long | DualAE05 | DualAE05 | LALA, N297A, Act5 | SG1321kV11Fc | SG1321hV11Fc |
| DualAE05-FF091 | Dual/LINC | | | | | (M428L, N434A, | | |
| | | | | | | Q438R, S440E) | | |
| Ctrl-DualAE05/ | (1 + 2) | Ctrl | Long | DualAE05 | DualAE05 | LALA, N297A, Act5 | SG1321kV11Fc | SG1321hV11Fc |
| DualAE05-FF091 | Dual/LINC | | | | | (M428L, N434A, | | |
| | | | | | | Q438R, S440E) | | |
| DLL3-DualAE05/ | (1 + 2) | Anti-DLL3 | Long | DualAE05 | DualAE05 | LALA, N297A, Act5 | SG1372kV11Fc | SG1372hV11Fc |
| DualAE05-FF102 | Dual/LINC | | | | | (M428L, N434A, | | |
| | | | | | | Q438R, S440E) | | |
| DLL3-DualAE05/ | (1 + 2) | Anti-DLL3 | Mid | DualAE05 | DualAE05 | LALA, N297A, Act5 | SG1372kV11Fc | SG1372hV11Fc |
| DualAE05-FF110 | Dual/LINC | | | | | (M428L, N434A, | | |
| | | | | | | Q438R, S440E) | | |
| DLL3-DualAE05/ | (1 + 2) | Anti-DLL3 | Short | DualAE05 | DualAE05 | LALA, N297A, Act5 | SG1372kV11Fc | SG1372hV11Fc |
| DualAE05-FF111 | Dual/LINC | | | | | (M428L, N434A, | | |
| | | | | | | Q438R, S440E) | | |
| DLL3-DualAE05/ | (1 + 2) | Anti-DLL3 | Long | DualAE05 | DualAE05 | L235R, S239K, N297A | SG1176kV11Fc | SG1176hV11Fc |
| DualAE05-FF056 | Dual/LINC | | | | | | | |
| DLL3-DualAE15/ | (1 + 2) | Anti-DLL3 | Long | DualAE15 | DualAE15 | LALA, N297A, Act5 | SG1321kV11Fc | SG1321hV11Fc |
| DualAE15-FF119 | Dual/LINC | | | | | (M428L, N434A, | | |
| | | | | | | Q438R, S440E) | | |
| Ctrl-DualAE15/ | (1 + 2) | Ctrl | Long | DualAE15 | DualAE15 | LALA, N297A, Act5 | SG1321kV11Fc | SG1321hV11Fc |
| DualAE15-FF119 | Dual/LINC | | | | | (M428L, N434A, | | |
| | | | | | | Q438R, S440E) | | |
| DLL3-DualAE15/ | (1 + 2) | Anti-DLL3 | Long | DualAE15 | DualAE15 | LALA, N297A, Act5 | SG1372kV11Fc | SG1372hV11Fc |
| DualAE15-FF120 | Dual/LINC | | | | | (M428L, N434A, | | |
| | | | | | | Q438R, S440E) | | |
| DLL3-DualAE15/ | (1 + 2) | Anti-DLL3 | Mid | DualAE15 | DualAE15 | LALA, N297A, Act5 | SG1372kV11Fc | SG1372hV11Fc |
| DualAE15-FF121 | Dual/LINC | | | | | (M428L, N434A, | | |
| | | | | | | Q438R, S440E) | | |
| DLL3-DualAE15/ | (1 + 2) | Anti-DLL3 | Short | DualAE15 | DualAE15 | LALA, N297A, Act5 | SG1372kV11Fc | SG1372hV11Fc |
| DualAE15-FF122 | Dual/LINC | | | | | (M428L, N434A, | | |
| | | | | | | Q438R, S440E) | | |
| DLL3-DualAE15/ | (1 + 2) | Anti-DLL3 | Long | DualAE15 | DualAE15 | L235R, S239K, N297A | SG1176kV11Fc | SG1176hV11Fc |
| DualAE15-FF123 | Dual/LINC | | | | | | | |
| DLL3-DualAE16/ | (1 + 2) | Anti-DLL3 | Long | DualAE16 | DualAE16 | LALA, N297A, Act5 | SG1321kV11Fc | SG1321hV11Fc |
| DualAE16-FF124 | Dual/LINC | | | | | (M428L, N434A, | | |
| | | | | | | Q438R, S440E) | | |
| Ctrl-DualAE16/ | (1 + 2) | Ctrl | Long | DualAE16 | DualAE16 | LALA, N297A, Act5 | SG1321kV11Fc | SG1321hV11Fc |
| DualAE16-FF124 | Dual/LINC | | | | | (M428L, N434A, | | |
| | | | | | | Q438R, S440E) | | |
| DLL3-DualAE16/ | (1 + 2) | Anti-DLL3 | Long | DualAE16 | DualAE16 | LALA, N297A, Act5 | SG1372kV11Fc | SG1372hV11Fc |
| DualAE16-FF125 | Dual/LINC | | | | | (M428L, N434A, | | |
| | | | | | | Q438R, S440E) | | |
| DLL3-DualAE16/ | (1 + 2) | Anti-DLL3 | Mid | DualAE16 | DualAE16 | LALA, N297A, Act5 | SG1372kV11Fc | SG1372hV11Fc |
| DualAE16-FF126 | Dual/LINC | | | | | (M428L, N434A, | | |
| | | | | | | Q438R, S440E) | | |
| DLL3-DualAE16/ | (1 + 2) | Anti-DLL3 | Short | DualAE16 | DualAE16 | LALA, N297A, Act5 | SG1372kV11Fc | SG1372hV11Fc |
| DualAE16-FF127 | Dual/LINC | | | | | (M428L, N434A, | | |
| | | | | | | Q438R, S440E) | | |
| DLL3-DualAE16/ | (1 + 2) | Anti-DLL3 | Long | DualAE16 | DualAE16 | L235R, S239K, N297A | SG1176kV11Fc | SG1176hV11Fc |
| DualAE16-FF128 | Dual/LINC | | | | | | | |

TABLE 10-2
Bi- and Trispecific Ab (1 + 1)

| Antibody name | Fv A | Fv B |
|---|---|---|
| DLL3/CD3ε | DLL3 | CD3ε |
| DLL3/DualAE05 | DLL3 | DualAE05 |

TABLE 10-3
Bispecific Ab (BiTE)

| Antibody name | Fv A | Fv B |
|---|---|---|
| DLL3CD3BiTE | Anti-DLL3 | Anti-CD3 |

TABLE 11-1
Antibody chain number and Sequence ID

| | | | | | Chain 4 or |
|---|---|---|---|---|---|
| Variant name | Linker | Chain 1 | Chain 2 | Chain 3 | Chain 5 |
| DLL3-DualAE05/DualAE05-FF091 | 249 | 201 | 206 | 208 | 214 |
| Ctrl-DualAE05/DualAE05-FF091 | 249 | 202 | 207 | 208 | 214 |
| DLL3-DualAE05/DualAE05-FF102 | 249 | 203 | 206 | 209 | 214 |
| DLL3-DualAE05/DualAE05-FF110 | 248 | 204 | 206 | 209 | 214 |
| DLL3-DualAE05/DualAE05-FF111 | 259 | 205 | 206 | 209 | 214 |
| DLL3-DualAE05/DualAE05-FF114 | 248 | 153 | 154 | 155 | 156 |
| DLL3-DualAE05/DualAE05-FF056 | 249 | 216 | 206 | 229 | 214 |
| DLL3-DualAE15/DualAE15-FF119 | 249 | 217 | 206 | 210 | 214 |
| Ctrl-DualAE15/DualAE15-FF119 | 249 | 218 | 207 | 210 | 214 |
| DLL3-DualAE15/DualAE15-FF120 | 249 | 219 | 206 | 211 | 214 |
| DLL3-DualAE15/DualAE15-FF121 | 248 | 220 | 206 | 211 | 214 |
| DLL3-DualAE15/DualAE15-FF122 | 259 | 221 | 206 | 211 | 214 |
| DLL3-DualAE15/DualAE15-FF123 | 249 | 222 | 206 | 230 | 214 |
| DLL3-DualAE16/DualAE16-FF124 | 249 | 223 | 206 | 212 | 215 |
| Ctrl-DualAE16/DualAE16-FF124 | 249 | 224 | 207 | 212 | 215 |
| DLL3-DualAE16/DualAE16-FF125 | 249 | 225 | 206 | 213 | 215 |
| DLL3-DualAE16/DualAE16-FF126 | 248 | 226 | 206 | 213 | 215 |
| DLL3-DualAE16/DualAE16-FF127 | 259 | 227 | 206 | 213 | 215 |
| DLL3-DualAE16/DualAE16-FF128 | 249 | 228 | 206 | 231 | 215 |

TABLE 11-2
Antibody chain number and Sequence ID

| Antibody name | Chain 1 |
|---|---|
| DLL3CD3BiTE | 250 |

TABLE 12
Variable region and their CDR 1-3 Sequence ID

| | | | | VHR_{—} | VHR_{—} | VHR_{—} | | VLR_{—} | VLR_{—} | VLR_{—} |
|---|---|---|---|---|---|---|---|---|---|---|
| VR name | VHR name | VLR name | VHR | CDR1 | CDR2 | CDR3 | VLR | CDR1 | CDR2 | CDR3 |
| DLL3 | D08410053H0118 | D084101L0000 | 232 | 233 | 234 | 235 | 236 | 237 | 238 | 239 |
| (D30841AE13) | | | | | | | | | | |
| DualAE05 | dBBDu183H1643 | dBBDu072L0581 | 6 | 20 | 34 | 48 | 58 | 63 | 68 | 73 |
| DualAE15 | dBBDu183H2594 | dBBDu072L0581 | 14 | 28 | 42 | 56 | 58 | 63 | 68 | 73 |
| DualAE16 | dBBDu183H1644 | dBBDu072L0939 | 81 | 82 | 83 | 84 | 60 | 65 | 70 | 75 |
| CD3ε | CD3εVH | CD3εVL | 251 | 252 | 253 | 254 | 255 | 256 | 257 | 258 |
| Ctrl | IC17HdK | IC17L | 240 | 24 | 242 | 243 | 244 | 245 | 246 | 247 |

TABLE 13
Sequence ID NOs. of Fc regions in Table 14

| Fc Name | SED ID NOs |
|---|---|
| SG1321kV11Fc | 99 |
| SG1372kV11Fc | 100 |
| SG1176kV11Fc | 108 |
| SG1321hV11Fc | 109 |
| SG1372hV11Fc | 111 |
| SG1176hV11Fc | 112 |

TABLE 14

| Sequence Name | SEQ ID NOS | Amino Acid Sequence |
|---|---|---|
| | 201 | DIQLTQSPSFLSASVGDRVTITCOSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCGGGGSGGGGSQVOLVESGGGLVQPGRSLRLSCAASGFKFSNVWFH |
| | | WVRQAPGKGLEWVAQIKDYYNAYAAYYAPSVKGRFTISRDDSKNSIYLQMNSLKTEDTAVYYCHYVHYASASTLLPAEGVDAWG |
| | | QGTTVTVSSASTKGPSVFPLAPSSKSTSGGTAALGCLVEDYFPEPVTVSWNSGALTSGVHTFPAVLQSSGLYSLSSVVTVPSCSLGT |
| | | QTYICNVNHKPSNTKVDEKVEPKSCDKTHTCPPCPAPEAAGGPSVFLFPPKPKDTLMISRTPEVTCVVVDVSHEDPEVKFNWYVD |
| | | GVEVHNAKTKPREEQYASTYRVVSVLTVLHQDWLNGKEYKCKVSNKALPAPIEKTISKAKGQPREPQVYTLPPCRDELTKNQVSL |
| | | 201\|WCLVKGFYPSDIAVEWESNGQPENNYKTTPPVLDSDGSFFLYSKLTVDKSRWQEGNVFSCSVLHEALHAHYTRKELSLSP |
| | 202 | DIQMTQSSSSFSVSLGDRVTITCKASEDIYNRLAWYQQKPGNAPRLLISGATSLETGVPSRFSGSGSGKDYTLSITSLQTEDVATYYC |
| | | QQYWSTPYTFGGGTKLEVKSSASTKGPSVFPLAPSSRSTSESTAALGCLVKDYFPEPVTVSWNSGALTSGVHTFPAVLQSSGLYSLS |
| | | SVVTVPSSSLGTKTYTCNVDHKPSNTKVDKRVEPKSCGGGGSGGGGSQVQLVESGGGLVQPGRSLRLSCAASGFKFSNVWFHW |
| | | VRQAPGKGLEWVAQIKDYYNAYAAYYAPSVKGRFTISRDDSKNSIYLQMNSLKTEDTAVYYCHYVHYASASTLLPAEGVDAWGQ |
| | | GTTVTVSSASTKGPSVFPLAPSSKSTSGGTAALGCLVEDYFPEPVTVSWNSGALTSGVHTFPAVLQSSGLYSLSSVVTVPSCSLGTQ |
| | | TYICNVNHKPSNTKVDEKVEPKSCDKTHTCPPCPAPEAAGGPSVFLFPPKPKDTLMISRTPEVTCVVVDVSHEDPEVKFNWYVDG |
| | | VEVHNAKTKPREEQYASTYRVVSVLTVLHQDWLNGKEYKCKVSNKALPAPIEKTISKAKGQPREPQVYTLPPCRDELTKNQVSLW |
| | | CLVKGFYPSDIAVEWESNGQPENNYKTTPPVLDSDGSFFLYSKLTVDKSRWQEGNVFSCSVLHEALHAHYTRKELSLSP |
| | 203 | DIQLTQSPSFLSASVGDRVTITCOSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCGGGGSGGGGSQVOLVESGGGLVQPGRSLRLSCAASGFKFSNVWFH |
| | | WVRQAPGKGLEWVAQIKDYYNAYAAYYAPSVKGRFTISRDDSKNSIYLQMNSLKTEDTAVYYCHYVHYASASTLLPAEGVDAWG |
| | | QGTTVTVSSASTKGPSVFPLAPSSKSTSGGTAALGCLVEDYFPEPVTVSWNSGALTSGVHTFPAVLQSSGLYSLSSVVTVPSCSLGT |
| | | QTYICNVNHKPSNTKVDEKVEPKSCDKTHTCPPCPAPEAAGGPSVFLFPPKPKDTLMISRTPEVTCVVVDVSHEDPEVKFNWYVD |
| | | GVEVHNAKTKPREEQYASTYRVVSVLTVLHQDWLNGKEYKCKVSNKALPAPIEKTISKAKGQPREPQVYTLPPCRDELTKNQVSL |
| | | WCLVKGFYPSDIAVEWESNGQPENNYKTTPPVLDSDGSFFLYSKLTVDKSRWQQGNVFSCSVLHEALHAHYTRKELSLSP |
| | 204 | DIQLTQSPSFLSASVGDRVTITCOSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCGGGGSQVQLVESGGGLVQPGRSLRLSCAASGFKFSNVWFHWVRQA |
| | | PGKGLEWVAQIKDYYNAYAAYYAPSVKGRFTISRDDSKNSIYLQMNSLKTEDTAVYYCHYVHYASASTLLPAEGVDAWGQGTTVT |
| | | VSSASTKGPSVFPLAPSSKSTSGGTAALGCLVEDYFPEPVTVSWNSGALTSGVHTFPAVLQSSGLYSLSSVVTVPSCSLGTQTYICNV |
| | | NHKPSNTKVDEKVEPKSCDKTHTCPPCPAPEAAGGPSVFLFPPKPKDTLMISRTPEVTCVVVDVSHEDPEVKFNWYVDGVEVHN |
| | | AKTKPREEQYASTYRVVSVLTVLHQDWLNGKEYKCKVSNKALPAPIEKTISKAKGQPREPQVYTLPPCRDELTKNQVSLWCLVKGF |
| | | 204YPSDIAVEWESNGQPENNYKTTPPVLDSDGSFFLYSKLTVDKSRWQQGNVFSCSVLHEALHAHYTRKELSLSP |
| | 205 | DIQLTQSPSFLSASVGDRVTITCOSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCQVQLVESGGGLVQPGRSLRLSCAASGFKFSNVWFHWVRQAPGKGL |
| | | EWVAQIKDYYNAYAAYYAPSVKGRFTISRDDSKNSIYLQMNSLKTEDTAVYYCHYVHYASASTLLPAEGVDAWGQGTTVTVSSAS |
| | | TKGPSVFPLAPSSKSTSGGTAALGCLVEDYFPEPVTVSWNSGALTSGVHTFPAVLQSSGLYSLSSVVTVPSCSLGTQTYICNVNHKP |
| | | SNTKVDEKVEPKSCDKTHTCPPCPAPEAAGGPSVFLFPPKPKDTLMISRTPEVTCVVVDVSHEDPEVKFNWYVDGVEVHNAKTKP |
| | | REEQYASTYRVVSVLTVLHQDWLNGKEYKCKVSNKALPAPIEKTISKAKGQPREPQVYTLPPCRDELTKNQVSLWCLVKGFYPSDI |
| | | AVEWESNGQPENNYKTTPPVLDSDGSFFLYSKLTVDKSRWQQGNVFSCSVLHEALHAHYTRKELSLSP |
| | 206 | QVTLRESGPALVKPTQTLTLTCTFSGFSLSSSYDMGWVRQAPGQGLEWMGTIYTGDYSTDYASWAKGRVTISVDRSKNQFSLKLS |
| | | SVTAADTAVYYCARHTGYGYFGLWGQGTLVTVSSASVAAPSVFIFPPSDEQLKSGTASVVCLLNNFYPREAKVQWKVDNALQSG |
| | | NSQESVTEQDSKDSTYSLSSTLTLSKADYEKHKVYACEVTHQGLSSPVTKSFNRGEC |
| | 207 | QVQLQQSGPQLVRPGASVKISCKASGYSFTSYWMHWVNQRPGQGLEWIGMIDPSYSETRLNQKFKDKATLTVDKSSSTAYMQL |
| | | SSPTSEDSAVYYCALYGNYFDYWGQGTTLTVSSASVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRGEC |
| | 208 | QVQLVESGGGLVQPGRSLRLSCAASGFKFSNVWFHWVRQAPGKGLEWVAQIKDYYNAYAAYYAPSVKGRFTISRDDSKNSIYLQ |
| | | MNSLKTEDTAVYYCHYVHYASASTLLPAEGVDAWGQGTTVTVSSASTKGPSVFPLAPSSKSTSGGTAALGCLVEDYFPEPVTVSW |
| | | NSGALTSGVHTFPAVLQSSGLYSLSSVVTVPSCSLGTQTYICNVNHKPSNTKVDEKVEPKSCDKTHTCPPCPAPEAAGGPSVFLFPP |
| | | KPKDTLMISRTPEVTCVVVDVSHEDPEVKFNWYVDGVEVHNAKTKPREEQYASTYRVVSVLTVLHQDWLNGKEYKCKVSNKALP |
| | | APIEKTISKAKGQPREPQVCTLPPSRDELTKNQVSLSCAVKGFYPSDIAVEWESNGQPENNYKTTPPVLDSDGSFFLVSKLTVDKSR |
| | | WQEGNVFSCSVLHEALHAHYTRKELSLSP |
| | 209 | QVQLVESGGGLVQPGRSLRLSCAASGFKFSNVWFHWVRQAPGKGLEWVAQIKDYYNAYAAYYAPSVKGRFTISRDDSKNSIYLQ |
| | | MNSLKTEDTAVYYCHYVHYASASTLLPAEGVDAWGQGTTVTVSSASTKGPSVFPLAPSSKSTSGGTAALGCLVEDYFPEPVTVSW |
| | | NSGALTSGVHTFPAVLQSSGLYSLSSVVTVPSCSLGTQTYICNVNHKPSNTKVDEKVEPKSCDKTHTCPPCPAPEAAGGPSVFLFPP |
| | | KPKDTLMISRTPEVTCVVVDVSHEDPEVKFNWYVDGVEVHNAKTKPREEQYASTYRVVSVLTVLHQDWLNGKEYKCKVSNKALP |
| | | APIEKTISKAKGQPREPQVCTLPPSRDELTKNQVSLSCAVKGFYPSDIAVEWESNGQPENNYKTTPPVLDSDGSFFLVSKLTVDKSR |
| | | WQQGNVFSCSVLHEALHAHYTRKELSLSP |
| | 210 | QVQLVESGGGLVQPGRSLRLSCAASGFKFSNVWFHWVRQAPGKGLEWVAQIKDYYNAYAGYYHPSVKGRFTISRDDSKNSIYLQ |
| | | MNSLKTEDTAVYYCHYVHYAAASQLLPAEGVDAWGQGTTVTVSSASTKGPSVFPLAPSSKSTSGGTAALGCLVEDYFPEPVTVSW |
| | | NSGALTSGVHTFPAVLQSSGLYSLSSVVTVPSCSLGTQTYICNVNHKPSNTKVDEKVEPKSCDKTHTCPPCPAPEAAGGPSVFLFPP |
| | | KPKDTLMISRTPEVTCVVVDVSHEDPEVKFNWYVDGVEVHNAKTKPREEQYASTYRVVSVLTVLHQDWLNGKEYKCKVSNKALP |
| | | APIEKTISKAKGQPREPQVCTLPPSRDELTKNQVSLSCAVKGFYPSDIAVEWESNGQPENNYKTTPPVLDSDGSFFLVSKLTVDKSR |
| | | WQEGNVFSCSVLHEALHAHYTRKELSLSP |
| | 211 | QVQLVESGGGLVQPGRSLRLSCAASGFKFSNVWFHWVRQAPGKGLEWVAQIKDYYNAYAGYYHPSVKGRFTISRDDSKNSIYLQ |
| | | MNSLKTEDTAVYYCHYVHYAAASQLLPAEGVDAWGQGTTVTVSSASTKGPSVFPLAPSSKSTSGGTAALGCLVEDYFPEPVTVSW |
| | | NSGALTSGVHTFPAVLQSSGLYSLSSVVTVPSCSLGTQTYICNVNHKPSNTKVDEKVEPKSCDKTHTCPPCPAPEAAGGPSVFLFPP |
| | | KPKDTLMISRTPEVTCVVVDVSHEDPEVKFNWYVDGVEVHNAKTKPREEQYASTYRVVSVLTVLHQDWLNGKEYKCKVSNKALP |
| | | APIEKTISKAKGQPREPQVCTLPPSRDELTKNQVSLSCAVKGFYPSDIAVEWESNGQPENNYKTTPPVLDSDGSFFLVSKLTVDKSR |
| | | WQQGNVFSCSVLHEALHAHYTRKELSLSP |
| | 212 | QVQLVESGGGLVQPGRSLRLSCAASGFVFSNVWFHWVRQAPGKGLEWVAQIKDYYNAYAAYYAPSVKGRFTISRDDSKNSIYLQ |
| | | MNSLKTEDTAVYYCHYVHYASASTLLPAEGVDAWGQGTTVTVSSASTKGPSVFPLAPSSKSTSGGTAALGCLVEDYFPEPVTVSW |
| | | NSGALTSGVHTFPAVLQSSGLYSLSSVVTVPSCSLGTQTYICNVNHKPSNTKVDEKVEPKSCDKTHTCPPCPAPEAAGGPSVFLFPP |
| | | KPKDTLMISRTPEVTCVVVDVSHEDPEVKFNWYVDGVEVHNAKTKPREEQYASTYRVVSVLTVLHQDWLNGKEYKCKVSNKALP |
| | | APIEKTISKAKGQPREPQVCTLPPSRDELTKNQVSLSCAVKGFYPSDIAVEWESNGQPENNYKTTPPVLDSDGSFFLVSKLTVDKSR |
| | | WQEGNVFSCSVLHEALHAHYTRKELSLSP |
| | 213 | QVQLVESGGGLVQPGRSLRLSCAASGFVFSNVWFHWVRQAPGKGLEWVAQIKDYYNAYAAYYAPSVKGRFTISRDDSKNSIYLQ |
| | | MNSLKTEDTAVYYCHYVHYASASTLLPAEGVDAWGQGTTVTVSSASTKGPSVFPLAPSSKSTSGGTAALGCLVEDYFPEPVTVSW |
| | | NSGALTSGVHTFPAVLQSSGLYSLSSVVTVPSCSLGTQTYICNVNHKPSNTKVDEKVEPKSCDKTHTCPPCPAPEAAGGPSVFLFPP |
| | | KPKDTLMISRTPEVTCVVVDVSHEDPEVKFNWYVDGVEVHNAKTKPREEQYASTYRVVSVLTVLHQDWLNGKEYKCKVSNKALP |
| | | APIEKTISKAKGQPREPQVCTLPPSRDELTKNQVSLSCAVKGFYPSDIAVEWESNGQPENNYKTTPPVLDSDGSFFLVSKLTVDKSR |
| | | WQQGNVFSCSVLHEALHAHYTRKELSLSP |
| | 214 | DIVMTQSPLSLPVTPGEPASISCQPSQEVVHMNRNTYLHWYQQKPGQAPRLLIYKVSNRFPGVPDRFSGSGSGTDFTLKISRVEAEDVGVYYC |
| | | AQGTSHPFTFGQGTKLEIKRTVAAPSVFIFPPSDRKLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | 215 | DIVMTQSPLSLPVTPGEPASISCQPSQEVVHMNRNTYLHWYQQKPGQAPRLLIYKVSNVFPGVPDRFSGSGSGTDFTLKISRVEAEDVGVYYC |
| | | AQGTHHPFTFGQGTKLEIKRTVAAPSVFIFPPSDRKLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | GNVFSCSVMHEALHNHYTQKSLSLSP |
| | 216 | DIQLTQSPSFLSASVGDRVTITCOSTESVYGSDWLSWYQQKPGQPPKLLIYQASNLEIGVPSRFSGSGSGTDFTLTINSLEAEDAATYYCQGYYS |
| | | GYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKT |
| | | YTCNVDHKPSNTKVDKRVEPKSCGGGGSGGGGSQVQLVESGGGLVQPGRSLRLSCAASGFKFSNVWFHWVRQAPGKGLEWVAQIKDYYN |
| | | AYAAYYAPSVKGRFTISRDDSKNSIYLQMNSLKTEDTAVYYCHYVHYASASTLLPAEGVDAWGQGTTVTVSSASTKGPSVFPLAPSSKSTSGGT |
| | | AALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEL |
| | | RGGPKVFLFPPKPKDTLMISRTPEVTCVVVDVSHEDPEVKFNWYVDGVEVHNAKTKPREEQYASTYRVVSVLTVLHQDWLNGKEYKCKVSNK |
| | | ALPAPIEKTISKAKGQPREPQVYTLPPCREEMTKNQVSLWCLVKGFYPSDIAVEWESNGQPENNYKTTPPVLDSDGSFFLYSKLTVDKSRWQE |
| | | GNVFSCSVLHEALHAHYTRKELSLSP |
| | 217 | DIQLTQSPSFLSASVGDRVTITCOSTESVYGSDWLSWYQQKPGQPPKLLIYQASNLEIGVPSRFSGSGSGTDFTLTINSLEAEDAATYYCQGYYS |
| | | GYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKT |
| | | YTCNVDHKPSNTKVDKRVEPKSCGGGGSGGGGSQVOLVESGGGLVQPGRSLRLSCAASGFKFSNVWFHWVRQAPGKGLEWVAQIKDYYN |
| | | AYAGYYHPSVKGRFTISRDDSKNSIYLQMNSLKTEDTAVYYCHYVHYAAASQLLPAEGVDAWGQGTTVTVSSASTKGPSVFPLAPSSKSTSGGT |
| | | AALGCLVEDYFPEPVTVSWNSGALTSGVHTFPAVLQSSGLYSLSSVVTVPSCSLGTQTYICNVNHKPSNTKVDEKVEPKSCDKTHTCPPCPAPE |
| | | AAGGPSVFLFPPKPKDTLMISRTPEVTCVVVDVSHEDPEVKFNWYVDGVEVHNAKTKPREEQYASTYRVVSVLTVLHQDWLNGKEYKCKVSN |
| | | KALPAPIEKTISKAKGQPREPQVYTLPPCRDELTKNQVSLWCLVKGFYPSDIAVEWESNGQPENNYKTTPPVLDSDGSFFLYSKLTVDKSRWQE |
| | | VFSCSVLHEALHAHYTRKELSLSP |
| | 218 | DIQMTQSSSSFSVSLGDRVTITCKASEDIYNRLAWYQQKPGNAPRLLISGATSLETGVPSRFSGSGSGKDYTLSITSLQTEDVATYYCQQYWSTP |
| | | YTFGGGTKLEVKSSASTKGPSVFPLAPSSRSTSESTAALGCLVKDYFPEPVTVSWNSGALTSGVHTFPAVLQSSGLYSLSSVVTVPSSSLGTKTYTC |
| | | NVDHKPSNTKVDKRVEPKSCGGGGSGGGGSQVOLVESGGGLVQPGRSLRLSCAASGFKFSNVWFHWVRQAPGKGLEWVAQIKDYYNAYA |
| | | GYYHPSVKGRFTISRDDSKNSIYLQMNSLKTEDTAVYYCHYVHYAAASQLLPAEGVDAWGQGTTVTVSSASTKGPSVFPLAPSSKSTSGGTAAL |
| | | GCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAG |
| | | GPSVFLFPPKPKDTLMISRTPEVTCVVVDVSHEDPEVKFNWYVDGVEVHNAKTKPREEQYASTYRVVSVLTVLHQDWLNGKEYKCKVSNKAL |
| | | PAPIEKTISKAKGQPREPQVYTLPPCRDELTKNQVSLWCLVKGFYPSDIAVEWESNGQPENNYKTTPPVLDSDGSFFLYSKLTVDKSRWQEGN |
| | 219 | DIQLTQSPSFLSASVGDRVTITCQSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCGGGGSGGGGSQVOLVESGGGLVQPGRSLRLSCAASGFKFSNVWFH |
| | | WVRQAPGKGLEWVAQIKDYYNAYAGYYHPSVKGRFTISRDDSKNSIYLQMNSLKTEDTAVYYCHYVHYAAASQLLPAEGVDAW |
| | | GQGTTVTVSSASTKGPSVFPLAPSSKSTSGGTAALGCLVEDYFPEPVTVSWNSGALTSGVHTFPAVLQSSGLYSLSSVVTVPSCSLG |
| | | TQTYICNVNHKPSNTKVDEKVEPKSCDKTHTCPPCPAPEAAGGPSVFLFPPKPKDTLMISRTPEVTCVVVDVSHEDPEVKFNWYV |
| | | DGVEVHNAKTKPREEQYASTYRVVSVLTVLHQDWLNGKEYKCKVSNKALPAPIEKTISKAKGQPREPQVYTLPPCRDELTKNQVSL |
| | | WCLVKGFYPSDIAVEWESNGQPENNYKTTPPVLDSDGSFFLYSKLTVDKSRWQQGNVFSCSVLHEALHAHYTRKELSLSP |
| | 220 | DIQLTQSPSFLSASVGDRVTITCQSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCGGGGSQVQLVESGGGLVQPGRSLRLSCAASGFKFSNVWFHWVRQA |
| | | PGKGLEWVAQIKDYYNAYAGYYHPSVKGRFTISRDDSKNSIYLQMNSLKTEDTAVYYCHYVHYAAASQLLPAEGVDAWGQGTTV |
| | | TVSSASTKGPSVFPLAPSSKSTSGGTAALGCLVEDYFPEPVTVSWNSGALTSGVHTFPAVLQSSGLYSLSSVVTVPSCSLGTQTYICN |
| | | VNHKPSNTKVDEKVEPKSCDKTHTCPPCPAPEAAGGPSVFLFPPKPKDTLMISRTPEVTCVVVDVSHEDPEVKFNWYVDGVEVH |
| | | NAKTKPREEQYASTYRVVSVLTVLHQDWLNGKEYKCKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGNVFSCSVLHEALHAHYTRKELSLSP |
| | 221 | DIQLTQSPSFLSASVGDRVTITCQSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCQVQLVESGGGLVQPGRSLRLSCAASGFKFSNVWFHWVRQAPGKGL |
| | | EWVAQIKDYYNAYAGYYHPSVKGRFTISRDDSKNSIYLQMNSLKTEDTAVYYCHYVHYAAASQLLPAEGVDAWGQGTTVTVSSA |
| | | STKGPSVFPLAPSSKSTSGGTAALGCLVEDYFPEPVTVSWNSGALTSGVHTFPAVLQSSGLYSLSSVVTVPSCSLGTQTYICNVNHK |
| | | PSNTKVDEKVEPKSCDKTHTCPPCPAPEAAGGPSVFLFPPKPKDTLMISRTPEVTCVVVDVSHEDPEVKFNWYVDGVEVHNAKTK |
| | | PREEQYASTYRVVSVLTVLHQDWLNGKEYKCKVSNKALPAPIEKTISKAKGQPREPQVYTLPPCRDELTKNQVSLWCLVKGFYPSDI |
| | | AVEWESNGQPENNYKTTPPVLDSDGSFFLYSKLTVDKSRWQQGNVFSCSVLHEALHAHYTRKELSLSP |
| | 222 | DIQLTQSPSFLSASVGDRVTITCOSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCGGGGSGGGGSQVOLVESGGGLVQPGRSLRLSCAASGFKFSNVWFH |
| | | WVRQAPGKGLEWVAQIKDYYNAYAGYYHPSVKGRFTISRDDSKNSIYLQMNSLKTEDTAVYYCHYVHYAAASQLLPAEGVDAW |
| | | GQGTTVTVSSASTKGPSVFPLAPSSKSTSGGTAALGCLVEDYFPEPVTVSWNSGALTSGVHTFPAVLQSSGLYSLSSVVTVPSCSLG |
| | | TQTYICNVNHKPSNTKVDEKVEPKSCDKTHTCPPCPAPELRGGPKVFLFPPKPKDTLMISRTPEVTCVVVDVSHEDPEVKFNWYV |
| | | DGVEVHNAKTKPREEQYASTYRVVSVLTVLHQDWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRWQEGNVFSCSVMHEALHNHYTQKSLSLSP |
| | 223 | DIQLTQSPSFLSASVGDRVTITCQSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCGGGGSGGGGSQVQLVESGGGLVQPGRSLRLSCAASGFVFSNVWFH |
| | | WVRQAPGKGLEWVAQIKDYYNAYAAYYAPSVKGRFTISRDDSKNSIYLQMNSLKTEDTAVYYCHYVHYASASTLLPAEGVDAWG |
| | | QGTTVTVSSASTKGPSVFPLAPSSKSTSGGTAALGCLVEDYFPEPVTVSWNSGALTSGVHTFPAVLQSSGLYSLSSVVTVPSCSLGT |
| | | QTYICNVNHKPSNTKVDEKVEPKSCDKTHTCPPCPAPEAAGGPSVFLFPPKPKDTLMISRTPEVTCVVVDVSHEDPEVKFNWYVD |
| | | GVEVHNAKTKPREEQYASTYRVVSVLTVLHQDWLNGKEYKCKVSNKALPAPIEKTISKAKGQPREPQVYTLPPCRDELTKNQVSL |
| | | WCLVKGFYPSDIAVEWESNGQPENNYKTTPPVLDSDGSFFLYSKLTVDKSRWQEGNVFSCSVLHEALHAHYTRKELSLSP |
| | 224 | DIQMTQSSSSFSVSLGDRVTITCKASEDIYNRLAWYQQKPGNAPRLLISGATSLETGVPSRFSGSGSGKDYTLSITSLQTEDVATYYC |
| | | QQYWSTPYTFGGGTKLEVKSSASTKGPSVFPLAPSSRSTSESTAALGCLVKDYFPEPVTVSWNSGALTSGVHTFPAVLQSSGLYSLS |
| | | SVVTVPSSSLGTKTYTCNVDHKPSNTKVDKRVEPKSCGGGGSGGGGSQVOLVESGGGLVQPGRSLRLSCAASGFVFSNVWFHW |
| | | VRQAPGKGLEWVAQIKDYYNAYAAYYAPSVKGRFTISRDDSKNSIYLQMNSLKTEDTAVYYCHYVHYASASTLLPAEGVDAWGQ |
| | | GTTVTVSSASTKGPSVFPLAPSSKSTSGGTAALGCLVEDYFPEPVTVSWNSGALTSGVHTFPAVLQSSGLYSLSSVVTVPSCSLGTQ |
| | | TYICNVNHKPSNTKVDEKVEPKSCDKTHTCPPCPAPEAAGGPSVFLFPPKPKDTLMISRTPEVTCVVVDVSHEDPEVKFNWYVDG |
| | | VEVHNAKTKPREEQYASTYRVVSVLTVLHQDWLNGKEYKCKVSNKALPAPIEKTISKAKGQPREPQVYTLPPCRDELTKNQVSLW |
| | | CLVKGFYPSDIAVEWESNGQPENNYKTTPPVLDSDGSFFLYSKLTVDKSRWQEGNVFSCSVLHEALHAHYTRKELSLSP |
| | 225 | DIQLTQSPSFLSASVGDRVTITCOSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCGGGGSGGGGSQVOLVESGGGLVQPGRSLRLSCAASGFVFSNVWFH |
| | | WVRQAPGKGLEWVAQIKDYYNAYAAYYAPSVKGRFTISRDDSKNSIYLQMNSLKTEDTAVYYCHYVHYASASTLLPAEGVDAWG |
| | | QGTTVTVSSASTKGPSVFPLAPSSKSTSGGTAALGCLVEDYFPEPVTVSWNSGALTSGVHTFPAVLQSSGLYSLSSVVTVPSCSLGT |
| | | QTYICNVNHKPSNTKVDEKVEPKSCDKTHTCPPCPAPEAAGGPSVFLFPPKPKDTLMISRTPEVTCVVVDVSHEDPEVKFNWYVD |
| | | GVEVHNAKTKPREEQYASTYRVVSVLTVLHQDWLNGKEYKCKVSNKALPAPIEKTISKAKGQPREPQVYTLPPCRDELTKNQVSL |
| | | WCLVKGFYPSDIAVEWESNGQPENNYKTTPPVLDSDGSFFLYSKLTVDKSRWQQGNVFSCSVLHEALHAHYTRKELSLSP |
| | 226 | DIQLTQSPSFLSASVGDRVTITCQSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCGGGGSQVQLVESGGGLVQPGRSLRLSCAASGFVFSNVWFHWVRQA |
| | | PGKGLEWVAQIKDYYNAYAAYYAPSVKGRFTISRDDSKNSIYLQMNSLKTEDTAVYYCHYVHYASASTLLPAEGVDAWGQGTTVT |
| | | VSSASTKGPSVFPLAPSSKSTSGGTAALGCLVEDYFPEPVTVSWNSGALTSGVHTFPAVLQSSGLYSLSSVVTVPSCSLGTQTYICNV |
| | | NHKPSNTKVDEKVEPKSCDKTHTCPPCPAPEAAGGPSVFLFPPKPKDTLMISRTPEVTCVVVDVSHEDPEVKFNWYVDGVEVHN |
| | | AKTKPREEQYASTYRVVSVLTVLHQDWLNGKEYKCKVSNKALPAPIEKTISKAKGQPREPQVYTLPPCRDELTKNQVSLWCLVKGF |
| | | YPSDIAVEWESNGQPENNYKTTPPVLDSDGSFFLYSKLTVDKSRWQQGNVFSCSVLHEALHAHYTRKELSLSP |
| | 227 | DIQLTQSPSFLSASVGDRVTITCOSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCQVQLVESGGGLVQPGRSLRLSCAASGFVFSNVWFHWVRQAPGKGL |
| | | EWVAQIKDYYNAYAAYYAPSVKGRFTISRDDSKNSIYLQMNSLKTEDTAVYYCHYVHYASASTLLPAEGVDAWGQGTTVTVSSAS |
| | | TKGPSVFPLAPSSKSTSGGTAALGCLVEDYFPEPVTVSWNSGALTSGVHTFPAVLQSSGLYSLSSVVTVPSCSLGTQTYICNVNHKP |
| | | SNTKVDEKVEPKSCDKTHTCPPCPAPEAAGGPSVFLFPPKPKDTLMISRTPEVTCVVVDVSHEDPEVKFNWYVDGVEVHNAKTKP |
| | | REEQYASTYRVVSVLTVLHQDWLNGKEYKCKVSNKALPAPIEKTISKAKGQPREPQVYTLPPCRDELTKNQVSLWCLVKGFYPSDI |
| | | AVEWESNGQPENNYKTTPPVLDSDGSFFLYSKLTVDKSRWQQGNVFSCSVLHEALHAHYTRKELSLSP |
| | 228 | DIQLTQSPSFLSASVGDRVTITCQSTESVYGSDWLSWYQQKPGQPPKLLIYQASNLEIGVPSRFSGSGSGTDFTLTINSLEAEDAATY |
| | | YCQGYYSGYIYAFGGGTKVEIKSSASTKGPSVFPLAPSSRSTSESTAALGCLVKDYFPEPVTVSWNSGALTSGVHTFPAVLQSSGLYS |
| | | LSSVVTVPSSSLGTKTYTCNVDHKPSNTKVDKRVEPKSCGGGGSGGGGSQVQLVESGGGLVQPGRSLRLSCAASGFVFSNVWFH |
| | | WVRQAPGKGLEWVAQIKDYYNAYAAYYAPSVKGRFTISRDDSKNSIYLQMNSLKTEDTAVYYCHYVHYASASTLLPAEGVDAWG |
| | | QGTTVTVSSASTKGPSVFPLAPSSKSTSGGTAALGCLVEDYFPEPVTVSWNSGALTSGVHTFPAVLQSSGLYSLSSVVTVPSCSLGT |
| | | QTYICNVNHKPSNTKVDEKVEPKSCDKTHTCPPCPAPELRGGPKVFLFPPKPKDTLMISRTPEVTCVVVDVSHEDPEVKFNWYVD |
| | | GVEVHNAKTKPREEQYASTYRVVSVLTVLHQDWLNGKEYKCKVSNKALPAPIEKTISKAKGQPREPQVYTLPPCREEMTKNQVSL |
| | | WCLVKGFYPSDIAVEWESNGQPENNYKTTPPVLDSDGSFFLYSKLTVDKSRWQEGNVFSCSVMHEALHNHYTQKSLSLSP |
| | 229 | QVQLVESGGGLVQPGRSLRLSCAASGFKFSNVWFHWVRQAPGKGLEWVAQIKDYYNAYAAYYAPSVKGRFTISRDDSKNSIYLQ |
| | | MNSLKTEDTAVYYCHYVHYASASTLLPAEGVDAWGQGTTVTVSSASTKGPSVFPLAPSSKSTSGGTAALGCLVEDYFPEPVTVSW |
| | | NSGALTSGVHTFPAVLQSSGLYSLSSVVTVPSCSLGTQTYICNVNHKPSNTKVDEKVEPKSCDKTHTCPPCPAPELRGGPKVFLFPP |
| | | KPKDTLMISRTPEVTCVVVDVSHEDPEVKFNWYVDGVEVHNAKTKPREEQYASTYRVVSVLTVLHQDWLNGKEYKCKVSNKALP |
| | | APIEKTISKAKGQPREPQVCTLPPSREEMTKNQVSLSCAVKGFYPSDIAVEWESNGQPENNYKTTPPVLDSDGSFFLVSKLTVDKSR |
| | | WQEGNVFSCSVMHEALHNHYTQKSLSLSP |
| | 230 | QVQLVESGGGLVQPGRSLRLSCAASGFKFSNVWFHWVRQAPGKGLEWVAQIKDYYNAYAGYYHPSVKGRFTISRDDSKNSIYLQ |
| | | MNSLKTEDTAVYYCHYVHYAAASQLLPAEGVDAWGQGTTVTVSSASTKGPSVFPLAPSSKSTSGGTAALGCLVEDYFPEPVTVSW |
| | | NSGALTSGVHTFPAVLQSSGLYSLSSVVTVPSCSLGTQTYICNVNHKPSNTKVDEKVEPKSCDKTHTCPPCPAPELRGGPKVFLFPP |
| | | KPKDTLMISRTPEVTCVVVDVSHEDPEVKFNWYVDGVEVHNAKTKPREEQYASTYRVVSVLTVLHQDWLNGKEYKCKVSNKALP |
| | | APIEKTISKAKGQPREPQVCTLPPSREEMTKNQVSLSCAVKGFYPSDIAVEWESNGQPENNYKTTPPVLDSDGSFFLVSKLTVDKSR |
| | | WQEGNVFSCSVMHEALHNHYTQKSLSLSP |
| | 231 | QVQLVESGGGLVQPGRSLRLSCAASGFVFSNVWFHWVRQAPGKGLEWVAQIKDYYNAYAAYYAPSVKGRFTISRDDSKNSIYLQ |
| | | MNSLKTEDTAVYYCHYVHYASASTLLPAEGVDAWGQGTTVTVSSASTKGPSVFPLAPSSKSTSGGTAALGCLVEDYFPEPVTVSW |
| | | NSGALTSGVHTFPAVLQSSGLYSLSSVVTVPSCSLGTQTYICNVNHKPSNTKVDEKVEPKSCDKTHTCPPCPAPELRGGPKVFLFPP |
| | | KPKDTLMISRTPEVTCVVVDVSHEDPEVKFNWYVDGVEVHNAKTKPREEQYASTYRVVSVLTVLHQDWLNGKEYKCKVSNKALP |
| | | APIEKTISKAKGQPREPQVCTLPPSREEMTKNQVSLSCAVKGFYPSDIAVEWESNGQPENNYKTTPPVLDSDGSFFLVSKLTVDKSR |
| | | WQEGNVFSCSVMHEALHNHYTQKSLSLSP |
| D08410053H0118 | 232 | QVTLRESGPALVKPTQTLTLTCTFSGFSLSSSYDMGWVRQAPGQGLEWMGTIYTGDYSTDYASWAKGRVTI |
| | | SVDRSKNQFSLKLSSVTAADTAVYYCARHTGYGYFGLWGQGTLVTVSS |
| D08410053H0118_VHR_CDR1 | 233 | SSYDMG |
| D08410053H0118_VHR_CDR2 | 234 | TIYTGDYSTDYASWAKG |
| D08410053H0118_VHR_CDR3 | 235 | HTGYGYFGL |
| D084101L0000 | 236 | DIQLTQSPSFLSASVGDRVTITCOSTESVYGSDWLSWYQQKPGQPPKLLIYQASNLEIGVPSRFSGSGSGTDFT |
| | | LTINSLEAEDAATYYCQGYYSGYIYAFGGGTKVEIK |
| D084101L0000_VLR_CDR1 | 237 | QSTESVYGSDWLS |
| D084101L0000_VLR_CDR2 | 238 | QASNLEI |
| D084101L0000_VLR_CDR3 | 239 | QGYYSGYIYA |
| IC17HdK | 240 | QVQLQQSGPQLVRPGASVKISCKASGYSFTSYWMHWVNQRPGQGLEWIGMIDPSYSETRLNQKFKDKATL |
| | | TVDKSSSTAYMQLSSPTSEDSAVYYCALYGNYFDYWGQGTTLTVSS |
| IC17HdK_VHR_CDR1 | 241 | SYWMH |
| IC17HdK_VHR_CDR2 | 242 | MIDPSYSETRLNQKFKD |
| IC17HdK_VHR_CDR3 | 243 | YGNYFDY |
| IC17L | 244 | DIQMTQSSSSFSVSLGDRVTITCKASEDIYNRLAWYQQKPGNAPRLLISGATSLETGVPSRFSGSGSGKDYTLSI |
| | | TSLQTEDVATYYCQQYWSTPYTFGGGTKLEVK |
| IC17L_VLR_CDR1 | 245 | KASEDIYNRLA |
| IC17L_VLR_CDR2 | 246 | GATSLET |
| IC17L_VLR_CDR3 | 247 | QQYWSTPYT |
| | 248 | VEPKSCGGGGS |
| | 249 | VEPKSCGGGGSGGGGS |
| DLL3CD3BITE | 250 | QVQLQESGPGLVKPSETLSLTCTVSGGSISSYYWSWIRQPPGKGLEWIGYVYYSGTTNYNPSLKSRVTISVDTSK |
| | | NQFSLKLSSVTAADTAVYYCASIAVTGFYFDYWGQGTLVTVSSGGGGSGGGGSGGGGSEIVLTQSPGTLSLSP |
| | | GERVTLSCRASQRVNNNYLAWYQQRPGQAPRLLIYGASSRATGIPDRFSGSGSGTDFTLTISRLEPEDFAVYYC |
| | | QQYDRSPLTFGGGTKLEIKSGGGGSEVOLVESGGGLVQPGGSLKLSCAASGFTFNKYAMNWVRQAPGKGLE |
| | | WVARIRSKYNNYATYYADSVKDRFTISRDDSKNTAYLQMNNLKTEDTAVYYCVRHGNFGNSYISYWAYWGQ |
| | | GTLVTVSSGGGGSGGGGSGGGGSQTVVTQEPSLTVSPGGTVTLTCGSSTGAVTSGNYPNWVQQKPGQAPR |
| | | GLIGGTKFLAPGTPARFSGSLLGGKAALTLSGVQPEDEAEYYCVLWYSNRWVFGGGTKLTVLGGGGDKTHTCP |
| | | PCPAPELLGGPSVFLFPPKPKDTLMISRTPEVTCVVVDVSHEDPEVKFNWYVDGVEVHNAKTKPCEEQYGSTY |
| | | RCVSVLTVLHQDWLNGKEYKCKVSNKALPAPIEKTISKAKGQPREPQVYTLPPSREEMTKNQVSLTCLVKGFYP |
| | | SDIAVEWESNGQPENNYKTTPPVLDSDGSFFLYSKLTVDKSRWQQGNVFSCSVMHEALHNHYTQKSLSLSPG |
| | | KGGGGSGGGGSGGGGSGGGGSGGGGSGGGGSDKTHTCPPCPAPELLGGPSVFLFPPKPKDTLMISRTPEVT |
| | | CVVVDVSHEDPEVKFNWYVDGVEVHNAKTKPCEEQYGSTYRCVSVLTVLHQDWLNGKEYKCKVSNKALPAPI |
| | | EKTISKAKGQPREPQVYTLPPSREEMTKNQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| CD3εVH | 251 | QVQLVESGGGVVQPGGSLRLSCAASGFTFSNAWMHWVRQAPGKGLEWVAQIKDKSQNYATYVAESVKGR |
| | | FTISRADSKNSIYLQMNSLKTEDTAVYYCRYVHYAAGYGVDIWGQGTTVTVSS |
| CD3εVH_VHR_CDR1 | 252 | NAWMH |
| CD3εVH_VHR_CDR2 | 253 | QIKDKSQNYATYVAESVKG |
| CD3εVH_VHR_CDR3 | 254 | VHYAAGYGVDI |
| CD3εVL | 255 | DIVMTQSPLSLPVTPGEPASISCRSSQPLVHSNRNTYLHWYQQKPGQAPRLLIYKVSNRFSGVPDRFSGSGSGT |
| | | DFTLKISRVEAEDVGVYYCGQGTQVPYTFGQGTKLEIK |
| CD3εVL_VLR_CDR1 | 256 | RSSQPLVHSNRNTYLH |
| CD3EVL_VLR_CDR2 | 257 | KVSNRFS |
| CD3EVL_VLR_CDR3 | 258 | GQGTQVPYT |
| | 259 | VEPKSC |
| SG1321kV11Fc | 99 | CPPCPAPEAAGGPSVFLFPPKPKDTLMISRTPEVTCVVVDVSHEDPEVKFNWYVDGVEVHNAKTKPREEQYA |
| | | STYRVVSVLTVLHQDWLNGKEYKCKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQEGNVFSCSVLHEALHAHYTRKELSLSP |
| SG1372kV11Fc | 100 | CPPCPAPEAAGGPSVFLFPPKPKDTLMISRTPEVTCVVVDVSHEDPEVKFNWYVDGVEVHNAKTKPREEQYA |
| | | STYRVVSVLTVLHQDWLNGKEYKCKVSNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQQGNVFSCSVLHEALHAHYTRKELSLSP |
| SG1176kV11Fc | 108 | CPPCPAPELRGGPKVFLFPPKPKDTLMISRTPEVTCVVVDVSHEDPEVKFNWYVDGVEVHNAKTKPREEQYAS |
| | | TYRVVSVLTVLHQDWLNGKEYKCKVSNKALPAPIEKTISKAKGQPREPQVYTLPPCREEMTKNQVSLWCLVKG |
| | | FYPSDIAVEWESNGQPENNYKTTPPVLDSDGSFFLYSKLTVDKSRWQEGNVFSCSVMHEALHNHYTQKSLSLS |
| | | P |
| SG1321hV11Fc | 109 | CPPCPAPEAAGGPSVFLFPPKPKDTLMISRTPEVTCVVVDVSHEDPEVKFNWYVDGVEVHNAKTKPREEQYA |
| | | STYRVVSVLTVLHQDWLNGKEYKCKVSNKALPAPIEKTISKAKGQPREPQVCTLPPSRDELTKNQVSLSCAVKG |
| | | FYPSDIAVEWESNGQPENNYKTTPPVLDSDGSFFLVSKLTVDKSRWQEGNVFSCSVLHEALHAHYTRKELSLSP |
| SG1372hV11Fc | 111 | CPPCPAPEAAGGPSVFLFPPKPKDTLMISRTPEVTCVVVDVSHEDPEVKFNWYVDGVEVHNAKTKPREEQYA |
| | | STYRVVSVLTVLHQDWLNGKEYKCKVSNKALPAPIEKTISKAKGQPREPQVCTLPPSRDELTKNQVSLSCAVKG |
| | | FYPSDIAVEWESNGQPENNYKTTPPVLDSDGSFFLVSKLTVDKSRWQQGNVFSCSVLHEALHAHYTRKELSLS |
| | | P |
| SG1176hV11Fc | 112 | CPPCPAPELRGGPKVFLFPPKPKDTLMISRTPEVTCVVVDVSHEDPEVKFNWYVDGVEVHNAKTKPREEQYAS |
| | | TYRVVSVLTVLHQDWLNGKEYKCKVSNKALPAPIEKTISKAKGQPREPQVCTLPPSREEMTKNQVSLSCAVKGF |
| | | YPSDIAVEWESNGQPENNYKTTPPVLDSDGSFFLVSKLTVDKSRWQEGNVFSCSVMHEALHNHYTQKSLSLSP |

### Example 3

### In Vitro Efficacy of DLL3-Dual-LINC Antibody Towards DLL3 Expression Cell Lines

### 3.1. Measurement of TDCC Activity Using Anti-DLL3/Dual-Linc (1+2) Antibody, Dual (1+1) Antibody, DLL3CD3BiTE, and DLL3/CD3 Epsilon (1+1) Antibody

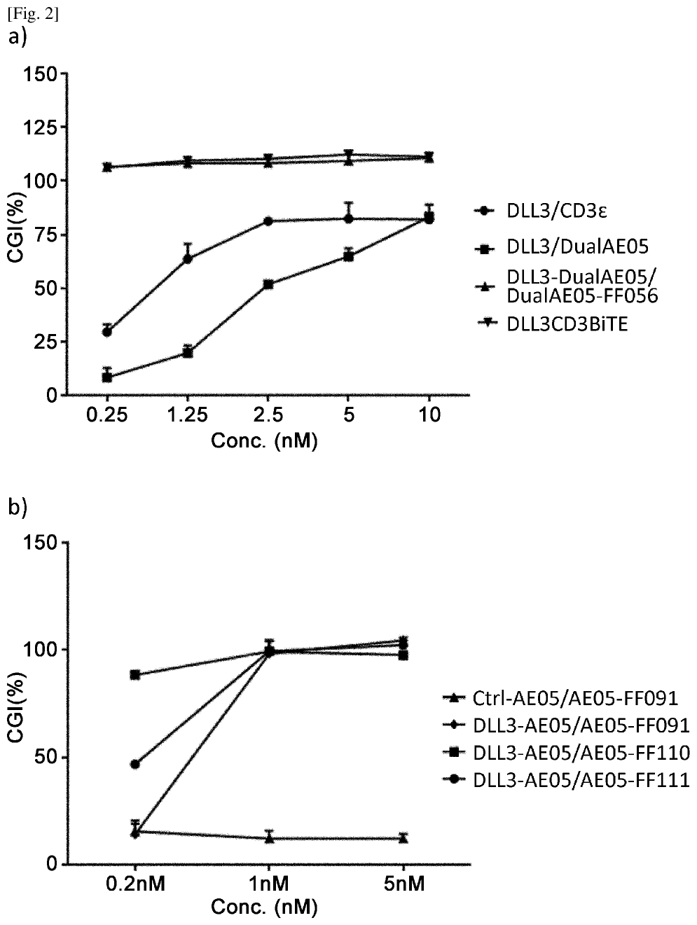

Cytotoxic activity was assessed by observing the rate of tumor cell growth inhibition using xCELLigence Real-Time Cell Analyzer (Roche Diagnostics) in the presence of PBMCs. FIG. 2 shows the TDCC activity of anti-DLL3/Dual-Linc antibody (DLL3-DualAE05/DualAE05, 1+2), Dual (1+1) antibody (DLL3/DualAE05), DLL3CD3BiTE, and DLL3/CD3 epsilon (1+1) antibody prepared according to Tables 10-13. SK-MEL30 cell line was used as target cells. Target cells were detached from the dish and cells were plated into E-plate 96 (Roche Diagnostics) in aliquots of 100 micro L/well by adjusting the cells to 5×10³ cells/well, and measurement of cell growth was initiated using the xCELLigence Real-Time Cell Analyzer. 24 hours later, the plate was removed and 50 micro L of the respective antibodies prepared at each concentration (serial dilutions starting from 10 nM i.e., 0.25, 1.25, 2.5, 5, and 10 nM) were added to the plate. After 15 minutes of reaction at room temperature, 50 micro L of the fresh human PBMC solution was added in effector:target ratio of 2 (i.e. 1×10⁴ cells/well) and measurement of cell growth was resumed using xCELLigence Real-Time Cell Analyzer. The reaction was carried out under the conditions of 5% carbon dioxide gas at 37 degrees C. As CD137 signaling enhances T-cell survival and prevents activation induced cell death, TDCC assay was conducted at a low E:T ratio. An extended period of time may be required to observe superior cytotoxicity contributed by CD137 activation. As such, approximately 68 hours after the addition of PBMCs, Cell Growth Inhibition (CGI) rate (%) was determined using the equation below. The Cell Index Value obtained from xCELLigence Real-Time Cell Analyzer used in the calculation was a normalized value where the Cell Index value immediately at the time point before antibody addition was defined as 1.

$$Cell Growth Inhibition rate ( \% ) = ( A - B ) \times 100 / ( A - 1 )$$

A represents the mean value of Cell Index values in wells without antibody addition (containing only target cells and human PBMCs), and B represents the mean value of the Cell Index values of target wells. The examinations were performed in duplicates.

As shown in FIG. 2 a), anti-DLL3/Dual-Linc antibody (DLL3-DualAE05/DualAE05, 1+2) showed stronger TDCC activity than those of Dual (1+1) antibody (DLL3/DualAE05) and DLL3/CD3 epsilon (1+1) antibody. This suggests that Dual-Line molecular format contributes to improved cytotoxicity. In addition, at these antibody concentrations, TDCC activity of anti-DLL3/Dual-Linc antibody are comparable to DLL3/CD3BiTE.

### 3.2. Evaluation of Linker Length Effect for Cytotoxicity

To improve cytotoxicity against tumor cells, the closer proximity and more rigid binding between tumor cells and effector cells would be critical. This means that the shorter distance may help to bring the T cell and tumor cell in close proximity, which may help for the TDCC. Therefore, we generated three anti-DLL3/Dual-Linc antibody variants which have linkers of different lengths. The linker length of DLL3-DualAE05/DualAE05-FF110 (mid linker) and DLL3-DualAE05/DualAE05-FF111 (short linker) are shorter than that of DLL3-DualAE05/DualAE05-FF091 (long linker) (herein below these variants may be abbreviated as DLL3-AE05/AE05-FF110, DLL3-AE05/AE05-FF111, and DLL3-AE05/AE05-FF091, respectively). To evaluate the linker length effect for cytotoxicity, TDCC measurement was conducted as described in Example 3.1 using 0.2, 1, and 5 nM of antibodies.

As shown in FIG. 2 b), DLL3-AE05/AE05-FF110 (mid linker) and DLL3-AE05/AE05-FF111 (short linker) showed stronger TDCC activity than that of DLL3-AE05/AE05-FF091 (long linker) at 0.2 nM. DLL3-AE05/AE05-FF110 (mid linker) showed the strongest TDCC activity. These results suggest that the linker length is important for TDCC activity and the mid-length linker is most appropriate for in vitro TDCC activity.

### Example 4

Assessment of In Vivo Efficacy of DLL3-DualAE05/DualAE05-FF091 Tri-Specific Antibody with Compared to DLL3CD3BiTE.

The anti-tumor activity of DLL3-DualAE05/DualAE05-FF091 antibody and DLL3CD3BiTE prepared in Example 2 were tested in a human small cell lung cancer NCI-H1436 cancer model. NCI-H1436 cells were subcutaneously transplanted to NOG humanized mice. NOG female mice were purchased from In-Vivo Science. For humanization, mice were sub lethally irradiated followed 1 day later by injection of 100,000 human cord blood cells (ALLCELLS). After humanization, NCI-H1436 (5×10⁶ cells) were mixed with Matrigel Basement Membrane Matrix (Corning) and transplanted to the right flank of humanized NOG mice. The day of transplantation was defined as day 0. On day 11, the mice were randomized on the basis of tumor volume and body weight. On the following day, mice were injected i.v. with either vehicle (PBS containing 0.05% Tween), 6.5 mg/kg DLL3-DualAE05/DualAE05-FF091, and 3.5 mg/kg DLL3CD3BiTE.

For T cell infiltration analysis, the xenografted tumors beard on the mice were harvested at indicated time points after the antibody administration. The tumors were weighted, minced by using human Tumor Dissosication Kit (Miltenyi) according to the manufacturer's protocol and CountBright Absolute Counting Beads (ThermoFisher scientific) was added after dissociation. Intratumor CD8+ T cell numbers was evaluated using LSRFortessa X-20 (BD).

For T cell exhaustion analysis, the xenografted tumors beard on the mice were harvested day 7 after the antibody administration. The tumors were minced by human Tumor Dissosication Kit (Miltenyi) according to the manufacturer's protocol and the intratumor T cells were evaluated for the expression of T cell exhaustion markers, Tim-3, PD-1, and Lag-3.

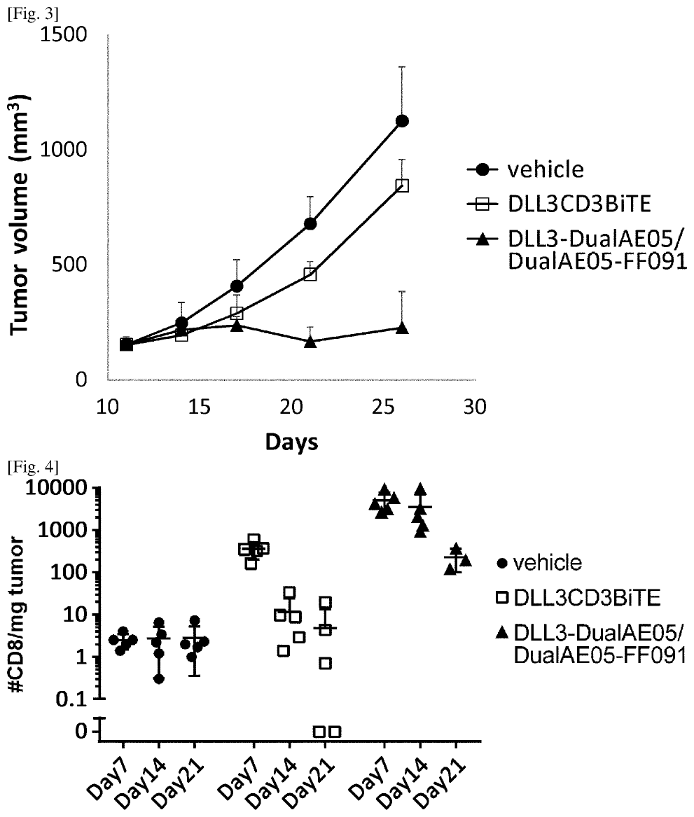

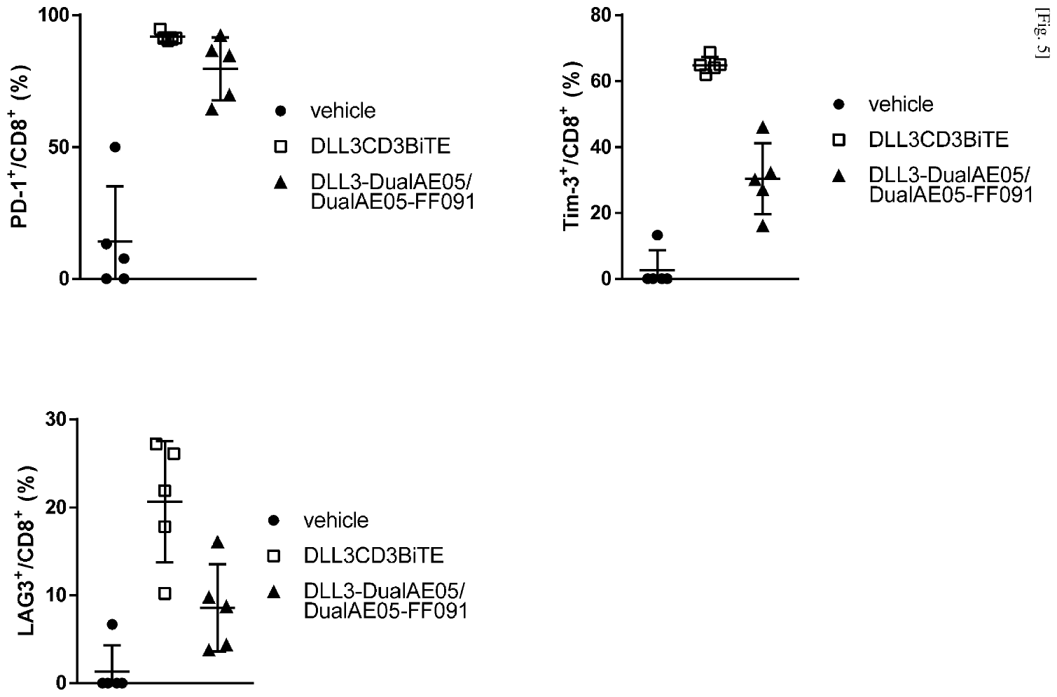

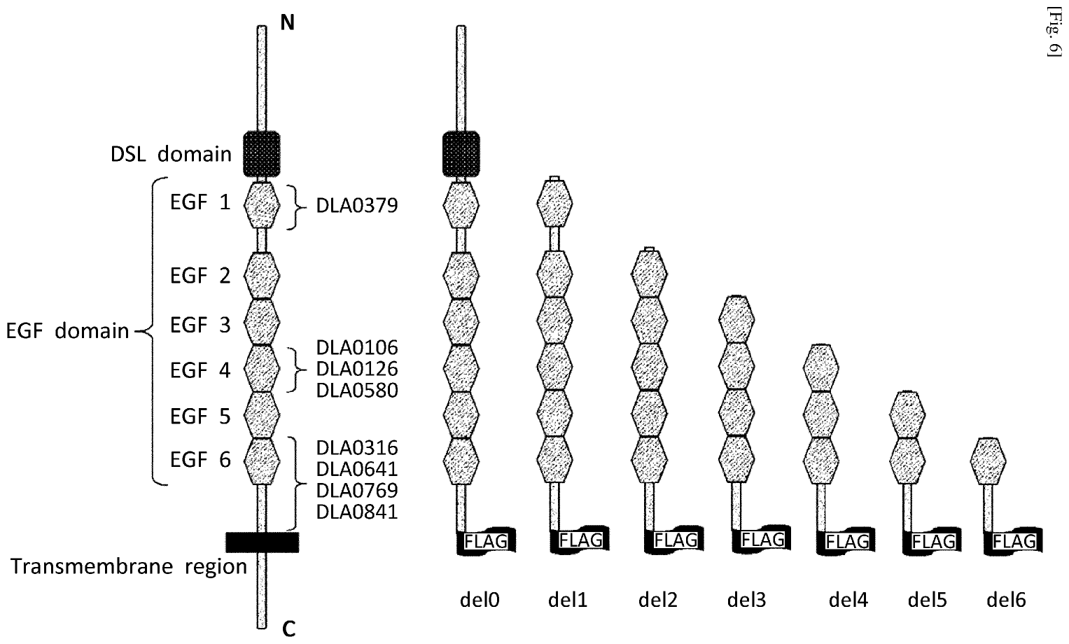

As a result, DLL3-DualAE05/DualAE05-FF091 showed stronger efficacy and higher T cell infiltration than DLL3CD3BiTE (FIG. 3 and FIG. 4). Furthermore, T cells of DLL3-DualAE05/DualAE05-FF091-treated group had lower expression of PD-1, Tim-3, and LAG3 (FIG. 5), suggesting that DLL3-DualAE05/DualAE05-FF091 induces less T cell exhaustion.

### Example 5

### In Vitro Efficacy of Anti-DLL3-Dual-LINC Antibody Towards Non-SCLC Cell Lines

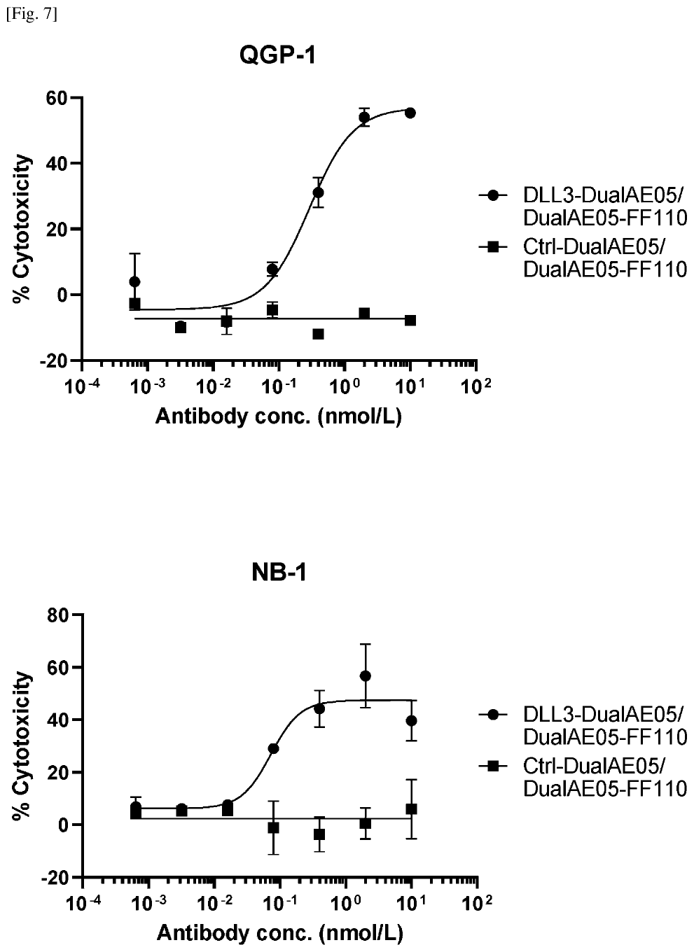

In vitro cytotoxicity of anti-DLL3-Dual-antibody (DLL3-DualAE05/DualAE05-FF110) and untargeted control Dual-LINC antibody (Ctrl-DualAE05/DualAE05-FF110) towards non-SCLC cell lines was evaluated by LDH Cytotoxicity Detection Kit (Takara Bio). NB-1 cell line (neuroblastoma, JCRB cell bank IF050295) and QGP-1 cell line (pancreatic islet cell carcinoma, JCRB cell bank JCRB0183) were used as target cells. Target cells were seeded at 1×10⁴ cells/well in 96 well culture plates, and antibodies prepared at each concentration (5-fold serial dilutions from 10 nM to 0.00064 nM) were added. Thereafter, 2×10⁵ cells of human PBMCs were added and incubated at 37 degrees Celsius. 24 hours later, high control samples were lysed by Triton-X 100 and 50 micro-L of supernatant was collected from all wells. Cytotoxicity (%) was calculated according to the manufacturer's protocol. As shown in FIG. 7, anti-DLL3-Dual-LINC antibody (DLL3-DualAE05/DualAE05-FF110) showed in vitro cell killing activity towards non-SCLC cell lines including NB-1 cell line and QGP-1 cell line.

### Example 6

In Vivo Efficacy of Anti-DLL3-Dual-LINC Antibody in Combination with Chemotherapeutic Reagents

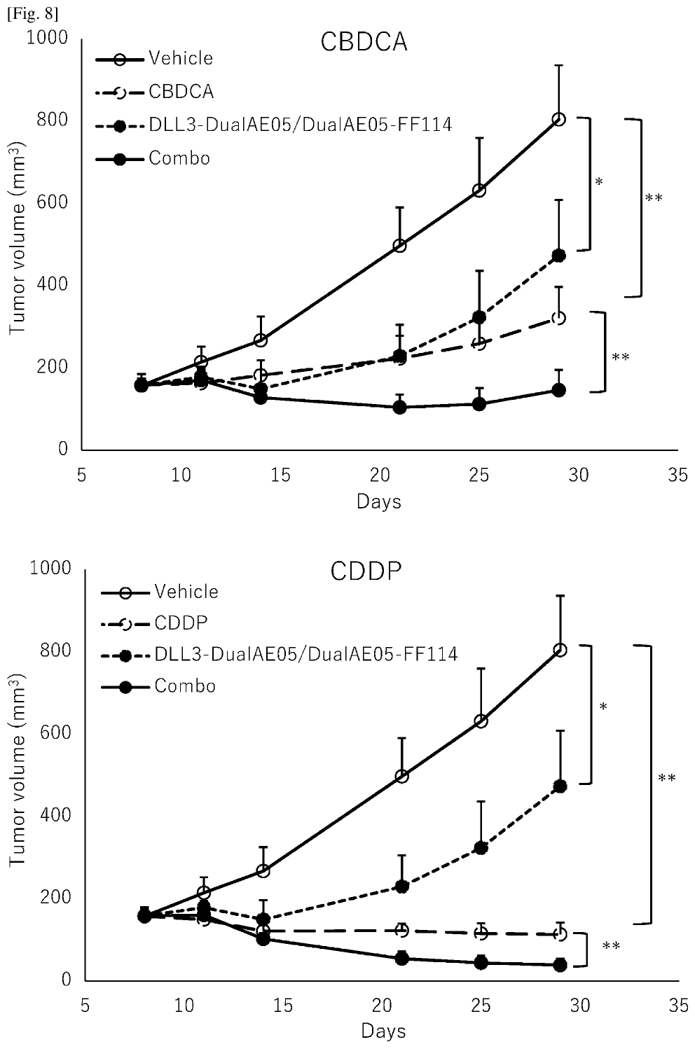

Anti-tumor efficacy of the combination treatment with anti-DLL3-Dual-LINC antibody (DLL3-DualAE05/DualAE05-FF114) and platinum reagent was evaluated in DMS53 small cell lung cancer model. NOG female mice were purchased from In-Vivo Science. For humanization, mice were sublethally irradiated followed 1 day later by injection of 100,000 human cord blood cells (LONZA). DMS53 cells (ATCC CRL-2062) were mixed with Matrigel Basement Membrane Matrix (Corning) and inoculated in the right flank of humanized NOG mice. On day 9 when the tumor volume was around 150 mm³, the mice were randomized on the basis of tumor volume and body weight, and injected intravenously (i.v.) with vehicle (PBS containing 0.05% Tween), 1.3 mg/kg anti-DLL3-Dual-LINC antibody (DLL3-DualAE05/DualAE05-FF114), 7.5 mg/kg cisplatin (CDDP), and 40 mg/kg carboplatin (CBDCA) as monotherapy or in combination. As a result, the combination of anti-DLL3-Dual-LINC antibody DLL3-DualAE05/DualAE05-FF114 and platinum reagents showed significantly greater efficacy than monotherapy of CDDP, CBDCA or anti-DLL3-Dual-LINC antibody alone (FIG. 8).

### Example 7

### In Vivo Efficacy of Anti-DLL3-Dual-LINC Antibody Towards NCI-H1436 Bearing Humanized Model

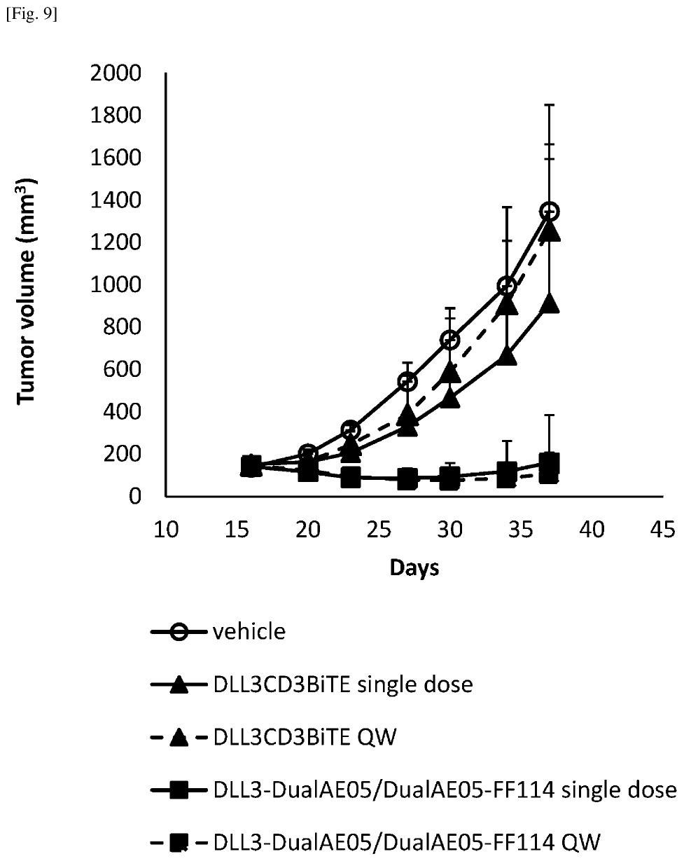

Anti-tumor efficacy of anti-DLL3-Dual-LINC antibody DLL3-DualAE05/DualAE05-FF114 was evaluated in NCI-H1436 small cell lung cancer model. NOG female mice were purchased from In-Vivo Science. For humanization, mice were sublethally irradiated followed 1 day later by injection of 100,000 human cord blood cells (LONZA). NCI-H1436 cells (ATCC CRL-5871) were mixed with Matrigel Basement Membrane Matrix (Corning) and inoculated in the right flank of humanized NOG mice. On day 16 when the tumor volume was around 150 mm³, the mice were randomized based on tumor volume and body weight, and injected i.v. with vehicle (PBS containing 0.05% Tween), 6.5 mg/kg anti-DLL3-Dual-LINC antibody (DLL3-DualAE05/DualAE05-FF114), and 3.5 mg/kg DLL3CD3BiTE. Anti-DLL3-Dual-LINC antibody DLL3-DualAE05/DualAE05-FF114 and DLL3CD3BiTE were administered in a single dose or every week (QW). As a result, Anti-DLL3-Dual-LINC antibody DLL3-DualAE05/DualAE05-FF114 showed stronger efficacy than DLL3CD3BiTE in both treatment regimens (FIG. 9).

### Example 8

### In Vitro Efficacy of Anti-DLL3-Dual-LINC Antibody Towards Other Non-SCLC Cell Lines

In vitro cytotoxicity of anti-DLL3-Dual-LINC antibody (DLL3-DualAE05/DualAE05-FF110 or DLL3-DualAE05/DualAE05-FF114) and untargeted control Dual LINC antibody towards other non-SCLC cell lines is evaluated as described in Example 5. Examples of non-SCLC cell lines which are to be evaluated are cell lines of pancreatic neuroendocrine tumor (PNET), small-cell type NEC (SCNEC), large-cell type NEC (LCNEC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC), neuroblastoma, glioma or glioblastoma (GBM), melanoma, and medullary thyroid cancer.

### Example 9

In Vitro and In Vivo Efficacy of Anti-DLL3-Dual-LINC Antibody in Combination with Other Chemotherapeutic Reagents or Checkpoint Inhibitors

In vitro cytotoxicity of anti-DLL3-Dual-LINC antibody (DLL3-DualAE05/DualAE05-FF110 or DLL3-DualAE05/DualAE05-FF114) in combination with other chemotherapeutic reagents or checkpoint inhibitors (anti-PD-L1 antibody or anti-PD1 antibody) towards SCLC cell line or other non-SCLC cell lines is evaluated using a method as described in Example 5.

In vivo anti-tumor efficacy of the anti-DLL3-Dual-LINC antibody (DLL3-DualAE05/DualAE05-FF110 or DLL3-DualAE05/DualAE05-FF114) in combination with other chemotherapeutic reagents or checkpoint inhibitors (anti-PD-L1 antibody or anti-PD1 antibody) is evaluated in DMS53 small cell lung cancer model and other non-SCLC cancer models as described in Example 6.

In this example, the non-SCLC cell lines (or non-SCLC cancer models) that are to be evaluated are cell lines (or cancer models) of a cancer selected from the group consisting of pancreatic neuroendocrine tumor (PNET), small-cell type NEC (SCNEC), large-cell type NEC (LCNEC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC), neuroblastoma, glioma or glioblastoma (GBM), melanoma, and medullary thyroid cancer.

In this example, the checkpoint inhibitor that is to be evaluated for combination therapy with anti-DLL3-Dual-LINC antibody (DLL3-DualAE05/DualAE05-FF110 or DLL3-DualAE05/DualAE05-FF114) is selected from the group consisting of pembrolizumab, pidilizumab (pidilizumab), nimotuzumab (nivolumab), atezolizumab and durvalumab, and in particular, is atezolizumab.

### Example 10

In Vivo Efficacy of Anti-DLL3-Dual-LINC Antibody in Combination with Steroid or Tocilizumab

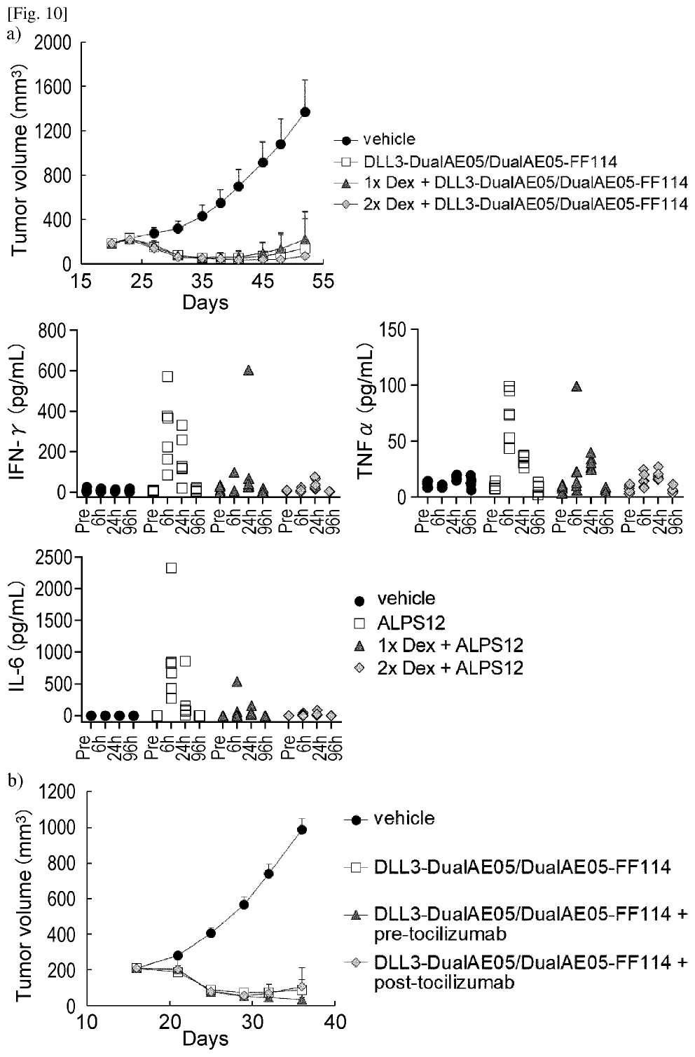

Cytokine release syndrome (CRS) is a common adverse event of T cell-associated therapeutics such as T cell engagers and CAR-T cells. In clinic, treatment with steroid and/or tocilizumab is widely used to control CRS. Thus, in vivo efficacy of anti-DLL3-Dual-LINC antibody (DLL3-DualAE05/DualAE05-FF114) in combination with steroid or tocilizumab was evaluated. Immune humanized NOG mice were generated as described in Example 7. PC-10 cells (Immuno-Biological Laboratories) expressing human DLL3 were engrafted into the right flank of humanized NOG mice. When the tumor volume was around 200 mm³, the mice were randomized based on tumor volume and body weight. In the premedication experiments with steroid, 33 mg/kg dexamethasone was intraperitoneally administered 1 and 24 hours, or 1 hour prior to antibody injection. In the experiments with tocilizumab, 10 mg/kg tocilizumab was administered one day before or 6 hours after injection of anti-DLL3-Dual-LINC antibody. Plasma samples were collected before and 6, 24, and 96 hours after injection of anti-DLL3-Dual-LINC antibody and plasma concentration of cytokines was measured using Human Cytokine/Chemokine Magnetic Bead Panel (HCYTMAG-60K-PX38, Millipore) according to the manufacturer's instruction. As a result, pre-medication with dexamethasone induced marked reduction of cytokine production without affecting the tumor growth inhibition (FIG. 10a). Likewise, pre- and post-medication of tocilizumab did not have any impact on the antitumor efficacy of anti-DLL3-Dual-LINC antibody (FIG. 10b).

### INDUSTRIAL APPLICABILITY

The present invention provides multispecific antigen-binding molecules exhibit enhanced T-cell dependent cytotoxicity activity in a DLL3-dependent manner through binding to the CD3/CD37 and DLL3. The antigen-binding molecules and pharmaceutical compositions thereof can be used for targeting cells expressing DLL3, for use in immunotherapy for treating various cancers, especially those associated with DLL3 such as DLL3-positive cancers.

1. A pharmaceutical formulation comprising an antibody and a pharmaceutically acceptable carrier for use in treatment of cancer, wherein the antibody comprises:
(a) a first antigen-binding moiety and a second antigen-binding moiety, each of which comprises an antibody variable region that can be the same or different and is independently selected from the group consisting of:
(a1) an antibody variable region comprising the heavy chain complementarity determining region (CDR) 1 of SEQ ID NO: 20, the heavy chain CDR 2 of SEQ ID NO: 34, the heavy chain CDR 3 of SEQ ID NO: 48, light chain CDR 1 of SEQ ID NO: 63, light chain CDR 2 of SEQ ID NO: 68 and light chain CDR 3 of SEQ ID NO: 73;
(a2) an antibody variable region comprising the heavy chain complementarity determining region (CDR) 1 of SEQ ID NO: 28, the heavy chain CDR 2 of SEQ ID NO: 42, the heavy chain CDR 3 of SEQ ID NO: 56, light chain CDR 1 of SEQ ID NO: 63, light chain CDR 2 of SEQ ID NO: 68 and light chain CDR 3 of SEQ ID NO: 73;
(a3) an antibody variable region comprising heavy chain complementarity determining region (CDR) 1 of SEQ ID NO: 82, heavy chain CDR 2 of SEQ ID NO: 83, heavy chain CDR 3 of SEQ ID NO: 84, light chain CDR 1 of SEQ ID NO: 65, light chain CDR 2 of SEQ ID NO: 70 and light chain CDR 3 of SEQ ID NO: 75; and
(b) a third antigen-binding moiety that binds to human Delta-like 3 (DLL3) and comprises an antibody variable region comprising a heavy chain CDR1 comprising SEQ ID NO: 233, a heavy chain CDR 2 comprising SEQ ID NO: 234, a heavy chain CDR 3 comprising SEQ ID NO: 235, a light chain CDR 1 comprising SEQ ID NO: 237, a light chain CDR 2 comprising SEQ ID NO: 238, and a light chain CDR 3 comprising SEQ ID NO: 239.

2. The pharmaceutical formulation of claim 1, wherein each of the first and second antigen-binding moieties comprises an antibody variable region that can be the same or different and is independently selected from the group consisting of:
(a1) a heavy chain variable region (VH) comprising an amino acid sequence of SEQ ID NO: 6, and a light chain variable region (VL) comprising an amino acid sequence of SEQ ID NO: 58;
(a2) a VH comprising an amino acid sequence of SEQ ID NO: 14, and a VL comprising an amino acid sequence of SEQ ID NO: 58; and
(a3) a VH comprising an amino acid sequence of SEQ ID NO: 81, and a VL comprising an amino acid sequence of SEQ ID NO: 60.

3. The pharmaceutical formulation of claim 1 or claim 2, wherein each of the first antigen-binding moiety and the second antigen-binding moiety has one, two or more of the following properties:
(i) binds to human CD3;
(ii) binds to human CD137; or
(iii) capable of binding to human CD3 and human CD137, wherein the first antigen-binding moiety binds to either one of human CD3 and human CD137.

4. The pharmaceutical formulation of claim 1 or claim 2, wherein the third antigen-binding moiety comprises an antibody variable region comprising a VH comprising SEQ ID NO: 232 and a VL comprising SEQ ID NO: 236.

5. The pharmaceutical formulation of claim 1 or claim 2, wherein each of the first and second antigen-binding moieties is a Fab that has a cysteine residue at position 191 of the CH1 domain (EU numbering), and wherein there is a disulfide bond linking the two cysteine residues.

6. The pharmaceutical formulation of claim 1 or claim 2, wherein each of the first, second and third antigen binding moieties is a Fab comprising a heavy chain comprising a VH and a CH1 domain and a light chain comprising a VL and a light chain constant (CL) domain, and wherein the C-terminus of the heavy chain of the third antigen binding moiety is fused, directly or via a peptide linker, to the N-terminus of the Fab heavy chain of either the first antigen binding moiety or the second antigen binding moiety.

7. The pharmaceutical formulation of claim 6, wherein the C-terminus of the heavy chain of the third antigen binding moiety is fused, via a peptide linker, to the N-terminus of the Fab heavy chain of either the first antigen binding moiety or the second antigen binding moiety, and wherein the peptide linker has an amino acid sequence selected from the group consisting of SEQ ID NO: 248, SEQ ID NO: 249, and SEQ ID NO: 259.

8. The pharmaceutical formulation of claim 7, wherein the third antigen binding moiety is a crossover Fab molecule in which the variable regions of the Fab light chain and the Fab heavy chain are exchanged, and wherein each of the first and second antigen binding moiety is a conventional Fab molecule.

9. The pharmaceutical formulation of claim 8, wherein, in the CL domain of each of the first and second antigen binding moieties, the amino acids at positions 123 and 124 (Kabat numbering) are arginine and lysine, respectively; and wherein, in the CH1 domain of each of the first and second antigen binding moieties, the amino acid at each of positions 147 and 213 (EU numbering) is glutamic acid.

10. The pharmaceutical formulation of claim 1 or claim 2, further comprising an Fc domain.

11. A multispecific antibody for use in the treatment of cancer, wherein the antibody comprises five polypeptide chains in a combination selected from the group consisting of (A) to (C) below:
(A) a polypeptide chain comprising the amino acid sequence of SEQ ID NO: 201 (chain 1), a polypeptide chain comprising the amino acid sequence of SEQ ID NO: 206 (chain 2), a polypeptide chain comprising the amino acid sequence of SEQ ID NO: 208 (chain 3), and two polypeptide chains each comprising the amino acid sequence of SEQ ID NO: 214 (chains 4 and 5);
(B) a polypeptide chain comprising the amino acid sequence of SEQ ID NO: 203 (chain 1), a polypeptide chain comprising the amino acid sequence of SEQ ID NO: 206 (chain 2), a polypeptide chain comprising the amino acid sequence of SEQ ID NO: 209 (chain 3), and two polypeptide chains each comprising the amino acid sequence of SEQ ID NO: 214 (chains 4 and 5); and
(C) a polypeptide chain comprising the amino acid sequence of SEQ ID NO: 204 (chain 1), a polypeptide chain comprising the amino acid sequence of SEQ ID NO: 206 (chain 2), a polypeptide chain comprising the amino acid sequence of SEQ ID NO: 209 (chain 3), and two polypeptide chains each comprising the amino acid sequence of SEQ ID NO: 214 (chains 4 and 5).

12. The pharmaceutical formulation of claim 1 or claim 2 or the multispecific antibody of claim 11 for use in treatment of cancer, wherein the cancer is a DLL3-expressing or DLL3-positive cancer, preferably selected from the group consisting of neuroendocrine neoplasm (NEN), neuroendocrine tumor (NET), neuroendocrine carcinoma (NEC) or other solid tumors of non-neuroendocrine origin.

13. The pharmaceutical formulation of claim 1 or claim 2 or the multispecific antibody of claim 11 for use in treatment of cancer, wherein the cancer is selected from the group consisting of pancreatic neuroendocrine tumor (PNET), small-cell type NEC (SCNEC), large-cell type NEC (LCNEC), small cell lung cancer (SCLC), large cell-neuroendocrine lung cancer, neuroendocrine prostate cancer (NEPC), Merkel cell carcinoma, small cell urinary bladder cancer, GI neuroendocrine carcinoma, medullary thyroid carcinoma (MTC), gastroenteropancreatic neuroendocrine carcinoma (GEP NEC), neuroblastoma, glioma or glioblastoma (GBM), melanoma, and medullary thyroid cancer.

14. The pharmaceutical formulation of claim 1 or claim 2 or the multispecific antibody of claim 11 for use in treatment of cancer in combination with an additional therapeutic agent, preferably a chemotherapeutic agent, or an immune checkpoint inhibitor.

15. The pharmaceutical formulation or the multispecific antibody of claim 14, for use in treatment of cancer in combination with an immune checkpoint inhibitor, wherein the immune checkpoint inhibitor is a PD-1 axis binding antagonist, preferably an anti-PD-1 antibody or an anti-PD-L1 antibody.

16. The pharmaceutical formulation or the multispecific antibody of claim 14, for use in treatment of cancer in combination with a chemotherapeutic agent, wherein the chemotherapeutic agent is selected from the group consisting of a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, an antiangiogenic agent, Etoposide, Irinotecan, Lurbinectedin, Amrubicin, and platinum agents (such as cisplatin and carboplatin).


SEQUENCE LISTING (320 sequences)

SEQ ID NO: 1 - protein, 128 aa, synthetic construct
  REGION 1..128: note = an artificially synthesized sequence
QVQLVESGGGLVQPGRSLRLSCAASGFTFSNAWMHWVRQAPGKGLEWVAQIKDKGNAYAAYYAPSVKGRFTISRDDSKNSIYLQMNSLKTEDTAVYYCHYVHYASASTVLPAFGVDAWGQGTTVTVSS

SEQ ID NO: 2 - protein, 128 aa, synthetic construct
  REGION 1..128: note = an artificially synthesized sequence
QVQLVESGGGLVQPGRSLRLSCAASGFKFSNVWMHWVRQAPGKGLEWVAQIKDKYNAYAAYYAPSVKGRFTISRDDSKNSIYLQMNSLKTEDTAVYYCHYVHYASASTLLPAFGVDAWGQGTTVTVSS

SEQ ID NO: 3 - protein, 128 aa, synthetic construct
  REGION 1..128: note = an artificially synthesized sequence
QVQLVESGGGLVQPGRSLRLSCAASGFKFSNVWMHWVRQAPGKGLEWVAQIKDKYNAYAAYYAPSVKGRFTISRDDSKNSIYLQMNSLKTEDTAVYYCHYIHYASASTLLPAFGVDAWGQGTTVTVSS

SEQ ID NO: 4 - protein, 128 aa, synthetic construct
  REGION 1..128: note = an artificially synthesized sequence
QVQLVESGGGLVQPGRSLRLSCAASGFKFSNVWFHWVRQAPGKGLEWVAQIKDKYNAYATYYAPSVKGRFTISRDDSKNSIYLQMNSLKTEDTAVYYCHYVHYASASTLLPAFGVDAWGQGTTVTVSS

SEQ ID NO: 5 - protein, 128 aa, synthetic construct
  REGION 1..128: note = an artificially synthesized sequence
QVQLVESGGGLVQPGRSLRLSCAASGFVFSNVWMHWVRQAPGKGLEWVAQIKDKWNAYAAYYAPSVKGRFTISRDDSKNSIYLQMNSLKTEDTAVYYCHYIHYASASTLLPAEGIDAWGQGTTVTVSS

SEQ ID NO: 6 - protein, 128 aa, synthetic construct
  REGION 1..128: note = an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSS

SEQ ID NO: 7 - protein, 207 aa, synthetic construct
  REGION 1..207: note = an artificially synthesized sequence
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 8 - protein, 128 aa, synthetic construct
  REGION 1..128: note = an artificially synthesized sequence
QVQLVESGGGLVQPGRSLRLSCAASGFKFSNTWFHWVRQAPGKGLEWVAQIKDYYNDYAAYYAPSVKGRFTISRDDSKNSIYLQMNSLKTEDTAVYYCHYVHYASASTLLPAEGVDAWGQGTTVTVSS

SEQ ID NO: 9 - protein, 128 aa, synthetic construct
  REGION 1..128: note = an artificially synthesized sequence
QVQLVESGGGLVQPGRSLRLSCAASGFVFSNVWFHWVRQAPGKGLEWVAQIKDKYNAYADYYAPSVKERFTISRDDSKNSIYLQMNSLKTEDTAVYYCHYVHYASASTLLPAEGVDAWGQGTTVTVSS

SEQ ID NO: 10 - protein, 128 aa, synthetic construct
  REGION 1..128: note = an artificially synthesized sequence
QVQLVESGGGLVQPGRSLRLSCAASGFVFSNVWFHWVRQAPGKGLEWVAQIKDKYNAYADYYAPSVEGRFTISRDDSKNSIYLQMNSLKTEDTAVYYCHYVHYASASTLLPAEGVDAWGQGTTVTVSS

SEQ ID NO: 11 - protein, 128 aa, synthetic construct
  REGION 1..128: note = an artificially synthesized sequence
QVQLVESGGGLVQPGRSLRLSCAASGFVFSNVWFHWVRQAPGKGLEWVAQIKDYYNAYADYYAPSVEGRFTISRDDSKNSIYLQMNSLKTEDTAVYYCHYVHYASASTLLPAEGVDAWGQGTTVTVSS

SEQ ID NO: 12 - protein, 128 aa, synthetic construct
  REGION 1..128: note = an artificially synthesized sequence
QVQLVESGGGLVQPGRSLRLSCAASGFVFSNVWFHWVRQAPGKGLEWVAQIKDKWNAYADYYAPSVKERFTISRDDSKNSIYLQMNSLKTEDTAVYYCHYIHYASASTLLPAEGIDAWGQGTTVTVSS

SEQ ID NO: 13 - protein, 128 aa, synthetic construct
  REGION 1..128: note = an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASTLLPAEGVDAWGQGTTVTVSS

SEQ ID NO: 14 - protein, 128 aa, synthetic construct
  REGION 1..128: note = an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSS

SEQ ID NO: 15 - protein, 5 aa, synthetic construct
  REGION 1..5: note = an artificially synthesized sequence
NAWMH

SEQ ID NO: 16 - protein, 5 aa, synthetic construct
  REGION 1..5: note = an artificially synthesized sequence
NVWMH

SEQ ID NO: 17 - protein, 5 aa, synthetic construct
  REGION 1..5: note = an artificially synthesized sequence
NVWMH

SEQ ID NO: 18 - protein, 5 aa, synthetic construct
  REGION 1..5: note = an artificially synthesized sequence
NVWFH

SEQ ID NO: 19 - protein, 5 aa, synthetic construct
  REGION 1..5: note = an artificially synthesized sequence
NVWMH

SEQ ID NO: 20 - protein, 5 aa, synthetic construct
  REGION 1..5: note = an artificially synthesized sequence
NVWFH

SEQ ID NO: 21 - protein, 72 aa, synthetic construct
  REGION 1..72: note = an artificially synthesized sequence
SPCPPNSFSSAGGQRTCDICRQCKGVFRTRKECSSTSNAECDCTPGFHCLGAGCSMCEQDCKQGQELTKKGC

SEQ ID NO: 22 - protein, 5 aa, synthetic construct
  REGION 1..5: note = an artificially synthesized sequence
NTWFH

SEQ ID NO: 23 - protein, 5 aa, synthetic construct
  REGION 1..5: note = an artificially synthesized sequence
NVWFH

SEQ ID NO: 24 - protein, 5 aa, synthetic construct
  REGION 1..5: note = an artificially synthesized sequence
NVWFH

SEQ ID NO: 25 - protein, 5 aa, synthetic construct
  REGION 1..5: note = an artificially synthesized sequence
NVWFH

SEQ ID NO: 26 - protein, 5 aa, synthetic construct
  REGION 1..5: note = an artificially synthesized sequence
NVWFH

SEQ ID NO: 27 - protein, 5 aa, synthetic construct
  REGION 1..5: note = an artificially synthesized sequence
NVWFH

SEQ ID NO: 28 - protein, 5 aa, synthetic construct
  REGION 1..5: note = an artificially synthesized sequence
NVWFH

SEQ ID NO: 29 - protein, 19 aa, synthetic construct
  REGION 1..19: note = an artificially synthesized sequence
QIKDKGNAYAAYYAPSVKG

SEQ ID NO: 30 - protein, 19 aa, synthetic construct
  REGION 1..19: note = an artificially synthesized sequence
QIKDKYNAYAAYYAPSVKG

SEQ ID NO: 31 - protein, 19 aa, synthetic construct
  REGION 1..19: note = an artificially synthesized sequence
QIKDKYNAYAAYYAPSVKG

SEQ ID NO: 32 - protein, 19 aa, synthetic construct
  REGION 1..19: note = an artificially synthesized sequence
QIKDKYNAYATYYAPSVKG

SEQ ID NO: 33 - protein, 19 aa, synthetic construct
  REGION 1..19: note = an artificially synthesized sequence
QIKDKWNAYAAYYAPSVKG

SEQ ID NO: 34 - protein, 19 aa, synthetic construct
  REGION 1..19: note = an artificially synthesized sequence
QIKDYYNAYAAYYAPSVKG

SEQ ID NO: 35 - protein, 31 aa, synthetic construct
  REGION 1..31: note = an artificially synthesized sequence
DCTPGFHCLGAGCSMCEQDCKQGQELTKKGC

SEQ ID NO: 36 - protein, 19 aa, synthetic construct
  REGION 1..19: note = an artificially synthesized sequence
QIKDYYNDYAAYYAPSVKG

SEQ ID NO: 37 - protein, 19 aa, synthetic construct
  REGION 1..19: note = an artificially synthesized sequence
QIKDKYNAYADYYAPSVKE

SEQ ID NO: 38 - protein, 19 aa, synthetic construct
  REGION 1..19: note = an artificially synthesized sequence
QIKDKYNAYADYYAPSVEG

SEQ ID NO: 39 - protein, 19 aa, synthetic construct
  REGION 1..19: note = an artificially synthesized sequence
QIKDYYNAYADYYAPSVEG

SEQ ID NO: 40 - protein, 19 aa, synthetic construct
  REGION 1..19: note = an artificially synthesized sequence
QIKDKWNAYADYYAPSVKE

SEQ ID NO: 41 - protein, 19 aa, synthetic construct
  REGION 1..19: note = an artificially synthesized sequence
QIKDYYNAYAGYYHPSVKG

SEQ ID NO: 42 - protein, 19 aa, synthetic construct
  REGION 1..19: note = an artificially synthesized sequence
QIKDYYNAYAGYYHPSVKG

SEQ ID NO: 43 - protein, 17 aa, synthetic construct
  REGION 1..17: note = an artificially synthesized sequence
VHYASASTVLPAFGVDA

SEQ ID NO: 44 - protein, 17 aa, synthetic construct
  REGION 1..17: note = an artificially synthesized sequence
VHYASASTLLPAFGVDA

SEQ ID NO: 45 - protein, 17 aa, synthetic construct
  REGION 1..17: note = an artificially synthesized sequence
IHYASASTLLPAFGVDA

SEQ ID NO: 46 - protein, 17 aa, synthetic construct
  REGION 1..17: note = an artificially synthesized sequence
VHYASASTLLPAFGVDA

SEQ ID NO: 47 - protein, 17 aa, synthetic construct
  REGION 1..17: note = an artificially synthesized sequence
IHYASASTLLPAEGIDA

SEQ ID NO: 48 - protein, 17 aa, synthetic construct
  REGION 1..17: note = an artificially synthesized sequence
VHYASASTLLPAEGVDA

SEQ ID NO: 49 - protein, 63 aa, synthetic construct
  REGION 1..63: note = an artificially synthesized sequence
LQDPCSNCPAGTFCDNNRNQICSPCPPNSFSSAGGQRTCDICRQCKGVFRTRKECSSTSNAEC

SEQ ID NO: 50 - protein, 17 aa, synthetic construct
  REGION 1..17: note = an artificially synthesized sequence
VHYASASTLLPAEGVDA

SEQ ID NO: 51 - protein, 17 aa, synthetic construct
  REGION 1..17: note = an artificially synthesized sequence
VHYASASTLLPAEGVDA

SEQ ID NO: 52 - protein, 17 aa, synthetic construct
  REGION 1..17: note = an artificially synthesized sequence
VHYASASTLLPAEGVDA

SEQ ID NO: 53 - protein, 17 aa, synthetic construct
  REGION 1..17: note = an artificially synthesized sequence
VHYASASTLLPAEGVDA

SEQ ID NO: 54 - protein, 17 aa, synthetic construct
  REGION 1..17: note = an artificially synthesized sequence
IHYASASTLLPAEGIDA

SEQ ID NO: 55 - protein, 17 aa, synthetic construct
  REGION 1..17: note = an artificially synthesized sequence
VHYAAASTLLPAEGVDA

SEQ ID NO: 56 - protein, 17 aa, synthetic construct
  REGION 1..17: note = an artificially synthesized sequence
VHYAAASQLLPAEGVDA

SEQ ID NO: 57 - protein, 112 aa, synthetic construct
  REGION 1..112: note = an artificially synthesized sequence
DIVMTQSPLSLPVTPGEPASISCQASQELVHMNRNTYLHWYQQKPGQAPRLLIYKVSNRFPGVPDRFSGSGSGTDFTLKISRVEAEDVGVYYCAQGTSVPFTFGQGTKLEIK

SEQ ID NO: 58 - protein, 112 aa, synthetic construct
  REGION 1..112: note = an artificially synthesized sequence
DIVMTQSPLSLPVTPGEPASISCQPSQEVVHMNRNTYLHWYQQKPGQAPRLLIYKVSNRFPGVPDRFSGSGSGTDFTLKISRVEAEDVGVYYCAQGTSHPFTFGQGTKLEIK

SEQ ID NO: 59 - protein, 112 aa, synthetic construct
  REGION 1..112: note = an artificially synthesized sequence
DIVMTQSPLSLPVTPGEPASISCQPSQEVVHMNNVVYLHWYQQKPGQAPRLLIYKVSNRFPGVPDRFSGSGSGTDFTLKISRVEAEDVGVYYCAQGTSHPFTFGQGTKLEIK

SEQ ID NO: 60 - protein, 112 aa, synthetic construct
  REGION 1..112: note = an artificially synthesized sequence
DIVMTQSPLSLPVTPGEPASISCQPSQEVVHMNRNTYLHWYQQKPGQAPRLLIYKVSNVFPGVPDRFSGSGSGTDFTLKISRVEAEDVGVYYCAQGTHHPFTFGQGTKLEIK

SEQ ID NO: 61 - protein, 112 aa, synthetic construct
  REGION 1..112: note = an artificially synthesized sequence
DIVMTQSPLSLPVTPGEPASISCQPSEEVVHMNRNTYLHWYQQKPGQAPRLLIYKVSNLFPGVPDRFSGSGSGTDFTLKISRVEAEDVGVYYCAQGTHHPFTFGQGTKLEIK

SEQ ID NO: 62 - protein, 16 aa, synthetic construct
  REGION 1..16: note = an artificially synthesized sequence
QASQELVHMNRNTYLH

SEQ ID NO: 63 - protein, 16 aa, synthetic construct
  REGION 1..16: note = an artificially synthesized sequence
QPSQEVVHMNRNTYLH

SEQ ID NO: 64 - protein, 16 aa, synthetic construct
  REGION 1..16: note = an artificially synthesized sequence
QPSQEVVHMNNVVYLH

SEQ ID NO: 65 - protein, 16 aa, synthetic construct
  REGION 1..16: note = an artificially synthesized sequence
QPSQEVVHMNRNTYLH

SEQ ID NO: 66 - protein, 16 aa, synthetic construct
  REGION 1..16: note = an artificially synthesized sequence
QPSEEVVHMNRNTYLH

SEQ ID NO: 67 - protein, 7 aa, synthetic construct
  REGION 1..7: note = an artificially synthesized sequence
KVSNRFP

SEQ ID NO: 68 - protein, 7 aa, synthetic construct
  REGION 1..7: note = an artificially synthesized sequence
KVSNRFP

SEQ ID NO: 69 - protein, 7 aa, synthetic construct
  REGION 1..7: note = an artificially synthesized sequence
KVSNRFP

SEQ ID NO: 70 - protein, 7 aa, synthetic construct
  REGION 1..7: note = an artificially synthesized sequence
KVSNVFP

SEQ ID NO: 71 - protein, 7 aa, synthetic construct
  REGION 1..7: note = an artificially synthesized sequence
KVSNLFP

SEQ ID NO: 72 - protein, 9 aa, synthetic construct
  REGION 1..9: note = an artificially synthesized sequence
AQGTSVPFT

SEQ ID NO: 73 - protein, 9 aa, synthetic construct
  REGION 1..9: note = an artificially synthesized sequence
AQGTSHPFT

SEQ ID NO: 74 - protein, 9 aa, synthetic construct
  REGION 1..9: note = an artificially synthesized sequence
AQGTSHPFT

SEQ ID NO: 75 - protein, 9 aa, synthetic construct
  REGION 1..9: note = an artificially synthesized sequence
AQGTHHPFT

SEQ ID NO: 76 - protein, 9 aa, synthetic construct
  REGION 1..9: note = an artificially synthesized sequence
AQGTHHPFT

SEQ ID NO: 77 - protein, 122 aa, synthetic construct
  REGION 1..122: note = an artificially synthesized sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSS

SEQ ID NO: 78 - protein, 112 aa, synthetic construct
  REGION 1..112: note = an artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIK

SEQ ID NO: 79 - protein, 121 aa, synthetic construct [sequence omitted]

SEQ ID NO: 80 - protein, 109 aa, synthetic construct [sequence omitted]

SEQ ID NO: 81 - protein, 128 aa, synthetic construct
  REGION 1..128: note = an artificially synthesized sequence
QVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSS

SEQ ID NO: 82 - protein, 5 aa, synthetic construct
  REGION 1..5: note = an artificially synthesized sequence
NVWFH

SEQ ID NO: 83 - protein, 19 aa, synthetic construct
  REGION 1..19: note = an artificially synthesized sequence
QIKDYYNAYAAYYAPSVKG

SEQ ID NO: 84 - protein, 17 aa, synthetic construct
  REGION 1..17: note = an artificially synthesized sequence
VHYASASTLLPAEGVDA

SEQ ID NO: 85 - protein, 330 aa, synthetic construct [sequence omitted]

SEQ ID NO: 86 - protein, 326 aa, synthetic construct
  REGION 1..326: note = an artificially synthesized sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 87 - protein, 377 aa, synthetic construct
  REGION 1..377: note = an artificially synthesized sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 88 - protein, 327 aa, synthetic construct
  REGION 1..327: note = an artificially synthesized sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 88 aa, synthetic construct
  REGION 1..88: note = an artificially synthesized sequence
MVLASSTTSIHTMLLLLLMLAQPAMARADPCAARPCAHGGRCYAHFSGLVCACAPGYMGARCEFPVHPDGASALPAAPPGLRPGDPQR

SEQ ID NO: 90 - protein, 618 aa, synthetic construct
  REGION 1..618: note = an artificially synthesized sequence
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 91 - protein, 4 aa, synthetic construct
  REGION 1..4: note = an artificially synthesized sequence
GGGS

SEQ ID NO: 92 - protein, 4 aa, synthetic construct
  REGION 1..4: note = an artificially synthesized sequence
SGGG

SEQ ID NO: 93 - protein, 5 aa, synthetic construct
  REGION 1..5: note = an artificially synthesized sequence
GGGGS

SEQ ID NO: 94 - protein, 5 aa, synthetic construct
  REGION 1..5: note = an artificially synthesized sequence
SGGGG

SEQ ID NO: 95 - protein, 6 aa, synthetic construct
  REGION 1..6: note = an artificially synthesized sequence
GGGGGS

SEQ ID NO: 96 - protein, 6 aa, synthetic construct
  REGION 1..6: note = an artificially synthesized sequence
SGGGGG

SEQ ID NO: 97 - protein, 7 aa, synthetic construct
  REGION 1..7: note = an artificially synthesized sequence
GGGGGGS

SEQ ID NO: 98 - protein, 7 aa, synthetic construct
  REGION 1..7: note = an artificially synthesized sequence
SGGGGGG

SEQ ID NO: 99 - protein, 220 aa, synthetic construct
  REGION 1..220: note = an artificially synthesized sequence
CPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 100 - protein, 220 aa, synthetic construct
  REGION 1..220: note = an artificially synthesized sequence
CPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 101 - protein, 128 aa, synthetic construct
  REGION 1..128: note = an artificially synthesized sequence
QVQLVESGGGLVQPGRSLRLSCAASGFKFSNVWMHWVRQAPGKGLEWVAQIKDKWNAYAAYYAPSVKGRFTISRDDSKNSIYLQMNSLKTEDTAVYYCHYIHYASASTLLPAFGIDAWGQGTTVTVSS

SEQ ID NO: 102 - protein, 212 aa, synthetic construct
  REGION 1..212: note = an artificially synthesized sequence
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVGSADDAKKDAAKKDDAKKDDAKKDGSQSIKGNHLVKVYDYQEDGSVLLTCDAEAKNITWFKDGKMIGFLTEDKKKWNLGSNAKDPRGMYQCKGSQNKSKPLQVYYRMDYKDDDDK

SEQ ID NO: 103 - protein, 189 aa, synthetic construct
  REGION 1..189: note = an artificially synthesized sequence
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQHHHHHHGGGGSGLNDIFEAQKIEWHE

SEQ ID NO: 104 - protein, 128 aa, synthetic construct
  REGION 1..128: note = an artificially synthesized sequence
QVQLVESGGGLVQPGRSLRLSCAASGFKFSNTWMHWVRQAPGKGLEWVAQIKDKYNAYAAYYAPSVKGRFTISRDDSKNSIYLQMNSLKTEDTAVYYCHYIHYASASTLLPAFGVDAWGQGTTVTVSS

SEQ ID NO: 105 - protein, 22 aa, synthetic construct
  REGION 1..22: note = an artificially synthesized sequence
LQDPCSNCPAGTFCDNNRNQIC

SEQ ID NO: 106 - protein, 128 aa, synthetic construct
  REGION 1..128: note = an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAFGVDAWGQGTTVTVSS

SEQ ID NO: 107 - protein, 128 aa, synthetic construct
  REGION 1..128: note = an artificially synthesized sequence
QVQLVESGGGLVQPGRSLRLSCAASGFKFSHVWFHWVRQAPGKGLEWVAQIKDKYNAYAAYYAPSVKGRFTISRDDSKNSIYLQMNSLKTEDTAVYYCHYVHYASASTLLPAFGVDAWGQGTTVTVSS

SEQ ID NO: 108 - protein, 220 aa, synthetic construct
  REGION 1..220: note = an artificially synthesized sequence
CPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 109 - protein, 220 aa, synthetic construct
  REGION 1..220: note = an artificially synthesized sequence
CPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQEGNVFSCSVLHEALHAHYTRKELSLSP

SEQ ID NO: 110 - protein, 128 aa, synthetic construct
  REGION 1..128: note = an artificially synthesized sequence
QVQLVESGGGLVQPGRSLRLSCAASGFKFSNVWFHWVRQAPGKGLEWVAQIKDKYNAYAAYYAPSVKGRFTISRDDSKNSIYLQMNSLKTEDTAVYYCHYVHYASASTLLPAEGVDAWGQGTTVTVSS

SEQ ID NO: 111 - protein, 220 aa, synthetic construct
  REGION 1..220: note = an artificially synthesized sequence
CPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 112 - protein, 220 aa, synthetic construct
  REGION 1..220: note = an artificially synthesized sequence
CPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 113 - protein, 128 aa, synthetic construct
  REGION 1..128: note = an artificially synthesized sequence
QVQLVESGGGLVQPGRSLRLSCAASGFTFSNAWMHWVRQAPGKGLEWVAQIKDKGNAYAAYYAPSVKGRFTISRDDSKNSIYLQMNSLKTEDTAVYYCRYVHYASASTLLPAFGVDAWGQGTTVTVSS

SEQ ID NO: 114 - protein, 5 aa, synthetic construct
  REGION 1..5: note = an artificially synthesized sequence
NVWMH

SEQ ID NO: 115-116 - skipped (2 entries)

SEQ ID NO: 117 - protein, 5 aa, synthetic construct
  REGION 1..5: note = an artificially synthesized sequence
NTWMH

SEQ ID NO: 118 - skipped

SEQ ID NO: 119 - protein, 5 aa, synthetic construct
  REGION 1..5: note = an artificially synthesized sequence
NVWFH

SEQ ID NO: 120 - protein, 5 aa, synthetic construct
  REGION 1..5: note = an artificially synthesized sequence
HVWFH

SEQ ID NO: 121-122 - skipped (2 entries)

SEQ ID NO: 123 - protein, 5 aa, synthetic construct
  REGION 1..5: note = an artificially synthesized sequence
NVWFH

SEQ ID NO: 124-125 - skipped (2 entries)

SEQ ID NO: 126 - protein, 5 aa, synthetic construct
  REGION 1..5: note = an artificially synthesized sequence
NAWMH

SEQ ID NO: 127 - protein, 19 aa, synthetic construct
  REGION 1..19: note = an artificially synthesized sequence
QIKDKWNAYAAYYAPSVKG

SEQ ID NO: 128-129 - skipped (2 entries)

SEQ ID NO: 130 - protein, 19 aa, synthetic construct
  REGION 1..19: note = an artificially synthesized sequence
QIKDKYNAYAAYYAPSVKG

SEQ ID NO: 131 - skipped

SEQ ID NO: 132 - protein, 19 aa, synthetic construct
  REGION 1..19: note = an artificially synthesized sequence
QIKDYYNAYAAYYAPSVKG

SEQ ID NO: 133 - protein, 19 aa, synthetic construct
  REGION 1..19: note = an artificially synthesized sequence
QIKDKYNAYAAYYAPSVKG

SEQ ID NO: 134-135 - skipped (2 entries)

SEQ ID NO: 136 - protein, 19 aa, synthetic construct
  REGION 1..19: note = an artificially synthesized sequence
QIKDKYNAYAAYYAPSVKG

SEQ ID NO: 137-138 - skipped (2 entries)

SEQ ID NO: 139 - protein, 19 aa, synthetic construct
  REGION 1..19: note = an artificially synthesized sequence
QIKDKGNAYAAYYAPSVKG

SEQ ID NO: 140 - protein, 17 aa, synthetic construct
  REGION 1..17: note = an artificially synthesized sequence
IHYASASTLLPAFGIDA

SEQ ID NO: 141-142 - skipped (2 entries)

SEQ ID NO: 143 - protein, 17 aa, synthetic construct
  REGION 1..17: note = an artificially synthesized sequence
IHYASASTLLPAFGVDA

SEQ ID NO: 144 - skipped

SEQ ID NO: 145 - protein, 17 aa, synthetic construct
  REGION 1..17: note = an artificially synthesized sequence
VHYASASTLLPAFGVDA

SEQ ID NO: 146 - protein, 17 aa, synthetic construct
  REGION 1..17: note = an artificially synthesized sequence
VHYASASTLLPAFGVDA

SEQ ID NO: 147-148 - skipped (2 entries)

SEQ ID NO: 149 - protein, 17 aa, synthetic construct
  REGION 1..17: note = an artificially synthesized sequence
VHYASASTLLPAEGVDA

SEQ ID NO: 150-151 - skipped (2 entries)

SEQ ID NO: 152 - protein, 17 aa, synthetic construct
  REGION 1..17: note = an artificially synthesized sequence
VHYASASTLLPAFGVDA

SEQ ID NO: 153 - protein, 676 aa, synthetic construct
  REGION 1..676: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 154 - protein, 226 aa, synthetic construct
  REGION 1..226: note = an artificially synthesized sequence
QVTLRESGPALVKPTQTLTLTCTFSGFSLSSSYDMGWVRQAPGQGLEWMGTIYTGDYSTDYASWAKGRVTISVDRSKNQFSLKLSSVTAADTAVYYCARHTGYGYFGLWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 456 aa, synthetic construct
  REGION 1..456: note = an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 156 - protein, 219 aa, synthetic construct
  REGION 1..219: note = an artificially synthesized sequence
DIVMTQSPLSLPVTPGEPASISCQPSQEVVHMNRNTYLHWYQQKPGQAPRLLIYKVSNRFPGVPDRFSGSGSGTDFTLKISRVEAEDVGVYYCAQGTSHPFTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157-200 - skipped (44 entries)

SEQ ID NO: 201 - protein, 681 aa, synthetic construct
  REGION 1..681: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 202 - protein, 678 aa, synthetic construct
  REGION 1..678: note = an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 203 - protein, 681 aa, synthetic construct
  REGION 1..681: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 204 - protein, 676 aa, synthetic construct
  REGION 1..676: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 205 - protein, 671 aa, synthetic construct
  REGION 1..671: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 206 - protein, 226 aa, synthetic construct; identical to SEQ ID NO: 154
  REGION 1..226: note = an artificially synthesized sequence

SEQ ID NO: 207 - protein, 223 aa, synthetic construct
  REGION 1..223: note = an artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 208 - protein, 456 aa, synthetic construct
  REGION 1..456: note = an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQEGNVFSCSVLHEALHAHYTRKELSLSP

SEQ ID NO: 209 - protein, 456 aa, synthetic construct
  REGION 1..456: note = an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 210 - protein, 456 aa, synthetic construct
  REGION 1..456: note = an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQEGNVFSCSVLHEALHAHYTRKELSLSP

SEQ ID NO: 211 - protein, 456 aa, synthetic construct
  REGION 1..456: note = an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 212 - protein, 456 aa, synthetic construct
  REGION 1..456: note = an artificially synthesized sequence
QVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQEGNVFSCSVLHEALHAHYTRKELSLSP

SEQ ID NO: 213 - protein, 456 aa, synthetic construct
  REGION 1..456: note = an artificially synthesized sequence
QVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 214 - protein, 219 aa, synthetic construct; identical to SEQ ID NO: 156
  REGION 1..219: note = an artificially synthesized sequence

SEQ ID NO: 215 - protein, 219 aa, synthetic construct
  REGION 1..219: note = an artificially synthesized sequence
DIVMTQSPLSLPVTPGEPASISCQPSQEVVHMNRNTYLHWYQQKPGQAPRLLIYKVSNVFPGVPDRFSGSGSGTDFTLKISRVEAEDVGVYYCAQGTHHPFTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 681 aa, synthetic construct
  REGION 1..681: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 217 - protein, 681 aa, synthetic construct
  REGION 1..681: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 218 - protein, 678 aa, synthetic construct
  REGION 1..678: note = an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 219 - protein, 681 aa, synthetic construct
  REGION 1..681: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 220 - protein, 676 aa, synthetic construct
  REGION 1..676: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 221 - protein, 671 aa, synthetic construct
  REGION 1..671: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 222 - protein, 681 aa, synthetic construct
  REGION 1..681: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 223 - protein, 681 aa, synthetic construct
  REGION 1..681: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 224 - protein, 678 aa, synthetic construct
  REGION 1..678: note = an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 225 - protein, 681 aa, synthetic construct
  REGION 1..681: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 226 - protein, 676 aa, synthetic construct
  REGION 1..676: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 227 - protein, 671 aa, synthetic construct
  REGION 1..671: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 228 - protein, 681 aa, synthetic construct
  REGION 1..681: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 229 - protein, 456 aa, synthetic construct
  REGION 1..456: note = an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 230 - protein, 456 aa, synthetic construct
  REGION 1..456: note = an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 231 - protein, 456 aa, synthetic construct
  REGION 1..456: note = an artificially synthesized sequence
QVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 232 - protein, 119 aa, synthetic construct
  REGION 1..119: note = an artificially synthesized sequence
QVTLRESGPALVKPTQTLTLTCTFSGFSLSSSYDMGWVRQAPGQGLEWMGTIYTGDYSTDYASWAKGRVTISVDRSKNQFSLKLSSVTAADTAVYYCARHTGYGYFGLWGQGTLVTVSS

SEQ ID NO: 233 - protein, 6 aa, synthetic construct
  REGION 1..6: note = an artificially synthesized sequence
SSYDMG

SEQ ID NO: 234 - protein, 17 aa, synthetic construct
  REGION 1..17: note = an artificially synthesized sequence
TIYTGDYSTDYASWAKG

SEQ ID NO: 235 - protein, 9 aa, synthetic construct
  REGION 1..9: note = an artificially synthesized sequence
HTGYGYFGL

SEQ ID NO: 236 - protein, 110 aa, synthetic construct
  REGION 1..110: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIK

SEQ ID NO: 237 - protein, 13 aa, synthetic construct
  REGION 1..13: note = an artificially synthesized sequence
QSTESVYGSDWLS

SEQ ID NO: 238 - protein, 7 aa, synthetic construct
  REGION 1..7: note = an artificially synthesized sequence
QASNLEI

SEQ ID NO: 239 - protein, 10 aa, synthetic construct
  REGION 1..10: note = an artificially synthesized sequence
QGYYSGYIYA

SEQ ID NO: 240 - protein, 116 aa, synthetic construct
  REGION 1..116: note = an artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSS

SEQ ID NO: 241 - protein, 5 aa, synthetic construct
  REGION 1..5: note = an artificially synthesized sequence
SYWMH

SEQ ID NO: 242 - protein, 17 aa, synthetic construct
  REGION 1..17: note = an artificially synthesized sequence
MIDPSYSETRLNQKFKD

SEQ ID NO: 243 - protein, 7 aa, synthetic construct
  REGION 1..7: note = an artificially synthesized sequence
YGNYFDY

SEQ ID NO: 244 - protein, 107 aa, synthetic construct
  REGION 1..107: note = an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVK

SEQ ID NO: 245 - protein, 11 aa, synthetic construct
  REGION 1..11: note = an artificially synthesized sequence
KASEDIYNRLA

SEQ ID NO: 246 - protein, 7 aa, synthetic construct
  REGION 1..7: note = an artificially synthesized sequence
GATSLET

SEQ ID NO: 247 - protein, 9 aa, synthetic construct
  REGION 1..9: note = an artificially synthesized sequence
QQYWSTPYT

SEQ ID NO: 248 - protein, 11 aa, synthetic construct
  REGION 1..11: note = an artificially synthesized sequence
VEPKSCGGGGS

SEQ ID NO: 249 - protein, 16 aa, synthetic construct
  REGION 1..16: note = an artificially synthesized sequence
VEPKSCGGGGSGGGGS

SEQ ID NO: 250 - protein, 984 aa, synthetic construct
  REGION 1..984: note = an artificially synthesized sequence
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 122 aa, synthetic construct; identical to SEQ ID NO: 77
  REGION 1..122: note = an artificially synthesized sequence

SEQ ID NO: 252 - protein, 5 aa, synthetic construct
  REGION 1..5: note = an artificially synthesized sequence
NAWMH

SEQ ID NO: 253 - protein, 19 aa, synthetic construct
  REGION 1..19: note = an artificially synthesized sequence
QIKDKSQNYATYVAESVKG

SEQ ID NO: 254 - protein, 11 aa, synthetic construct
  REGION 1..11: note = an artificially synthesized sequence
VHYAAGYGVDI

SEQ ID NO: 255 - protein, 112 aa, synthetic construct; identical to SEQ ID NO: 78
  REGION 1..112: note = an artificially synthesized sequence

SEQ ID NO: 256 - protein, 16 aa, synthetic construct
  REGION 1..16: note = an artificially synthesized sequence
RSSQPLVHSNRNTYLH

SEQ ID NO: 257 - protein, 7 aa, synthetic construct
  REGION 1..7: note = an artificially synthesized sequence
KVSNRFS

SEQ ID NO: 258 - protein, 9 aa, synthetic construct
  REGION 1..9: note = an artificially synthesized sequence
GQGTQVPYT

SEQ ID NO: 259 - protein, 6 aa, synthetic construct
  REGION 1..6: note = an artificially synthesized sequence
VEPKSC

SEQ ID NO: 260 - protein, 122 aa, synthetic construct
  REGION 1..122: note = an artificially synthesized sequence
QSVEESGGRLVTPGTPLTLTCTVSGIDLSSYAMSWVRQAPGKGLEWIGIISNSGSGFYANWAKGRFTISKTSSTTVDVKISSPTTEDTATYFCARVFDADSSGYYYYGMDPWGPGTLVTVSS

SEQ ID NO: 261 - protein, 111 aa, synthetic construct
  REGION 1..111: note = an artificially synthesized sequence
DVVMTQTPSSVSEPVGGTVTIKCQASEDIESYLAWYQQKPGQPPKLLIYAASTLASGVSSRFRGSGSGTEYTLTISDLECADAATYYCQSTYYDSSGVDWAFGGGTEVVVK

SEQ ID NO: 262 - protein, 123 aa, synthetic construct
  REGION 1..123: note = an artificially synthesized sequence
QSVEESGGRLVTPGTPLTLTCTVSGFSLSRYAMSWVRQAPGKGLEWIGIITGGGDPYYASWAKGRFTISKTSTTMDLKITSPTTEDTATYFCARMIGSEYASSSEYYDAFDPWGPGTLVTVSS

SEQ ID NO: 263 - protein, 112 aa, synthetic construct
  REGION 1..112: note = an artificially synthesized sequence
ALVMTQTPSSVSAAVGGTVTINCQASQNIYSNLAWYQQKPGQRPKLLIYGASNLESGVPSRFKGSGSGTEYTLTISDLECDDAAIYYCQSAYYTTSVDVLYPFGGGTEVVVK

SEQ ID NO: 264 - protein, 124 aa, synthetic construct
  REGION 1..124: note = an artificially synthesized sequence
QEQLVESGGGLVQPGGSLKLSCKVSGFDFSNYGVSWVRQAPGKGLEWIGYIDPAFGRTYYASWVNGRFTISSHNAQNTLYLQLNSLTAADTATYFCVRVALVVAGVAYYGMDVWGPGTLVTVSS

SEQ ID NO: 265 - protein, 114 aa, synthetic construct
  REGION 1..114: note = an artificially synthesized sequence
ADVVMTQTPASVEAAVGGTVTINCQASQNIYSYLSWYQQKPGQPPKLLIYRTSTLASGVPSRFKGSGSGTQFTLTISDLECADAATYYCQSYYYSSGSSYGAFAFGGGTEVVVK

SEQ ID NO: 266 - protein, 114 aa, synthetic construct
  REGION 1..114: note = an artificially synthesized sequence
QSLEESGGRLVTPGTPLTLTCTASGFSLNTYHMIWVRQAPGKGLEWIGIVDSGGSTYYASWAKGRFTISRTSTTVDLKATSLTTEDTATYFCARGGSWDGFNIWGPGTLVTVSS

SEQ ID NO: 267 - protein, 112 aa, synthetic construct
  REGION 1..112: note = an artificially synthesized sequence
DVVMTQTPSSVSAAVGGTVTINCQSSQSVYNSDFLAWYQQKPGQPPKLLIYAASTLASGVPSRFKGSGSGTQFTLTISDLECDDAATYYCQGTYEDVGWFNDFGGGTEVVVK

SEQ ID NO: 268 - protein, 128 aa, synthetic construct
  REGION 1..128: note = an artificially synthesized sequence
QEQLVESGGGLVQPGASLTLTCKASGFDFTSNAMCWVRQAPGKGPEWIACIYNDDGSAYSASWAKGRFTVSKTSSTAVTLQVTGLTAADTATYFCARSPYYTYGGAPSAYASGYFNLWGPGTLVTVSS

SEQ ID NO: 269 - protein, 113 aa, synthetic construct
  REGION 1..113: note = an artificially synthesized sequence
ADIVMTQTPASVEAAVGGTVTIKCQASENIYNSLAWYQQKPGQPPKLLMHDASVLTSGVPSRFSGSGYGTEFTLTISDLECADAATYYCQSDYYSTGGSYGFAFGGGTEVVVK

SEQ ID NO: 270 - protein, 121 aa, synthetic construct
  REGION 1..121: note = an artificially synthesized sequence
QEQLVESGGGLVQPGASLTLTCKASGFSFNTSYCPCWVRQVPGKGPEWIACIDAGYSGATWYANWAKGRFTISKTSSTTVTLQMTSLTGADTATYFCVRCDAAGSGAFNLWGPGTLVTVSS

SEQ ID NO: 271 - protein, 110 aa, synthetic construct
  REGION 1..110: note = an artificially synthesized sequence
AYDMTQTPASVEVPVGGTVTIKCQASQSIYSYLSWYQQKPGQPPKLLIYEASKLTSGVPSRFSGSGSGTEFTLTISGVQCDDAATYYCQQGSGYMDVDNVFGGGTEVVVK

SEQ ID NO: 272 - protein, 123 aa, synthetic construct
  REGION 1..123: note = an artificially synthesized sequence
QEQLVESGGGLVQPEGSLTLTCTASGFSFSSSGYMCWVRQAPGKGLEWIACISGGSSGDTDYANWAKGRFSISKTSSTTVTLQMTSLTVADTATYFCARDVYIDSTIFNFNLWGPGTLVTVSS

SEQ ID NO: 273 - protein, 111 aa, synthetic construct
  REGION 1..111: note = an artificially synthesized sequence
QVLTQTPSPVSAAVGGTVTISCQASQSVYNNNNLAWYQQKPGQPPKLLIYAASSLASGVPSRFKGSGAGTQFTLTISDLECDDAATYYCQGTYYSSGWYSTFGGGTEVVVK

SEQ ID NO: 274 - protein, 118 aa, synthetic construct
  REGION 1..118: note = an artificially synthesized sequence
QEQLVESGGGLVQPEGSLTLTCTASGFSFSSSYDMCWVRQAPGKGPEWIACIYTGDGSTDYASWAKGRFTVSKASSTTVTLQMTSLAVADTATYFCARNSGYGYFGLWGPGTLVTVSS

SEQ ID NO: 275 - protein, 109 aa, synthetic construct
  REGION 1..109: note = an artificially synthesized sequence
QVLTQTPSPVSAAVGGTVTISCQSTESVYGSDWLSWYQQKPGQPPKVLIYQASNLEIGVPSRFKGSGSGTQFTLTISGAQCDDAATYYCQGYYSGYIYAFGGGTEVVVK

SEQ ID NO: 276 - protein, 5 aa, synthetic construct
  REGION 1..5: note = an artificially synthesized sequence
NYGVS

SEQ ID NO: 277 - protein, 17 aa, synthetic construct
  REGION 1..17: note = an artificially synthesized sequence
YIDPAFGRTYYASWVNG

SEQ ID NO: 278 - protein, 15 aa, synthetic construct
  REGION 1..15: note = an artificially synthesized sequence
VALVVAGVAYYGMDV

SEQ ID NO: 279 - protein, 11 aa, synthetic construct
  REGION 1..11: note = an artificially synthesized sequence
QASQNIYSYLS

SEQ ID NO: 280 - protein, 7 aa, synthetic construct
  REGION 1..7: note = an artificially synthesized sequence
RTSTLAS

SEQ ID NO: 281 - protein, 15 aa, synthetic construct
  REGION 1..15: note = an artificially synthesized sequence
QSYYYSSGSSYGAFA

SEQ ID NO: 282 - protein, 6 aa, synthetic construct
  REGION 1..6: note = an artificially synthesized sequence
SSYDMC

SEQ ID NO: 283 - protein, 17 aa, synthetic construct
  REGION 1..17: note = an artificially synthesized sequence
CIYTGDGSTDYASWAKG

SEQ ID NO: 284 - protein, 9 aa, synthetic construct
  REGION 1..9: note = an artificially synthesized sequence
NSGYGYFGL

SEQ ID NO: 285 - protein, 5 aa, synthetic construct
  REGION 1..5: note = an artificially synthesized sequence
SNAMC

SEQ ID NO: 286 - protein, 17 aa, synthetic construct
  REGION 1..17: note = an artificially synthesized sequence
CIYNDDGSAYSASWAKG

SEQ ID NO: 287 - protein, 20 aa, synthetic construct
  REGION 1..20: note = an artificially synthesized sequence
SPYYTYGGAPSAYASGYFNL

SEQ ID NO: 288 - protein, 11 aa, synthetic construct
  REGION 1..11: note = an artificially synthesized sequence
QASENIYNSLA

SEQ ID NO: 289 - protein, 7 aa, synthetic construct
  REGION 1..7: note = an artificially synthesized sequence
DASVLTS

SEQ ID NO: 290 - protein, 14 aa, synthetic construct
  REGION 1..14: note = an artificially synthesized sequence
QSDYYSTGGSYGFA

SEQ ID NO: 291 - protein, 6 aa, synthetic construct
  REGION 1..6: note = an artificially synthesized sequence
SSGYMC

SEQ ID NO: 292 - protein, 18 aa, synthetic construct
  REGION 1..18: note = an artificially synthesized sequence
CISGGSSGDTDYANWAKG

SEQ ID NO: 293 - protein, 13 aa, synthetic construct
  REGION 1..13: note = an artificially synthesized sequence
DVYIDSTIFNFNL

SEQ ID NO: 294 - protein, 13 aa, synthetic construct
  REGION 1..13: note = an artificially synthesized sequence
QASQSVYNNNNLA

SEQ ID NO: 295 - protein, 7 aa, synthetic construct
  REGION 1..7: note = an artificially synthesized sequence
AASSLAS

SEQ ID NO: 296 - protein, 12 aa, synthetic construct
  REGION 1..12: note = an artificially synthesized sequence
QGTYYSSGWYST

SEQ ID NO: 297 - protein, 119 aa, synthetic construct
  REGION 1..119: note = an artificially synthesized sequence
QVQLKESGPALVKPTQTLTLTCTFSGFSLSSSYDMGWVRQAPGQGLEWMGTIYTGDYSTDYASWAKGRVTITADESTSTAYMELSSLRSEDTAVYYCARNTGYGYFGLWGQGTLVTVSS

SEQ ID NO: 298 - protein, 119 aa, synthetic construct
  REGION 1..119: note = an artificially synthesized sequence
QVTLRESGPALVKPTQTLTLTCTFSGFSLSSSYDMGWVRQAPGQGLEWMGTIYTGDYSTDYASWAKGRVTISVDTSKNQFSLKLSSVTAADTAVYYCARHTGYGYFGLWGQGTLVTVSS

SEQ ID NO: 299 - protein, 119 aa, synthetic construct
  REGION 1..119: note = an artificially synthesized sequence
QVTLRESGPALVKPTQTLTLTCAFSGFSLSSSYDMGWVRQAPGQGLEWMGTIYTGDYSTDYASWAKGRVTISVDTSKNQFSLKLSSVTAADTAVYYCARHTGYGYFGLWGQGTLVTVSS

SEQ ID NO: 300 - protein, 119 aa, synthetic construct
  REGION 1..119: note = an artificially synthesized sequence
QVTLRESGPALVKPTQTLTLTCTFSGFSLSSSYDMGWVRQAPGQGLEWMGTIYTGDYSTDYASWAKGRVTISVDSSKNQFSLKLSSVTAADTAVYYCARHTGYGYFGLWGQGTLVTVSS

SEQ ID NO: 301 - protein, 119 aa, synthetic construct
  REGION 1..119: note = an artificially synthesized sequence
QVTLRESGPALVKPTQTLTLTCTFSGFSLSSSYDMGWVRQAPGQGLEWMGTIYTGDYSTDYASWAKGRVTISVDKSKNQFSLKLSSVTAADTAVYYCARHTGYGYFGLWGQGTLVTVSS

SEQ ID NO: 302 - protein, 110 aa, synthetic construct
  REGION 1..110: note = an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYSYAFGGGTKVEIK

SEQ ID NO: 303 - protein, 9 aa, synthetic construct
  REGION 1..9: note = an artificially synthesized sequence
NTGYGYFGL

SEQ ID NO: 304 - protein, 10 aa, synthetic construct
  REGION 1..10: note = an artificially synthesized sequence
QGYYSGYSYA

SEQ ID NO: 305 - protein, 121 aa, synthetic construct
  REGION 1..121: note = an artificially synthesized sequence
QVQLQQSGADLVRPGTSVKVSCKASGYAFTNYLIEWIKQRPGQGLEWIGVMNPGSGGTHYSEKFRGKATLTADKSSSTAYMQLISLTSDDSAVYFCARSDYDYVTYAMDYWGQGTSVTVSS

SEQ ID NO: 306 - protein, 118 aa, synthetic construct
  REGION 1..118: note = an artificially synthesized sequence
EVQLQQSGPELVKPGASVKMSCRASGYTFTDYYMKWVKQSHGKSLEWIGDINPNNGDTFYNQKFKGKATLTIDKSSSTAYMQLNSLTSDDSAVYYCARDGNYAYFDYWGQGTTLTVSS

SEQ ID NO: 307 - protein, 119 aa, synthetic construct
  REGION 1..119: note = an artificially synthesized sequence
QVQLQQSGGDLMKPGASVKISCKAAGYTFSNYYIEWVKQRPGHGLEWIGEILPGSGSTTYNEKFKGKASFTADTSSNTAYMQLSSLTSEDSAVYYCARWGAREPGFPYWGQGTLVTVSA

SEQ ID NO: 308 - protein, 123 aa, synthetic construct
  REGION 1..123: note = an artificially synthesized sequence
EVKLVESGGGLVQPGGSLSLSCAASGFTFTDYYMNWVRQPPGKALEWLALIRNKANGYTTEYNASVKGRFTISRDNSQNILYLQMNALRAEDSATYYCARDSDGYYEYYFDYWGQGTTLTVSS

SEQ ID NO: 309 - protein, 123 aa, synthetic construct
  REGION 1..123: note = an artificially synthesized sequence
QVQLVQSGAEVKKPGASVKVSCKASGYTFTNYYMHWVRQAPGLGLEWMGIINPSDGSTSYAQKFQGRVTMTRDTSTNTVYMDLSSLRSEDTAVYYCARGGNSAFYSYYDMDVWGQGTTVTVSS

SEQ ID NO: 310 - protein, 118 aa, synthetic construct
  REGION 1..118: note = an artificially synthesized sequence
QVQLQESGPGLVKPSETLSLTCTVSGDSISSYYWTWIRQPPGKGLEWIGYIYYSGTTNYNPSLKSRVTISVDTSKSQFSLKLSSVTAADTAVYYCASIAVRGFFFDYWGQGTLVTVSS

SEQ ID NO: 311 - protein, 118 aa, synthetic construct
  REGION 1..118: note = an artificially synthesized sequence
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSS

SEQ ID NO: 312 - protein, 118 aa, synthetic construct
  REGION 1..118: note = an artificially synthesized sequence
QVQLQESGPGLVKPSETLSLTCTVSGASISSFYWSWIRQPPGKGLEWIGYIYYSGTTNYNPSLKSRVTISVDTSKNQFSLKLSSVTAADTAVYYCARIAVAGFFFDYWGQGTLVTVSS

SEQ ID NO: 313 - protein, 107 aa, synthetic construct
  REGION 1..107: note = an artificially synthesized sequence
DIKMTQSPSSMYASLGERVTITCKASQDINSYLIWFQQKPGKSPKTLIYRTNRLVDGVPSRFSGSGSGQDYSLTISSLEYGDMGIYYCLQYDEFPFTFGSGTKLEIK

SEQ ID NO: 314 - protein, 111 aa, synthetic construct
  REGION 1..111: note = an artificially synthesized sequence
DIVLTQSPASLAVSLGQRATISCRASKSVSTSGYSYMHWYQQKPGQPPKLLIFLASNLESGVPARFSGSGSGTDFTLNIHPVEEEDAATYYCQHSRHLPWTFGGGTKLEIK

SEQ ID NO: 315 - protein, 107 aa, synthetic construct
  REGION 1..107: note = an artificially synthesized sequence
DIVMTQSQKFMSTSVGDRVSVTCKASQNVGTNVAWYQQKPGQSPKALIYSASYRYSGVPDRFTGSGSGTDFTLTISNVQSEDLAEYFCQQYNNYPLTFGAGTKLELK

SEQ ID NO: 316 - protein, 107 aa, synthetic construct
  REGION 1..107: note = an artificially synthesized sequence
DIQMTQSPSSLSASLGERVSLTCRASQEISDYLSWLQQKPDGTIKRLIFAASTLDSGVPKRFSGSRSGSDFSLSISSLESEDFADYYCLQYASYPYTFGSGTKLEIK

SEQ ID NO: 317 - protein, 112 aa, synthetic construct
  REGION 1..112: note = an artificially synthesized sequence
DVVMTQTPLSLPVTLGQPASISCRSSQSLVYRDGNTYLSWFQQRPGQSPRRLIYKVSNWQSGVPDRFSGGGSGTDFTLKISRVEAEDVGVYYCMQGTHWPPTFGQGTKVEIK

SEQ ID NO: 318 - protein, 108 aa, synthetic construct
  REGION 1..108: note = an artificially synthesized sequence
EIVLTQSPGTLSLSPGERATLSCRASQSVSSSYLAWYQQKPGQAPRLLIYGASTRATGIPDRFSGSGSGTDFTLTISRLEPEDFAVYYCQQYGTSPLTFGGGTKVEIK

SEQ ID NO: 319 - protein, 108 aa, synthetic construct
  REGION 1..108: note = an artificially synthesized sequence
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIK

SEQ ID NO: 320 - protein, 108 aa, synthetic construct
  REGION 1..108: note = an artificially synthesized sequence
EIVLTQSPGTLSLSPGERATLSCRASQSVNKNYLAWYQQKPGQAPRLLIYGASSRATGIPDRFSGSGSGTDFTLTISRLEPEDFAVYYCQQYDRSPLTFGGGTKVEIK